(19) United States
(12) Patent Application Publication
(10) Pub. No.: US 2002/0065404 A1
(43) Pub. Date: May 30, 2002

(54) Cytidine monophosphate-sialic acid transporter, and hexosaminidase polynucleotides and polypeptides, and uses based thereon.

(76) Inventors: Michael J. Betenbaugh, Baltimore, MD (US); Karen Palter, Philadelphia, PA (US)

Correspondence Address: McGUIREWOODS LLP, Suite 1800, 1750 Tysons Boulevard, McLean, VA, 22102, US

(21) Appl. No.: 09/750,703

(22) Filed: Jan. 2, 2001

Related U.S. Application Data
(60) Provisional application No. 60/174,612, filed on Jan. 5, 2000.

(51) Int. Cl. C07H 21/02; C07H 21/04; C12N 1/20

(52) U.S. Cl. 536/23.1; 435/252.3

(57) ABSTRACT

The present invention relates to novel Drosophila genes and proteins called cytidine monophosphate-sialic acid transporter (CMP-SAT), hexosaminidase-1 (Hex-1), and hexosaminidase-2 (Hex-2), and isolated polynucleotides encoding these polypeptides. Also provided are vectors, host cells, antibodies, and recombinant methods for producing CMP-SAT, Hex-1, and Hex-2 polypeptides as well as sense and anti-sense polynucleotides of Hex-1 and Hex-2. The invention further relates to production of complex glycoproteins in insect cell expression systems.

### CROSS REFERENCE TO RELATED APPLICATION

This application claims the benefit under 35 U.S.C.§ 119(e) of prior provisional application No. 60/174,612, filed Jan. 5, 2000, which is hereby incorporated by reference in its entirety.

### STATEMENT REGARDING FEDERALLY SPONSORED RESEARCH OR DEVELOPMENT

Part of the work performed during the development of this invention utilized U.S. Government funds in the form of grants from the National Science Foundation, Grant Number BES981400. The U.S. Government has certain rights in this invention.

### FIELD OF THE INVENTION

The present invention relates to novel genes and proteins which participate in post-translational processing events. In specific embodiments, the present invention provides nucleic acid molecules encoding cytidine monophosphate-sialic acid transporter (CMP-SA transporter or CMP-SAT), hexosaminidase-1 (Hex-1), and/or hexosaminidase-2 (Hex-2) polypeptides, and fragments, variants, or derivatives thereof. In additional embodiments the present invention provides for nucleic acid molecules complementary to these nucleic acid molecules (including fragments, variants, or derivatives thereof). The present invention also relates to polypeptides encoded by these nucleic acid molecules, and vectors, host cells and recombinant methods for producing the same.

### BACKGROUND OF THE INVENTION

During the last decade, numerous processes and procedures have been developed for genetically engineering cells in order to produce a wide variety of proteins and glycoproteins. These procedures involve utilizing recombinant DNA technology to prepare a vector which includes genetic material that codes for a specific protein or glycoprotein. Upon introduction of the vector into the host cell, the inserted genetic material instructs the host cell's biochemical machinery to manufacture a specific protein or glycoprotein.

Glycoproteins are proteins having carbohydrate groups attached at various points along the protein's amino acid backbone. The carbohydrate groups are commonly attached to asparagine, serine or threonine. The genetic sequence introduced into the host cell usually includes instructions with respect to the amino acid sequence of the protein and the location and structure of the carbohydrate groups. Most of the cell lines which are commonly used as host cells are capable of following the vector's instructions with respect to preparing a protein having a specific amino acid sequence. However, many host cells are not capable of following instructions with respect to glycosylation of the protein. For example, lepidopteran insect cells are a common host cell used for producing a wide variety of proteins via baculovirus expression systems. However, insect cells do not necessarily contain the same, or sufficient quantities of, the glycosylation machinery found in mammalian cells necessary to produce glycoprotein structures similar or identical to those produced by mammalian cells. Additionally, insect cells may also contain glycosylation machinery that interferes with processing events that would otherwise permit production of “mammalian-like” glycoproteins.

More specifically, while mammalian cells often generate complex oligosaccharides terminating in sialic acid (SA), insect cells typically produce truncated (paucimannosidic) and hybrid structures terminating in mannose (Man) or N-acetylglucosamine (GlcNAc). The inability of insect cell lines to generate complex carbohydrates comprising sialic acid significantly limits the wider application of this expression system.

The carbohydrate composition of an attached oligosaccharide, especially sialic acid, can affect a glycoprotein's solubility, structural stability, resistance to protease degradation, biological activity, and in vivo circulation (Goochee et al. (1991) Bio/technology 9:1347-1355, Cumming et al. (1991) Glycobiology 1:115-130, Opdenakker et al. (1993) FASEB J. 7:1330, Rademacher et al. (1988) Ann. Rev. Biochem., Lis et al. (1993) Eur. J Biochem. 218:1-27). The terminal residues of a carbohydrate are particularly important for therapeutic proteins since the final sugar moiety often controls its in vivo circulatory half-life (Cumming et al. (1991) Glycobiology 1:115-130). Glycoproteins with oligosaccharides terminating in sialic acid typically remain in circulation longer due to the presence of receptors in hepatocytes and macrophages that bind and rapidly remove structures terminating in mannose (Man), N-acetylglucosamine (GlcNAc), and galactose (Gal), from the bloodstream (Ashwell et al. (1974) Giochem. Soc. Symp. 40:117-124, Goochee et al (1991) Bio/technology 9:1347-1355, Opdenakker et al. (1993) FASEB J. 7:1330). Unfortunately, Man and GlcNAc are the residues most commonly found on the termini of glycoproteins produced by insect cells. The presence of sialic acid can also be important to the structure and function of a glycoprotein since sialic acid is one of the few sugars that is charged at physiological pH. The sialic acid residue is often involved in biological recognition events such as protein targeting, viral infection, cell adhesion, tissue targeting, and tissue organization (Brandley et al. (1986) J. of Leukocyte bio. 40:97-111, Varki et al. (1997) FASEB 11:248-255, Goochee et al. (1991) Bio/technology 9:1347-1355, Lopez et al. (1997) Glycobiology 7:635-651, Opdenakker et al. (1993) FASEB J. 7:1330).

The composition of the attached oligosaccharide for a secreted or membrane-bound glycoprotein is dictated by the structure of the protein and by the post-translational processing events that occur in the endoplasmic reticulum and Golgi apparatus of the host cell. Since the secretory processing machinery in mammalian cells differs from that in insect cells, glycoproteins with very different carbohydrate structures are produced by these two host cells (Jarvis et al (1995) Virology 212:500-511, Maru et al. (1996) J. Biol. Chem. 271:16294-16299, Altmann etal. (1996) Trends in Glycoscience and Glycotechnology 8:101-114). These differences in carbohydrate structure can have dramatic effects on the in vitro and in vivo properties of the resulting glycoprotein. For example, the in vitro activity of human thyrotropin (hTSH) expressed in insect cells was five times higher than the activity of the same glycoprotein produced from mammalian Chinese hamster ovary (CHO) cells (Grossman et al. (1997) Endocrinology 138:92-100). However, the in vivo activity of the insect cell-derived product was substantially lower due to its rapid clearance from injected rats. The drop in in vivo hTSH activity was linked to the absence of complex-type oligosaccharides terminating in sialic acid in the insect cell product (Grossman et al. (1997) EndocrinologyI 138:92-100).

N-glycosylation is highly significant to glycoprotein structure and function. In insect and mammalian cells N-glycosylation begins in the endoplasmic reticulum (ER) with the addition of the oligosaccharide, Glc₃Man₉GlcNAc₂ onto the asparagine (Asn) residue in the consensus sequence Asn-X-Ser/Thr (Moremen, et al. (1994) Glycobiology 4:113-125, Varki et al. (1993) Glycobiology 3(2):97-130, Altmann et al (1996) Trends in Glycoscience and Glycotechnology 8:101-114). As the glycoprotein passes through the ER and Golgi apparatus, enzymes trim and add different sugars to this N-linked glycan. These carbohydrate modification steps can differ in mammalian and insect hosts.

In mammalian cell lines, the initial trimming steps are followed by the enzyme-catalyzed addition of sugars including N-acetylglucosamine (GlcNAc), galactose (Gal), and sialic acid (SA) by steps as described in Goochee et al. (1991) Bio/technology 9:1347-1355.

In insect cells, N-linked glycans attached to glycoproteins comprise either high-mannose (Man₉₋₅GlcNAc₂) or truncated (paucimannosidic) (Man₃₋₂GlcNAc₂) oligosaccharides; occasionally comprising alpha(1,6)-fucose (FIG. 3; Jarvis et al. (1989) Mol. Cell. Biol. 9:214-223, Kuroda et al. (1990) Virology 174:418-329, Marz et al. (1995) Glycoproteins 543-563, Altmann et al. (1996) Trends in Glycoscience and Glycotechnology 8:101-114). Reports primarily directed to Sf-9 or Sf-21 cells from Spodoptera frugiperda indicated that insect cells could trim N-linked oligosaccharides but could not elongate these trimmed structures to produce complex carbohydrates. Reports from other insect cell lines, including Tricoplusia ni (T. ni; High Five™) and Estigmena acrea (Ea-4), indicated the presence of limited levels of partially elongated hybrid (structures with one terminal Man branch and one branch with terminal Gal, GlcNAc, or another sugar) and complex (structures with two non-man termini) N-linked oligosaccharides (Oganah et al. (1996) Bio/Technology 14:197-202, Hsu et al. (1997) J. Biol. Chem. 272:9062-9070). Low levels of GlcNAc transferase I and II (GlcNAc TI and TII), fucosyltransferase, mannosidases I and II, and Gal transferase (Gal T) have been reported in these insect cells; indicating a limited capability for production of these hybrid and complex N-linked oligosaccharides in these cells (Velardo et al (1993) J. Biol. Chem. 268:17902-17907, Altmann et al. (1996) Trends in Glycoscience and Glycotechnology 8:101-114, van Die et al. (1996) Glycobiology 6:157-164).

Manipulating carbohydrate processing in insect cells has been attempted by others; and in mammalian cells, the expression of sialyltransferases, galactosyltransferases and other enzymes is well established in order to enhance the level of oligosaccharide attachment (see U.S. Pat. No. 5,047,335). However, in these cases, the presence of the necessary donor nucleotide substrates, most significantly the sialylation nucleotide, CMP-sialic acid, in the proper subcellular compartment has been assumed.

CMP-SA must be delivered into the Golgi apparatus in order for sialylation to occur, and this transport process depends on the presence of the CMP-SA transporter protein (Deutscher et al. (1984) Cell 39:295-299). Where the native enzymatic transport is rate-limiting, a CMP-SA transporter enzyme is cloned and expressed in the cells of interest to supply or enhance CMP-SA transport.

Moreover, most insect cell derived glycoproteins lack complex N-glycans. This absence is likely, at least in part, due to the presence of glucosaminidases that cleave GlcNAc attached to the alpha(1,3) Man branch to generate paucimannosidic oligosaccharides (Licari et al. (1993) Biotech. Prog. 9:146-152, Altmann et al. (1995) J. Biol. Chem. 270:17344-17349). Therefore, inhibition of glucosaminidase activity in insect cells would permit persistence and elongation of carbohydrate substrates such that more “mammalian-like” glycoproteins are produced in these cells.

From the above, it is apparent that there is a need to identify polynucleotides and polypeptides which can be used to alter the glycosylation machinery of non-human host cells in order to control the structure of carbohydrates attached during glycosylation. Glycoproteins containing sialylated oligosaccharides would have improved in vivo circulatory half-lives that could lead to their increased utilization as vaccines and therapeutics. In particular, complex sialylated glycoproteins from insect cells would be more appropriate biological mimics of native mammalian glycoproteins in molecular recognition events where sialic acid plays a role. Thus, manipulating carbohydrate processing pathways in insect cells would be useful not only in expressing glycoproteins which accurately mimic naturally occurring proteins, but would also be useful in preparing glycoproteins having selected altered carbohydrate structures for medical and research uses.

### SUMMARY OF THE INVENTION

The present invention provides nucleic acid molecules encoding CMP-SA transporter and hexosaminidase polynucleotides and polypeptides, and fragments, variants, or derivatives thereof. These compositions are useful for production of glycosylated proteins in heterologous expression systems. In particular, these compostions are useful for manipulating the carbohydrate processing pathways of insect cell lines to enhance production of complex sialylated glycoproteins. Such sialylated glycoproteins find use as pharmaceutical compositions, vaccines, diagnostics, therapeutics, and the like.

Since CMP-SA is not membrane-soluble it must be delivered into the Golgi apparatus in order for sialylation to occur. This transport process depends on the presence of a CMP-SA transporter protein (Deutscher et al. (1984) Cell 39:295-299). Where the native enzymatic transport is lower than desired, a CMP-SA transporter enzyme is cloned and expressed in the cells of interest. For example, polynucleotides encoding the CMP-SA transporter, disclosed herein (SEQ ID NO:1 and SEQ ID NO:2), may be cloned and expressed in cells to provide or supplement CMP-SA transport activity.

According to the methods of the present invention, production of the acceptor substrate glycan GalGlcNAcMan-R, is particularly desirable for the sialylation reaction of N-linked glycoproteins, moreover the terminal Gal is required. Thus, in one embodiment of the invention the cells of interest are manipulated (using techniques described herein or otherwise known in the art) to contain this substrate. For example, for insect cells which principally produce truncated carbohydrates terminating in Man or GlcNAc, such cells may routinely be manipulated to produce a significant fraction of complex oligosaccharides terminating in Gal. One non-limiting, non-exclusive approach that may be routinely applied to produce a significant fraction of complex oligosaccharides terminating in Gal includes suppressing carbohydrate processing events which produce truncated N-linked glycans that cannot serve as acceptors in downstream glycosyltransferase reactions.

Thus, in one embodiment, concentrations of acceptor substrates are increased by using methods described herein or otherwise known in the art to suppress the activity of one or more endogenous glycosidases. By way of example, an endogenous glycosidase, the activity of which may be suppressed according to the methods of the invention includes Hex-1 and/or Hex-2. Thus, the invention encompasses suppressing enzymatic pathways that produce truncated acceptor carbohydrate structures in insect cell lines.

The methods of the present invention provides for manipulation of glycoprotein production in cells to desired levels by enhancing transport of the donor nucleotide sugar substrate (CMP-SA) to the Golgi apparatus and/or by introducing expression constructs which inhibit or eliminate endogenous glucosaminidase activity (for example, Hex-1 and/or Hex-2 anti-sense, ribozyme, or gene targeting (“knockout”) constructs). By “desired level” is intended that the quantity of a biochemical comprised by the cell of interest is altered subsequent to subjecting the cell to the methods of the invention. In this manner, the invention comprises enhancing expression of CMP-SAT (to increase CMP-SAT concentration in the Golgi lumen) and/or reduction of glucosaminidase activity (to suppress unfavorable cleavage reactions that generate truncated carbohydrate acceptors). In a preferred embodiment of the invention, enhancing production of sialylated glycoproteins comprise increasing the levels to above endogenous levels. It is recognized that the increase can be from a non-detectable level to any detectable level; or the increase can be from a detected endogenous level to a higher level.

For purposes of the present invention, by “enhancing expression” is intended to mean that the translated product of a nucleic acid encoding a desired protein is higher than the endogenous level of that protein in the host cell in which the nucleic acid is expressed. In a preferred embodiment of the invention, the biological activity of a desired carbohydrate processing enzyme is increased by enhancing expression of the enzyme.

For the purposes of the invention, by “suppressing activity” is intended to mean decreasing the biological activity of an enzyme. In this aspect, the invention encompasses reducing the endogenous expression of the enzyme protein, for example, by using antisense, and/or ribozyme nucleic acid sequences, and/or gene knock-out mutagenesis.

By “endogenous” is intended to mean the type and/or quantity of a biological function or a biochemical composition that is present in a naturally occurring or recombinant cell prior to manipulation of that cell according to the methods of the invention.

By “heterologous” is intended to mean the type and/or quantity of a biological function or a biochemical composition that is not present in a naturally occurring or recombinant cell prior to manipulation of that cell by the methods of the invention.

For purposes the present invention, by “a heterologous polypeptide or protein” is meant as a polypeptide or protein expressed (i.e. synthesized) in a cell species of interest that is different from the cell species in which the polypeptide or protein is normally expressed (i.e. expressed in nature).

Methods for determining endogenous and heterologous functions and compositions relevant to the invention are provided herein; and otherwise encompass those methods known in the art.

In insect cells, paucimannosidic structures are produced by a membrane-bound glucosaminidase, which remove terminal GlcNAc residues from the alpha(1,3) arm of the trimannosyl core structure (Altmann et al (1995) J. Biol. Chem. 270:17344-17349). This trimannosyl core structure lacks the proper termini required for conversion of side chains to sialylated complex structures. Therefore, in the present invention, production of these undesired oligosaccharide structures is suppressed by inhibiting Hex-1 and/or Hex-2 activity.

### BRIEF DESCRIPTION OF THE DRAWINGS

FIG. 1 depicts the nucleotide and amino acid sequence of Drosophila CMP-SA transporter (SEQ ID NO:1 and (SEQ ID NO:2).

FIG. 2 depicts the nucleotide and amino acid sequence of Drosophila Hexosaminidase-1 (SEQ ID NO:3 and SEQ ID NO:4).

FIG. 3 depicts the nucleotide and amino acid sequence of Drosophila Hexosaminidase-2 (SEQ ID NO:5 and SEQ ID NO:6).

### DETAILED DESCRIPTION OF THE INVENTION

In accordance with one embodiment of the present invention, there is provided polynucleotides encoding a novel cytidine monophosphate-sialic acid transporter (CMP-SAT) (SEQ ID NO:1 and SEQ ID NO:2) as well as biologically active and/or diagnostically and/or therapeutically useful fragments, analogs, variants and/or derivatives thereof.

As used herein, CMP-SAT “polynucleotide” refers to a molecule having a nucleic acid sequence contained in SEQ ID NO:1 or the cDNA contained within the clone deposited with the ATCC. For example, the CMP-SAT polynucleotide can contain the nucleotide sequence of the full length cDNA sequence, including the 5′ and 3′ untranslated sequences, the coding region, as well as fragments, epitopes, domains, and variants of the nucleic acid sequence.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the CMP-SAT gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

In another embodiment of the present invention, there is provided polynucleotides encoding a novel hexosaminidase (Hex-1) (SEQ ID NO:3 and SEQ ID NO:4) as well as biologically active and/or diagnostically and/or therapeutically useful fragments, analogs, variants and/or derivatives thereof.

As used herein, Hex-1 “polynucleotide” refers to a molecule having a nucleic acid sequence contained in SEQ ID NO:3 or the cDNA contained within the clone deposited with the ATCC. For example, the Hex-1 polynucleotide can contain the nucleotide sequence of the full length cDNA sequence, including the 5′ and 3′ untranslated sequences, the coding region, as well as fragments, epitopes, domains, and variants of the nucleic acid sequence.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the Hex-1 gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

In another embodiment of the present invention, there is provided polynucleotides encoding another novel hexosaminidase (Hex-2) (SEQ ID NO:5 and SEQ ID NO:6) as well as biologically active and/or diagnostically and/or therapeutically useful fragments, analogs, variants and/or derivatives thereof. The present invention further includes polypeptides encoded by these nucleic acid molecules.

As used herein, Hex-2 “polynucleotide” refers to a molecule having a nucleic acid sequence contained in SEQ ID NO:5 or the cDNA contained within the clone deposited with the ATCC. For example, the Hex-2 polynucleotide can contain the nucleotide sequence of the full length cDNA sequence, including the 5′ and 3′ untranslated sequences, the coding region, as well as fragments, epitopes, domains, and variants of the nucleic acid sequence.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the Hex-2 gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

In accordance with another embodiment of the present invention, there is provided isolated nucleic acid molecules encoding CMP-SAT (SEQ ID NO:1), including mRNAs, cDNAs, reverse complement cDNAs, as well as analogs and biologically active and diagnostically or therapeutically useful fragments and derivatives thereof.

In accordance with another embodiment of the present invention, there is provided isolated nucleic acid molecules encoding Hex-1 (SEQ ID NO:3), including mRNAs, cDNAs, reverse complement cDNAs, as well as analogs and biologically active and diagnostically or therapeutically useful fragments and derivatives thereof.

In accordance with another embodiment of the present invention, there is provided isolated nucleic acid molecules encoding Hex-2 (SEQ ID NO:5), including mRNAs, cDNAs, reverse complement cDNAs, as well as analogs and biologically active and diagnostically or therapeutically useful fragments and derivatives thereof.

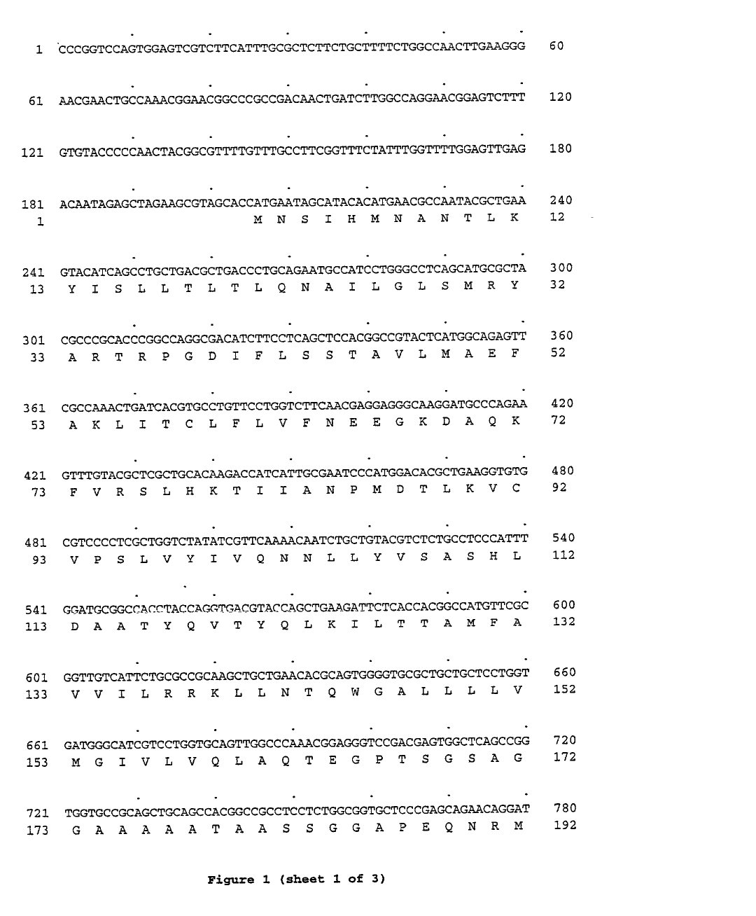

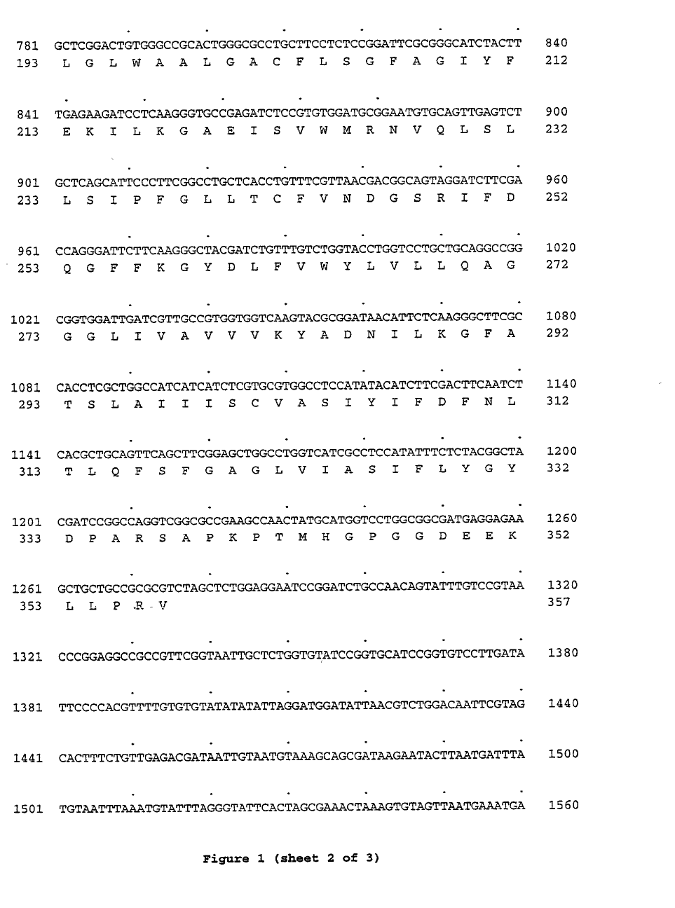

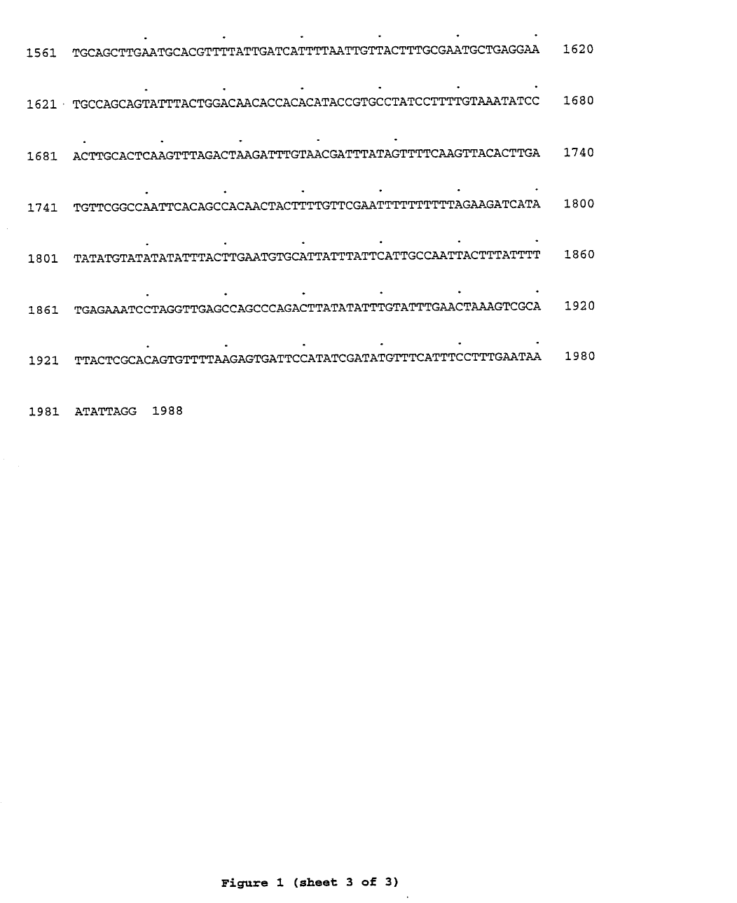

The present invention provides isolated nucleic acid molecules comprising or, alternatively, consisting of, a polynucleotide sequence encoding the CMP-SAT polypeptide disclosed in FIG. 1, and/or encoding a polypeptide having the amino acid sequence encoded by the cDNA disclosed in FIG. 1, or a fragment thereof. The invention also encompasses polypeptides comprising or, alternatively, consisting of, at least a portion of the amino acid sequence in FIG. 1 (SEQ ID NO:2) or amino acid sequence encoded by the cDNA clone (______) deposited in a ______ host on ______ and assigned ATCC number ______. The nucleotide sequence determined by sequencing the deposited CMP-SAT clone, which is shown in FIG. 1 (SEQ ID NO:1), contains an open reading frame encoding a complete polypeptide of about 357 amino acid residues including an N-terminal methionine and multiple predicted transmembrane domains centered at about amino acid residues 145-161, 265-281, 291-307, 47-63, 228-244, 312-328, 192-208, and/or 119-135 (FIG. 1; SEQ ID NO:2). In another embodiment multiple predicted transmembrane domains of CMP-SAT are located at about amino acid residues 140-165, 261-284, 286-314, 45-64, 228-244, 312-330, 192-211, and/or 119-135 (FIG. 1; SEQ ID NO:2). In this context “about” includes the particularly recited ranges or values, and ranges or values larger or smaller by several (5, 4, 3, 2, or 1) amino acids, at either extreme or at both extremes. The CMP-SAT polypeptide also has a predicted molecular weight for the complete protein of approximately 38.6 kDa.

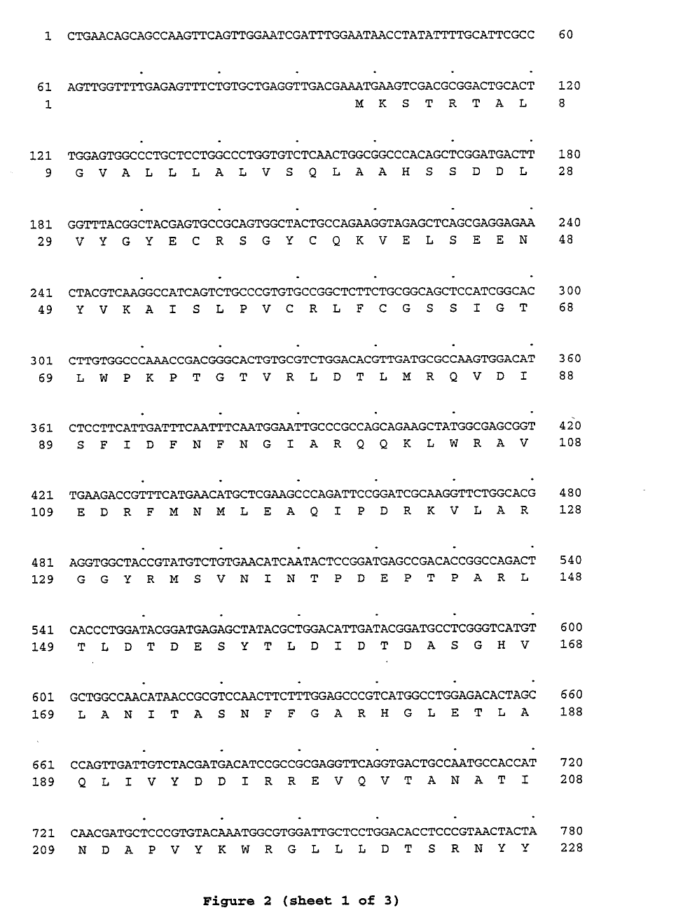

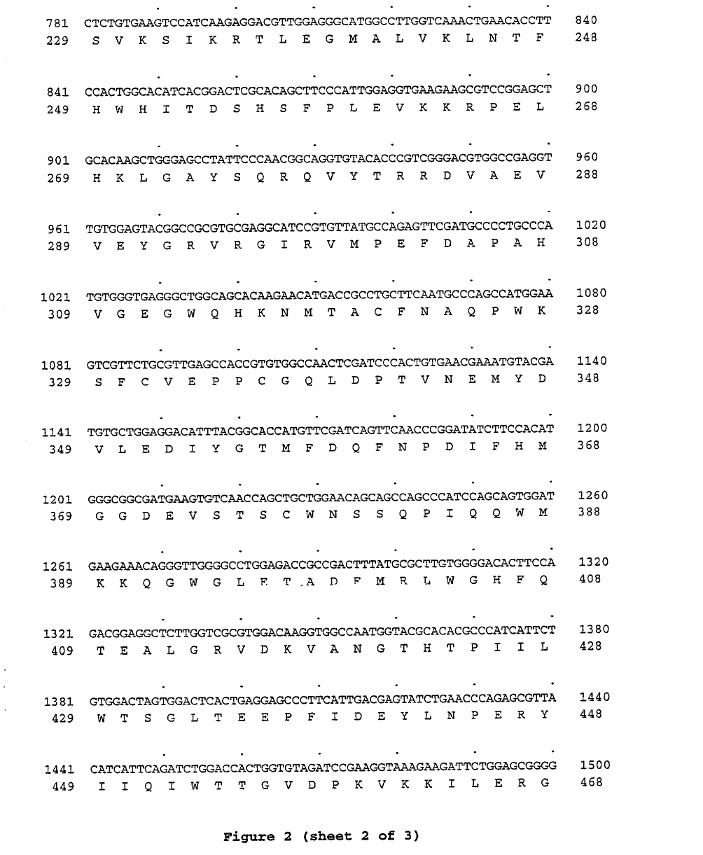

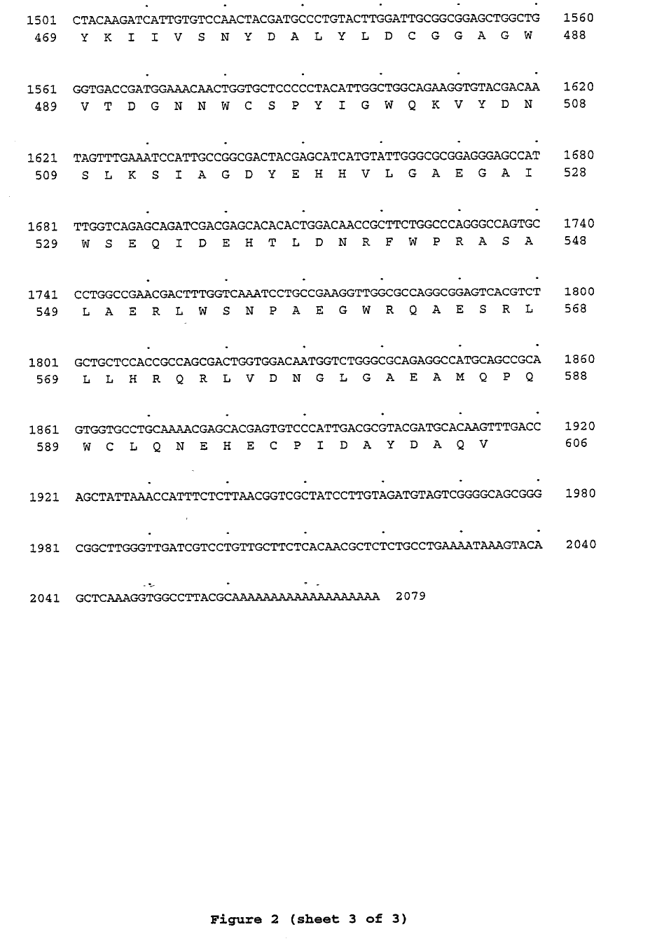

The present invention provides isolated nucleic acid molecules comprising or, alternatively, consisting of a polynucleotide sequence encoding the Hex-1 polypeptide disclosed in FIG. 2, and/or encoding a polypeptide having the amino acid sequence encoded by the cDNA disclosed in FIG. 2, or a fragment thereof. In preferred embodiments, the polynucleotide sequences of the invention are complementary to the polynucleotide sequence of FIG. 2 (SEQ ID NO:3). The invention also encompasses polypeptides comprising or, alternatively, consisting of, at least a portion of the amino acid sequence in FIG. 2 (SEQ ID NO:4) or amino acid sequence encoded by the cDNA clone (______) deposited in a ______ host on ______ and assigned ATCC number ______. The nucleotide sequence determined by sequencing the deposited Hex-1 clone, which is shown in FIG. 2 (SEQ ID NO:3), contains an open reading frame encoding a complete polypeptide of about 606 amino acid residues including an N-terminal methionine. The Hex-1 polypeptide also appears to contain an amino-terminal signal sequence from about amino acid residues 1-22 (FIG. 2; SEQ ID NO:4). In this context “about” includes the particularly recited ranges or values, and ranges or values larger or smaller by several (5, 4, 3, 2, or 1) amino acids, at either extreme or at both extremes. The Hex-1 polypeptide (SEQ ID NO:4) has a predicted molecular weight for the complete protein of approximately 69 kDa.

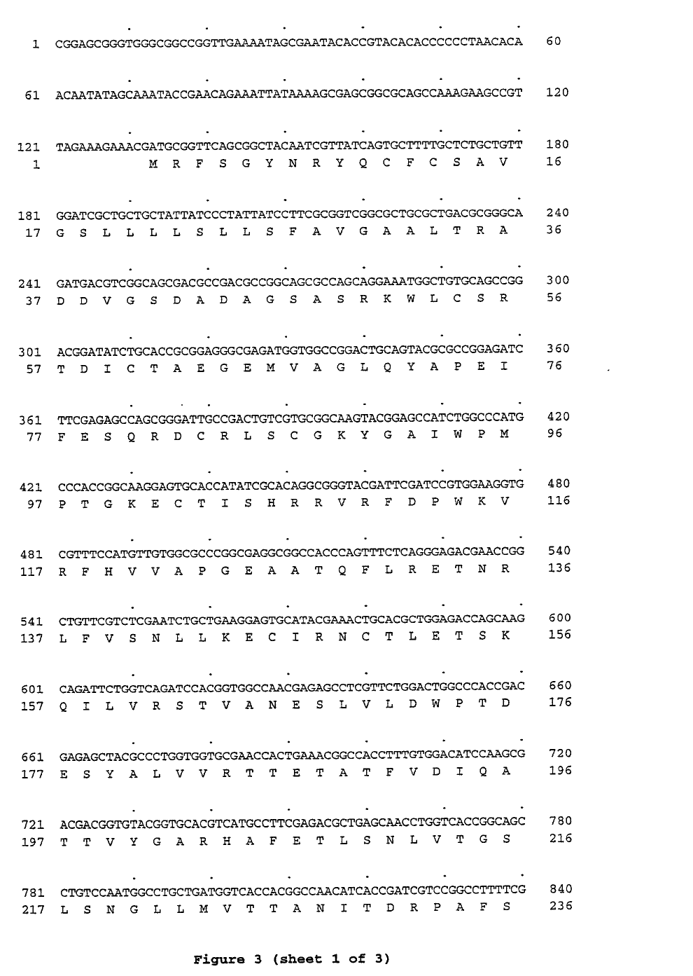

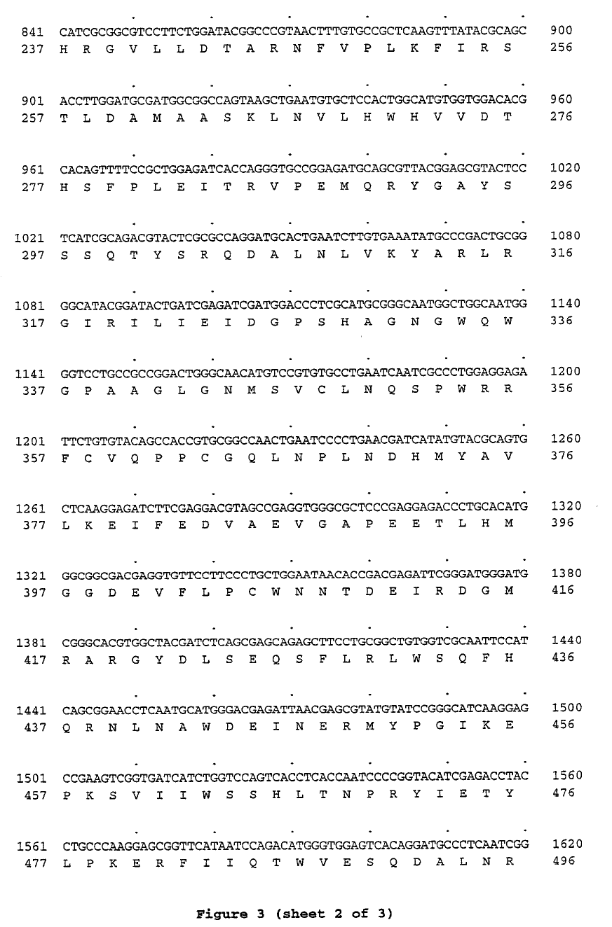

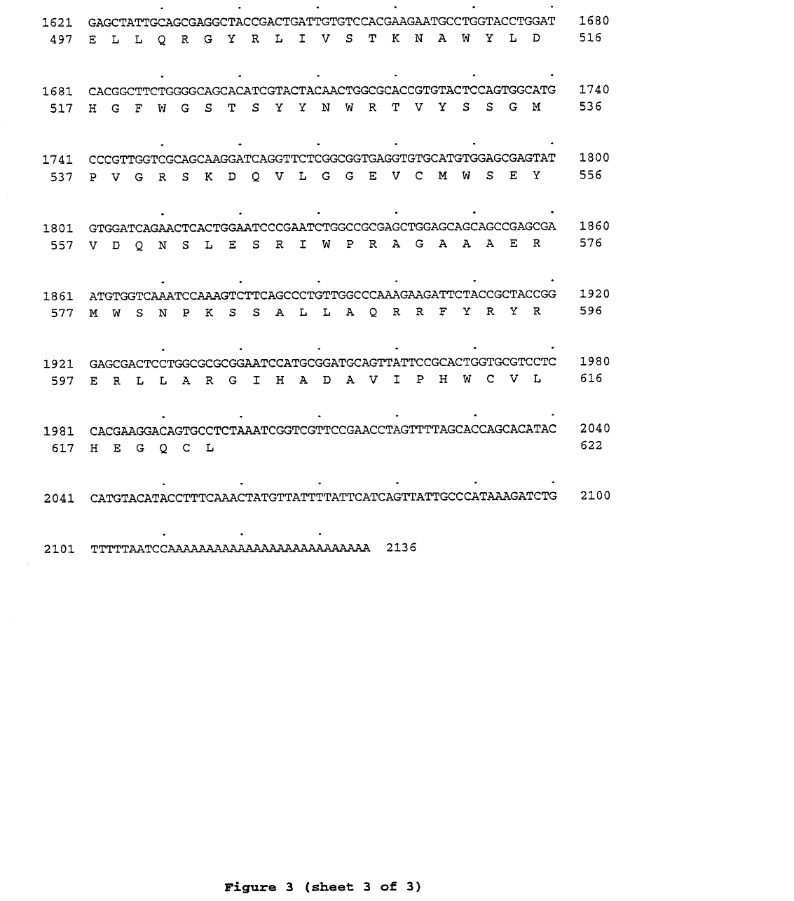

The present invention provides isolated nucleic acid molecules comprising or, alternatively, consisting of a polynucleotide sequence encoding the Hex-2 polypeptide disclosed in FIG. 3, and/or encoding a polypeptide having the amino acid sequence encoded by the cDNA disclosed in FIG. 3, or a fragment thereof. In preferred embodiments, the polynucleotide sequences of the invention are complementary to the polynucleotide sequence of FIG. 3 (SEQ ID NO:5). The invention also encompasses polypeptides comprising, or alternatively, consisting of, at least a portion of the amino acid sequence in FIG. 3 (SEQ ID NO:6) or amino acid sequence encoded by the cDNA clone (______) deposited in a ______ host on ______ and assigned ATCC number ______. The nucleotide sequence determined by sequencing the deposited Hex-2 clone, which is shown in FIG. 3 (SEQ ID NO:5), contains an open reading frame encoding a complete polypeptide of about 622 amino acid residues including an N-terminal methionine. The Hex-2 polypeptide also appears to contain an amino-terminal signal sequence from about amino acid residues 1-31 (FIG. 3; SEQ ID NO:6). In this context “about” includes the particularly recited ranges or values, and ranges or values larger or smaller by several (5, 4, 3, 2, or 1) amino acids, at either extreme or at both extremes. The Hex-2 polypeptide has a predicted molecular weight for the complete protein of approximately 70.7 kDa.

Thus, one aspect of the invention provides for use of isolated nucleic acid molecules comprising or, alternatively, consisting of polynucleotides a having nucleotide sequence selected from the group consisting of: (a) a nucleotide sequence encoding a polypeptide having the amino acid sequence shown in FIG. 1 (SEQ ID NO:2); (b) a nucleotide sequence encoding a biologically active fragment of the CMP-SAT polypeptide having the amino acid sequence shown in FIG. 1 (SEQ ID NO:2); (c) a nucleotide sequence encoding an antigenic fragment of the CMP-SAT polypeptide having the amino acid sequence shown in FIG. 1 (SEQ ID NO:2); (d) a nucleotide sequence encoding the CMP-SAT polypeptide comprising the complete amino acid sequence encoded by the plasmid contained in ATCC Deposit ______; (e) a nucleotide sequence encoding a biologically active fragment of the CMP-SAT polypeptide having the amino acid sequence encoded by the plasmid contained in ATCC Deposit ______; (f) a nucleotide sequence encoding an antigenic fragment of the CMP-SAT polypeptide having the amino acid sequence encoded by the plasmid contained in ATCC Deposit ______; and (g) a nucleotide sequence complementary to any of the nucleotide sequences in (a) through (f), above. Polypeptides encoded by such nucleic acids may also be used according to the methods of the present invention. Further embodiments of the invention include use of isolated nucleic acid molecules that comprise a polynucleotide having a nucleotide sequence at least 80%, 85%, or 90% identical, and more preferably at least 95%, 97%, 98% or 99% identical, to any of the nucleotide sequences in (a), (b), (c), (d), (e), (f), or (g), above, or a polynucleotide which hybridizes under stringent hybridization conditions to a polynucleotide in (a), (b), (c), (d), (e), (f), or (g), above. This polynucleotide which hybridizes does not hybridize under stringent hybridization conditions to a polynucleotide having a nucleotide sequence consisting of only A residues or of only T residues. Polypeptides encoded by such nucleic acids may also be used according to the methods of the present invention.

Another aspect of the invention provides for use of isolated nucleic acid molecules comprising or, alternatively, consisting of polynucleotides a having nucleotide sequence selected from the group consisting of: (a) a nucleotide sequence encoding a polypeptide having the amino acid sequence shown in FIG. 2 (SEQ ID NO:4); (b) a nucleotide sequence encoding a biologically active fragment of the Hex-1 polypeptide having the amino acid sequence shown in FIG. 2 (SEQ ID NO:4); (c) a nucleotide sequence encoding an antigenic fragment of the Hex-1 polypeptide having the amino acid sequence shown in FIG. 2 (SEQ ID NO:4); (d) a nucleotide sequence encoding the Hex-1 polypeptide comprising the complete amino acid sequence encoded by the plasmid contained in ATCC Deposit ______; (e) a nucleotide sequence encoding a biologically active fragment of the Hex-1 polypeptide having the amino acid sequence encoded by the plasmid contained in ATCC Deposit ______; (f) a nucleotide sequence encoding an antigenic fragment of the Hex-1 polypeptide having the amino acid sequence encoded by the plasmid contained in ATCC Deposit ______; and (g) a nucleotide sequence complementary to any of the nucleotide sequences in (a) through (f), above. Polypeptides encoded by such nucleic acids may also be used according to the methods of the present invention. Further embodiments of the invention include use of isolated nucleic acid molecules that comprise a polynucleotide having a nucleotide sequence at least 80%, 85%, or 90% identical, and more preferably at least 95%, 97%, 98% or 99% identical, to any of the nucleotide sequences in (a), (b), (c), (d), (e), (i), or (g), above, or a polynucleotide which hybridizes under stringent hybridization conditions to a polynucleotide in (a), (b), (c), (d), (e), (f), or (g), above. This polynucleotide which hybridizes does not hybridize under stringent hybridization conditions to a polynucleotide having a nucleotide sequence consisting of only A residues or of only T residues. Polypeptides encoded by such nucleic acids may also be used according to the methods of the present invention.

Another aspect of the invention provides for use of isolated nucleic acid molecules comprising or, alternatively, consisting of polynucleotides a having nucleotide sequence selected from the group consisting of: (a) a nucleotide sequence encoding a polypeptide having the amino acid sequence shown in FIG. 3 (SEQ ID NO:6); (b) a nucleotide sequence encoding a biologically active fragment of the Hex-2 polypeptide having the amino acid sequence shown in FIG. 3 (SEQ ID NO:6); (c) a nucleotide sequence encoding an antigenic fragment of the Hex-2 polypeptide having the amino acid sequence shown in FIG. 3 (SEQ ID NO:6); (d) a nucleotide sequence encoding the Hex-2 polypeptide comprising the complete amino acid sequence encoded by the plasmid contained in ATCC Deposit ______ (e) a nucleotide sequence encoding a biologically active fragment of the Hex-2 polypeptide having the amino acid sequence encoded by the plasmid contained in ATCC Deposit ______ ; (f) a nucleotide sequence encoding an antigenic fragment of the Hex-2 polypeptide having the amino acid sequence encoded by the plasmid contained in ATCC Deposit ______; and (g) a nucleotide sequence complementary to any of the nucleotide sequences in (a) through (f), above. Polypeptides encoded by such nucleic acids may also be used according to the methods of the present invention. Further embodiments of the invention include use of isolated nucleic acid molecules that comprise a polynucleotide having a nucleotide sequence at least 80%, 85%, or 90% identical, and more preferably at least 95%, 97%, 98% or 99% identical, to any of the nucleotide sequences in (a), (b), (c), (d), (e), (f), or (g), above, or a polynucleotide which hybridizes under stringent hybridization conditions to a polynucleotide in (a), (b), (c), (d), (e), (f), or (g), above. This polynucleotide which hybridizes does not hybridize under stringent hybridization conditions to a polynucleotide having a nucleotide sequence consisting of only A residues or of only T residues. Polypeptides encoded by such nucleic acids may also be used according to the methods of the present invention.

To reduce glucosaminidase activity in the target insect cell line(s), the invention provides vectors encoding Hex-1 and/or Hex-2 cDNAs in the antisense orientation and/or, vectors encoding ribozymes and/or, vectors containing sequences capable of “knocking out” the Hex-1 and/or Hex-2 genes via homologous recombination. Expression plasmids described herein or otherwise known in the art are constructed using techniques known in the art to produce stably-transformed insect cells that constitutively express the antisense construct and/or ribozyme construct to suppress translation of Hex-1 and/or Hex-2 or alternatively, to use homologous recombination techniques known in the art are to “knock-out” the Hex-I and/or Hex-2 genes. Techniques known in the art may be routinely applied to analyze N-linked oligosaccharide structures and to determine if N-glycan processing is altered and of the number of paucimannosidic structures in these cells is reduced.

Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′ coding portion of a polynucleotide that encodes the amino terminal portion of Hex-1 (SEQ ID NO:3) may be used to design antisense RNA oligonucleotides of from about 10 to 40 base pairs in length. A DNA oligonucleotide is designed to be complementary to a region of the gene involved in transcription thereby preventing transcription and the production of Hex-1 (SEQ ID NO:4). The antisense RNA oligonucleotide hybridizes to the mRNA in vivo and blocks translation of the mRNA molecule into Hex-1 polypeptide. The oligonucleotides described above can also be delivered to cells such that the antisense RNA or DNA may be expressed in vivo to inhibit production of Hex-1.

In another example, the 5′ coding portion of a polynucleotide that encodes the amino terminal portion of Hex-2 (SEQ ID NO:5) may be used to design antisense RNA oligonucleotides of from about 10 to 40 base pairs in length. A DNA oligonucleotide is designed to be complementary to a region of the gene involved in transcription thereby preventing transcription and the production of Hex-2 (SEQ ID NO:6). The antisense RNA oligonucleotide hybridizes to the mRNA in vivo and blocks translation of the mRNA molecule into Hex-2 polypeptide. The oligonucleotides described above can also be delivered to cells such that the antisense RNA or DNA may be expressed in vivo to inhibit production of Hex-2.

In one embodiment, the Hex-1 and/or Hex-2 antisense nucleic acids of the invention are produced intracellularly by transcription from an exogenous sequence. For example, a vector or a portion thereof, is transcribed, producing an antisense nucleic acid (RNA) of Hex-1 (SEQ ID NO:3). Such a vector would contain a sequence encoding Hex-1 (SEQ ID NO:3) antisense nucleic acids. In another example, a vector or a portion thereof, is transcribed, producing an antisense nucleic acid (RNA) of Hex-2 (SEQ ID NO:5). Such a vector would contain a sequence encoding Hex-2 (SEQ ID NO:5) antisense nucleic acids. Such anti-sense vectors can remain episomal or become chromosomally integrated, as long as it can be transcribed to produce the desired antisense RNA. Such vectors can be constructed by recombinant DNA technology methods standard in the art. Vectors can be plasmid, viral, or others know in the art used for replication and expression in insect cells. Expression of the sequences encoding Hex-1 and/or Hex-2, or fragments thereof, can be by any promoter known in the art to act in insect cells. Such promoters can be inducible or constitutive. Such promoters include, but are not limited to, the baculovirus polyhedrin promoter (Luckow et al. (1993) Curr. Opin. Biotech. 4:564-572, Luckow et al. (1995)).

The antisense nucleic acids of the invention comprise sequences complementary to at least a portion of an RNA transcript of Hex-1 (SEQ ID NO:3) genes. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded Hex-1 (SEQ ID NO:3) antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with Hex-1 RNAs it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of Hex-1 (SEQ ID NO:3) could be used in an antisense approach to inhibit translation of endogenous Hex-1 (SEQ ID NO:3) mRNAs. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of Hex-1 (SEQ ID NO:3) mRNAs, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The antisense nucleic acids of the invention comprise sequences complementary to at least a portion of an RNA transcript of Hex-2 (SEQ ID NO:5) genes. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded Hex-2 (SEQ ID NO:5) antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with Hex-2 RNAs it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of Hex-2 (SEQ ID NO:5) could be used in an antisense approach to inhibit translation of endogenous Hex-2 (SEQ ID NO:5) mRNAs. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of Hex-2 (SEQ ID NO:5) mRNAs, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), agents facilitating transport across the cell membrane (see, e.g., Letsinger et al., 1989, Proc. Natl. Acad. Sci. U.S.A. 86:6553-6556; Lemaitre et al., Proc. Natl. Acad. Sci. 84:648-652 (1987); PCT Publication No. WO88/09810, published Dec. 15, 1988), or hybridization-triggered cleavage agents (See, e.g., Krol et al., BioTechniques 6:958-976 (1988)) or intercalating agents. (See, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res. 15:6625-6641 (1987)). The oligonucleotide is a 2-0-methylribonucleotide (Inoue et al., Nucl. Acids Res. 15:6131-6148 (1987)), or a chimeric RNA-DNA analogue (Inoue et al., FEBS Lett. 215:327-330 (1997)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res. 16:3209 (1988)), methylphosphonate oligonucleotides can be prepared by use of controlled pore glass polymer supports (Sarin et al., Proc. Natl. Acad. Sci. U.S.A. 85:7448-7451 (1988)), etc.

While antisense nucleotides complementary to Hex-1 (SEQ ID NO:3) coding region sequences could be used, those complementary to the transcribed untranslated region are most preferred.

Potential Hex-1 (SEQ ID NO:3) suppressors according to the invention also include catalytic RNA, or a ribozyme (See, e.g., PCT International Publication WO 90/11364, published Oct. 4, 1990; Sarver et al, Science 247:1222-1225 (1990). While ribozymes that cleave mRNA at site specific recognition sequences can be used to destroy Hex-1 (SEQ ID NO:3) mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the Hex-1 (SEQ ID NO:3) mRNAs; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express Hex-1 (SEQ ID NO:3) in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter so that transfected cells will produce sufficient quantities of the ribozyme to destroy endogenous Hex-1 (SEQ ID NO:3) messages and inhibit translation. Since ribozymes unlike antisense molecules, are catalytic, a lower intracellular concentration is required for efficiency.

Endogenous gene expression can also be reduced by inactivating or “knocking out” the Hex-1 (SEQ ID NO:3) gene and/or their promoters using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention, or a completely unrelated DNA sequence (such as for example, CMP-SAT) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo.

In another embodiment, antisense nucleotides complementary to Hex-2 (SEQ ID NO:5) coding region sequences could be used, those complementary to the transcribed untranslated region are most preferred.

Potential Hex-2 (SEQ ID NO:5) suppressors according to the invention also include catalytic RNA, or a ribozyme (See, e.g., PCT International Publication WO 90/11364, published Oct. 4, 1990; Sarver et al, Science 247:1222-1225 (1990). While ribozymes that cleave mRNA at site specific recognition sequences can be used to destroy Hex-2 (SEQ ID NO:5) mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the Hex-2 (SEQ ID NO:5) mRNAs; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express Hex-2 (SEQ ID NO:5) in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter so that transfected cells will produce sufficient quantities of the ribozyme to destroy endogenous Hex-2 (SEQ ID NO:5) messages and inhibit translation. Since ribozyrnes unlike antisense molecules, are catalytic, a lower intracellular concentration is required for efficiency.

Endogenous gene expression can also be reduced by inactivating or “knocking out” the Hex-2 (SEQ ID NO:5) gene and/or their promoters using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention, or a completely unrelated DNA sequence (such as for example, CMP-SAT) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo.

In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). The contents of each of the documents recited in this paragraph is herein incorporated by reference in its entirety.

Polynucleotide and Polypeptide Variants

The present invention is directed to variants of the polynucleotide sequences disclosed in SEQ ID NO: 1, SEQ ID NO:3, or SEQ ID NO:5, and the complementary strands thereto, and/or the cDNA sequences contained in deposited clones.

The present invention also encompasses variants of the polypeptide sequences disclosed in SEQ ID NO:2, SEQ ID NO:4, or SEQ ID NO:6, and/or encoded by deposited clones.

“Variant” refers to a polynucleotide or polypeptide differing from the CMP-SAT (FIG. 1), Hex-1 (FIG. 2), or Hex-2 (FIG. 3) polynucleotides or polypeptides, but retaining essential properties thereof. Generally, variants are overall closely similar, and, in many regions, identical to the CMP-SAT (FIG. 1), Hex-1 (FIG. 2), or Hex-2 (FIG. 3) polynucleotides or polypeptides.

The present invention is also directed to nucleic acid molecules which comprise, or alternatively consist of, a nucleotide sequence which is at least 80%, 85%, 90%, 95%, 96%, 97%, 98% or 99% identical to, for example, the nucleotide coding sequence in SEQ ID NO: 1 or the complementary strand thereto, the nucleotide coding sequence contained in the deposited cDNA clone or the complementary strand thereto, nucleotide sequence encoding the polypeptide of SEQ ID NO:2, nucleotide sequence encoding the polypeptide encoded by the cDNA contained in the deposited clone, and/or polynucleotide fragments of any of these nucleic acid molecules (e.g., those fragments described herein). Polynucleotides which hybridize to these nucleic acid molecules under stringent hybridization conditions or lower stringency conditions are also encompassed by the invention, as are polypeptides encoded by these polynucleotides.

In another embodiment, the present invention is also directed to nucleic acid molecules which comprise, or alternatively consist of, a nucleotide sequence which is at least 80%, 85%, 90%, 95%, 96%, 97%, 98% or 99% identical to, for example, the nucleotide coding sequence in SEQ ID NO:3 or the complementary strand thereto, the nucleotide coding sequence contained in the deposited cDNA clone or the complementary strand thereto, nucleotide sequence encoding the polypeptide of SEQ ID NO:4, nucleotide sequence encoding the polypeptide encoded by the cDNA contained in the deposited clone, and/or polynucleotide fragments of any of these nucleic acid molecules (e.g., those fragments described herein). Polynucleotides which hybridize to these nucleic acid molecules under stringent hybridization conditions or lower stringency conditions are also encompassed by the invention, as are polypeptides encoded by these polynucleotides.

In another embodiment, the present invention is also directed to nucleic acid molecules which comprise, or alternatively consist of, a nucleotide sequence which is at least 80%, 85%, 90%, 95%, 96%, 97%, 98% or 99% identical to, for example, the nucleotide coding sequence in SEQ ID NO:5 or the complementary strand thereto, the nucleotide coding sequence contained in the deposited cDNA clone or the complementary strand thereto, nucleotide sequence encoding the polypeptide of SEQ ID NO:6, nucleotide sequence encoding the polypeptide encoded by the cDNA contained in the deposited clone, and/or polynucleotide fragments of any of these nucleic acid molecules (e.g., those fragments described herein). Polynucleotides which hybridize to these nucleic acid molecules under stringent hybridization conditions or lower stringency conditions are also encompassed by the invention, as are polypeptides encoded by these polynucleotides.

The present invention is also directed to polypeptides which comprise, or alternatively consist of, an amino acid sequence which is at least 80%, 85%, 90%, 95%, 96%, 97%, 98%, 99% identical to, for example, the polypeptide sequence shown in SEQ ID NO:2 the polypeptide sequence encoded by the cDNA contained in the deposited clone, and/or polypeptide fragments of any of these polypeptides (e.g., those fragments described herein).

In another embodiment, the present invention is also directed to polypeptides which comprise, or alternatively consist of, an amino acid sequence which is at least 80%, 85%, 90%, 95%, 96%, 97%, 98%, 99% identical to, for example, the polypeptide sequence shown in SEQ ID NO:4 the polypeptide sequence encoded by the cDNA contained in the deposited clone, and/or polypeptide fragments of any of these polypeptides (e.g., those fragments described herein).

In another embodiment, the present invention is also directed to polypeptides which comprise, or alternatively consist of, an amino acid sequence which is at least 80%, 85%, 90%, 95%, 96%, 97%, 98%, 99% identical to, for example, the polypeptide sequence shown in SEQ ID NO:6 the polypeptide sequence encoded by the cDNA contained in the deposited clone, and/or polypeptide fragments of any of these polypeptides (e.g., those fragments described herein).

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding CMP-SAT polypeptides (FIG. 1). In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in SEQ ID NO:1, the ORF (open reading frame), or any fragment specified as described herein.

In another embodiment, by a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding Hex-1 polypeptides (FIG. 2). In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence maybe an entire sequence shown in SEQ ID NO:3, the ORF (open reading frame), or any fragment specified as described herein.

In another embodiment, a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding Hex-2 polypeptides (FIG. 3). In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in SEQ ID NO:5, the ORF (open reading frame), or any fragment specified as described herein.

As a practical matter, whether any particular nucleic acid molecule or polypeptide is at least 80%, 85%, 90%, 95%, 96%, 97%, 98% or 99% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequences of SEQ ID NO:2 or to the amino acid sequence encoded by the cDNA contained in the deposited clone can be determined conventionally using known computer programs.

Likewise, whether any particular polypeptide is at least 80%, 85%, 90%, 95%, 96%, 97%, 98% or 99% identical to, for instance, the amino acid sequences of SEQ ID NO:4 or to the amino acid sequence encoded by the cDNA contained in the deposited clone can also be determined conventionally using known computer programs.

And furthermore, whether any particular polypeptide is at least 80%, 85%, 90%, 95%, 96%, 97%, 98% or 99% identical to, for instance, the amino acid sequences of SEQ ID NO:6 or to the amino acid sequence encoded by the cDNA contained in the deposited clone can also be determined conventionally using known computer programs.

A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245(1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequnce are manually corrected for. No other manual corrections are to made for the purposes of the present invention.

The CMP-SAT (SEQ ID NO:1 and SEQ ID NO:2) polynucleotide or polypeptide variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. CMP-SAT polynucleotide variants can be produced for a variety of reasons, e.g., to optimize codon expression for a particular host (change codons in the human mRNA to those preferred by a bacterial host such as E. coli).

The Hex-1 (SEQ ID NO:3 and SEQ ID NO:4) polynucleotide or polypeptide variants may also contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Hex-1 polynucleotide variants can be produced for a variety of reasons, e.g., to optimize codon expression for a particular host (change codons in the human mRNA to those preferred by a bacterial host such as E. coli).

The Hex-2 (SEQ ID NO:5 and SEQ ID NO:6) polynucleotide or polypeptide variants may also contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Hex-2 polynucleotide variants can be produced for a variety of reasons, e.g., to optimize codon expression for a particular host (change codons in the human mRNA to those preferred by a bacterial host such as E. coli).

Naturally occurring CMP-SAT (SEQ ID NO:1 and SEQ ID NO:2) variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

In another embodiment, naturally occurring Hex-1 (SEQ ID NO:3 and SEQ ID NO:4) variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

In another embodiment, naturally occurring Hex-2 (SEQ ID NO:5 and SEQ ID NO:6) variants are called “allelic variants,” and refer to one of several alternate forms of a gene occupying a given locus on a chromosome of an organism. (Genes II, Lewin, B., ed., John Wiley & Sons, New York (1985).) These alletic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the CMP-SAT polypeptides (SEQ ID NO:2). For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

In another embodiment, using known methods of protein engineering and recombinant DNA technology, variants may be generated to improve or alter the characteristics of the Hex-1 polypeptides (SEQ ID NO:4). For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

In another embodiment, using known methods of protein engineering and recombinant DNA technology, variants may be generated to improve or alter the characteristics of the Hex-2 polypeptides (SEQ ID NO:6). For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes CMP-SAT polypeptide (SEQ ID NO:2) variants which show substantial biological activity. Such variants include deletions, insertions, inversions, repeats, and substitutions selected according to general rules known in the art so as have little effect on activity.

The present application is directed to nucleic acid molecules at least 90%, 95%, 96%, 97%, 98% or 99% identical to the nucleic acid sequences disclosed herein, (e.g., encoding a polypeptide having the amino acid sequence of an N and/or C terminal deletion disclosed below as m-n of SEQ ID NO:2), irrespective of whether they encode a polypeptide having CMP-SA transport activity. This is because even where a particular nucleic acid molecule does not encode a polypeptide having CMP-SA transport activity, one of skill in the art would still know how to use the nucleic acid molecule, for instance, as a hybridization probe or a polymerase chain reaction (PCR) primer. Uses of the nucleic acid molecules of the present invention that do not encode a polypeptide having CMP-SA transport activity include, inter alia, (1) isolating a CMP-SA transporter or allelic or splice variants thereof in a cDNA library; (2) in situ hybridization (e.g., “FISH”) to metaphase chromosomal spreads to provide precise chromosomal location of the CMP-SA transporter, as described in Verma et al., Human Chromosomes: A Manual of Basic Techniques, Pergamon Press, New York (1988); and (3) Northern Blot analysis for detecting CMP-SA transporter mRNA expression in specific tissues.

Preferred, however, are nucleic acid molecules having sequences at least 90%, 95%, 96%, 97%, 98% or 99% identical to the nucleic acid sequences disclosed herein, which do, in fact, encode a polypeptide having CMP-SA transport activity. By “a polypeptide having CMP-SA transport activity” is intended polypeptides exhibiting activity similar, but not necessarily identical, to a functional activity of the CMP-SAT polypeptides (SEQ ID NO:2) of the present invention (e.g., complete (full-length) CMP-SAT, mature CMP-SAT, and soluble CMP-SAT as measured, for example, in a particular immunoassay or biological assay.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequences of the deposited cDNAs, the nucleic acid sequences shown in FIG. 1 (SEQ ID NO:1), or fragments thereof, will encode polypeptides “having CMP-SA transport activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having CMP-SA transport functional activity. This is because the skilled artisan is fully aware of amino acid substitutions that are either less likely or not likely to significantly effect protein function (e.g., replacing one aliphatic amino acid with a second aliphatic amino acid), as further described below.

In another embodiment, the invention further includes Hex-1 polypeptide (SEQ ID NO:4) variants which show substantial biological activity. Such variants include deletions, insertions, inversions, repeats, and substitutions selected according to general rules known in the art so as have little effect on activity.

The present application is directed to nucleic acid molecules at least 90%, 95%, 96%, 97%, 98% or 99% identical to the nucleic acid sequences disclosed herein, (e.g., encoding a polypeptide having the amino acid sequence of an N and/or C terminal deletion disclosed below as m-n of SEQ ID NO:4), irrespective of whether they encode a polypeptide having hexosaminidase activity. This is because even where a particular nucleic acid molecule does not encode a polypeptide having hexosaminidase activity, one of skill in the art would still know how to use the nucleic acid molecule, for instance, as a hybridization probe or a polymerase chain reaction (PCR) primer. Uses of the nucleic acid molecules of the present invention that do not encode a polypeptide having hexosaminidase activity include, inter alia, (1) isolating a Hex-1 or allelic or splice variants thereof in a cDNA library; (2) in situ hybridization (e.g., “FISH”) to metaphase chromosomal spreads to provide precise chromosomal location of the Hex-1, as described in Verma et al., Human Chromosomes: A Manual of Basic Techniques, Pergamon Press, New York (1988); and (3) Northern Blot analysis for detecting Hex-1 mRNA expression in specific tissues.

Preferred, however, are nucleic acid molecules having sequences at least 90%, 95%, 96%, 97%, 98% or 99% identical to the nucleic acid sequences disclosed herein, which do, in fact, encode a polypeptide having hexosaminidase activity. By “a polypeptide having hexosaminidase activity” is intended polypeptides exhibiting activity similar, but not necessarily identical, to a functional activity of the hexosaminidase polypeptides (SEQ ID NO:4) of the present invention (e.g., complete (full-length) Hex-1, mature Hex-1, and soluble Hex-1 as measured, for example, in a particular immunoassay or biological assay.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequences of the deposited cDNAs, the nucleic acid sequences shown in FIG. 2 (SEQ ID NO:3), or fragments thereof, will encode polypeptides “having hexosaminidase activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having hexosaminidase functional activity. This is because the skilled artisan is fully aware of amino acid substitutions that are either less likely or not likely to significantly effect protein function (e.g., replacing one aliphatic amino acid with a second aliphatic amino acid), as further described below.

In another embodiment, the invention further includes Hex-2 polypeptide (SEQ ID NO:6) variants which show substantial biological activity. Such variants include deletions, insertions, inversions, repeats, and substitutions selected according to general rules known in the art so as have little effect on activity.

The present application is directed to nucleic acid molecules at least 90%, 95%, 96%, 97%, 98% or 99% identical to the nucleic acid sequences disclosed herein, (e.g., encoding a polypeptide having the amino acid sequence of an N and/or C terminal deletion disclosed below as m-n of SEQ ID NO:6), irrespective of whether they encode a polypeptide having hexosaminidase activity. This is because even where a particular nucleic acid molecule does not encode a polypeptide having hexosaminidase activity, one of skill in the art would still know how to use the nucleic acid molecule, for instance, as a hybridization probe or a polymerase chain reaction (PCR) primer. Uses of the nucleic acid molecules of the present invention that do not encode a polypeptide having hexosaminidase activity include, inter alia, (1) isolating a Hex-2 or allelic or splice variants thereof in a cDNA library; (2) in situ hybridization (e.g., “FISH”) to metaphase chromosomal spreads to provide precise chromosomal location of the Hex-2, as described in Verma et al., Human Chromosomes: A Manual of Basic Techniques, Pergamon Press, New York (1988); and (3) Northern Blot analysis for detecting Hex-2 mRNA expression in specific tissues.

Preferred, however, are nucleic acid molecules having sequences at least 90%, 95%, 96%, 97%, 98% or 99% identical to the nucleic acid sequences disclosed herein, which do, in fact, encode a polypeptide having hexosaminidase activity. By “a polypeptide having hexosaminidase activity” is intended polypeptides exhibiting activity similar, but not necessarily identical, to a functional activity of the hexosaminidase polypeptides (SEQ ID NO:6) of the present invention (e.g., complete (full-length) Hex-2, mature Hex-2, and soluble Hex-2 as measured, for example, in a particular immunoassay or biological assay.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequences of the deposited cDNAs, the nucleic acid sequences shown in FIG. 3 (SEQ ID NO:5), or fragments thereof, will encode polypeptides “having hexosaminidase activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having hexosaminidase functional activity. This is because the skilled artisan is fully aware of amino acid substitutions that are either less likely or not likely to significantly effect protein function (e.g., replacing one aliphatic amino acid with a second aliphatic amino acid), as further described below.

Guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

For example, site directed changes at the amino acid level of CMP-SAT (SEQ ID NO:2) can be made by replacing a particular amino acid with a conservative amino acid. Preferred conservative mutations include: M1 replaced with A, G, I, L, S, T, or V; N2 replaced with Q; S3 replaced with A, G, I, L, T, M, or V; 14 replaced with A, G, L, S, T, M, or V; H5 replaced with K, or R; M6 replaced with A, G, I, L, S, T, or V; N7 replaced with Q; A8 replaced with G, I, L, S, T, M, or V; N9 replaced with Q; T10 replaced with A, G, I, L, S, M, or V; L11 replaced with A, G, I, S, T, M, or V; K12 replaced with H, or R; Y13 replaced with F, or W; I14 replaced with A, G, L, S, T, M, or V; S15 replaced with A, G, I, L, T, M, or V; L16 replaced with A, G, I, S, T, M, or V; L17 replaced with A, G, I, S, T, M, or V; T18 replaced with A, G, I, L, S, M, or V; L19 replaced with A, G, I, S, T, M, or V; T20 replaced with A, G, I, L, S, M, or V; L21 replaced with A, G, I, S, T, M, or V; Q22 replaced with N; N23 replaced with Q; A24 replaced with G, I, L, S, T, M, or V; I25 replaced with A, G, L, S, T, M, or V; L26 replaced with A, G, I, S, T, M, or V; G27 replaced with A, I, L, S, T, M, or V; L28 replaced with A, G, I, S, T, M, or V; S29 replaced with A, G, I, L, T, M, or V; M30 replaced with A, G, I, L, S, T, or V; R31 replaced with H, or K; Y32 replaced with F, or W; A33 replaced with G, I, L, S, T, M, or V; R34 replaced with H, or K; T35 replaced with A, G, I, L, S, M, or V; R36 replaced with H, or K; G38 replaced with A, I, L, S, T, M, or V; D39 replaced with E; I40 replaced with A, G, L, S, T, M, or V; F41 replaced with W, or Y; L42 replaced with A, G, I, S, T, M, or V; S43 replaced with A, G, I, L, T, M, or V; S44 replaced with A, G, I, L, T, M, or V; T45 replaced with A, G, I, L, S, M, or V; A46 replaced with G, I, L, S, T, M, or V; V47 replaced with A, G, I, L, S, T, or M; L48 replaced with A, G, I, S, T, M, or V; M49 replaced with A, G, I, L, S, T, or V; A50 replaced with G, I, L, S, T, M, or V; E51 replaced with D; F52 replaced with W, or Y; A53 replaced with G, I, L, S, T, M, or V; K54 replaced with H, or R; L55 replaced with A, G, I, S, T, M, or V; 156 replaced with A, G, L, S, T, M, or V; T57 replaced with A, G, I, L, S, M, or V; L59 replaced with A, G, I, S, T, M, or V; F60 replaced with W, or Y; L61 replaced with A, G, I, S, T, M, or V; V62 replaced with A, G, I, L, S, T, or M; F63 replaced with W, or Y; N64 replaced with Q; E65 replaced with D; E66 replaced with D; G67 replaced with A, I, L, S, T, M, or V; K68 replaced with H, or R; D69 replaced with E; A70 replaced with G, I, L, S, T, M, or V; Q71 replaced with N; K72 replaced with H, or R; F73 replaced with W, or Y; V74 replaced with A, G, I, L, S, T, or M; R75 replaced with H, or K; S76 replaced with A, G, I, L, T, M, or V; L77 replaced with A, G, I, S, T, M, or V; H78 replaced with K, or R; K79 replaced with H, or R; T80 replaced with A, G, I, L, S, M, or V; I81 replaced with A, G, L, S, T, M, or V; I82 replaced with A, G, L, S, T, M, or V; A83 replaced with G, I, L, S, T, M, or V; N84 replaced with Q; M86 replaced with A, G, I, L, S, T, or V; D87 replaced with E; T88 replaced with A, G, I, L, S, M, or V; L89 replaced with A, G, I, S, T, M, or V; K90 replaced with H, or R; V91 replaced with A, G, I, L, S, T, or M; V93 replaced with A, G, I, L, S, T, or M; S95 replaced with A, G, I, L, T, M, or V; L96 replaced with A, G, I, S, T, M, or V; V97 replaced with A, G, I, L, S, T, or M; Y98 replaced with F, or W; 199 replaced with A, G, L, S, T, M, or V; V100 replaced with A, G, I, L, S, T, or M; Q101 replaced with N; N102 replaced with Q; N103 replaced with Q; L104 replaced with A, G, I, S, T, M, or V; L105 replaced with A, G, I, S, T, M, or V; Y106 replaced with F, or W; V107 replaced with A, G, I, L, S, T, or M; S108 replaced with A, G, I, L, T, M, or V; A109 replaced with G, I, L, S, T, M, or V; S110 replaced with A, G, I, L, T, M, or V; H111 replaced with K, or R; L112 replaced with A, G, I, S, T, M, or V; D113 replaced with E; A114 replaced with G, I, L, S, T, M, or V; A115 replaced with G, I, L, S, T, M, or V; T116 replaced with A, G, I, L, S, M, or V; Y117 replaced with F, or W; Q118 replaced with N; V 19 replaced with A, G, I, L, S, T, or M; T120 replaced with A, G, I, L, S, M, or V; Y121 replaced with F, or W; Q122 replaced with N; L123 replaced with A, G, I, S, T, M, or V; K124 replaced with H, or R; I125 replaced with A, G, L, S, T, M, or V; L126 replaced with A, G, I, S, T, M, or V; T127 replaced with A, G, I, L, S, M, or V; T128 replaced with A, G, I, L, S, M, or V; A129 replaced with G, I, L, S, T, M, or V; M130 replaced with A, G, I, L, S, T, or V; F131 replaced with W, or Y; A132 replaced with G, I, L, S, T, M, or V; V133 replaced with A, G, I, L, S, T, or M; V134 replaced with A, G, I, L, S, T, or M; I135 replaced with A, G, L, S, T, M, or V; L136 replaced with A, G, I, S, T, M, or V; R137 replaced with H, or K;

R138 replaced with H, or K; K139 replaced with H, or R; L140 replaced with A, G, I, S, T, M, or V; L141 replaced with A, G, I, S, T, M, or V; N142 replaced with Q; T143 replaced with A, G, I, L, S, M, or V; Q144 replaced with N; W145 replaced with F, or Y; G146 replaced with A, I, L, S, T, M, or V; A147 replaced with G, I, L, S, T, M, or V; L148 replaced with A, G, I, S, T, M, or V; L149 replaced with A, G, I, S, T, M, or V; L150 replaced with A, G, I, S, T, M, or V; L151 replaced with A, G, I, S, T, M, or V; V152 replaced with A, G, I, L, S, T, or M; M153 replaced with A, G, I, L, S, T, or V; G154 replaced with A, I, L, S, T, M, or V; I155 replaced with A, G, L, S, T, M, or V; V156 replaced with A, G, I, L, S, T, or M; L157 replaced with A, G, I, S, T, M, or V; V158 replaced with A, G, I, L, S, T, or M; Q159 replaced with N; L160 replaced with A, G, I, S, T, M, or V; A161 replaced with G, I, L, S, T, M, or V; Q162 replaced with N; T163 replaced with A, G, I, L, S, M, or V; E164 replaced with D; G165 replaced with A, I, L, S, T, M, or V; T167 replaced with A, G, I, L, S, M, or V; S168 replaced with A, G, I, L, T, M, or V; G169 replaced with A, I, L, S, T, M, or V; S170 replaced with A, G, I, L, T, M, or V; A171 replaced with G, I, L, S, T, M, or V; G172 replaced with A, I, L, S, T, M, or V; G173 replaced with A, I, L, S, T, M, or V; A174 replaced with G, I, L, S, T, M, or V; A175 replaced with G, I, L, S, T, M, or V; A176 replaced with G, I, L, S, T, M, or V; A177 replaced with G, I, L, S, T, M, or V; A178 replaced with G, I, L, S, T, M, or V; T179 replaced with A, G, I, L, S, M, orV; A180 replaced with G, I, L, S, T, M, or V; A181 replaced with G, I, L, S, T, M, or V; S182 replaced with A, G, I, L, T, M, or V; S183 replaced with A, G, I, L, T, M, or V; G184 replaced with A, I, L, S, T, M, or V; G185 replaced with A, I, L, S, T, M, or V; A186 replaced with G, I, L, S, T, M, or V; E188 replaced with D; Q189 replaced with N; N190 replaced with Q; R191 replaced with H, or K; M192 replaced with A, G, I, L, S, T, or V; L193 replaced with A, G, I, S, T, M, or V; G194 replaced with A, I, L, S, T, M, or V; L195 replaced with A, G, I, S, T, M, or V; W196 replaced with F, or Y; A197 replaced with G, I, L, S, T, M, or V; A198 replaced with C, I, L, S, T, M, or V; L199 replaced with A, G, I, S, T, M, or V; G200 replaced with A, I, L, S, T, M, or V; A201 replaced with G, I, L, S, T, M, or V; F203 replaced with W, or Y; L204 replaced with A, G, I, S, T, M, or V; S205 replaced with A, G, I, L, T, M, or V; G206 replaced with A, I, L, S, T, M, or V; F207 replaced with W, or Y; A208 replaced with G, I, L, S, T, M, or V; G209 replaced with A, I, L, S, T, M, or V; I210 replaced with A, G, L, S, T, M, or V; Y211 replaced with F, or W; F212 replaced with W, or Y; E213 replaced with D; K214 replaced with H, or R; I215 replaced with A, G, L, S, T, M, or V; L216 replaced with A, G, I, S, T, M, or V; K217 replaced with H, or R; G218 replaced with A, I, L, S, T, M, or V; A219 replaced with G, I, L, S, T, M, or V; E220 replaced with D; I221 replaced with A, G, L, S, T, M, or V; S222 replaced with A, G, I, L, T, M, or V; V223 replaced with A, G, I, L, S, T, or M; W224 replaced with F, or Y; M225 replaced with A, G, I, L, S, T, or V; R226 replaced with H, or K; N227 replaced with Q; V228 replaced with A, G, I, L, S, T, or M; Q229 replaced with N; L230 replaced with A, G, I, S, T, M, or V; S231 replaced with A, G, I, L, T, M, or V; L232 replaced with A, G, I, S, T, M, or V; L233 replaced with A, G, I, S, T, M, or V; S234 replaced with A, G, I, L, T, M, or V; I235 replaced with A, G, L, S, T, M, or V; F237 replaced with W, or Y; G238 replaced with A, I, L, S, T, M, or V; L239 replaced with A, G, I, S, T, M, or V; L240 replaced with A, G, I, S, T, M, or V; T241 replaced with A, G, I, L, S, M, or V; F243 replaced with W, or Y; V244 replaced with A, G, I, L, S, T, or M; N245 replaced with Q; D246 replaced with E; G247 replaced with A, I, L, S, T, M, or V; S248 replaced with A, G, I, L, T, M, or V; 249 replaced with H, or K; I250 replaced with A, G, I, L, S, T, M, or V; F251 replaced with W, or Y; D252 replaced with E; Q253 replaced with N; G254 replaced with A, I, L, S, T, M, or V; F255 replaced with W, or Y; F256 replaced with W, or Y; K257 replaced with H, or R; G258 replaced with A, I, L, S, T, M, or V; Y259 replaced with F, or W; D260 replaced with E; L261 replaced with A, G, I, S, T, M, or V; F262 replaced with W, or Y; V263 replaced with A, G, I, L, S, T, or M; W264 replaced with F, or Y; Y265 replaced with F, or W; L266 replaced with A, G, I, S, T, M, or V; V267 replaced with A, G, I, L, S, T, or M; L268 replaced with A, G, I, L, S, T, M, or V; L269 replaced with A, G, I, S, T, M, or V; Q270 replaced with N; A271 replaced with G, I, L, S, T, M, or V; G272 replaced with A, I, L, S, T, M, or V; G273 replaced with A, I, L, S, T, M, or V; G274 replaced with A, I, L, S, T, M, or V; L275 replaced with A, I, L, S, T, M, or V; I276 replaced with A, G, L, S, T, M, or V; V277 replaced with A, G, I, L, S, T, or M; A278 replaced with G, I, L, S, T, M, or V; V279 replaced with A, G, I, L, S, T, or M; V280 replaced with A, G, I, L, S, T, or M; V281 replaced with A, G, I, L, S, T, or M; K282 replaced with H, or R; Y283 replaced with F, or W; A284 replaced with G, I, L, S, T, M, or V; D285 replaced with E; N286 replaced with Q; 1287 replaced with A, G, L, S, T, M, or V; L288 replaced with A, G, I, S, T, M, or V; K289 replaced with H, or R; G290 replaced with A, I, L, S, T, M, or V; F291 replaced with W, or Y; A292 replaced with G, I, L, S, T, M, or V; T293 replaced with A, G, I, L, S, M, or V; S294 replaced with A, G, I, L, T, M, or V; L295 replaced with A, G, I, S, T, M, or V; A296 replaced with G, I, L, S, T, M, or V; 1297 replaced with A, G, L, S, T, M, or V; I298 replaced with A, G, L, S, T, M, or V; I299 replaced with A, G, L, S, T, M, or V; S300 replaced with A, G, I, L, T, M, or V; V302 replaced with A, G, I, L, S, T, or M; A303 replaced with G, I, L, S, T, M, or V; S304 replaced with A, G, I, L, T, M, or V; I305 replaced with A, G, L, S, T, M, or V; Y306 replaced with F, or W; I307 replaced with A, G, L, S, T, M, or V; F308 replaced with W, or Y; D309 replaced with E; F310 replaced with W, or Y; N311 replaced with Q; L312 replaced with A, G, I, S, T, M, or V; T313 replaced with A, G, I, L, S, M, or V; L314 replaced with A, G, I, S, T, M, or V; Q315 replaced with N; F316 replaced with W, or Y; S317 replaced with A, G, I, L, T, M, or V; F318 replaced with W, or Y; G319 replaced with A, I, L, S, T, M, or V; A320 replaced with G, I, L, S, T, M, or V; G321 replaced with A, I, L, S, T, M, or V; L322 replaced with A, G, I, S, T, M, or V; V323 replaced with A, G, I, L, S, T, or M; I324 replaced with A, G, L, S, T, M, or V; A325 replaced with G, I, L, S, T, M, or V; S326 replaced with A, G, I, L, T, M, or V; I327 replaced with A, G, L, S, T, M, or V; F328 replaced with W, or Y; L329 replaced with A, G, I, S, T, M, or V; Y330 replaced with F, or W; G331 replaced with A, I, L, S, T, M, or V; Y332 replaced with F, or W; D333 replaced with E; A335 replaced with G, I, L, S, T, M, or V; R336 replaced with H, or K; S337 replaced with A, G, I, L, T, M, or V; A338 replaced with G, I, L, S, T, M, or V; K340 replaced with H, or R; T342 replaced with A, G, I, L, S, M, or V; M343 replaced with A, G, I, L, S, T, or V; H344 replaced with K, or R; G345 replaced with A, I, L, S, T, M, or V; G347 replaced with A, I, L, S, T, M, or V; G348 replaced with A, I, L, S, T, M, or V; D349 replaced with E; E350 replaced with D; E351 replaced with D; K352 replaced with H, or R; L353 replaced with A, G, I, S, T, M, or V; L354 replaced with A, G, I, S, T, M, or V; R356 replaced with H, or K; and/or V357 replaced with A, G, I, L, S, T, or M.

The resulting constructs can be routinely screened for activities or functions described throughout the specification and known in the art. Preferably, the resulting constructs have an increased CMP-SA transport activity or function, while the remaining CMP-SAT activities or functions are maintained. More preferably, the resulting constructs have more than one increased biological activity or function, while the remaining biological activities or functions are maintained.

In another example, site directed changes at the amino acid level of Hex-1 (SEQ ID NO:4) can be made by replacing a particular amino acid with a conservative amino acid. Preferred conservative mutations include: M1 replaced with A, G, I, L, S, T, or V; K2 replaced with H, or R; S3 replaced with A, G, I, L, T, M, or V; T4 replaced with A, G, I, L, S, M, or V; R5 replaced with H, or K; T6 replaced with A, G, I, L, S, M, or V; A7 replaced with G, I, L, S, T, M, or V; L8 replaced with A, G, I, S, T, M, or V; G9 replaced with A, I, L, S, T, M, or V; V10 replaced with A, G, I, L, S, T, or M; A11 replaced with G, I, L, S, T, M, or V; L12 replaced with A, G, I, S, T, M, or V; L 13 replaced with A, G, I, S, T, M, or V; L14 replaced with A, G, I, S, T, M, or V; A15 replaced with G, I, L, S, T, M, or V; L16 replaced with A, G, I, S, T, M, or V; V17 replaced with A, G, I, L, S, T, or M; S18 replaced with A, G, I, L, T, M, or V; Q 19 replaced with N; L20 replaced with A, G, I, S, T, M, or V; A21 replaced with G, I, L, S, T, M, or V; A22 replaced with G, I, L, S, T, M, or V; H23 replaced with K, or R; S24 replaced with A, G, I, L, T, M, or V; S25 replaced with A, G, I, L, T, M, or V; D26 replaced with E; D27 replaced with E; L28 replaced with A, G, I, S, T, M, or V; V29 replaced with A, G, I, L, S, T, or M; Y30 replaced with F, or W; G31 replaced with A, I, L, S, T, M, or V; Y32 replaced with F, or W; E33 replaced with D; R35 replaced with H, or K; S36 replaced with A, G, I, L, T, M, or V; G37 replaced with A, I, L, S, T, M, or V; Y38 replaced with F, or W; Q40 replaced with N; K41 replaced with H, or R; V42 replaced with A, G, I, L, S, T, or M; E43 replaced with D; L44 replaced with A, G, I, S, T, M, or V; S45 replaced with A, G, I, L, T, M, or V; E46 replaced with D; E47 replaced with D; N48 replaced with Q; Y49 replaced with F, or W; V50 replaced with A, G, I, L, S, T, or M; K51 replaced with H, or R; A52 replaced with G, I, L, S, T, M, or V; I53 replaced with A, G, L, S, T, M, or V; S54 replaced with A, G, I, L, T, M, or V; L55 replaced with A, G, I, S, T, M, or V; V57 replaced with A, G, I, L, S, T, or M; R59 replaced with H, or K; L60 replaced with A, G, I, S, T, M, or V; F61 replaced with W, or Y; G63 replaced with A, I, L, S, T, M, or V; S64 replaced with A, G, I, L, T, M, or V; S65 replaced with A, G, I, L, T, M, or V; 166 replaced with A, G, L, S, T, M, or V; G67 replaced with A, I, L, S, T, M, or V; T68 replaced with A, G, I, L, S, M, or V; L69 replaced with A, G, I, S, T, M, or V; W70 replaced with F, or Y; K72 replaced with H, or R; T74 replaced with A, G, I, L, S, M, or V; G75 replaced with A, I, L, S, T, M, or V; T76 replaced with A, G, I, L, S, M, or V; V77 replaced with A, G, I, L, S, T, or M; R78 replaced with H, or K; L79 replaced with A, G, I, S, T, M, or V; D80 replaced with E; T81 replaced with A, G, I, L, S, M, or V; L82 replaced with A, G, I, S, T, M, or V; M83 replaced with A, G, I, L, S, T, or V; R84 replaced with H, or K; Q85 replaced with N; V86 replaced with A, G, I, L, S, T, or M; D87 replaced with E; 188 replaced with A, G, L, S, T, M, or V; S89 replaced with A, G, I, L, T, M, or V; F90 replaced with W, or Y; 191 replaced with A, G, L, S, T, M, or V; D92 replaced with E; F93 replaced with W, or Y; N94 replaced with Q; F95 replaced with W, or Y; N96 replaced with Q; G97 replaced with A, I, L, S, T, M, or V; 198 replaced with A, G, L, S, T, M, or V; A99 replaced with G, 1, L, S, T, M, or V; R100 replaced with H, or K; Q10 replaced with N; Q102 replaced with N; K103 replaced with H, or R; L104 replaced with A, G, I, S, T, M, or V; W105 replaced with F, or Y; R106 replaced with H, or K; A107 replaced with G, I, L, S, T, M, or V; V108 replaced with A, G, I, L, S, T, or M; E109 replaced with D; D110 replaced with E; R111 replaced with H, or K; F112 replaced with W, or Y; M113 replaced with A, G, I, L, S, T, or V; N114 replaced with Q; M115 replaced with A, G, I, L, S, T, or V; L116 replaced with A, G, I, S, T, M, or V; El 17 replaced with D; A118 replaced with G, I, L, S, T, M, or V; Q119 replaced with N; I120 replaced with A, G, L, S, T, M, or V; D122 replaced with E; R123 replaced with H, or K; K124 replaced with H, or R; V125 replaced with A, G, I, L, S, T, or M; L126 replaced with A, G, I, S, T, M, or V; A127 replaced with G, I, L, S, T, M, or V; R128 replaced with H, or K; G129 replaced with A, I, L, S, T, M, or V; G130 replaced with A, I, L, S, T, M, or V; Y131 replaced with F, or W; R132 replaced with H, or K; M133 replaced with A, G, I, L, S, T, or V; S134 replaced with A, G, I, L, T, M, or V; V135 replaced with A, G, I, L, S, T, or M; N136 replaced with Q; I137 replaced with A, G, L, S, T, M, or V; N138 replaced with Q; T139 replaced with A, G, I, L, S, M, or V; D141 replaced with E; E142 replaced with D; T144 replaced with A, G, I, L, S, M, or V; A146 replaced with G, I, L, S, T, M, or V; R147 replaced with H, or K; L148 replaced with A, G, I, S, T, M, or V; T149 replaced with A, G, I, L, S, M, or V; L150 replaced with A, G, I, S, T, M, or V; D151 replaced with E; T152 replaced with A, G, I, L, S, M, or V; D153 replaced with E; E154 replaced with D; S155 replaced with A, G, I, L, T, M, or V; Y156 replaced with F, or W; T157 replaced with A, G, I, L, S, M, or V; L158 replaced with A, G, I, S, T, M, or V; D159 replaced with E; I160 replaced with A, G, L, S, T, M, or V; D161 replaced with E; T162 replaced with A, G, I, L, S, M, or V; D 163 replaced with E; A164 replaced with G, I, L, S, T, M, or V; S 165 replaced with A, G, I, L, T, M, or V; G166 replaced with A, I, L, S, T, M, or V; I167 replaced with K, or R; V168 replaced with A, G, I, L, S, T, or M; L169 replaced with A, G, I, S, T, M, or V; A170 replaced with G, I, L, S, T, M, or V; N171 replaced with Q; I172 replaced with A, G, L, S, T, M, or V; T173 replaced with A, G, I, L, S, M, or V; A174 replaced with G, I, L, S, T, M, or V; S175 replaced with A, G, I, L, T, M, or V; N176 replaced with Q; F177 replaced with W, or Y; F178 replaced with W, or Y; G179 replaced with A, I, L, S, T, M, or V; A180 replaced with G, I, L, S, T, M, or V; R181 replaced with H, or K; H182 replaced with K, or R; G183 replaced with A, I, L, S, T, M, or V; L184 replaced with A, G, I, S, T, M, or V; E185 replaced with D; T186 replaced with A, G, I, L, S, M, or V; L187 replaced with A, G, I, S, T, M, or V; A188 replaced with G, I, L, S, T, M, or V; Q189 replaced with N; L190 replaced with A, G, I, S, T, M, or V; 1191 replaced with A, G, L, S, T, M, or V; V192 replaced with A, G, I, L, S, T, or M; Y193 replaced with F, or W; D194 replaced with E; D195 replaced with E; I196 replaced with A, G, L, S, T, M, or V; R197 replaced with H, or K; R198 replaced with H, or K; E199 replaced with D; V200 replaced with A, G, I, L, S, T, or M; Q201 replaced with N; V202 replaced with A, G, I, L, S, T, or M; T203 replaced with A, G, I, L, S, M, or V; A204 replaced with G, I, L, S, T, M, or V; N205 replaced with Q; A206 replaced with G, I, L, S, T, M, or V; T207 replaced with A, G, I, L, S, M, or V; I208 replaced with A, G, L, S, T, M, or V; N209 replaced with Q; D210 replaced with E; A211 replaced with G, I, L, S, T, M, or V; V213 replaced with A, G, I, L, S, T, or M; Y214 replaced with F, or W; K215 replaced with H, or R; W216 replaced with F, or Y; R217 replaced with H, or K; G218 replaced with A, I, L, S, T, M, or V; L219 replaced with A, G, I, S, T, M, or V; L220 replaced with A, G, I, S, T, M, or V; L221 replaced with A, G, I, S, T, M, or V; D222 replaced with E; T223 replaced with A, G, I, L, S, M, or V; S224 replaced with A, G, I, L, T, M, or V; R225 replaced with H, or K; N226 replaced with Q; Y227 replaced with F, or W; Y228 replaced with F, or W; S229 replaced with A, G, I, L, T, M, or V; V230 replaced with A, G, I, L, S, T, or M; K231 replaced with H, or R; S232 replaced with A, G, I, L, T, M, or V; I233 replaced with A, G, L, S, T, M, or V; K234 replaced with H, or R; R235 replaced with H, or K; T236 replaced with A, G, I, L, S, M, or V; L237 replaced with A, G, I, S, T, M, or V; E238 replaced with D; G239 replaced with A, I, L, S, T, M, or V; M240 replaced with A, G, I, L, S, T, or V; A241 replaced with G, I, L, S, T, M, or V; L242 replaced with A, G, I, S, T, M, or V; V243 replaced with A, G, I, L, S, T, or M; K244 replaced with H, or R; L245 replaced with A, G, I, S, T, M, or V; N246 replaced with Q; T247 replaced with A, G, I, L, S, M, or V; F248 replaced with W, or Y; H249 replaced with K, or R; W250 replaced with F, or Y; H251 replaced with K, or R; 1252 replaced with A, G, L, S, T, M, or V; T253 replaced with A, G, I, L, S, M, or V; D254 replaced with E; S255 replaced with A, G, I, L, T, M, or V; H256 replaced with K, or R; S257 replaced with A, G, I, L, T, M, or V; F258 replaced with W, or Y; L260 replaced with A, G, I, S, T, M, or V; E261 replaced with D; V262 replaced with A, G, I, L, S, T, or M; K263 replaced with H, or R; K264 replaced with H, or R; R265 replaced with H, or K; E267 replaced with D; L268 replaced with A, G, I, S, T, M, or V; H269 replaced with K, or R; K270 replaced with H, or R; L271 replaced with A, G, I, S, T, M, or V; G272 replaced with A, I, L, S, T, M, or V; A273 replaced with G, I, L, S, T, M, or V; Y274 replaced with F, or W; S275 replaced with A, G, I, L, T, M, or V; Q276 replaced with N; R277 replaced with H, or K; Q278 replaced with N; V279 replaced with A, G, I, L, S, T, or M; Y280 replaced with F, or W; T281 replaced with A, G, I, L, S, M, or V; R282 replaced with H, or K; R283 replaced with H, or K; D284 replaced with E; V285 replaced with A, G, I, L, S, T, or M; A286 replaced with G, I, L, S, T, M, or V; E287 replaced with D; V288 replaced with A, G, I, L, S, T, or M; V289 replaced with A, G, I, L, S, T, or M; E290 replaced with D; Y291 replaced with F, or W; G292 replaced with A, I, L, S, T, M, or V; R293 replaced with H, or K; V294 replaced with A, G, I, L, S, T, or M; R295 replaced with H, or K; G296 replaced with A, I, L, S, T, M, or V; 1297 replaced with A, G, L, S, T, M, or V; R298 replaced with H, or K; V299 replaced with A, G, I, L, S, T, or M; M300 replaced with A, G, I, L, S, T, or V; E302 replaced with D; F303 replaced with W, or Y; D304 replaced with E; A305 replaced with G, I, L, S, T, M, or V; A307 replaced with G, I, L, S, T, M, or V; H308 replaced with K, or R; V309 replaced with A, G, I, L, S, T, or M; G310 replaced with A, I, L, S, T, M, or V; E311 replaced with D; G312 replaced with A, I, L, S, T, M, or V; W313 replaced with F, or Y; Q314 replaced with N; H315 replaced with K, or R; K316 replaced with H, or R; N317 replaced with Q; M318 replaced with A, G, I, L, S, T, or V; T319 replaced with A, G, I, L, S, M, or V; A320 replaced with G, I, L, S, T, M, or V; F322 replaced with W, or Y; N323 replaced with Q; A324 replaced with G, I, L, S, T, M, or V; Q325 replaced with N; W327 replaced with F, or Y; K328 replaced with H, or R; S329 replaced with A, G, I, L, T, M, or V; F330 replaced with W, or Y; V332 replaced with A, G, I, L, S, T, or M; E333 replaced with D; G337 replaced with A, I, L, S, T, M, or V; Q338 replaced with N; L339 replaced with A, G, I, S, T, M, or V; D340 replaced with E; T342 replaced with A, G, I, L, S, M, or V; V343 replaced with A, G, I, L, S, T, or M; N344 replaced with Q; E345 replaced with D; M346 replaced with A, G, I, L, S, T, or V; Y347 replaced with F, or W; D348 replaced with E; V349 replaced with A, G, I, L, S, T, or M; L350 replaced with A, G, I, S, T, M, or V; E351 replaced with D; D352 replaced with E; 1353 replaced with A, G, L, S, T, M, or V; Y354 replaced with F, or W; G355 replaced with A, I, L, S, T, M, or V; T356 replaced with A, G, I, L, S, M, or V; M357 replaced with A, G, I, L, S, T, or V; F358 replaced with W, or Y; D359 replaced with E; Q360 replaced with N; F361 replaced with W, or Y; N362 replaced with Q; D364 replaced with E; I365 replaced with A, G, L, S, T, M, or V; F366 replaced with W, or Y; H367 replaced with K, or R; M368 replaced with A, G, I, L, S, T, or V; G369 replaced with A, I, L, S, T, M, or V; G370 replaced with A, I, L, S, T, M, or V; D371 replaced with E; E372 replaced with D; V373 replaced with A, G, I, L, S, T, or M; S374 replaced with A, G, I, L, T, M, or V; T375 replaced with A, G, I, L, S, M, or V; S376 replaced with A, G, I, L, T, M, or V; W378 replaced with F, or Y; N379 replaced with Q; S380 replaced with A, G, I, L, T, M, or V; S381 replaced with A, G, I, L, T, M, or V; Q382 replaced with N; I384 replaced with A, G, L, S, T, M, or V; Q385 replaced with N; Q386 replaced with N; W387 replaced with F, or Y; M388 replaced with A, G, I, L, S, T, or V; K389 replaced with H, or R; K390 replaced with H, or R; Q391 replaced with N; G392 replaced with A, I, L, S, T, M, or V; W393 replaced with F, or Y; G394 replaced with A, I, L, S, T, M, or V; L395 replaced with A, G, I, S, T, M, or V; E396 replaced with D; T397 replaced with A, G, I, L, S, M, or V; A398 replaced with G, I, L, S, T, M, or V; D399 replaced with E; F400 replaced with W, or Y; M401 replaced with A, G, I, L, S, T, or V; R402 replaced with H, or K; L403 replaced with A, G, I, S, T, M, or V; W404 replaced with F, or Y; G405 replaced with A, I, L, S, T, M, or V; H406 replaced with K, or R; F407 replaced with W, or Y; Q408 replaced with N; T409 replaced with A, G, I, L, S, M, or V; E410 replaced with D; A411 replaced with G, I, L, S, T, M, or V; L412 replaced with A, G, I, S, T, M, or V; G413 replaced with A, I, L, S, T, M, or V; R414 replaced with H, or K; V415 replaced with A, G, I, L, S, T, or M; D416 replaced with E; K417 replaced with H, or R; V418 replaced with A, G, I, L, S, T, or M; A419 replaced with G, I, L, S, T, M, or V; N420 replaced with Q; G421 replaced with A, I, L, S, T, M, or V; T422 replaced with A, G, I, L, S, M, or V; H423 replaced with K, or R; T424 replaced with A, G, I, L, S, M, or V; I426 replaced with A, G, L, S, T, M, or V; I427 replaced with A, G, L, S, T, M, or V; L428 replaced with A, G, I, S, T, M, or V; W429 replaced with F, or Y; T430 replaced with A, G, I, L, S, M, or V; S431 replaced with A, G, I, L, T, M, or V; G432 replaced with A, I, L, S, T, M, or V; L433 replaced with A, G, I, S, T, M, or V; T434 replaced with A, G, I, L, S, M, or V; E435 replaced with D; E436 replaced with D; F438 replaced with W, or Y; I439 replaced with A, G, L, S, T, M, or V; D440 replaced with E; E441 replaced with D; Y442 replaced with F, or W; L443 replaced with A, G, I, S, T, M, or V; N444 replaced with Q; E446 replaced with D; R447 replaced with H, or K; Y448 replaced with F, or W; I449 replaced with A, G, L, S, T, M, or V; I450 replaced with A, G, L, S, T, M, or V; Q451 replaced with N; I452 replaced with A, G, L, S, T, M, or V; W453 replaced with F, or Y; T454 replaced with A, G, I, L, S, M, or V; T455 replaced with A, G, I, L, S, M, or V; G456 replaced with A, I, L, S, T, M, or V; V457 replaced with A, G, I, L, S, T, or M; D458 replaced with E; K460 replaced with H, or R; V461 replaced with A, G, I, L, S, T, or M; K462 replaced with H, or R; K463 replaced with H, or R; I464 replaced with A, G, L, S, T, M, or V; L465 replaced with A, G, I, S, T, M, or V; E466 replaced with D; R467 replaced with H, or K; G468 replaced with A, I, L, S, T, M, or V; Y469 replaced with F, or W; K470 replaced with H, or R; I471 replaced with A, G, L, S, T, M, or V; I472 replaced with A, G, L, S, T, M, or V; V473 replaced with A, G, I, L, S, T, or M; S474 replaced with A, G, I, L, T, M, or V; N475 replaced with Q; Y476 replaced with F, or W; D477 replaced with E; A478 replaced with G, I, L, S, T, M, or V; L479 replaced with A, G, I, S, T, M, or V; Y480 replaced with F, or W; L481 replaced with A, G, I, S, T, M, or V; D482 replaced with E; G484 replaced with A, I, L, S, T, M, or V; G485 replaced with A, I, L, S, T, M, or V; A486 replaced with G, I, L, S, T, M, or V; G487 replaced with A, I, L, S, T, M, or V; W488 replaced with F, or Y; V489 replaced with A, G, I, L, S, T, or M; T490 replaced with A, G, I, L, S, M, or V; D491 replaced with E; G492 replaced with A, I, L, S, T, M, or V; N493 replaced with Q; N494 replaced with Q; W495 replaced with F, or Y; S497 replaced with A, G, I, L, T, M, or V; Y499 replaced with F, or W; I500 replaced with A, G, L, S, T, M, or V; G501 replaced with A, I, L, S, T, M, or V; W502 replaced with F, or Y; Q503 replaced with N; K504 replaced with H, or R; V505 replaced with A, G, I, L, S, T, or M; Y506 replaced with F, or W; D507 replaced with E; N508 replaced with Q; S509 replaced with A, G, I, L, T, M, or V; L510 replaced with A, G, I, S, T, M, or V; K511 replaced with H, or R; S512 replaced with A, G, I, L, T, M, or V; I513 replaced with A, G, L, S, T, M, or V; A514 replaced with G, I, L, S, T, M, or V; G515 replaced with A, I, L, S, T, M, or V; D516 replaced with E; Y517 replaced with F, or W; E518 replaced with D; H519 replaced with K, or R; H520 replaced with K, or R; V521 replaced with A, G, I, L, S, T, or M; L522 replaced with A, G, I, S, T, M, or V; G523 replaced with A, I, L, S, T, M, or V; A524 replaced with G, I, L, S, T, M, or V; E525 replaced with D; G526 replaced with A, I, L, S, T, M, or V; A527 replaced with G, I, L, S, T, M, or V; I528 replaced with A, G, L, S, T, M, or V; W529 replaced with F, or Y; S530 replaced with A, G, I, L, T, M, or V; E531 replaced with D; Q532 replaced with N; I533 replaced with A, G, L, S, T, M, or V; D534 replaced with E; E535 replaced with D; H536 replaced with K, or R; T537 replaced with A, G, I, L, S, M, or V; L538 replaced with A, G, I, S, T, M, or V; D539 replaced with E; N540 replaced with Q; R541 replaced with H, or K; F542 replaced with W, or Y; W543 replaced with F, or Y; R545 replaced with H, or K; A546 replaced with G, I, L, S, T, M, or V; S547 replaced with A, G, I, L, T, M, or V; A548 replaced with G, I, L, S, T, M, or V; L549 replaced with A, G, I, S, T, M, or V; A550 replaced with G, I, L, S, T, M, or V; E551 replaced with D; R552 replaced with H, or K; L553 replaced with A, G, I, S, T, M, or V; W554 replaced with F, or Y; S555 replaced with A, G, I, L, T, M, or V; N556 replaced with Q; A558 replaced with G, I, L, S, T, M, or V; E559 replaced with D; G560 replaced with A, I, L, S, T, M, or V; W561 replaced with F, or Y; R562 replaced with H, or K; Q563 replaced with N; A564 replaced with G, I, L, S, T, M, or V; E565 replaced with D; S566 replaced with A, G, I, L, T, M, or V; R567 replaced with H, or K; L568 replaced with A, G, I, S, T, M, or V; L569 replaced with A, G, I, S, T, M, or V; L570 replaced with A, G, I, S, T, M, or V; H571 replaced with K, or R; R572 replaced with H, or K; Q573 replaced with N; R574 replaced with H, or K; L575 replaced with A, G, I, S, T, M, or V; V576 replaced with A, G, I, L, S, T, or M; D577 replaced with E; N578 replaced with Q; G579 replaced with A, I, L, S, T, M, or V; L580 replaced with A, G, I, S, T, M, or V; G581 replaced with A, I, L, S, T, M, or V; A582 replaced with G, I, L, S, T, M, or V; E583 replaced with D; A584 replaced with G, I, L, S, T, M, or V; M585 replaced with A, G, I, L, S, T, or V; Q586 replaced with N; Q588 replaced with N; W589 replaced with F, or Y; L591 replaced with A, G, I, S, T, M, or V; Q592 replaced with N; N593 replaced with Q; E594 replaced with D; H595 replaced with K, or R; E596 replaced with D; I599 replaced with A, G, L, S, T, M, or V; D600 replaced with E; A601 replaced with G, I, L, S, T, M, or V; Y602 replaced with F, or W; D603 replaced with E; A604 replaced with G, I, L, S, T, M, or V; Q605 replaced with N; and/or V606 replaced with A, G, I, L, S, T, or M.

The resulting constructs can be routinely screened for activities or functions described throughout the specification and known in the art. Preferably, the resulting constructs have an increased glucosaminidase activity or function, while the remaining Hex-1 activities or functions are maintained. More preferably, the resulting constructs have more than one increased biological activity or function, while the remaining biological activities or functions are maintained.

And in another example, site directed changes at the amino acid level of Hex-2 (SEQ ID NO:6) can be made by replacing a particular amino acid with a conservative amino acid. Preferred conservative mutations include: M1 replaced with A, G, I, L, S, T, or V; R2 replaced with H, or K; F3 replaced with W, or Y; S4 replaced with A, G, I, L, T, M, or V; G5 replaced with A, I, L, S, T, M, or V; Y6 replaced with F, or W; N7 replaced with Q; R8 replaced with H, or K; Y9 replaced with F, or W; Q10 replaced with N; F12 replaced with W, or Y; S14 replaced with A, G, I, L, T, M, or V; A15 replaced with G, I, L, S, T, M, or V; V16 replaced with A, G, I, L, S, T, or M; G17 replaced with A, I, L, S, T, M, or V; S18 replaced with A, G, I, L, T, M, or V; L19 replaced with A, G, I, S, T, M, or V; L20 replaced with A, G, I, S, T, M, or V; L21 replaced with A, G, I, S, T, M, or V; L22 replaced with A, G, I, S, T, M, or V; S23 replaced with A, G, I, L, T, M, or V; L24 replaced with A, G, I, S, T, M, or V; L25 replaced with A, G, I, S, T, M, or V; S26 replaced with A, G, I, L, T, M, or V; F27 replaced with W, or Y; A28 replaced with G, I, L, S, T, M, or V; V29 replaced with A, G, I, L, S, T, or M; G30 replaced with A, I, L, S, T, M, or V; A31 replaced with G, I, L, S, T, M, or V; A32 replaced with G, I, L, S, T, M, or V; L33 replaced with A, G, I, S, T, M, or V; T34 replaced with A, G, I, L, S, M, or V; R35 replaced with H, or K; A36 replaced with G, I, L, S, T, M, or V; D37 replaced with E; D38 replaced with E; V39 replaced with A, G, I, L, S, T, or M; G40 replaced with A, I, L, S, T, M, or V; S41 replaced with A, G, I, L, T, M, or V; D42 replaced with E; A43 replaced with G, I, L, S, T, M, or V; D44 replaced with E; A45 replaced with G, I, L, S, T, M, or V; G46 replaced with A, I, L, S, T, M, or V; S47 replaced with A, G, I, L, T, M, or V; A48 replaced with G, I, L, S, T, M, or V; S49 replaced with A, G, I, L, T, M, or V; R50 replaced with H, or K; K51 replaced with H, or R; W52 replaced with F, or Y; L53 replaced with A, G, I, S, T, M, or V; S55 replaced with A, G, I, L, T, M, or V; R56 replaced with H, or K; T57 replaced with A, G, I, L, S, M, or V; D58 replaced with E; I59 replaced with A, G, L, S, T, M, or V; T61 replaced with A, G, I, L, S, M, or V; A62 replaced with G, I, L, S, T, M, or V; E63 replaced with D; G64 replaced with A, I, L, S, T, M, or V; E65 replaced with D; M66 replaced with A, G, I, L, S, T, or V; V67 replaced with A, G, I, L, S, T, or M; A68 replaced with G, I, L, S, T, M, or V; G69 replaced with A, I, L, S, T, M, or V; L70 replaced with A, G, I, S, T, M, or V; Q71 replaced with N; Y72 replaced with F, or W; A73 replaced with G, I, L, S, T, M, or V; E75 replaced with D; 176 replaced with A, G, L, S, T, M, or V; F77 replaced with W, or Y; E78 replaced with D; S79 replaced with A, G, I, L, T, M, or V; Q80 replaced with N; R81 replaced with H, or K; D82 replaced with E; R84 replaced with H, or K; L85 replaced with A, G, I, S, T, M, or V; S86 replaced with A, G, I, L, T, M, or V; G88 replaced with A, I, L, S, T, M, or V; K89 replaced with H, or R; Y90 replaced with F, or W; G91 replaced with A, I, L, S, T, M, or V; A92 replaced with G, I, L, S, T, M, or V; 193 replaced with A, G, L, S, T, M, or V; W94 replaced with F, or Y; M96 replaced with A, G, I, L, S, T, or V; T98 replaced with A, G, I, L, S, M, or V; G99 replaced with A, I, L, S, T, M, or V; K100 replaced with H, or R; E101 replaced with D; T103 replaced with A, G, I, L, S, M, or V; I104 replaced with A, G, L, S, T, M, or V; S105 replaced with A, G, I, L, T, M, or V; H106 replaced with K, or R; R107 replaced with H, or K; R108 replaced with H, or K; V109 replaced with A, G, I, L, S, T, or M; R110 replaced with H, or K; F111 replaced with W, or Y; D112 replaced with E; W114 replaced with F, or Y; K115 replaced with H, or R; V116 replaced with A, G, I, L, S, T, or M; R117 replaced with H, or K; F118 replaced with W, or Y; H119 replaced with K, or R; V120 replaced with A, G, I, L, S, T, or M; V121 replaced with A, G, I, L, S, T, or M; A122 replaced with G, I, L, S, T, M, or V; G124 replaced with A, I, L, S, T, M, or V; E125 replaced with D; A126 replaced with G, I, L, S, T, M, or V; A127 replaced with G, I, L, S, T, M, or V; T128 replaced with A, G, I, L, S, M, or V; Q129 replaced with N; F130 replaced with W, or Y; L131 replaced with A, G, I, S, T, M, or V; R132 replaced with H, or K; E133 replaced with D; T134 replaced with A, G, I, L, S, M, or V; N135 replaced with Q; R136 replaced with H, or K; L137 replaced with A, G, I, S, T, M, or V; F138 replaced with W, or Y; V139 replaced with A, G, I, L, S, T, or M; S140 replaced with A, G, I, L, T, M, or V; N141 replaced with Q; L142 replaced with A, G, I, S, T, M, or V; L143 replaced with A, G, I, S, T, M, or V; K144 replaced with H, or R; E145 replaced with D; I147 replaced with A, G, L, S, T, M, or V; R148 replaced with H, or K; N149 replaced with Q; TI51 replaced with A, G, I, L, S, M, or V; L152 replaced with A, G, I, S, T, M, or V; E153 replaced with D; T154 replaced with A, G, I, L, S, M, or V; S155 replaced with A, G, I, L, T, M, or V; K156 replaced with H, or R; Q157 replaced with N; I158 replaced with A, G, L, S, T, M, or V; L159 replaced with A, G, I, S, T, M, or V; V160 replaced with A, G, I, L, S, T, or M; R161 replaced with H, or K; S162 replaced with A, G, I, L, T, M, or V; T163 replaced with A, G, I, L, S, M, or V; V164 replaced with A, G, I, L, S, T, or M; A165 replaced with G, I, L, S, T, M, or V; N166 replaced with Q; E167 replaced with D; S168 replaced with A, G, I, L, T, M, or V; L169 replaced with A, G, I, S, T, M, or V; V170 replaced with A, G, I, L, S, T, or M; L171 replaced with A, G, I, S, T, M, or V; D172 replaced with E; W173 replaced with F, or Y; T175 replaced with A, G, I, L, S, M, or V; D176 replaced with E; E177 replaced with D; S178 replaced with A, G, I, L, T, M, or V; Y179 replaced with F, or W; A180 replaced with G, I, L, S, T, M, or V; L181 replaced with A, G, I, S, T, M, or V; V182 replaced with A, G, I, L, S, T, or M; V183 replaced with A, G, I, L, S, T, or M; R184 replaced with H, or K; T185 replaced with A, G, I, L, S, M, or V; T186 replaced with A, G, I, L, S, M, or V; E187 replaced with D; T188 replaced with A, G, I, L, S, M, or V; A189 replaced with G, I, L, S, T, M, or V; T190 replaced with A, G, I, L, S, M, or V; F191 replaced with W, or Y; V192 replaced with A, G, I, L, S, T, or M; D193 replaced with E; I194 replaced with A, G, L, S, T, M, or V; Q195 replaced with N; A196 replaced with G, I, L, S, T, M, or V; T197 replaced with A, G, I, L, S, M, or V; T198 replaced with A, G, I, L, S, M, or V; V199 replaced with A, G, I, L, S, T, or M; Y200 replaced with F, or W; G201 replaced with A, I, L, S, T, M, or V; A202 replaced with G, I, L, S, T, M, or V; R203 replaced with H, or K; H204 replaced with K, or R; A205 replaced with G, I, L, S, T, M, or V; F206 replaced with W, or Y; E207 replaced with D; T208 replaced with A, G, I, L, S, M, or V; L209 replaced with A, G, I, S, T, M, or V; S210 replaced with A, G, I, L, T, M, or V; N211 replaced with Q; L212 replaced with A, G, I, S, T, M, or V; V213 replaced with A, G, I, L, S, T, or M; T214 replaced with A, G, I, L, S, M, or V; G215 replaced with A, I, L, S, T, M, or V; S216 replaced with A, G, I, L, T, M, or V; L217 replaced with A, G, I, S, T, M, or V; S218 replaced with A, G, I, L, T, M, or V; N219 replaced with Q; G220 replaced with A, , L, S, T, M, or V; L221 replaced with A, G, , S, T, M, or V; L222 replaced with A, G, I, S, T, M, or V; M223 replaced with A, G, I, L, S, T, or V; V224 replaced with A, G, I, L, S, T, or M; T225 replaced with A, G, I, L, S, M, or V; T226 replaced with A, G, I, L, S, M, or V; A227 replaced with G, I, L, S, T, M, or V; N228 replaced with Q; I229 replaced with A, G, L, S, T, M, or V; T230 replaced with A, G, I, L, S, M, or V; D231 replaced with E; R232 replaced with H, or K; A234 replaced with G, I, L, S, T, M, or V; F235 replaced with W, or Y; S236 replaced with A, G, I, L, T, M, or V; H237 replaced with K, or R; R238 replaced with H, or K; G239 replaced with A, I, L, S, T, M, or V; V240 replaced with A, G, I, L, S, T, or M; L241 replaced with A, G, I, S, T, M, or V; L242 replaced with A, G, I, S, T, M, or V; D243 replaced with E; T244 replaced with A, G, I, L, S, M, or V; A245 replaced with G, I, L, S, T, M, or V; R246 replaced with H, or K; N247 replaced with Q; F248 replaced with W, or Y; V249 replaced with A, G, I, L, S, T, or M; L251 replaced with A, G, I, S, T, M, or V; K252 replaced with H, or R; F253 replaced with W, or Y; I254 replaced with A, G, L, S, T, M, or V; R255 replaced with H, or K; S256 replaced with A, G, I, L, T, M, or V; T257 replaced with A, G, I, L, S, M, or V; L258 replaced with A, G, I, S, T, M, or V; D259 replaced with E; A260 replaced with G, I, L, S, T, M, or V; M261 replaced with A, G, I, L, S, T, or V; A262 replaced with G, I, L, S, T, M, or V; A263 replaced with G, I, L, S, T, M, or V; S264 replaced with A, G, I, L, T, M, or V; K265 replaced with H, or R; L266 replaced with A, G, I, S, T, M, or V; N267 replaced with Q; V268 replaced with A, G, I, L, S, T, or M; L269 replaced with A, G, I, S, T, M, or V; H270 replaced with K, or R; W271 replaced with F, or Y; H272 replaced with K, or R; V273 replaced with A, G, I, L, S, T, or M; V274 replaced with A, G, I, L, S, T, or M; D275 replaced with E; T276 replaced with A, G, I, L, S, M, or V; H277 replaced with K, or R; S278 replaced with A, G, I, L, T, M, or V; F279 replaced with W, or Y; L281 replaced with A, G, I, S, T, M, or V; E282 replaced with D; I283 replaced with A, G, L, S, T, M, or V; T284 replaced with A, G, I, L, S, M, or V; R285 replaced with H, or K; V286 replaced with A, G, I, L, S, T, or M; E288 replaced with D; M289 replaced with A, G, I, L, S, T, or V; Q290 replaced with N; R291 replaced with H, or K; Y292 replaced with F, or W; G293 replaced with A, I, L, S, T, M, or V; A294 replaced with G, I, L, S, T, M, or V; Y295 replaced with F, or W; S296 replaced with A, G, I, L, T, M, or V; S297 replaced with A, G, I, L, T, M, or V; S298 replaced with A, G, I, L, T, M, or V; Q299 replaced with N; T300 replaced with A, G, I, L, S, M, or V; Y301 replaced with F, or W; S302 replaced with A, G, I, L, T, M, or V; R303 replaced with H, or K; Q304 replaced with N; D305 replaced with E; A306 replaced with G, I, L, S, T, M, or V; L307 replaced with A, G, I, S, T, M, or V; N308 replaced with Q; L309 replaced with A, G, I, S, T, M, or V; V310 replaced with A, G, I, L, S, T, or M; K311 replaced with H, or R; Y312 replaced with F, or W; A313 replaced with G, I, L, S, T, M, or V; R314 replaced with H, or K; L315 replaced with A, G, I, S, T, M, or V; R316 replaced with H, or K; G317 replaced with A, I, L, S, T, M, or V; I318 replaced with A, G, L, S, T, M, or V; R319 replaced with H, or K; I320 replaced with A, G, L, S, T, M, or V; L321 replaced with A, G, I, S, T, M, or V; I322 replaced with A, G, L, S, T, M, or V; E323 replaced with D; I324 replaced with A, G, L, S, T, M, or V; D325 replaced with E; G326 replaced with A, I, L, S, T, M, or V; S328 replaced with A, G, I, L, T, M, or V; H329 replaced with K, or R; A330 replaced with G, I, L, S, T, M, or V; G331 replaced with A, I, L, S, T, M, or V; N332 replaced with Q; G333 replaced with A, I, L, S, T, M, or V; W334 replaced with F, or Y; Q335 replaced with N; W336 replaced with F, or Y; G337 replaced with A, I, L, S, T, M, or V; A339 replaced with G, I, L, S, T, M, or V; A340 replaced with G, I, L, S, T, M, or V; G341 replaced with A, I, L, S, T, M, or V; L342 replaced with A, G, I, S, T, M, or V; G343 replaced with A, I, L, S, T, M, or V; N344 replaced with Q; M345 replaced with A, G, I, L, S, T, or V; S346 replaced with A, G, I, L, T, M, or V; V347 replaced with A, G, I, L, S, T, or M; L349 replaced with A, G, I, S, T, M, or V; N350 replaced with Q; Q351 replaced with N; S352 replaced with A, G, I, L, T, M, or V; W354 replaced with F, or Y; R355 replaced with H, or K; R356 replaced with H, or K; F357 replaced with W, or Y; V359 replaced with A, G, I, L, S, T, or M; Q360 replaced with N; G364 replaced with A, I, L, S, T, M, or V; Q365 replaced with N; L366 replaced with A, G, I, S, T, M, or V; N367 replaced with Q; L369 replaced with A, G, I, S, T, M, or V; N370 replaced with Q; D371 replaced with E; H372 replaced with K, or R; M373 replaced with A, G, I, L, S, T, or V; Y374 replaced with F, or W; A375 replaced with G, I, L, S, T, M, or V; V376 replaced with A, G, I, L, S, T, or M; L377 replaced with A, G, I, S, T, M, or V; K378 replaced with H, or R; E379 replaced with D; I380 replaced with A, G, L, S, T, M, or V; F381 replaced with W, or Y; E382 replaced with D; D383 replaced with E; V384 replaced with A, G, I, L, S, T, or M; A385 replaced with G, I, L, S, T, M, or V; E386 replaced with D; V387 replaced with A, G, I, L, S, T, or M; G388 replaced with A, I, L, S, T, M, or V; A389 replaced with G, I, L, S, T, M, or V; E391 replaced with D; E392 replaced with D; T393 replaced with A, G, I, L, S, M, or V; L394 replaced with A, G, I, S, T, M, or V; H395 replaced with K, or R; M396 replaced with A, G, I, L, S, T, or V; G397 replaced with A, I, L, S, T, M, or V; G398 replaced with A, I, L, S, T, M, or V; D399 replaced with E; E400 replaced with D; V401 replaced with A, G, I, L, S, T, or M; F402 replaced with W, or Y; L403 replaced with A, G, I, S, T, M, or V; W406 replaced with F, or Y; N407 replaced with Q; N408 replaced with Q; T409 replaced with A, G, I, L, S, M, or V; D410 replaced with E; E411 replaced with D; I412 replaced with A, G, L, S, T, M, or V; R413 replaced with H, or K; D414 replaced with E; G415 replaced with A, I, L, S, T, M, or V; M416 replaced with A, G, I, L, S, T, or V; R417 replaced with H, or K; A418 replaced with G, I, L, S, T, M, or V; R419 replaced with H, or K; G420 replaced with A, I, L, S, T, M, or V; Y421 replaced with F, or W; D422 replaced with E; L423 replaced with A, G, I, S, T, M, or V; S424 replaced with A, G, I, L, T, M, or V; E425 replaced with D; Q426 replaced with N; S427 replaced with A, G, I, L, T, M, or V; F428 replaced with W, or Y; L429 replaced with A, G, I, S, T, M, or V; R430 replaced with H, or K; L431 replaced with A, G, I, S, T, M, or V; W432 replaced with F, or Y; S433 replaced with A, G, I, L, T, M, or V; Q434 replaced with N; F435 replaced with W, or Y; H436 replaced with K, or R; Q437 replaced with N; R438 replaced with H, or K; N439 replaced with Q; L440 replaced with A, G, I, S, T, M, or V; N441 replaced with Q; A442 replaced with G, I, L, S, T, M, or V; W443 replaced with F, or Y; D444 replaced with E; E445 replaced with D; I446 replaced with A, G, L, S, T, M, or V; N447 replaced with Q; E448 replaced with D; R449 replaced with H, or K; M450 replaced with A, G, I, L, S, T, or V; Y451 replaced with F, or W; G453 replaced with A, I, L, S, T, M, or V; I454 replaced with A, G, L, S, T, M, or V; K455 replaced with H, or R; E456 replaced with D; K458 replaced with H, or R; S459 replaced with A, G, I, L, T, M, or V; V460 replaced with A, G, I, L, S, T, or M; I461 replaced with A, G, L, S, T, M, or V; I462 replaced with A, G, L, S, T, M, or V; W463 replaced with F, or Y; S464 replaced with A, G, I, L, T, M, or V; S465 replaced with A, G, I, L, T, M, or V; H466 replaced with K, or R; L467 replaced with A, G, I, S, T, M, or V; T468 replaced with A, G, I, L, S, M, or V; N469 replaced with Q; R471 replaced with H, or K; Y472 replaced with F, or W; I473 replaced with A, G, L, S, T, M, or V; E474 replaced with D; T475 replaced with A, G, I, L, S, M, or V; Y476 replaced with F, or W; L477 replaced with A, G, I, S, T, M, or V; K479 replaced with H, or R; E480 replaced with D; R481 replaced with H, or K; F482 replaced with W, or Y; I483 replaced with A, G, L, S, T, M, or V; I484 replaced with A, G, L, S, T, M, or V; Q485 replaced with N; T486 replaced with A, G, I, L, S, M, or V; W487 replaced with F, or Y; V488 replaced with A, G, I, L, S, T, or M; E489 replaced with D; S490 replaced with A, G, I, L, T, M, or V; Q491 replaced with N; D492 replaced with E; A493 replaced with G, I, L, S, T, M, or V; L494 replaced with A, G, I, S, T, M, or V; N495 replaced with Q; R496 replaced with H, or K; E497 replaced with D; L498 replaced with A, G, I, S, T, M, or V; L499 replaced with A, G, I, S, T, M, or V; Q500 replaced with N; R501 replaced with H, or K; G502 replaced with A, I, L, S, T, M, or V; Y503 replaced with F, or W; R504 replaced with H, or K; L505 replaced with A, G, I, S, T, M, or V; I506 replaced with A, G, L, S, T, M, or V; V507 replaced with A, G, I, L, S, T, or M; S508 replaced with A, G, I, L, T, M, or V; T509 replaced with A, G, I, L, S, M, or V; K510 replaced with H, or R; N511 replaced with Q; A512 replaced with G, I, L, S, T, M, or V; W513 replaced with F, or Y; Y514 replaced with F, or W; L515 replaced with A, G, I, S, T, M, or V; D516 replaced with E; H517 replaced with K, or R; G518 replaced with A, I, L, S, T, M, or V; F519 replaced with W, or Y; W520 replaced with F, or Y; G521 replaced with A, I, L, S, T, M, or V; S522 replaced with A, G, I, L, T, M, or V; T523 replaced with A, G, I, L, S, M, or V; S524 replaced with A, G, I, L, T, M, or V; Y525 replaced with F, or W; Y526 replaced with F, or W; N527 replaced with Q; W528 replaced with F, or Y; R529 replaced with H, or K; T530 replaced with A, G, I, L, S, M, or V; V531 replaced with A, G, I, L, S, T, or M; Y532 replaced with F, or W; S533 replaced with A, G, I, L, T, M, or V; S534 replaced with A, G, I, L, T, M, or V; G535 replaced with A, I, L, S, T, M, or V; M536 replaced with A, G, I, L, S, T, or V; V538 replaced with A, G, I, L, S, T, or M; G539 replaced with A, I, L, S, T, M, or V; R540 replaced with H, or K; S541 replaced with A, G, I, L, T, M, or V; K542 replaced with H, or R; D543 replaced with E; Q544 replaced with N; V545 replaced with A, G, I, L, S, T, or M; L546 replaced with A, G, I, S, T, M, or V; G547 replaced with A, I, L, S, T, M, or V; G548 replaced with A, I, L, S, T, M, or V; E549 replaced with D; V550 replaced with A, G, I, L, S, T, or M; M552 replaced with A, G, I, L, S, T, or V; W553 replaced with F, or Y; S554 replaced with A, G, I, L, T, M, or V; E555 replaced with D; Y556 replaced with F, or W; V557 replaced with A, G, I, L, S, T, or M; D558 replaced with E; Q559 replaced with N; N560 replaced with Q; S561 replaced with A, G, I, L, T, M, or V; L562 replaced with A, G, I, S, T, M, or V; E563 replaced with D; S564 replaced with A, G, I, L, T, M, or V; R565 replaced with H, or K; I566 replaced with A, G, L, S, T, M, or V; W567 replaced with F, or Y; R569 replaced with H, or K; A570 replaced with G, I, L, S, T, M, or V; G571 replaced with A, I, L, S, T, M, or V; A572 replaced with G, I, L, S, T, M, or V; A573 replaced with G, I, L, S, T, M, or V; A574 replaced with G, I, L, S, T, M, or V; E575 replaced with D; R576 replaced with H, or K; M577 replaced with A, G, I, L, S, T, or V; W578 replaced with F, or Y; S579 replaced with A, G, I, L, T, M, or V; N580 replaced with Q; K582 replaced with H, or R; S583 replaced with A, G, I, L, T, M, or V; S584 replaced with A, G, I, L, T, M, or V; A585 replaced with G, I, L, S, T, M, or V; L586 replaced with A, G, I, S, T, M, or V; L587 replaced with A, G, I, S, T, M, or V; A588 replaced with G, I, L, S, T, M, or V; Q589 replaced with N; R590 replaced with H, or K; R591 replaced with H, or K; F592 replaced with W, or Y; Y593 replaced with F, or W; R594 replaced with H, or K; Y595 replaced with F, or W; R596 replaced with H, or K; E597 replaced with D; R598 replaced with H, or K; L599 replaced with A, G, I, S, T, M, or V; L600 replaced with A, G, I, S, T, M, or V; A601 replaced with G, I, L, S, T, M, or V; R602 replaced with H, or K; G603 replaced with A, I, L, S, T, M, or V; I604 replaced with A, G, L, S, T, M, or V; H605 replaced with K, or R; A606 replaced with G, I, L, S, T, M, or V; D607 replaced with E; A608 replaced with G, I, L, S, T, M, or V; V609 replaced with A, G, I, L, S, T, or M; I610 replaced with A, G, L, S, T, M, or V; H612 replaced with K, or R; W613 replaced with F, or Y; V615 replaced with A, G, I, L, S, T, or M; L616 replaced with A, G, I, S, T, M, or V; H617 replaced with K, or R; E618 replaced with D; G619 replaced with A, I, L, S, T, M, or V; Q620 replaced with N; and/or L622 replaced with A, G, I, S, T, M, or V.

The resulting constructs can be routinely screened for activities or functions described throughout the specification and known in the art. Preferably, the resulting constructs have an increased glucosaminidase activity or function, while the remaining Hex-2 activities or functions are maintained. More preferably, the resulting constructs have more than one increased biological activity or function, while the remaining biological activities or functions are maintained.

Besides conservative amino acid substitution, variants of the present invention include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

In one example, preferred non-conservative substitutions of CMP-SAT (SEQ ID NO:2) include: M1 replaced with D, E, H, K, R, N, Q, F, W, Y, P, or C; N2 replaced with D, E, H, K, R, A, G, I, L, S, T, M, V, F, W, Y, P, or C; S3 replaced with D, E, H, K, R, N, Q, F, W, Y, P, or C; I4 replaced with D, E, H, K, R, N, Q, F, W, Y, P, or C; H5 replaced with D, E, A, G, I, L, S, T, M, V, N, Q, F, W, Y, P, or C; M6 replaced with D, E, H, K, R, N, Q, F, W, Y, P, or C; N7 replaced with D, E, H, K, R, A, G, I, L, S, T, M, V, F, W, Y, P, or C; A8 replaced with D, E, H, K, R, N, Q, F, W, Y, P, or C; N9 replaced with D, E, H, K, R, A, G, I, L, S, T, M, V, F, W, Y, P, or C; T10 replaced with D, E, H, K, R, N, Q, F, W, Y, P, or C; L11 replaced with D, E, H, K, R, N, Q, F, W, Y, P, or C; K12 replaced with D, E, A, G, I, L, S, T, M, V, N, Q, F, W, Y, P, or C; Y13 replaced with D, E, H, K, R, N, Q, A, G, I, L, S, T, M, V, P, or C; I14 replaced with D, E, H, K, R, N, Q, F, W, Y, P, or C; S15 replaced with D, E, H, K, R, N, Q, F, W, Y, P, or C; L16 replaced with D, E, H, K, R, N, Q, F, W, Y, P, or C; L17 replaced with D, E, H, K, R, N, Q, F, W, Y, P, or C; T18 replaced with D, E, H, K, R, N, Q, F, W, Y, P, or C; L19 replaced with D, E, H, K, R, N, Q, F, W, Y, P, or C; T20 replaced with D, E, H, K, R, N, Q, F, W, Y, P, or C; L21 replaced with D, E, H, K, R, N, Q, F, W, Y, P, or C; Q22 replaced with D, E, H, K, R, A, G, I, L, S, T, M, V, F, W, Y, P, or C; N23 replaced with D, E, H, K, R, A, G, I, L, S, T, M, V, F, W, Y, P, or C; A24 replaced with D, E, H, K, R, N, Q, F, W, Y, P, or C; I25 replaced with D, E, H, K, R, N, Q, F, W, Y, P, or C; L26 replaced with D, E, H, K, R, N, Q, F, W, Y, P, or C; G27 replaced with D, E, H, K, R, N, Q, F, W, Y, P, or C; L28 replaced with D, E, H, K, R, N, Q, F, W, Y, P, or C; S29 replaced with D, E, H, K, R, N, Q, F, W, Y, P, or C; M30 replaced with D, E, H, K, R, N, Q, F, W, Y, P, or C; R31 replaced with D, E, A, G, I, L, S, T, M, V, N, Q, F, W, Y, P, or C; Y32 replaced with D, E, H, K, R, N, Q, A, G, I, L, S, T, M, V, P, or C; A33 replaced with D, E, H, K, R, N, Q, F, W, Y, P, or C; R34 replaced with D, E, A, G, I, L, S, T, M, V, N, Q, F, W, Y, P, or C; T35 replaced with D, E, H, K, R, N, Q, F, W, Y, P, or C; R36 replaced with D, E, A, G, I, L, S, T, M, V, N, Q, F, W, Y, P, or C; P37 replaced with D, E, H, K, R, A, G, I, L, S, T, M, V, N, Q, F, W, Y, or C; G38 replaced with D, E, H, K, R, N, Q, F, W, Y, P, or C; D39 replaced with H, K, R, A, G, I, L, S, T, M, V, N, Q, F, W, Y, P, or C; I40 replaced with D, E, H, K, R, N, Q, F, W, Y, P, or C; F41 replaced with D, E, H, K, R, N, Q, A, G, I, L, S, T, M, V, P, or C; L42 replaced with D, E, H, K, R, N, Q, F, W, Y, P, or C; S43 replaced with D, E, H, K, R, N, Q, F, W, Y, P, or C; S44 replaced with D, E, H, K, R, N, Q, F, W, Y, P, or C; T45 replaced with D, E, H, K, R, N, Q, F, W, Y, P, or C; A46 replaced with D, E, H, K, R, N, Q, F, W, Y, P, or C; V47 replaced with D, E, H, K, R, N, Q, F, W, Y, P, or C; L48 replaced with D, E, H, K, R, N, Q, F, W, Y, P, or C; M49 replaced with D, E, H, K, R, N, Q, F, W, Y, P, or C; A50 replaced with D, E, H, K, R, N, Q, F, W, Y, P, or C; E51 replaced with H, K, R, A, G, I, L, S, T, M, V, N, Q, F, W, Y, P, or C; F52 replaced with D, E, H, K, R, N, Q, A, G, I, L, S, T, M, V, P, or C; A53 replaced with D, E, H, K, R, N, Q, F, W, Y, P, or C; K54 replaced with D, E, A, G, I, L, S, T, M, V, N, Q, F, W, Y, P, or C; L55 replaced with D, E, H, K, R, N, Q, F, W, Y, P, or C; I56 replaced with D, E, H, K, R, N, Q, F, W, Y, P, or C; T57 replaced with D, E, H, K, R, N, Q, F, W, Y, P, or C; C58 replaced with D, E, H, K, R, A, G, I, L, S, T, M, V, N, Q, F, W, Y, or P; L59 replaced with D, E, H, K, R, N, Q, F, W, Y, P, or C; F60 replaced with D, E, H, K, R, N, Q, A, G, I, L, S, T, M, V, P, or C; L61 replaced with D, E, H, K, R, N, Q, F, W, Y, P, or C; V62 replaced with D, E, H, K, R, N, Q, F, W, Y, P, or C; F63 replaced with D, E, H, K, R, N, Q, A, G, I, L, S, T, M, V, P, or C; N64 replaced with D, E, H, K, R, A, G, I, L, S, T, M, V, F, W, Y, P, or C; E65 replaced with H, K, R, A, G, I, L, S, T, M, V, N, Q, F, W, Y, P, or C; E66 replaced with H, K, R, A, G, I, L, S, T, M, V, N, Q, F, W, Y, P, or C; G67 replaced with D, E, H, K, R, N, Q, F, W, Y, P, or C; K68 replaced with D, E, A, G, I, L, S, T, M, V, N, Q, F, W, Y, P, or C; D69 replaced with H, K, R, A, G, I, L, S, T, M, V, N, Q, F, W, Y, P, or C; A70 replaced with D, E, H, K, R, N, Q, F, W, Y, P, or C; Q71 replaced with D, E, H, K, R, A, G, I, L, S, T, M, V, F, W, Y, P, or C; K72 replaced with D, E, A, G, I, L, S, T, M, V, N, Q, F, W, Y, P, or C; F73 replaced with D, E, H, K, R, N, Q, A, G, I, L, S, T, M, V, P, or C; V74 replaced with D, E, H, K, R, N, Q, F, W, Y, P, or C; R75 replaced with D, E, A, G, I, L, S, T, M, V, N, Q, F, W, Y, P, or C; S76 replaced with D, E, H, K, R, N, Q, F, W, Y, P, or C; L77 replaced with D, E, H, K, R, N, Q, F, W, Y, P, or C; H78 replaced with D, E, A, G, I, L, S, T, M, V, N, Q, F, W, Y, P, or C; K79 replaced with D, E, A, G, I, L, S, T, M, V, N, Q, F, W, Y, P, or C; T80 replaced with D, E, H, K, R, N, Q, F, W, Y, P, or C; I81 replaced with D, E, H, K, R, N, Q, F, W, Y, P, or C; I82 replaced with D, E, H, K, R, N, Q, F, W, Y, P, or C; A83 replaced with D, E, H, K, R, N, Q, F, W, Y, P, or C; N84 replaced with D, E, H, K, R, A, G, I, L, S, T, M, V, F, W, Y, P, or C; P85 replaced with D, E, H, K, R, A, G, I, L, S, T, M, V, N, Q, F, W, Y, or C; M86 replaced with D, E, H, K, R, N, Q, F, W, Y, P, or C; D87 replaced with H, K, R, A, G, I, L, S, T, M, V, N, Q, F, W, Y, P, or C; T88 replaced with D, E, H, K, R, N, Q, F, W, Y, P, or C; L89 replaced with D, E, H, K, R, N, Q, F, W, Y, P, or C; K90 replaced with D, E, A, G, I, L, S, T, M, V, N, Q, F, W, Y, P, or C; V91 replaced with D, E, H, K, R, N, Q, F, W, Y, P, or C; C92 replaced with D, E, H, K, R, A, G, I, L, S, T, M, V, N, Q, F, W, Y, or P; V93 replaced with D, E, H, K, R, N, Q, F, W, Y, P, or C; P94 replaced with D, E, H, K, R, A, G, I, L, S, T, M, V, N, Q, F, W, Y, or C; S95 replaced with D, E, H, K, R, N, Q, F, W, Y, P, or C; L96 replaced with D, E, H, K, R, N, Q, F, W, Y, P, or C; V97 replaced with D, E, H, K, R, N, Q, F, W, Y, P, or C; Y98 replaced with D, E, H, K, R, N, Q, A, G, I, L, S, T, M, V, P, or C; I99 replaced with D, E, H, K, R, N, Q, F, W, Y, P, or C; V100 replaced with D, E, H, K, R, N, Q, F, W, Y, P, or C; Q101 replaced with D, E, H, K, R, A, G, I, L, S, T, M, V, F, W, Y, P, or C; N102 replaced with D, E, H, K, R, A, G, I, L, S, T, M, V, F, W, Y, P, or C; N103 replaced with D, E, H, K, R, A, G, I, L, S, T, M, V, F, W, Y, P, or C; L104 replaced with D, E, H, K, R, N, Q, F, W, Y, P, or C; L105 replaced with D, E, H, K, R, N, Q, F, W, Y, P, or C; Y106 replaced with D, E, H, K, R, N, Q, A, G, I, L, S, T, M, V, P, or C; V107 replaced with D, E, H, K, R, N, Q, F, W, Y, P, or C; S108 replaced with D, E, H, K, R, N, Q, F, W, Y, P, or C; A109 replaced with D, E, H, K, R, N, Q, F, W, Y, P, or C; S110 replaced with D, E, H, K, R, N, Q, F, W, Y, P, or C; H111 replaced with D, E, A, G, I, L, S, T, M, V, N, Q, F, W, Y, P, or C; L112 replaced with D, E, H, K, R, N, Q, F, W, Y, P, or C; D113 replaced with H, K, R, A, G, I, L, S, T, M, V, N, Q, F, W, Y, P, or C; A114 replaced with D, E, H, K, R, N, Q, F, W, Y, P, or C; A115 replaced with D, E, H, K, R, N, Q, F, W, Y, P, or C; T116 replaced with D, E, H, K, R, N, Q, F, W, Y, P, or C; Y117 replaced with D, E, H, K, R, N, Q, A, G, I, L, S, T, M, V, P, or C; Q118 replaced with D, E, H, K, R, A, G, I, L, S, T, M, V, F, W, Y, P, or C; V119 replaced with D, E, H, K, R, N, Q, F, W, Y, P, or C; T120 replaced with D, E, H, K, R, N, Q, F, W, Y, P, or C; Y121 replacedwithD, E, H, K, R, N, Q, A, G, I, L, S, T, M, V, P, or C; Q122 replaced with D, E, H, K, R, A, G, I, L, S, T, M, V, F, W, Y, P, or C; L123 replaced with D, E, H, K, R, N, Q, F, W, Y, P, or C; K124 replaced with D, E, A, G, I, L, S, T, M, V, N, Q, F, W, Y, P, or C; I125 replaced with D, E, H, K, R, N, Q, F, W, Y, P, or C; L126 replaced with D, E, H, K, R, N, Q, F, W, Y, P, or C; T127 replaced with D, E, H, K, R, N, Q, F, W, Y, P, or C; T128 replaced with D, E, H, K, R, N, Q, F, W, Y, P, or C; A129 replaced with D, E, H, K, R, N, Q, F, W, Y, P, or C; M130 replaced with D, E, H, K, R, N, Q, F, W, Y, P, or C; F131 replaced with D, E, H, K, R, N, Q, A, G, I, L, S, T, M, V, P, or C; A132 replaced with D, E, H, K, R, N, Q, F, W, Y, P, or C; V133 replaced with D, E, H, K, R, N, Q, F, W, Y, P, or C; V134 replaced with D, E, H, K, R, N, Q, F, W, Y, P, or C; I135 replaced with D, E, H, K, R, N, Q, F, W, Y, P, or C; L136 replaced with D, E, H, K, R, N, Q, F, W, Y, P, or C; R137 replaced with D, E, A, G, I, L, S, T, M, V, N, Q, F, W, Y, P, or C; R138 replaced with D, E, A, G, I, L, S, T, M, V, N, Q, F, W, Y, P, or C; K139 replaced with D, E, A, G, I, L, S, T, M, V, N, Q, F, W, Y, P, or C; L140 replaced with D, E, H, K, R, N, Q, F, W, Y, P, or C; L141 replaced with D, E, H, K, R, N, Q, F, W, Y, P, or C; N142 replaced with D, E, H, K, R, A, G, I, L, S, T, M, V, F, W, Y, P, or C; T143 replaced with D, E, H, K, R, N, Q, F, W, Y, P, or C; Q144 replaced with D, E, H, K, R, A, G, I, L, S, T, M, V, F, W, Y, P, or C; W145 replaced with D, E, H, K, R, N, Q, A, G, I, L, S, T, M, V, P, or C; G146 replaced with D, E, H, K, R, N, Q, F, W, Y, P, or C; A147 replaced with D, E, H, K, R, N, Q, F, W, Y, P, or C; L148 replaced with D, E, H, K, R, N, Q, F, W, Y, P, or C; L149 replaced with D, E, H, K, R, N, Q, F, W, Y, P, or C; L150 replaced with D, E, H, K, R, N, Q, F, W, Y, P, or C; LiSi replaced with D, E, H, K, R, N, Q, F, W, Y, P, or C; V152 replaced with D, E, H, K, R, N, Q, F, W, Y, P, or C; M153 replaced with D, E, H, K, R, N, Q, F, W, Y, P, or C; G154 replaced with D, E, H, K, R, N, Q, F, W, Y, P, or C; I155 replaced with D, E, H, K, R, N, Q, F, W, Y, P, or C; V156 replaced with D, E, H, K, R, N, Q, F, W, Y, P, or C; L157 replaced with D, E, H, K, R, N, Q, F, W, Y, P, or C; V158 replaced with D, E, H, K, R, N, Q, F, W, Y, P, or C; Q159 replaced with D, E, H, K, R, A, G, I, L, S, T, M, V, F, W, Y, P, or C; L160 replaced with D, E, H, K, R, N, Q, F, W, Y, P, or C; A161 replaced with D, E, H, K, R, N, Q, F, W, Y, P, or C; Q162 replaced with D, E, H, K, R, A, G, I, L, S, T, M, V, F, W, Y, P, or C; T163 replaced with D, E, H, K, R, N, Q, F, W, Y, P, or C; E164 replaced with H, K, R, A, G, I, L, S, T, M, V, N, Q, F, W, Y, P, or C; G165 replaced with D, E, H, K, R, N, Q, F, W, Y, P, or C; P166 replaced with D, E, H, K, R, A, G, I, L, S, T, M, V, N, Q, F, W, Y, or C; T167 replaced with D, E, H, K, R, N, Q, F, W, Y, P, or C; S168 replaced with D, E, H, K, R, N, Q, F, W, Y, P, or C; G169 replaced with D, E, H, K, R, N, Q, F, W, Y, P, or C; S170 replaced with D, E, H, K, R, N, Q, F, W, Y, P, or C; A171 replaced with D, E, H, K, R, N, Q, F, W, Y, P, or C; G172 replaced with D, E, H, K, R, N, Q, F, W, Y, P, or C; G173 replaced with D, E, H, K, R, N, Q, F, W, Y, P, or C; A174 replaced with D, E, H, K, R, N, Q, F, W, Y, P, or C; A175 replaced with D, E, H, K, R, N, Q, F, W, Y, P, or C; A176 replaced with D, E, H, K, R, N, Q, F, W, Y, P, or C; A177 replaced with D, E, H, K, R, N, Q, F, W, Y, P, or C; A178 replaced with D, E, H, K, R, N, Q, F, W, Y, P, or C; T179 replaced with D, E, H, K, R, N, Q, F, W, Y, P, or C; A180 replaced with D, E, H, K, R, N, Q, F, W, Y, P, or C; A181 replaced with D, E, H, K, R, N, Q, F, W, Y, P, or C; S182 replaced with D, E, H, K, R, N, Q, F, W, Y, P, or C; S183 replaced with D, E, H, K, R, N, Q, F, W, Y, P, or C; G184 replaced with D, E, H, K, R, N, Q, F, W, Y, P, or C; G185 replaced with D, E, H, K, R, N, Q, F, W, Y, P, or C; A186 replaced with D, E, H, K, R, N, Q, F, W, Y, P, or C; P187 replaced with D, E, H, K, R, A, G, I, L, S, T, M, V, N, Q, F, W, Y, or C; E188 replaced with H, K, R, A, G, I, L, S, T, M, V, N, Q, F, W, Y, P, or C; Q189 replaced with D, E, H, K, R, A, G, I, L, S, T, M, V, F, W, Y, P, or C; N190 replaced with D, E, H, K, R, A, G, I, L, S, T, M, V, F, W, Y, P, or C; R191 replaced with D, E, A, G, I, L, S, T, M, V, N, Q, F, W, Y, P, or C; M192 replaced with D, E, H, K, R, N, Q, F, W, Y, P, or C; L193 replaced with D, E, H, K, R, N, Q, F, W, Y, P, or C; G194 replaced with D, E, H, K, R, N, Q, F, W, Y, P, or C; L195 replaced with D, E, H, K, R, N, Q, F, W, Y, P, or C; W196 replaced with D, E, H, K, R, N, Q, A, G, I, L, S, T, M, V, P, or C; A197 replaced with D, E, H, K, R, N, Q, F, W, Y, P, or C; A198 replaced with D, E, H, K, R, N, Q, F, W, Y, P, or C; L199 replaced with D, E, H, K, R, N, Q, F, W, Y, P, or C; G200 replaced with D, E, H, K, R, N, Q, F, W, Y, P, or C; A201 replaced with D, E, H, K, R, N, Q, F, W, Y, P, or C; C202 replaced with D, E, H, K, R, A, G, I, L, S, T, M, V, N, Q, F, W, Y, or P; F203 replaced with D, E, H, K, R, N, Q, A, G, I, L, S, T, M, V, P, or C; L204 replaced with D, E, H, K, R, N, Q, F, W, Y, P, or C; S205 replaced with D, E, H, K, R, N, Q, F, W, Y, P, or C; G206 replaced with D, E, H, K, R, N, Q, F, W, Y, P, or C; F207 replaced with D, E, H, K, R, N, Q, A, G, I, L, S, T, M, V, P, or C; A208 replaced with D, E, H, K, R, N, Q, F, W, Y, P, or C; G209 replaced with D, E, H, K, R, N, Q, F, W, Y, P, or C; I210 replaced with D, E, H, K, R, N, Q, F, W, Y, P, or C; Y211 replaced with D, E, H, K, R, N, Q, A, G, I, L, S, T, M, V, P, or C; F212 replaced with D, E, H, K, R, N, Q, A, G, I, L, S, T, M, V, P, or C; E213 replaced with H, K, R, A, G, I, L, S, T, M, V, N, Q, F, W, Y, P, or C; K214 replaced with D, E, A, G, I, L, S, T, M, V, N, Q, F, W, Y, P, or C; I215 replaced with D, E, H, K, R, N, Q, F, W, Y, P, or C; L216 replaced with D, E, H, K, R, N, Q, F, W, Y, P, or C; K217 replaced with D, E, A, G, I, L, S, T, M, V, N, Q, F, W, Y, P, or C; G218 replaced with D, E, H, K, R, N, Q, F, W, Y, P, or C; A219 replaced with D, E, H, K, R, N, Q, F, W, Y, P, or C; E220 replaced with H, K, R, A, G, I, L, S, T, M, V, N, Q, F, W, Y, P, or C; I221 replaced with D, E, H, K, R, N, Q, F, W, Y, P, or C; S222 replaced with D, E, H, K, R, N, Q, F, W, Y, P, or C; V223 replaced with D, E, H, K, R, N, Q, F, W, Y, P, or C; W224 replaced with D, E, H, K, R, N, Q, A, G, I, L, S, T, M, V, P, or C; M225 replaced with D, E, H, K, R, N, Q, F, W, Y, P, or C; R226 replaced with D, E, A, G, I, L, S, T, M, V, N, Q, F, W, Y, P, or C; N227 replaced with D, E, H, K, R, A, G, I, L, S, T, M, V, F, W, Y, P, or C; V228 replaced with D, E, H, K, R, N, Q, F, W, Y, P, or C; Q229 replaced with D, E, H, K, R, A, G, I, L, S, T, M, V, F, W, Y, P, or C; L230 replaced with D, E, H, K, R, N, Q, F, W, Y, P, or C; S231 replaced with D, E, H, K, R, N, Q, F, W, Y, P, or C; L232 replaced with D, E, H, K, R, N, Q, F, W, Y, P, or C; L233 replaced with D, E, H, K, R, N, Q, F, W, Y, P, or C; S234 replaced with D, E, H, K, R, N, Q, F, W, Y, P, or C; I235 replaced with D, E, H, K, R, N, Q, F, W, Y, P, or C; P236 replaced with D, E, H, K, R, A, G, I, L, S, T, M, V, N, Q, F, W, Y, or C; F237 replaced with D, E, H, K, R, N, Q, A, G, I, L, S, T, M, V, P, or C; G238 replaced with D, E, H, K, R, N, Q, F, W, Y, P, or C; L239 replaced with D, E, H, K, R, N, Q, F, W, Y, P, or C; L240 replaced with D, E, H, K, R, N, Q, F, W, Y, P, or C; T241 replaced with D, E, H, K, R, N, Q, F, W, Y, P, or C; C242 replaced with D, E, H, K, R, A, G, I, L, S, T, M, V, N, Q, F, W, Y, or P; F243 replaced with D, E, H, K, R, N, Q, A, G, I, L, S, T, M, V, P, or C; V244 replaced with D, E, H, K, R, N, Q, F, W, Y, P, or C; N245 replaced with D, E, H, K, R, A, G, I, L, S, T, M, V, F, W, Y, P, or C; D246 replaced with H, K, R, A, G, I, L, S, T, M, V, N, Q, F, W, Y, P, or C; G247 replaced with D, E, H, K, R, N, Q, F, W, Y, P, or C; S248 replaced with D, E, H, K, R, N, Q, F, W, Y, P, or C; R249 replaced with D, E, A, G, I, L, S, T, M, V, N, Q, F, W, Y, P, or C; I250 replaced with D, E, H, K, R, N, Q, F, W, Y, P, or C; F251 replaced with D, E, H, K, R, N, Q, A, G, I, L, S, T, M, V, P, or C; D252 replaced with H, K, R, A, G, I, L, S, T, M, V, N, Q, F, W, Y, P, or C; Q253 replaced with D, E, H, K, R, A, G, I, L, S, T, M, V, F, W, Y, P, or C; G254 replaced with D, E, H, K, R, N, Q, F, W, Y, P, or C; F255 replaced with D, E, H, K, R, N, Q, A, G, I, L, S, T, M, V, P, or C; F256 replaced with D, E, H, K, R, N, Q, A, G, I, L, S, T, M, V, P, or C; K257 replaced with D, E, A, G, I, L, S, T, M, V, N, Q, F, W, Y, P, or C; G258 replaced with D, E, H, K, R, N, Q, F, W, Y, P, or C; Y259 replaced with D, E, H, K, R, N, Q, A, G, I, L, S, T, M, V, P, or C; D260 replaced with H, K, R, A, G, I, L, S, T, M, V, N, Q, F, W, Y, P, or C; L261 replaced with D, E, H, K, R, N, Q, F, W, Y, P, or C; F262 replaced with D, E, H, K, R, N, Q, A, G, I, L, S, T, M, V, P, or C; V263 replaced with D, E, H, K, R, N, Q, F, W, Y, P, or C; W264 replaced with D, E, H, K, R, N, Q, A, G, I, L, S, T, M, V, P, or C; Y265 replaced with D, E, H, K, R, N, Q, A, G, I, L, S, T, M, V, P, or C; L266 replaced with D, E, H, K, R, N, Q, F, W, Y, P, or C; V267 replaced with D, E, H, K, R, N, Q, F, W, Y, P, orC; L268 replaced with D, E, H, K, R, N, Q, F, W, Y, P, or C; L269 replaced with D, E, H, K, R, N, Q, F, W, Y, P, or C; Q270 replaced with D, E, H, K, R, A, G, I, L, S, T, M, V, F, W, Y, P, or C; A271 replaced with D, E, H, K, R, N, Q, F, W, Y, P, or C; G272 replaced with D, E, H, K, R, N, Q, F, W, Y, P, or C; G273 replaced with D, E, H, K, R, N, Q, F, W, Y, P, or C; G274 replaced with D, E, H, K, R, N, Q, F, W, Y, P, or C; L275 replaced with D, E, H, K, R, N, Q, F, W, Y, P, or C; I276 replaced with D, E, H, K, R, N, Q, F, W, Y, P, or C; V277 replaced with D, E, H, K, R, N, Q, F, W, Y, P, or C; A278 replaced with D, E, H, K, R, N, Q, F, W, Y, P, or C; V279 replaced with D, E, H, K, R, N, Q, F, W, Y, P, or C; V280 replaced with D, E, H, K, R, N, Q, F, W, Y, P, or C; V281 replaced with D, E, H, K, R, N, Q, F, W, Y, P, or C; K282 replaced with D, E, A, G, I, L, S, T, M, V, N, Q, F, W, Y, P, or C; Y283 replaced with D, E, H, K, R, N, Q, A, G, I, L, S, T, M, V, P, or C; A284 replaced with D, E, H, K, R, N, Q, F, W, Y, P, or C; D285 replaced with H, K, R, A, G, I, L, S, T, M, V, N, Q, F, W, Y, P, or C; N286 replaced with D, E, H, K, R, A, G, I, L, S, T, M, V, F, W, Y, P, or C; I287 replaced with D, E, H, K, R, N, Q, F, W, Y, P, or C; L288 replaced with D, E, H, K, R, N, Q, F, W, Y, P, or C; K289 replaced with D, E, A, G, I, L, S, T, M, V, N, Q, F, W, Y, P, or C; G290 replaced with D, E, H, K, R, N, Q, F, W, Y, P, or C; F291 replaced with D, E, H, K, R, N, Q, A, G, I, L, S, T, M, V, P, or C; A292 replaced with D, E, H, K, R, N, Q, F, W, Y, P, or C; T293 replaced with D, E, H, K, R, N, Q, F, W, Y, P, or C; S294 replaced with D, E, H, K, R, N, Q, F, W, Y, P, or C; L295 replaced with D, E, H, K, R, N, Q, F, W, Y, P, or C; A296 replaced with D, E, H, K, R, N, Q, F, W, Y, P, or C; I297 replaced with D, E, H, K, R, N, Q, F, W, Y, P, or C; I298 replaced with D, E, H, K, R, N, Q, F, W, Y, P, or C; I299 replaced with D, E, H, K, R, N, Q, F, W, Y, P, or C; S300 replaced with D, E, H, K, R, N, Q, F, W, Y, P, or C; C301 replaced with D, E, H, K, R, A, G, I, L, S, T, M, V, N, Q, F, W, Y, or P; V302 replaced with D, E, H, K, R, N, Q, F, W, Y, P, or C; A303 replaced with D, E, H, K, R, N, Q, F, W, Y, P, or C; S304 replaced with D, E, H, K, R, N, Q, F, W, Y, P, or C; I305 replaced with D, E, H, K, R, N, Q, F, W, Y, P, or C; Y306 replaced with D, E, H, K, R, N, Q, A, G, I, L, S, T, M, V, P, or C; I307 replaced with D, E, H, K, R, N, Q, F, W, Y, P, or C; F308 replaced with D, E, H, K, R, N, Q, A, G, I, L, S, T, M, V, P, or C; D309 replaced with H, K, R, A, G, I, L, S, T, M, V, N, Q, F, W, Y, P, or C; F310 replaced with D, E, H, K, R, N, Q, A, G, I, L, S, T, M, V, P, or C; N311 replaced with D, E, H, K, R, A, G, I, L, S, T, M, V, F, W, Y, P, or C; L312 replaced with D, E, H, K, R, N, Q, F, W, Y, P, or C; T313 replaced with D, E, H, K, R, N, Q, F, W, Y, P, or C; L314 replaced with D, E, H, K, R, N, Q, F, W, Y, P, or C; Q315 replaced with D, E, H, K, R, A, G, I, L, S, T, M, V, F, W, Y, P, or C; F316 replaced with D, E, H, K, R, N, Q, A, G, I, L, S, T, M, V, P, or C; S317 replaced with D, E, H, K, R, N, Q, F, W, Y, P, or C; F318 replaced with D, E, H, K, R, N, Q, A, G, I, L, S, T, M, V, P, or C; G319 replaced with D, E, H, K, R, N, Q, F, W, Y, P, or C; A320 replaced with D, E, H, K, R, N, Q, F, W, Y, P, or C; G321 replaced with D, E, H, K, R, N, Q, F, W, Y, P, or C; L322 replaced with D, E, H, K, R, N, Q, F, W, Y, P, or C; V323 replaced with D, E, H, K, R, N, Q, F, W, Y, P, or C; I324 replaced with D, E, H, K, R, N, Q, F, W, Y, P, or C; A325 replaced with D, E, H, K, R, N, Q, F, W, Y, P, or C; S326 replaced with D, E, H, K, R, N, Q, F, W, Y, P, or C; I327 replaced with D, E, H, K, R, N, Q, F, W, Y, P, or C; F328 replaced with D, E, H, K, R, N, Q, A, G, I, L, S, T, M, V, P, or C; L329 replaced with D, E, H, K, R, N, Q, F, W, Y, P, or C; Y330 replaced with D, E, H, K, R, N, Q, A, G, I, L, S, T, M, V, P, or C; G331 replaced with D, E, H, K, R, N, Q, F, W, Y, P, or C; Y332 replaced with D, E, H, K, R, N, Q, A, G, I, L, S, T, M, V, P, or C; D333 replaced with H, K, R, A, G, I, L, S, T, M, V, N, Q, F, W, Y, P, or C; P334 replaced with D, E, H, K, R, A, G, I, L, S, T, M, V, N, Q, F, W, Y, or C; A335 replaced with D, E, H, K, R, N, Q F, W, Y, P, or C; R336 replaced with D, E, A, G, I, L, S, T, M, V, N, Q, F, W, Y, P, or C; S337 replaced with D, E, H, K, R, N, Q, F, W, Y, P, or C; A338 replaced with D, E, H, K, R, N, Q, F, W, Y, P, or C; P339 replaced with D, E, H, K, R, A, G, I, L, S, T, M, V, N, Q, F, W, Y, or C; K340 replaced with D, E, A, G, I, L, S, T, M, V, N, Q, F, W, Y, P, or C; P341 replaced with D, E, H, K, R, A, G, I, L, S, T, M, V, N, Q, F, W, Y, or C; T342 replaced with D, E, H, K, R, N, Q, F, W, Y, P, or C; M343 replaced with D, E, H, K, R, N, Q, F, W, Y, P, or C; H344 replaced with D, E, A, G, I, L, S, T, M, V, N, Q, F, W, Y, P, or C; G345 replaced with D, E, H, K, R, N, Q, F, W, Y, P, or C; P346 replaced with D, E, H, K, R, A, G, I, L, S, T, M, V, N, Q, F, W, Y, or C; G347 replaced with D, E, H, K, R, N, Q, F, W, Y, P, or C; G348 replaced with D, E, H, K, R, N, Q, F, W, Y, P, or C; D349 replaced with H, K, R, A, G, I, L, S, T, M, V, N, Q, F, W, Y, P, or C; E350 replaced with H, K, R, A, G, I, L, S, T, M, V, N, Q, F, W, Y, P, or C; E351 replaced with H, K, R, A, G, I, L, S, T, M, V, N, Q, F, W, Y, P, or C; K352 replaced with D, E, A, G, I, L, S, T, M, V, N, Q, F, W, Y, P, or C; L353 replaced with D, E, H, K, R, N, Q, F, W, Y, P, or C; L354 replaced with D, E, H, K, R, N, Q, F, W, Y, P, or C; P355 replaced with D, E, H, K, R, A, G, I, L, S, T, M, V, N, Q, F, W, Y, or C; R356 replaced with D, E, A, G, I, L, S, T, M, V, N, Q, F, W, Y, P, or C; and/or V357 replaced with D, E, H, K, R, N, Q, F, W, Y, P, or C.

The resulting constructs can be routinely screened for activities or functions described throughout the specification and known in the art. Preferably, the resulting constructs have loss of a CMP-SA transport activity or function, while the remaining biological activities or functions are maintained. More preferably, the resulting constructs have more than one loss of biological activity or function, while the remaining biological activities or functions are maintained.

Additionally, more than one amino acid (e.g., 2, 3, 4, 5, 6, 7, 8, 9 and 10) can be replaced with the substituted amino acids as described above (either conservative or nonconservative). The substituted amino acids can occur in the full length, mature, or proprotein form of CMP-SAT (SEQ ID NO:2) protein, as well as the N- and C-terminal deletion mutants, having the general formula m-n, listed below.

A further embodiment of the invention relates to a polypeptide which comprises the amino acid sequence of CMP-SAT (SEQ ID NO:2) polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of CMP-SAT polypeptide (SEQ ID NO:2), which contains at least one, but not more than 10, 9, 8, 7, 6, 5, 4, 3, 2 or 1 amino acid substitutions. In specific embodiments, the number of additions, substitutions, and/or deletions in the amino acid sequence of FIG. 1 or fragments thereof (e.g., the mature form and/or other fragments described herein), is 1-5, 5-10, 5-25, 5-50, 10-50 or 50-150, conservative amino acid substitutions are preferable.

In another example, preferred non-conservative substitutions of Hex-1 (SEQ ID NO:4) include: M1 replaced with D, E, H, K, R, N, Q, F, W, Y, P, or C; K2 replaced with D, E, A, G, I, L, S, T, M, V, N, Q, F, W, Y, P, or C; S3 replaced with D, E, H, K, R, N, Q, F, W, Y, P, or C; T4 replaced with D, E, H, K, R, N, Q, F, W, Y, P, or C; R5 replaced with D, E, A, G, I, L, S, T, M, V, N, Q, F, W, Y, P, or C; T6 replaced with D, E, H, K, R, N, Q, F, W, Y, P, or C; A7 replaced with D, E, H, K, R, N, Q, F, W, Y, P, or C; L8 replaced with D, E, H, K, R, N, Q, F, W, Y, P, or C; G9 replaced with D, E, H, K, R, N, Q, F, W, Y, P, or C; V10 replaced with D, E, H, K, R, N, Q, F, W, Y, P, or C; A11 replaced with D, E, H, K, R, N, Q, F, W, Y, P, or C; L12 replaced with D, E, H, K, R, N, Q, F, W, Y, P, or C; L13 replaced with D, E, H, K, R, N, Q, F, W, Y, P, or C; L14 replaced with D, E, H, K, R, N, Q, F, W, Y, P, or C; A15 replaced with D, E, H, K, R, N, Q, F, W, Y, P, or C; L16 replaced with D, E, H, K, R, N, Q, F, W, Y, P, or C; V17 replaced with D, E, H, K, R, N, Q, F, W, Y, P, or C; S18 replaced with D, E, H, K, R, N, Q, F, W, Y, P, or C; Q19 replaced with D, E, H, K, R, A, G, I, L, S, T, M, V, F, W, Y, P, or C; L20 replaced with D, E, H, K, R, N, Q, F, W, Y, P, or C; A21 replaced with D, E, H, K, R, N, Q, F, W, Y, P, or C; A22 replaced with D, E, H, K, R, N, Q, F, W, Y, P, or C; H23 replaced with D, E, A, G, I, L, S, T, M, V, N, Q, F, W, Y, P, or C; S24 replaced with D, E, H, K, R, N, Q, F, W, Y, P, or C; S25 replaced with D, E, H, K, R, N, Q, F, W, Y, P, or C; D26 replaced with H, K, R, A, G, I, L, S, T, M, V, N, Q, F, W, Y, P, or C; D27 replaced with H, K, R, A, G, I, L, S, T, M, V, N, Q, F, W, Y, P, or C; L28 replaced with D, E, H, K, R, N, Q, F, W, Y, P, or C; V29 replaced with D, E, H, K, R, N, Q, F, W, Y, P, or C; Y30 replaced with D, E, H, K, R, N, Q, A, G, I, L, S, T, M, V, P, or C; G31 replaced with D, E, H, K, R, N, Q, F, W, Y, P, or C; Y32 replaced with D, E, H, K, R, N, Q, A, G, I, L, S, T, M, V, P, or C; E33 replaced with H, K, R, A, G, I, L, S, T, M, V, N, Q, F, W, Y, P, or C; C34 replaced with D, E, H, K, R, A, G, I, L, S, T, M, V, N, Q, F, W, Y, or P; R35 replaced with D, E, A, G, I, L, S, T, M, V, N, Q, F, W, Y, P, or C; S36 replaced with D, E, H, K, R, N, Q, F, W, Y, P, or C; G37 replaced with D, E, H, K, R, N, Q, F, W, Y, P, or C; Y38 replaced with D, E, H, K, R, N, Q, A, G, I, L, S, T, M, V, P, or C; C39 replaced with D, E, H, K, R, A, G, I, L, S, T, M, V, N, Q, F, W, Y, or P; Q40 replaced with D, E, H, K, R, A, G, I, L, S, T, M, V, F, W, Y, P, or C; K41 replaced with D, E, A, G, I, L, S, T, M, V, N, Q, F, W, Y, P, or C; V42 replaced with D, E, H, K, R, N, Q, F, W, Y, P, or C; E43 replaced with H, K, R, A, G, I, L, S, T, M, V, N, Q, F, W, Y, P, or C; L44 replaced with D, E, H, K, R, N, Q, F, W, Y, P, or C; S45 replaced with D, E, H, K, R, N, Q, F, W, Y, P, or C; E46 replaced with H, K, R, A, G, I, L, S, T, M, V, N, Q, F, W, Y, P, or C; E47 replaced with H, K, R, A, G, I, L, S, T, M, V, N, Q, F, W, Y, P, or C; N48 replaced with D, E, H, K, R, A, G, I, L, S, T, M, V, F, W, Y, P, or C; Y49 replaced with D, E, H, K, R, N, Q, A, G, I, L, S, T, M, V, P, or C; V50 replaced with D, E, H, K, R, N, Q, F, W, Y, P, or C; K51 replaced with D, E, A, G, I, L, S, T, M, V, N, Q, F, W, Y, P, or C; A52 replaced with D, E, H, K, R, N, Q, F, W, Y, P, or C; 153 replaced with D, E, H, K, R, N, Q, F, W, Y, P, or C; S54 replaced with D, E, H, K, R, N, Q, F, W, Y, P, or C; L55 replaced with D, E, H, K, R, N, Q, F, W, Y, P, or C; P56 replaced with D, E, H, K, R, A, G, I, L, S, T, M, V, N, Q, F, W, Y, or C; V57 replaced with D, E, H, K, R, N, Q, F, W, Y, P, or C; C58 replaced with D, E, H, K, R, A, G, I, L, S, T, M, V, N, Q, F, W, Y, or P; R59 replaced with D, E, A, G, I, L, S, T, M, V, N, Q, F, W, Y, P, or C; L60 replaced with D, E, H, K, R, N, Q, F, W, Y, P, or C; F61 replaced with D, E, H, K, R, N, Q, A, G, I, L, S, T, M, V, P, or C; C62 replaced with D, E, H, K, R, A, G, I, L, S, T, M, V, N, Q, F, W, Y, or P; G63 replaced with D, E, H, K, R, N, Q, F, W, Y, P, or C; S64 replaced with D, E, H, K, R, N, Q, F, W, Y, P, or C; S65 replaced with D, E, H, K, R, N, Q, F, W, Y, P, or C; I66 replaced with D, E, H, K, R, N, Q, F, W, Y, P, or C; G67 replaced with D, E, H, K, R, N, Q, F, W, Y, P, or C; T68 replaced with D, E, H, K, R, N, Q, F, W, Y, P, or C; L69 replaced with D, E, H, K, R, N, Q, F, W, Y, P, or C; W70 replaced with D, E, H, K, R, N, Q, A, G, I, L, S, T, M, V, P, or C; P71 replaced with D, E, H, K, R, A, G, I, L, S, T, M, V, N, Q, F, W, Y, or C; K72 replaced with D, E, A, G, I, L, S, T, M, V, N, Q, F, W, Y, P, or C; P73 replaced with D, E, H, K, R, A, G, I, L, S, T, M, V, N, Q, F, W, Y, or C; T74 replaced with D, E, H, K, R, N, Q, F, W, Y, P, or C; G75 replaced with D, E, H, K, R, N, Q, F, W, Y, P, or C; T76 replaced with D, E, H, K, R, N, Q, F, W, Y, P, or C; V77 replaced with D, E, H, K, R, N, Q, F, W, Y, P, or C; R78 replaced with D, E, A, G, I, L, S, T, M, V, N, Q, F, W, Y, P, or C; L79 replaced with D, E, H, K, R, N, Q, F, W, Y, P, or C; D80 replaced with H, K, R, A, G, I, L, S, T, M, V, N, Q, F, W, Y, P, or C; T81 replaced with D, E, H, K, R, N, Q, F, W, Y, P, or C; L82 replaced with D, E, H, K, R, N, Q, F, W, Y, P, or C; M83 replaced with D, E, H, K, R, N, Q, F, W, Y, P, or C; R84 replaced with D, E, A, G, I, L, S, T, M, V, N, Q, F, W, Y, P, or C; Q85 replaced with D, E, H, K, R, A, G, I, L, S, T, M, V, F, W, Y, P, or C; V86 replaced with D, E, H, K, R, N, Q, F, W, Y, P, or C; D87 replaced with H, K, R, A, G, I, L, S, T, M, V, N, Q, F, W, Y, P, or C; I88 replaced with D, E, H, K, R, N, Q, F, W, Y, P, or C; S89 replaced with D, E, H, K, R, N, Q, F, W, Y, P, or C; F90 replaced with D, E, H, K, R, N, Q, A, G, I, L, S, T, M, V, P, or C; I91 replaced with D, E, H, K, R, N, Q, F, W, Y, P, or C; D92 replaced with H, K, R, A, G, I, L, S, T, M, V, N, Q, F, W, Y, P, or C; F93 replaced with D, E, H, K, R, N, Q, A, G, I, L, S, T, M, V, P, or C; N94 replaced with D, E, H, K, R, A, G, I, L, S, T, M, V, F, W, Y, P, or C; F95 replaced with D, E, H, K, R, N, Q, A, G, I, L, S, T, M, V, P, or C; N96 replaced with D, E, H, K, R, A, G, I, L, S, T, M, V, F, W, Y, P, or C; G97 replaced with D, E, H, K, R, N, Q, F, W, Y, P, or C; 198 replaced with D, E, H, K, R, N, Q, F, W, Y, P, or C; A99 replaced with D, E, H, K, R, N, Q, F, W, Y, P, or C; R100 replaced with D, E, A, G, I, L, S, T, M, V, N, Q, F, W, Y, P, or C; Q101 replaced with D, E, H, K, R, A, G, I, L, S, T, M, V, F, W, Y, P, or C; Q102 replaced with D, E, H, K, R, A, G, I, L, S, T, M, V, F, W, Y, P, or C; K103 replaced with D, E, A, G, I, L, S, T, M, V, N, Q, F, W, Y, P, or C; L104 replaced with D, E, H, K, R, N, Q, F, W, Y, P, or C; W105 replaced with D, E, H, K, R, N, Q, A, G, I, L, S, T, M, V, P, or C; R106 replaced with D, E, A, G, I, L, S, T, M, V, N, Q, F, W, Y, P, or C; A107 replaced with D, E, H, K, R, N, Q, F, W, Y, P, or C; V108 replaced with D, E, H, K, R, N, Q, F, W, Y, P, or C; E109 replaced with H, K, R, A, G, I, L, S, T, M, V, N, Q, F, W, Y, P, or C; D110 replaced with H, K, R, A, G, I, L, S, T, M, V, N, Q, F, W, Y, P, or C; R111 replaced with D, E, A, G, I, L, S, T, M, V, N, Q, F, W, Y, P, or C; F112 replaced with D, E, H, K, R, N, Q, A, G, I, L, S, T, M, V, P, or C; M113 replaced with D, E, H, K, R, N, Q, F, W, Y, P, or C; N114 replaced with D, E, H, K, R, A, G, I, L, S, T, M, V, F, W, Y, P, or C; M115 replaced with D, E, H, K, R, N, Q, F, W, Y, P, or C; L116 replaced with D, E, H, K, R, N, Q, F, W, Y, P, or C; E117 replaced with H, K, R, A, G, I, L, S, T, M, V, N, Q, F, W, Y, P, or C; A118 replaced with D, E, H, K, R, N, Q, F, W, Y, P, or C; Q119 replaced with D, E, H, K, R, A, G, I, L, S, T, M, V, F, W, Y, P, or C; I120 replaced with D, E, H, K, R, N, Q, F, W, Y, P, or C; P121 replaced with D, E, H, K, R, A, G, I, L, S, T, M, V, N, Q, F, W, Y, or C; D122 replaced with H, K, R, A, G, I, L, S, T, M, V, N, Q, F, W, Y, P, or C; R123 replaced with D, E, A, G, I, L, S, T, M, V, N, Q, F, W, Y, P, or C; K124 replaced with D, E, A, G, I, L, S, T, M, V, N, Q, F, W, Y, P, or C; V125 replaced with D, E, H, K, R, N, Q, F, W, Y, P, or C; L126 replaced with D, E, H, K, R, N, Q, F, W, Y, P, or C; A127 replaced with D, E, H, K, R, N, Q, F, W, Y, P, or C; R128 replaced with D, E, A, G, I, L, S, T, M, V, N, Q, F, W, Y, P, or C; G129 replaced with D, E, H, K, R, N, Q, F, W, Y, P, or C; G130 replaced with D, E, H, K, R, N, Q, F, W, Y, P, or C; Y131 replaced with D, E, H, K, R, N, Q, A, G, I, L, S, T, M, V, P, or C; R132 replaced with D, E, A, G, I, L, S, T, M, V, N, Q, F, W, Y, P, or C; M133 replaced with D, E, H, K, R, N, Q, F, W, Y, P, or C; S134 replaced with D, E, H, K, R, N, Q, F, W, Y, P, or C; V135 replaced with D, E, H, K, R, N, Q, F, W, Y, P, or C; N136 replaced with D, E, H, K, R, A, G, I, L, S, T, M, V, F, W, Y, P, or C; I137 replaced with D, E, H, K, R, N, Q, F, W, Y, P, or C; N138 replaced with D, E, H, K, R, A, G, I, L, S, T, M, V, F, W, Y, P, or C; T139 replaced with D, E, H, K, R, N, Q, F, W, Y, P, or C; P140 replaced with D, E, H, K, R, A, G, I, L, S, T, M, V, N, Q, F, W, Y, or C; D141 replaced with H, K, R, A, G, I, L, S, T, M, V, N, Q, F, W, Y, P, or C; E142 replaced with H, K, R, A, G, I, L, S, T, M, V, N, Q, F, W, Y, P, or C; P143 replaced with D, E, H, K, R, A, G, I, L, S, T, M, V, N, Q, F, W, Y, or C; T144 replaced with D, E, H, K, R, N, Q, F, W, Y, P, or C; P145 replaced with D, E, H, K, R, A, G, I, L, S, T, M, V, N, Q, F, W, Y, or C; A146 replaced with D, E, H, K, R, N, Q, F, W, Y, P, or C; R147 replaced with D, E, A, G, I, L, S, T, M, V, N, Q, F, W, Y, P, or C; L148 replaced with D, E, H, K, R, N, Q, F, W, Y, P, or C; T149 replaced with D, E, H, K, R, N, Q, F, W, Y, P, or C; L150 replaced with D, E, H, K, R, N, Q, F, W, Y, P, or C; D151 replaced with H, K, R, A, G, I, L, S, T, M, V, N, Q, F, W, Y, P, or C; T152 replaced with D, E, H, K, R, N, Q, F, W, Y, P, or C; D153 replaced with H, K, R, A, G, I, L, S, T, M, V, N, Q, F, W, Y, P, or C; E154 replaced with H, K, R, A, G, I, L, S, T, M, V, N, Q, F, W, Y, P, or C; S155 replaced with D, E, H, K, R, N, Q, F, W, Y, P, or C; Y156 replaced with D, E, H, K, R, N, Q, A, G, I, L, S, T, M, V, P, or C; T157 replaced with D, E, H, K, R, N, Q, F, W, Y, P, or C; L158 replaced with D, E, H, K, R, N, Q, F, W, Y, P, or C; D159 replaced with H, K, R, A, G, I, L, S, T, M, V, N, Q, F, W, Y, P, or C; I160 replaced with D, E, H, K, R, N, Q, F, W, Y, P, or C; D161 replaced with H, K, R, A, G, I, L, S, T, M, V, N, Q, F, W, Y, P, or C; T162 replaced with D, E, H, K, R, N, Q, F, W, Y, P, or C; D163 replaced with H, K, R, A, G, I, L, S, T, M, V, N, Q, F, W, Y, P, or C; A164 replaced with D, E, H, K, R, N, Q, F, W, Y, P, or C; S165 replaced with D, E, H, K, R, N, Q, F, W, Y, P, or C; G166 replaced with D, E, H, K, R, N, Q, F, W, Y, P, or C; H167 replaced with D, E, A, G, I, L, S, T, M, V, N, Q, F, W, Y, P, or C; V168 replaced with D, E, H, K, R, N, Q, F, W, Y, P, or C; L169 replaced with D, E, H, K, R, N, Q, F, W, Y, P, or C; A170 replaced with D, E, H, K, R, N, Q, F, W, Y, P, or C; N171 replaced with D, E, H, K, R, A, G, I, L, S, T, M, V, F, W, Y, P, or C; I172 replaced with D, E, H, K, R, N, Q, F, W, Y, P, or C; T173 replaced with D, E, H, K, R, N, Q, F, W, Y, P, or C; A174 replaced with D, E, H, K, R, N, Q, F, W, Y, P, or C; S175 replaced with D, E, H, K, R, N, Q, F, W, Y, P, or C; N176 replaced with D, E, H, K, R, A, G, I, L, S, T, M, V, F, W, Y, P, or C; F177 replaced with D, E, H, K, R, N, Q, A, G, I, L, S, T, M, V, P, or C; F178 replaced with D, E, H, K, R, N, Q, A, G, I, L, S, T, M, V, P, or C; G179 replaced with D, E, H, K, R, N, Q, F, W, Y, P, or C; A180 replaced with D, E, H, K, R, N, Q, F, W, Y, P, or C; R181 replaced with D, E, A, G, I, L, S, T, M, V, N, Q, F, W, Y, P, or C; H182 replaced with D, E, A, G, I, L, S, T, M, V, N, Q, F, W, Y, P, or C; G183 replaced with D, E, H, K, R, N, Q, F, W, Y, P, or C; L184 replaced with D, E, H, K, R, N, Q, F, W, Y, P, or C; E185 replaced with H, K, R, A, G, I, L, S, T, M, V, N, Q, F, W, Y, P, or C; T186 replaced with D, E, H, K, R, N, Q, F, W, Y, P, or C; L187 replaced with D, E, H, K, R, N, Q, F, W, Y, P, or C; A188 replaced with D, E, H, K, R, N, Q, F, W, Y, P, or C; Q189 replaced with D, E, H, K, R, A, G, I, L, S, T, M, V, F, W, Y, P, or C; L190 replaced with D, E, H, K, R, N, Q, F, W, Y, P, or C; I191 replaced with D, E, H, K, R, N, Q, F, W, Y, P, or C; V192 replaced with D, E, H, K, R, N, Q, F, W, Y, P, or C; Y193 replaced with D, E, H, K, R, N, Q, A, G, I, L, S, T, M, V, P, or C; D194 replaced with H, K, R, A, G, I, L, S, T, M, V, N, Q, F, W, Y, P, or C; D195 replaced with H, K, R, A, G, I, L, S, T, M, V, N, Q, F, W, Y, P, or C; I196 replaced with D, E, H, K, R, N, Q, F, W, Y, P, or C; R197 replaced with D, E, A, G, I, L, S, T, M, V, N, Q, F, W, Y, P, or C; R198 replaced with D, E, A, G, I, L, S, T, M, V, N, Q, F, W, Y, P, or C; E199 replaced with H, K, R, A, G, I, L, S, T, M, V, N, Q, F, W, Y, P, or C; V200 replaced with D, E, H, K, R, N, Q, F, W, Y, P, or C; Q201 replaced with D, E, H, K, R, A, G, I, L, S, T, M, V, F, W, Y, P, or C; V202 replaced with D, E, H, K, R, N, Q, F, W, Y, P, or C; T203 replaced with D, E, H, K, R, N, Q, F, W, Y, P, or C; A204 replaced with D, E, H, K, R, N, Q, F, W, Y, P, or C; N205 replaced with D, E, H, K, R, A, G, I, L, S, T, M, V, F, W, Y, P, or C; A206 replaced with D, E, H, K, R, N, Q, F, W, Y, P, or C; T207 replaced with D, E, H, K, R, N, Q, F, W, Y, P, or C; I208 replaced with D, E, H, K, R, N, Q, F, W, Y, P, or C; N209 replaced with D, E, H, K, R, A, G, I, L, S, T, M, V, F, W, Y, P, or C; D210 replaced with H, K, R, A, G, I, L, S, T, M, V, N, Q, F, W, Y, P, or C; A211 replaced with D, E, H, K, R, N, Q, F, W, Y, P, or C; P212 replaced with D, E, H, K, R, A, G, I, L, S, T, M, V, N, Q, F, W, Y, or C; V213 replaced with D, E, H, K, R, N, Q, F, W, Y, P, or C; Y214 replaced with D, E, H, K, R, N, Q, A, G, I, L, S, T, M, V, P, or C; K215 replaced with D, E, A, G, I, L, S, T, M, V, N, Q, F, W, Y, P, or C; W216 replaced with D, E, H, K, R, N, Q, A, G, I, L, S, T, M, V, P, or C; R217 replaced with D, E, A, G, I, L, S, T, M, V, N, Q, F, W, Y, P, or C; G218 replaced with D, E, H, K, R, N, Q, F, W, Y, P, or C; L219 replaced with D, E, H, K, R, N, Q, F, W, Y, P, or C; L220 replaced with D, E, H, K, R, N, Q, F, W, Y, P, or C; L221 replaced with D, E, H, K, R, N, Q, F, W, Y, P, or C; D222 replaced with H, K, R, A, G, I, L, S, T, M, V, N, Q, F, W, Y, P, or C; T223 replaced with D, E, H, K, R, N, Q, F, W, Y, P, or C; S224 replaced with D, E, H, K, R, N, Q, F, W, Y, P, or C; R225 replaced with D, E, A, G, I, L, S, T, M, V, N, Q, F, W, Y, P, or C; N226 replaced with D, E, H, K, R, A, G, I, L, S, T, M, V, F, W, Y, P, or C; Y227 replaced with D, E, H, K, R, N, Q, A, G, I, L, S, T, M, V, P, or C; Y228 replaced with D, E, H, K, R, N, Q, A, G, I, L, S, T, M, V, P, or C; S229 replaced with D, E, H, K, R, N, Q, F, W, Y, P, or C; V230 replaced with D, E, H, K, R, N, Q, F, W, Y, P, or C; K231 replaced with D, E, A, G, I, L, S, T, M, V, N, Q, F, W, Y, P, or C; S232 replaced with D, E, H, K, R, N, Q, F, W, Y, P, or C; I233 replaced with D, E, H, K, R, N, Q, F, W, Y, P, or C; K234 replaced with D, E, A, G, I, L, S, T, M, V, N, Q, F, W, Y, P, or C; R235 replaced with D, E, A, G, I, L, S, T, M, V, N, Q, F, W, Y, P, or C; T236 replaced with D, E, H, K, R, N, Q, F, W, Y, P, or C; L237 replaced with D, E, H, K, R, N, Q, F, W, Y, P, or C; E238 replaced with H, K, R, A, G, I, L, S, T, M, V, N, Q, F, W, Y, P, or C; G239 replaced with D, E, H, K, R, N, Q, F, W, Y, P, or C; M240 replaced with D, E, H, K, R, N, Q, F, W, Y, P, or C; A241 replaced with D, E, H, K, R, N, Q, F, W, Y, P, or C; L242 replaced with D, E, H, K, R, N, Q, F, W, Y, P, or C; V243 replaced with D, E, H, K, R, N, Q, F, W, Y, P, or C; K244 replaced with D, E, A, G, I, L, S, T, M, V, N, Q, F, W, Y, P, or C; L245 replaced with D, E, H, K, R, N, Q, F, W, Y, P, or C; N246 replaced with D, E, H, K, R, A, G, I, L, S, T, M, V, F, W, Y, P, or C; T247 replaced with D, E, H, K, R, N, Q, F, W, Y, P, or C; F248 replaced with D, E, H, K, R, N, Q, A, G, I, L, S, T, M, V, P, or C; H249 replaced with D, E, A, G, I, L, S, T, M, V, N, Q, F, W, Y, P, or C; W250 replaced with D, E, H, K, R, N, Q, A, G, I, L, S, T, M, V, P, or C; H251 replaced with D, E, A, G, I, L, S, T, M, V, N, Q, F, W, Y, P, or C; I252 replaced with D, E, H, K, R, N, Q, F, W, Y, P, or C; T253 replaced with D, E, H, K, R, N, Q, F, W, Y, P, or C; D254 replaced with H, K, R, A, G, I, L, S, T, M, V, N, Q, F, W, Y, P, or C; S255 replaced with D, E, H, K, R, N, Q, F, W, Y, P, or C; H256 replaced with D, E, A, G, I, L, S, T, M, V, N, Q, F, W, Y, P, or C; S257 replaced with D, E, H, K, R, N, Q, F, W, Y, P, or C; F258 replaced with D, E, H, K, R, N, Q, A, G, I, L, S, T, M, V, P, or C; P259 replaced with D, E, H, K, R, A, G, I, L, S, T, M, V, N, Q, F, W, Y, or C; L260 replaced with D, E, H, K, R, N, Q, F, W, Y, P, or C; E261 replaced with H, K, R, A, G, I, L, S, T, M, V, N, Q, F, W, Y, P, or C; V262 replaced with D, E, H, K, R, N, Q, F, W, Y, P, or C; K263 replaced with D, E, A, G, I, L, S, T, M, V, N, Q, F, W, Y, P, or C; K264 replaced with D, E, A, G, I, L, S, T, M, V, N, Q, F, W, Y, P, or C; R265 replaced with D, E, A, G, I, L, S, T, M, V, N, Q, F, W, Y, P, or C; P266 replaced with D, E, H, K, R, A, G, I, L, S, T, M, V, N, Q, F, W, Y, or C; E267 replaced with H, K, R, A, G, I, L, S, T, M, V, N, Q, F, W, Y, P, or C; L268 replaced with D, E, H, K, R, N, Q, F, W, Y, P, or C; H269 replaced with D, E, A, G, I, L, S, T, M, V, N, Q, F, W, Y, P, or C; K270 replaced with D, E, A, G, I, L, S, T, M, V, N, Q, F, W, Y, P, or C; L271 replaced with D, E, H, K, R, N, Q, F, W, Y, P, or C; G272 replaced with D, E, H, K, R, N, Q, F, W, Y, P, or C; A273 replaced with D, E, H, K, R, N, Q, F, W, Y, P, or C; Y274 replaced with D, E, H, K, R, N, Q, A, G, I, L, S, T, M, V, P, or C; S275 replaced with D, E, H, K, R, N, Q, F, W, Y, P, or C; Q276 replaced with D, E, H, K, R, A, G, I, L, S, T, M, V, F, W, Y, P, or C; R277 replaced with D, E, A, G, I, L, S, T, M, V, N, Q, F, W, Y, P, or C; Q278 replaced with D, E, H, K, R, A, G, I, L, S, T, M, V, F, W, Y, P, or C; V279 replaced with D, E, H, K, R, N, Q, F, W, Y, P, or C; Y280 replaced with D, E, H, K, R, N, Q, A, G, I, L, S, T, M, V, P, or C; T281 replaced with D, E, H, K, R, N, Q, F, W, Y, P, or C; R282 replaced with D, E, A, G, I, L, S, T, M, V, N, Q, F, W, Y, P, or C; R283 replaced with D, E, A, G, I, L, S, T, M, V, N, Q, F, W, Y, P, or C; D284 replaced with H, K, R, A, G, I, L, S, T, M, V, N, Q, F, W, Y, P, or C; V285 replaced with D, E, H, K, R, N, Q, F, W, Y, P, or C; A286 replaced with D, E, H, K, R, N, Q, F, W, Y, P, or C; E287 replaced with H, K, R, A, G, I, L, S, T, M, V, N, Q, F, W, Y, P, or C; V288 replaced with D, E, H, K, R, N, Q, F, W, Y, P, or C; V289 replaced with D, E, H, K, R, N, Q, F, W, Y, P, or C; E290 replaced with H, K, R, A, G, I, L, S, T, M, V, N, Q, F, W, Y, P, or C; Y291 replaced with D, E, H, K, R, N, Q, A, G, I, L, S, T, M, V, P, or C; G292 replaced with D, E, H, K, R, N, Q, F, W, Y, P, or C; R293 replaced with D, E, A, G, I, L, S, T, M, V, N, Q, F, W, Y, P, or C; V294 replaced with D, E, H, K, R, N, Q, F, W, Y, P, or C; R295 replaced with D, E, A, G, I, L, S, T, M, V, N, Q, F, W, Y, P, or C; G296 replaced with D, E, H, K, R, N, Q, F, W, Y, P, or C; I297 replaced with D, E, H, K, R, N, Q, F, W, Y, P, or C; R298 replaced with D, E, A, G, I, L, S, T, M, V, N, Q, F, W, Y, P, or C; V299 replaced with D, E, H, K, R, N, Q, F, W, Y, P, or C; M300 replaced with D, E, H, K, R, N, Q, F, W, Y, P, or C; P301 replaced with D, E, H, K, R, A, G, I, L, S, T, M, V, N, Q, F, W, Y, or C; E302 replaced with H, K, R, A, G, I, L, S, T, M, V, N, Q, F, W, Y, P, or C; F303 replaced with D, E, H, K, R, N, Q, A, G, I, L, S, T, M, V, P, or C; D304 replaced with H, K, R, A, G, I, L, S, T, M, V, N, Q, F, W, Y, P, or C; A305 replaced with D, E, H, K, R, N, Q, F, W, Y, P, or C; P306 replaced with D, E, H, K, R, A, G, I, L, S, T, M, V, N, Q, F, W, Y, or C; A307 replaced with D, E, H, K, R, N, Q, F, W, Y, P, or C; H308 replaced with D, E, A, G, I, L, S, T, M, V, N, Q, F, W, Y, P, or C; V309 replaced with D, E, H, K, R, N, Q, F, W, Y, P, or C; G310 replaced with D, E, H, K, R, N, Q, F, W, Y, P, or C; E311 replaced with H, K, R, A, G, I, L, S, T, M, V, N, Q, F, W, Y, P, or C; G312 replaced with D, E, H, K, R, N, Q, F, W, Y, P, or C; W313 replaced with D, E, H, K, R, N, Q, A, G, I, L, S, T, M, V, P, or C; Q314 replaced with D, E, H, K, R, A, G, I, L, S, T, M, V, F, W, Y, P, or C; H315 replaced with D, E, A, G, I, L, S, T, M, V, N, Q, F, W, Y, P, or C; K316 replaced with D, E, A, G, I, L, S, T, M, V, N, Q, F, W, Y, P, or C; N317 replaced with D, E, H, K, R, A, G, I, L, S, T, M, V, F, W, Y, P, or C; M318 replaced with D, E, H, K, R, N, Q, F, W, Y, P, or C; T319 replaced with D, E, H, K, R, N, Q, F, W, Y, P, or C; A320 replaced with D, E, H, K, R, N, Q, F, W, Y, P, or C; C321 replaced with D, E, H, K, R, A, G, I, L, S, T, M, V, N, Q, F, W, Y, or P; F322 replaced with D, E, H, K, R, N, Q, A, G, I, L, S, T, M, V, P, or C; N323 replaced with D, E, H, K, R, A, G, I, L, S, T, M, V, F, W, Y, P, or C; A324 replaced with D, E, H, K, R, N, Q, F, W, Y, P, or C; Q325 replaced with D, E, H, K, R, A, G, I, L, S, T, M, V, F, W, Y, P, or C; P326 replaced with D, E, H, K, R, A, G, I, L, S, T, M, V, N, Q, F, W, Y, or C; W327 replaced with D, E, H, K, R, N, Q, A, G, I, L, S, T, M, V, P, or C; K328 replaced with D, E, A, G, I, L, S, T, M, V, N, Q, F, W, Y, P, or C; S329 replaced with D, E, H, K, R, N, Q, F, W, Y, P, or C; F330 replaced with D, E, H, K, R, N, Q, A, G, I, L, S, T, M, V, P, or C; C331 replaced with D, E, H, K, R, A, G, I, L, S, T, M, V, N, Q, F, W, Y, or P; V332 replaced with D, E, H, K, R, N, Q, F, W, Y, P, or C; E333 replaced with H, K, R, A, G, I, L, S, T, M, V, N, Q, F, W, Y, P, or C; P334 replaced with D, E, H, K, R, A, G, I, L, S, T, M, V, N, Q, F, W, Y, or C; P335 replaced with D, E, H, K, R, A, G, I, L, S, T, M, V, N, Q, F, W, Y, or C; C336 replaced with D, E, H, K, R, A, G, I, L, S, T, M, V, N, Q, F, W, Y, or P; G337 replaced with D, E, H, K, R, N, Q, F, W, Y, P, or C; Q338 replaced with D, E, H, K, R, A, G, I, L, S, T, M, V, F, W, Y, P, or C; L339 replaced with D, E, H, K, R, N, Q, F, W, Y, P, or C; D340 replaced with H, K, R, A, G, I, L, S, T, M, V, N, Q, F, W, Y, P, or C; P341 replaced with D, E, H, K, R, A, G, I, L, S, T, M, V, N, Q, F, W, Y, or C; T342 replaced with D, E, H, K, R, N, Q, F, W, Y, P, or C; V343 replaced with D, E, H, K, R, N, Q, F, W, Y, P, or C; N344 replaced with D, E, H, K, R, A, G, I, L, S, T, M, V, F, W, Y, P, or C; E345 replaced with H, K, R, A, G, I, L, S, T, M, V, N, Q, F, W, Y, P, or C; M346 replaced with D, E, H, K, R, N, Q, F, W, Y, P, or C; Y347 replaced with D, E, H, K, R, N, Q, A, G, I, L, S, T, M, V, P, or C; D348 replaced with H, K, R, A, G, I, L, S, T, M, V, N, Q, F, W, Y, P, or C; V349 replaced with D, E, H, K, R, N, Q, F, W, Y, P, or C; L350 replaced with D, E, H, K, R, N, Q, F, W, Y, P, or C; E351 replaced with H, K, R, A, G, I, L, S, T, M, V, N, Q, F, W, Y, P, or C; D352 replaced with H, K, R, A, G, I, L, S, T, M, V, N, Q, F, W, Y, P, or C; I353 replaced with D, E, H, K, R, N, Q, F, W, Y, P, or C; Y354 replaced with D, E, H, K, R, N, Q, A, G, I, L, S, T, M, V, P, or C; G355 replaced with D, E, H, K, R, N, Q, F, W, Y, P, or C; T356 replaced with D, E, H, K, R, N, Q, F, W, Y, P, or C; M357 replaced with D, E, H, K, R, N, Q, F, W, Y, P, or C; F358 replaced with D, E, H, K, R, N, Q, A, G, I, L, S, T, M, V, P, or C; D359 replaced with H, K, R, A, G, I, L, S, T, M, V, N, Q, F, W, Y, P, or C; Q360 replaced with D, E, H, K, R, A, G, I, L, S, T, M, V, F, W, Y, P, or C; F361 replaced with D, E, H, K, R, N, Q, A, G, I, L, S, T, M, V, P, or C; N362 replaced with D, E, H, K, R, A, G, I, L, S, T, M, V, F, W, Y, P, or C; P363 replaced with D, E, H, K, R, A, G, I, L, S, T, M, V, N, Q, F, W, Y, or C; D364 replaced with H, K, R, A, G, I, L, S, T, M, V, N, Q, F, W, Y, P, or C; I365 replaced with D, E, H, K, R, N, Q, F, W, Y, P, or C; F366 replaced with D, E, H, K, R, N, Q, A, G, I, L, S, T, M, V, P, or C; H367 replaced with D, E, A, G, I, L, S, T, M, V, N, Q, F, W, Y, P, or C; M368 replaced with D, E, H, K, R, N, Q, F, W, Y, P, or C; G369 replaced with D, E, H, K, R, N, Q, F, W, Y, P, or C; G370 replaced with D, E, H, K, R, N, Q, F, W, Y, P, or C; D371 replaced with H, K, R, A, G, I, L, S, T, M, V, N, Q, F, W, Y, P, or C; E372 replaced with H, K, R, A, G, I, L, S, T, M, V, N, Q, F, W, Y, P, or C; V373 replaced with D, E, H, K, R, N, Q, F, W, Y, P, or C; S374 replaced with D, E, H, K, R, N, Q, F, W, Y, P, or C; T375 replaced with D, E, H, K, R, N, Q, F, W, Y, P, or C; S376 replaced with D, E, H, K, R, N, Q, F, W, Y, P, or C; C377 replaced with D, E, H, K, R, A, G, I, L, S, T, M, V, N, Q, F, W, Y, or P; W378 replaced with D, E, H, K, R, N, Q, A, G, I, L, S, T, M, V, P, or C; N379 replaced with D, E, H, K, R, A, G, I, L, S, T, M, V, F, W, Y, P, or C; S380 replaced with D, E, H, K, R, N, Q, F, W, Y, P, or C; S381 replaced with D, E, H, K, R, N, Q, F, W, Y, P, or C; Q382 replaced with D, E, H, K, R, A, G, I, L, S, T, M, V, F, W, Y, P, or C; P383 replaced with D, E, H, K, R, A, G, I, L, S, T, M, V, N, Q, F, W, Y, or C; I384 replaced with D, E, H, K, R, N, Q, F, W, Y, P, or C; Q385 replaced with D, E, H, K, R, A, G, I, L, S, T, M, V, F, W, Y, P, or C; Q386 replaced with D, E, H, K, R, A, G, I, L, S, T, M, V, F, W, Y, P, or C; W387 replaced with D, E, H, K, R, N, Q, A, G, I, L, S, T, M, V, P, or C; M388 replaced with D, E, H, K, R, N, Q, F, W, Y, P, or C; K389 replaced with D, E, A, G, I, L, S, T, M, V, N, Q, F, W, Y, P, or C; K390 replaced with D, E, A, G, I, L, S, T, M, V, N, Q, F, W, Y, P, or C; Q391 replaced with D, E, H, K, R, A, G, I, L, S, T, M, V, F, W, Y, P, or C; G392 replaced with D, E, H, K, R, N, Q, F, W, Y, P, or C; W393 replaced with D, E, H, K, R, N, Q, A, G, I, L, S, T, M, V, P, or C; G394 replaced with D, E, H, K, R, N, Q, F, W, Y, P, or C; L395 replaced with D, E, H, K, R, N, Q, F, W, Y, P, or C; E396 replaced with H, K, R, A, G, I, L, S, T, M, V, N, Q, F, W, Y, P, or C; T397 replaced with D, E, H, K, R, N, Q, F, W, Y, P, or C; A398 replaced with D, E, H, K, R, N, Q, F, W, Y, P, or C; D399 replaced with H, K, R, A, G, I, L, S, T, M, V, N, Q, F, W, Y, P, or C; F400 replaced with D, E, H, K, R, N, Q, A, G, I, L, S, T, M, V, P, or C; M401 replaced with D, E, H, K, R, N, Q, F, W, Y, P, or C; R402 replaced with D, E, A, G, I, L, S, T, M, V, N, Q, F, W, Y, P, or C; L403 replaced with D, E, H, K, R, N, Q, F, W, Y, P, or C; W404 replaced with D, E, H, K, R, N, Q, A, G, I, L, S, T, M, V, P, or C; G405 replaced with D, E, H, K, R, N, Q, F, W, Y, P, or C; H406 replaced with D, E, A, G, I, L, S, T, M, V, N, Q, F, W, Y, P, or C; F407 replaced with D, E, H, K, R, N, Q, A, G, I, L, S, T, M, V, P, or C; Q408 replaced with D, E, H, K, R, A, G, I, L, S, T, M, V, F, W, Y, P, or C; T409 replaced with D, E, H, K, R, N, Q, F, W, Y, P, or C; E410 replaced with H, K, R, A, G, I, L, S, T, M, V, N, Q, F, W, Y, P, or C; A411 replaced with D, E, H, K, R, N, Q, F, W, Y, P, or C; L412 replaced with D, E, H, K, R, N, Q, F, W, Y, P, or C; G413 replaced with D, E, H, K, R, N, Q, F, W, Y, P, or C; R414 replaced with D, E, A, G, I, L, S, T, M, V, N, Q, F, W, Y, P, or C; V415 replaced with D, E, H, K, R, N, Q, F, W, Y, P, or C; D416 replaced with H, K, R, A, G, I, L, S, T, M, V, N, Q, F, W, Y, P, or C; K417 replaced with D, E, A, G, I, L, S, T, M, V, N, Q, F, W, Y, P, or C; V418 replaced with D, E, H, K, R, N, Q, F, W, Y, P, or C; A419 replaced with D, E, H, K, R, N, Q, F, W, Y, P, or C; N420 replaced with D, E, H, K, R, A, G, I, L, S, T, M, V, F, W, Y, P, or C; G421 replaced with D, E, H, K, R, N, Q, F, W, Y, P, or C; T422 replaced with D, E, H, K, R, N, Q, F, W, Y, P, or C; H423 replaced with D, E, A, G, I, L, S, T, M, V, N, Q, F, W, Y, P, or C; T424 replaced with D, E, H, K, R, N, Q, F, W, Y, P, or C; P425 replaced with D, E, H, K, R, A, G, I, L, S, T, M, V, N, Q, F, W, Y, or C; I426 replaced with D, E, H, K, R, N, Q, F, W, Y, P, or C; I427 replaced with D, E, H, K, R, N, Q, F, W, Y, P, or C; L428 replaced with D, E, H, K, R, N, Q, F, W, Y, P, or C; W429 replaced with D, E, H, K, R, N, Q, A, G, I, L, S, T, M, V, P, or C; T430 replaced with D, E, H, K, R, N, Q, F, W, Y, P, or C; S431 replaced with D, E, H, K, R, N, Q, F, W, Y, P, or C; G432 replaced with D, E, H, K, R, N, Q, F, W, Y, P, or C; L433 replaced with D, E, H, K, R, N, Q, F, W, Y, P, or C; T434 replaced with D, E, H, K, R, N, Q, F, W, Y, P, or C; E435 replaced with H, K, R, A, G, I, L, S, T, M, V, N, Q, F, W, Y, P, or C; E436 replaced with H, K, R, A, G, I, L, S, T, M, V, N, Q, F, W, Y, P, or C; P437 replaced with D, E, H, K, R, A, G, I, L, S, T, M, V, N, Q, F, W, Y, or C; F438 replaced with D, E, H, K, R, N, Q, A, G, I, L, S, T, M, V, P, or C; I439 replaced with D, E, H, K, R, N, Q, F, W, Y, P, or C; D440 replaced with H, K, R, A, G, I, L, S, T, M, V, N, Q, F, W, Y, P, or C; E441 replaced with H, K, R, A, G, I, L, S, T, M, V, N, Q, F, W, Y, P, or C; Y442 replaced with D, E, H, K, R, N, Q, A, G, I, L, S, T, M, V, P, or C; L443 replaced with D, E, H, K, R, N, Q, F, W, Y, P, or C; N444 replaced with D, E, H, K, R, A, G, I, L, S, T, M, V, F, W, Y, P, or C; P445 replaced with D, E, H, K, R, A, G, I, L, S, T, M, V, N, Q, F, W, Y, or C; E446 replaced with H, K, R, A, G, I, L, S, T, M, V, N, Q, F, W, Y, P, or C; R447 replaced with D, E, A, G, I, L, S, T, M, V, N, Q, F, W, Y, P, or C; Y448 replaced with D, E, H, K, R, N, Q, A, G, I, L, S, T, M, V, P, or C; I449 replaced with D, E, H, K, R, N, Q, F, W, Y, P, or C; I450 replaced with D, E, H, K, R, N, Q, F, W, Y, P, or C; Q451 replaced with D, E, H, K, R, A, G, I, L, S, T, M, V, F, W, Y, P, or C; I452 replaced with D, E, H, K, R, N, Q, F, W, Y, P, or C; W453 replaced with D, E, H, K, R, N, Q, A, G, I, L, S, T, M, V, P, or C; T454 replaced with D, E, H, K, R, N, Q, F, W, Y, P, or C; T455 replaced with D, E, H, K, R, N, Q, F, W, Y, P, or C; G456 replaced with D, E, H, K, R, N, Q, F, W, Y, P, or C; V457 replaced with D, E, H, K, R, N, Q, F, W, Y, P, or C; D458 replaced with H, K, R, A, G, I, L, S, T, M, V, N, Q, F, W, Y, P, or C; P459 replaced with D, E, H, K, R, A, G, I, L, S, T, M, V, N, Q, F, W, Y, or C; K460 replaced with D, E, A, G, I, L, S, T, M, V, N, Q, F, W, Y, P, or C; V461 replaced with D, E, H, K, R, N, Q, F, W, Y, P, or C; K462 replaced with D, E, A, G, I, L, S, T, M, V, N, Q, F, W, Y, P, or C; K463 replaced with D, E, A, G, I, L, S, T, M, V, N, Q, F, W, Y, P, or C; I464 replaced with D, E, H, K, R, N, Q, F, W, Y, P, or C; L465 replaced with D, E, H, K, R, N, Q, F, W, Y, P, or C; E466 replaced with H, K, R, A, G, I, L, S, T, M, V, N, Q, F, W, Y, P, or C; R467 replaced with D, E, A, G, I, L, S, T, M, V, N, Q, F, W, Y, P, or C; G468 replaced with D, E, H, K, R, N, Q, F, W, Y, P, or C; Y469 replaced with D, E, H, K, R, N, Q, A, G, I, L, S, T, M, V, P, or C; K470 replaced with D, E, A, G, I, L, S, T, M, V, N, Q, F, W, Y, P, or C; I471 replaced with D, E, H, K, R, N, Q, F, W, Y, P, or C; I472 replaced with D, E, H, K, R, N, Q, F, W, Y, P, or C; V473 replaced with D, E, H, K, R, N, Q, F, W, Y, P, or C; S474 replaced with D, E, H, K, R, N, Q, F, W, Y, P, or C; N475 replaced with D, E, H, K, R, A, G, I, L, S, T, M, V, F, W, Y, P, or C; Y476 replaced with D, E, H, K, R, N, Q, A, G, I, L, S, T, M, V, P, or C; D477 replaced with H, K, R, A, G, I, L, S, T, M, V, N, Q, F, W, Y, P, or C; A478 replaced with D, E, H, K, R, N, Q, F, W, Y, P, or C; L479 replaced with D, E, H, K, R, N, Q, F, W, Y, P, or C; Y480 replaced with D, E, H, K, R, N, Q, A, G, I, L, S, T, M, V, P, or C; L481 replaced with D, E, H, K, R, N, Q, F, W, Y, P, or C; D482 replaced with H, K, R, A, G, I, L, S, T, M, V, N, Q, F, W, Y, P, or C; C483 replaced with D, E, H, K, R, A, G, I, L, S, T, M, V, N, Q, F, W, Y, or P; G484 replaced with D, E, H, K, R, N, Q, F, W, Y, P, or C; G485 replaced with D, E, H, K, R, N, Q, F, W, Y, P, or C; A486 replaced with D, E, H, K, R, N, Q, F, W, Y, P, or C; G487 replaced with D, E, H, K, R, N, Q, F, W, Y, P, or C; W488 replaced with D, E, H, K, R, N, Q, A, G, I, L, S, T, M, V, P, or C; V489 replaced with D, E, H, K, R, N, Q, F, W, Y, P, or C; T490 replaced with D, E, H, K, R, N, Q, F, W, Y, P, or C; D491 replaced with H, K, R, A, G, I, L, S, T, M, V, N, Q, F, W, Y, P, or C; G492 replaced with D, E, H, K, R, N, Q, F, W, Y, P, or C; N493 replaced with D, E, H, K, R, A, G, I, L, S, T, M, V, F, W, Y, P, or C; N494 replaced with D, E, H, K, R, A, G, I, L, S, T, M, V, F, W, Y, P, or C; W495 replaced with D, E, H, K, R, N, Q, A, G, I, L, S, T, M, V, P, or C; C496 replaced with D, E, H, K, R, A, G, I, L, S, T, M, V, N, Q, F, W, Y, or P; S497 replaced with D, E, H, K, R, N, Q, F, W, Y, P, or C; P498 replaced with D, E, H, K, R, A, G, I, L, S, T, M, V, N, Q, F, W, Y, or C; Y499 replaced with D, E, H, K, R, N, Q, A, G, I, L, S, T, M, V, P, or C; I500 replaced with D, E, H, K, R, N, Q, F, W, Y, P, or C; G501 replaced with D, E, H, K, R, N, Q, F, W, Y, P, or C; W502 replaced with D, E, H, K, R, N, Q, A, G, I, L, S, T, M, V, P, or C; Q503 replaced with D, E, H, K, R, A, G, I, L, S, T, M, V, F, W, Y, P, or C; K504 replaced with D, E, A, G, I, L, S, T, M, V, N, Q, F, W, Y, P, or C; V505 replaced with D, E, H, K, R, N, Q, F, W, Y, P, or C; Y506 replaced with D, E, H, K, R, N, Q, A, G, I, L, S, T, M, V, P, or C; D507 replaced with H, K, R, A, G, I, L, S, T, M, V, N, Q, F, W, Y, P, or C; N508 replaced with D, E, H, K, R, A, G, I, L, S, T, M, V, F, W, Y, P, or C; S509 replaced with D, E, H, K, R, N, Q, F, W, Y, P, or C; L510 replaced with D, E, H, K, R, N, Q, F, W, Y, P, or C; K511 replaced with D, E, A, G, I, L, S, T, M, V, N, Q, F, W, Y, P, or C; S512 replaced with D, E, H, K, R, N, Q, F, W, Y, P, or C; I513 replaced with D, E, H, K, R, N, Q, F, W, Y, P, or C; A514 replaced with D, E, H, K, R, N, Q, F, W, Y, P, or C; G515 replaced with D, E, H, K, R, N, Q, F, W, Y, P, or C; D516 replaced with H, K, R, A, G, I, L, S, T, M, V, N, Q, F, W, Y, P, or C; Y517 replaced with D, E, H, K, R, N, Q, A, G, I, L, S, T, M, V, P, or C; E518 replaced with H, K, R, A, G, I, L, S, T, M, V, N, Q, F, W, Y, P, or C; H519 replaced with D, E, A, G, I, L, S T, M, V, N, Q, F, W, Y, P, or C; H520 replaced with D, E, A, G, I, L, S, T, M, V, N, Q, F, W, Y, P, or C; V521 replaced with D, E, H, K, R, N, Q, F, W, Y, P, or C; L522 replaced with D, E, H, K, R, N, Q, F, W, Y, P, or C; G523 replaced with D, E, H, K, R, N, Q, F, W, Y, P, or C; A524 replaced with D, E, H, K, R, N, Q, F, W, Y, P, or C; E525 replaced with H, K, R, A, G, I, L, S, T, M, V, N, Q, F, W, Y, P, or C; G526 replaced with D, E, H, K, R, N, Q, F, W, Y, P, or C; A527 replaced with D, E, H, K, R, N, Q, F, W, Y, P, or C; I528 replaced with D, E, H, K, R, N, Q, F, W, Y, P, or C; W529 replaced with D, E, H, K, R, N, Q, A, G, I, L, S, T, M, V, P, or C; S530 replaced with D, E, H, K, R, N, Q, F, W, Y, P, or C; E531 replaced with H, K, R, A, G, I, L, S, T, M, V, N, Q, F, W, Y, P, or C; Q532 replaced with D, E, H, K, R, A, G, I, L, S, T, M, V, F, W, Y, P, or C; I533 replaced with D, E, H, K, R, N, Q, F, W, Y, P, or C; D534 replaced with H, K, R, A, G, I, L, S, T, M, V, N, Q, F, W, Y, P, or C; E535 replaced with H, K, R, A, G, I, L, S, T, M, V, N, Q, F, W, Y, P, or C; H536 replaced with D, E, A, G, I, L, S, T, M, V, N, Q, F, W, Y, P, or C; T537 replaced with D, E, H, K, R, N, Q, F, W, Y, P, or C; L538 replaced with D, E, H, K, R, N, Q, F, W, Y, P, or C; D539 replaced with H, K, R, A, G, I, L, S, T, M, V, N, Q, F, W, Y, P, or C; N540 replaced with D, E, H, K, R, A, G, I, L, S, T, M, V, F, W, Y, P, or C; R541 replaced with D, E, A, G, I, L, S, T, M, V, N, Q, F, W, Y, P, or C; F542 replaced with D, E, H, K, R, N, Q, A, G, I, L, S, T, M, V, P, or C; W543 replaced with D, E, H, K, R, N, Q, A, G, I, L, S, T, M, V, P, or C; P544 replaced with D, E, H, K, R, A, G, I, L, S, T, M, V, N, Q, F, W, Y, or C; R545 replaced with D, E, A, G, I, L, S, T, M, V, N, Q, F, W, Y, P, or C; A546 replaced with D, E, H, K, R, N, Q, F, W, Y, P, or C; S547 replaced with D, E, H, K, R, N, Q, F, W, Y, P, or C; A548 replaced with D, E, H, K, R, N, Q, F, W, Y, P, or C; L549 replaced with D, E, H, K, R, N, Q, F, W, Y, P, or C; A550 replaced with D, E, H, K, R, N, Q, F, W, Y, P, or C; E551 replaced with H, K, R, A, G, I, L, S, T, M, V, N, Q, F, W, Y, P, or C; R552 replaced with D, E, A, G, I, L, S, T, M, V, N, Q, F, W, Y, P, or C; L553 replaced with D, E, H, K, R, N, Q, F, W, Y, P, or C; W554 replaced with D, E, H, K, R, N, Q, A, G, I, L, S, T, M, V, P, or C; S555 replaced with D, E, H, K, R, N, Q, F, W, Y, P, or C; N556 replaced with D, E, H, K, R, A, G, I, L, S, T, M, V, F, W, Y, P, or C; P557 replaced with D, E, H, K, R, A, G, I, L, S, T, M, V, N, Q, F, W, Y, or C; A558 replaced with D, E, H, K, R, N, Q, F, W, Y, P, or C; E559 replaced with H, K, R, A, G, I, L, S, T, M, V, N, Q, F, W, Y, P, or C; G560 replaced with D, E, H, K, R, N, Q, F, W, Y, P, or C; W561 replaced with D, E, H, K, R, N, Q, A, G, I, L, S, T, M, V, P, or C; R562 replaced with D, E, A, G, I, L, S, T, M, V, N, Q, F, W, Y, P, or C; Q563 replaced with D, E, H, K, R, A, G, I, L, S, T, M, V, F, W, Y, P, or C; A564 replaced with D, E, H, K, R, N, Q, F, W, Y, P, or C; E565 replaced with H, K, R, A, G, I, L, S, T, M, V, N, Q, F, W, Y, P, or C; S566 replaced with D, E, H, K, R, N, Q, F, W, Y, P, or C; R567 replaced with D, E, A, G, I, L, S, T, M, V, N, Q, F, W, Y, P, or C; L568 replaced with D, E, H, K, R, N, Q, F, W, Y, P, or C; L569 replaced with D, E, H, K, R, N, Q, F, W, Y, P, or C; L570 replaced with D, E, H, K, R, N, Q, F, W, Y, P, or C; H571 replaced with D, E, A, G, I, L, S, T, M, V, N, Q, F, W, Y, P, or C; R572 replaced with D, E, A, G, I, L, S, T, M, V, N, Q, F, W, Y, P, or C; Q573 replaced with D, E, H, K, R, A, G, I, L, S, T, M, V, F, W, Y, P, or C; R574 replaced with D, E, A, G, I, L, S, T, M, V, N, Q, F, W, Y, P, or C; L575 replaced with D, E, H, K, R, N, Q, F, W, Y, P, or C; V576 replaced with D, E, H, K, R, N, Q, F, W, Y, P, or C; D577 replaced with H, K, R, A, G, I, L, S, T, M, V, N, Q, F, W, Y, P, or C; N578 replaced with D, E, H, K, R, A, G, I, L, S, T, M, V, F, W, Y, P, or C; G579 replaced with D, E, H, K, R, N, Q, F, W, Y, P, or C; L580 replaced with D, E, H, K, R, N, Q, F, W, Y, P, or C; G581 replaced with D, E, H, K, R, N, Q, F, W, Y, P, or C; A582 replaced with D, E, H, K, R, N, Q, F, W, Y, P, or C; E583 replaced with H, K, R, A, G, I, L, S, T, M, V, N, Q, F, W, Y, P, or C; A584 replaced with D, E, H, K, R, N, Q, F, W, Y, P, or C; M585 replaced with D, E, H, K, R, N, Q, F, W, Y, P, or C; Q586 replaced with D, E, H, K, R, A, G, I, L, S, T, M, V, F, W, Y, P, or C; P587 replaced with D, E, H, K, R, A, G, I, L, S, T, M, V, N, Q, F, W, Y, or C; Q588 replaced with D, E, H, K, R, A, G, I, L, S, T, M, V, F, W, Y, P, or C; W589 replaced with D, E, H, K, R, N, Q, A, G, I, L, S, T, M, V, P, or C; C590 replaced with D, E, H, K, R, A, G, I, L, S, T, M, V, N, Q, F, W, Y, or P; L591 replaced with D, E, H, K, R, N, Q, F, W, Y, P, or C; Q592 replaced with D, E, H, K, R, A, G, I, L, S, T, M, V, F, W, Y, P, or C; N593 replaced with D, E, H, K, R, A, G, I, L, S, T, M, V, F, W, Y, P, or C; E594 replaced with H, K, R, A, G, I, L, S, T, M, V, N, Q, F, W, Y, P, or C; H595 replaced with D, E, A, G, I, L, S, T, M, V, N, Q, F, W, Y, P, or C; E596 replaced with H, K, R, A, G, I, L, S, T, M, V, N, Q, F, W, Y, P, or C; C597 replaced with D, E, H, K, R, A, G, I, L, S, T, M, V, N, Q, F, W, Y, or P; P598 replaced with D, E, H, K, R, A, G, I, L, S, T, M, V, N, Q, F, W, Y, or C; I599 replaced with D, E, H, K, R, N, Q, F, W, Y, P, or C; D600 replaced with H, K, R, A, G, I, L, S, T, M, V, N, Q, F, W, Y, P, or C; A601 replaced with D, E, H, K, R, N, Q, F, W, Y, P, or C; Y602 replaced with D, E, H, K, R, N, Q, A, G, I, L, S, T, M, V, P, or C; D603 replaced with H, K, R, A, G, I, L, S, T, M, V, N, Q, F, W, Y, P, or C; A604 replaced with D, E, H, K, R, N, Q, F, W, Y, P, or C; Q605 replaced with D, E, H, K, R, A, G, I, L, S, T, M, V, F, W, Y, P, or C; and/or V606 replaced with D, E, H, K, R, N, Q, F, W, Y, P, or C.

The resulting constructs can be routinely screened for activities or functions described throughout the specification and known in the art. Preferably, the resulting constructs have loss of a hexosaminidase activity or function, while the remaining biological activities or functions are maintained. More preferably, the resulting constructs have more than one loss of biological activity or function, while the remaining biological activities or functions are maintained.

Additionally, more than one amino acid (e.g., 2, 3, 4, 5, 6, 7, 8, 9 and 10) can be replaced with the substituted amino acids as described above (either conservative or nonconservative). The substituted amino acids can occur in the full length, mature, or proprotein form of Hex-1 (SEQ ID NO:4) protein, as well as the N- and C-terminal deletion mutants, having the general formula m-n, listed below.

A further embodiment of the invention relates to a polypeptide which comprises the amino acid sequence of Hex-1 (SEQ ID NO:4) polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of Hex-1 polypeptide (SEQ ID NO:4), which contains at least one, but not more than 10, 9, 8, 7, 6, 5, 4, 3, 2 or 1 amino acid substitutions. In specific embodiments, the number of additions, substitutions, and/or deletions in the amino acid sequence of FIG. 2 or fragments thereof (e.g., the mature form and/or other fragments described herein), is 1-5, 5-10, 5-25, 5-50, 10-50 or 50-150, conservative amino acid substitutions are preferable.

In another example, preferred non-conservative substitutions of Hex-2 (SEQ ID NO:6) include: M1 replaced with D, E, H, K, R, N, Q, F, W, Y, P, or C; R2 replaced with D, E, A, G, I, L, S, T, M, V, N, Q, F, W, Y, P, or C; F3 replaced with D, E, H, K, R, N, Q, A, G, I, L, S, T, M, V, P, or C; S4 replaced with D, E, H, K, R, N, Q, F, W, Y, P, or C; G5 replaced with D, E, H, K, R, N, Q, F, W, Y, P, or C; Y6 replaced with D, E, H, K, R, N, Q, A, G, I, L, S, T, M, V, P, or C; N7 replaced with D, E, H, K, R, A, G, I, L, S, T, M, V, F, W, Y, P, or C; R8 replaced with D, E, A, G, I, L, S, T, M, V, N, Q, F, W, Y, P, or C; Y9 replaced with D, E, H, K, R, N, Q, A, G, I, L, S, T, M, V, P, or C; Q10 replaced with D, E, H, K, R, A, G, I, L, S, T, M, V, F, W, Y, P, or C; C11 replaced with D, E, H, K, R, A, G, I, L, S, T, M, V, N, Q, F, W, Y, or P; F12 replaced with D, E, H, K, R, N, Q, A, G, I, L, S, T, M, V, P, or C; C13 replaced with D, E, H, K, R, A, G, I, L, S, T, M, V, N, Q, F, W, Y, or P; S14 replaced with D, E, H, K, R, N, Q, F, W, Y, P, or C; A15 replaced with D, E, H, K, R, N, Q, F, W, Y, P, or C; V16 replaced with D, E, H, K, R, N, Q, F, W, Y, P, or C; G17 replaced with D, E, H, K, R, N, Q, F, W, Y, P, or C; S18 replaced with D, E, H, K, R, N, Q, F, W, Y, P, or C; L19 replaced with D, E, H, K, R, N, Q, F, W, Y, P, or C; L20 replaced with D, E, H, K, R, N, Q, F, W, Y, P, or C; L21 replaced with D, E, H, K, R, N, Q, F, W, Y, P, or C; L22 replaced with D, E, H, K, R, N, Q, F, W, Y, P, or C; S23 replaced with D, E, H, K, R, N, Q, F, W, Y, P, or C; L24 replaced with D, E, H, K, R, N, Q, F, W, Y, P, or C; L25 replaced with D, E, H, K, R, N, Q, F, W, Y, P, or C; S26 replaced with D, E, H, K, R, N, Q, F, W, Y, P, or C; F27 replaced with D, E, H, K, R, N, Q, A, G, I, L, S, T, M, V, P, or C; A28 replaced with D, E, H, K, R, N, Q, F, W, Y, P, or C; V29 replaced with D, E, H, K, R, N, Q, F, W, Y, P, or C; G30 replaced with D, E, H, K, R, N, Q, F, W, Y, P, or C; A31 replaced with D, E, H, K, R, N, Q, F, W, Y, P, or C; A32 replaced with D, E, H, K, R, N, Q, F, W, Y, P, or C; L33 replaced with D, E, H, K, R, N, Q, F, W, Y, P, or C; T34 replaced with D, E, H, K, R, N, Q, F, W, Y, P, or C; R35 replaced with D, E, A, G, I, L, S, T, M, V, N, Q, F, W, Y, P, or C; A36 replaced with D, E, H, K, R, N, Q, F, W, Y, P, or C; D37 replaced with H, K, R, A, G, I, L, S, T, M, V, N, Q, F, W, Y, P, or C; D38 replaced with H, K, R, A, G, I, L, S, T, M, V, N, Q, F, W, Y, P, or C; V39 replaced with D, E, H, K, R, N, Q, F, W, Y, P, or C; G40 replaced with D, E, H, K, R, N, Q, F, W, Y, P, or C; S41 replaced with D, E, H, K, R, N, Q, F, W, Y, P, or C; D42 replaced with H, K, R, A, G, I, L, S, T, M, V, N, Q, F, W, Y, P, or C; A43 replaced with D, E, H, K, R, N, Q, F, W, Y, P, or C; D44 replaced with H, K, R, A, G, I, L, S, T, M, V, N, Q, F, W, Y, P, or C; A45 replaced with D, E, H, K, R, N, Q, F, W, Y, P, or C; G46 replaced with D, E, H, K, R, N, Q, F, W, Y, P, or C; S47 replaced with D, E, H, K, R, N, Q, F, W, Y, P, or C; A48 replaced with D, E, H, K, R, N, Q, F, W, Y, P, or C; S49 replaced with D, E, H, K, R, N, Q, F, W, Y, P, or C; R50 replaced with D, E, A, G, I, L, S, T, M, V, N, Q, F, W, Y, P, or C; K51 replaced with D, E, A, G, I, L, S, T, M, V, N, Q, F, W, Y, P, or C; W52 replaced with D, E, H, K, R, N, Q, A, G, I, L, S, T, M, V, P, or C; L53 replaced with D, E, H, K, R, N, Q, F, W, Y, P, or C; C54 replaced with D, E, H, K, R, A, G, I, L, S, T, M, V, N, Q, F, W, Y, or P; S55 replaced with D, E, H, K, R, N, Q, F, W, Y, P, or C; R56 replaced with D, E, A, G, I, L, S, T, M, V, N, Q, F, W, Y, P, or C; T57 replaced with D, E, H, K, R, N, Q, F, W, Y, P, or C; D58 replaced with H, K, R, A, G, I, L, S, T, M, V, N, Q, F, W, Y, P, or C; I59 replaced with D, E, H, K, R, N, Q, F, W, Y, P, or C; C60 replaced with D, E, H, K, R, A, G, I, L, S, T, M, V, N, Q, F, W, Y, or P; T61 replaced with D, E, H, K, R, N, Q, F, W, Y, P, or C; A62 replaced with D, E, H, K, R, N, Q, F, W, Y, P, or C; E63 replaced with H, K, R, A, G, I, L, S, T, M, V, N, Q, F, W, Y, P, or C; G64 replaced with D, E, H, K, R, N, Q, F, W, Y, P, or C; E65 replaced with H, K, R, A, G, I, L, S, T, M, V, N, Q, F, W, Y, P, or C; M66 replaced with D, E, H, K, R, N, Q, F, W, Y, P, or C; V67 replaced with D, E, H, K, R, N, Q, F, W, Y, P, or C; A68 replaced with D, E, H, K, R, N, Q, F, W, Y, P, or C; G69 replaced with D, E, H, K, R, N, Q, F, W, Y, P, or C; L70 replaced with D, E, H, K, R, N, Q, F, W, Y, P, or C; Q71 replaced with D, E, H, K, R, A, G, I, L, S, T, M, V, F, W, Y, P, or C; Y72 replaced with D, E, H, K, R, N, Q, A, G, I, L, S, T, M, V, P, or C; A73 replaced with D, E, H, K, R, N, Q, F, W, Y, P, or C; P74 replaced with D, E, H, K, R, A, G, I, L, S, T, M, V, N, Q, F, W, Y, or C; E75 replaced with H, K, R, A, G, I, L, S, T, M, V, N, Q, F, W, Y, P, or C; I76 replaced with D, E, H, K, R, N, Q, F, W, Y, P, or C; F77 replaced with D, E, H, K, R, N, Q, A, G, I, L, S, T, M, V, P, or C; E78 replaced with H, K, R, A, G, I, L, S, T, M, V, N, Q, F, W, Y, P, or C; S79 replaced with D, E, H, K, R, N, Q, F, W, Y, P, or C; Q80 replaced with D, E, H, K, R, A, G, I, L, S, T, M, V, F, W, Y, P, or C; R81 replaced with D, E, A, G, I, L, S, T, M, V, N, Q, F, W, Y, P, or C; D82 replaced with H, K, R, A, G, I, L, S, T, M, V, N, Q, F, W, Y, P, or C; C83 replaced with D, E, H, K, R, A, G, I, L, S, T, M, V, N, Q, F, W, Y, or P; R84 replaced with D, E, A, G, I, L, S, T, M, V, N, Q, F, W, Y, P, or C; L85 replaced with D, E, H, K, R, N, Q, F, W, Y, P, or C; S86 replaced with D, E, H, K, R, N, Q, F, W, Y, P, or C; C87 replaced with D, E, H, K, R, A, G, I, L, S, T, M, V, N, Q, F, W, Y, or P; G88 replaced with D, E, H, K, R, N, Q, F, W, Y, P, or C; K89 replaced with D, E, A, G, I, L, S, T, M, V, N, Q, F, W, Y, P, or C; Y90 replaced with D, E, H, K, R, N, Q, A, G, I, L, S, T, M, V, P, or C; G91 replaced with D, E, H, K, R, N, Q, F, W, Y, P, or C; A92 replaced with D, E, H, K, R, N, Q, F, W, Y, P, or C; I93 replaced with D, E, H, K, R, N, Q, F, W, Y, P, or C; W94 replaced with D, E, H, K, R, N, Q, A, G, I, L, S, T, M, V, P, or C; P95 replaced with D, E, H, K, R, A, G, I, L, S, T, M, V, N, Q, F, W, Y, or C; M96 replaced with D, E, H, K, R, N, Q, F, W, Y, P, or C; P97 replaced with D, E, H, K, R, A, G, I, L, S, T, M, V, N, Q, F, W, Y, or C; T98 replaced with D, E, H, K, R, N, Q, F, W, Y, P, or C; G99 replaced with D, E, H, K, R, N, Q, F, W, Y, P, or C; K100 replaced with D, E, A, G, I, L, S, T, M, V, N, Q, F, W, Y, P, or C; E101 replaced with H, K, R, A, G, I, L, S, T, M, V, N, Q, F, W, Y, P, or C; C102 replaced with D, E, H, K, R, A, G, I, L, S, T, M, V, N, Q, F, W, Y, or P; T103 replaced with D, E, H, K, R, N, Q, F, W, Y, P, or C; I104 replaced with D, E, H, K, R, N, Q, F, W, Y, P, or C; S 105 replaced with D, E, H, K, R, N, Q, F, W, Y, P, or C; H106 replaced with D, E, A, G, I, L, S, T, M, V, N, Q, F, W, Y, P, or C; R107 replaced with D, E, A, G, I, L, S, T, M, V, N, Q, F, W, Y, P, or C; R108 replaced with D, E, A, G, I, L, S, T, M, V, N, Q, F, W, Y, P, or C; V109 replaced with D, E, H, K, R, N, Q, F, W, Y, P, or C; R110 replaced with D, E, A, G, I, L, S, T, M, V, N, Q, F, W, Y, P, or C; F111 replaced with D, E, H, K, R, N, Q, A, G, I, L, S, T, M, V, P, or C; D112 replaced with H, K, R, A, G, I, L, S, T, M, V, N, Q, F, W, Y, P, or C; P113 replaced with D, E, H, K, R, A, G, I, L, S, T, M, V, N, Q, F, W, Y, or C; W114 replaced with D, E, H, K, R, N, Q, A, G, I, L, S, T, M, V, P, or C; K115 replaced with D, E, A, G, I, L, S, T, M, V, N, Q, F, W, Y, P, or C; V116 replaced with D, E, H, K, R, N, Q, F, W, Y, P, or C; R117 replaced with D, E, A, G, I, L, S, T, M, V, N, Q, F, W, Y, P, or C; F118 replaced with D, E, H, K, R, N, Q, A, G, I, L, S, T, M, V, P, or C; H119 replaced with D, E, A, G, I, L, S, T, M, V, N, Q, F, W, Y, P, or C; V120 replaced with D, E, H, K, R, N, Q, F, W, Y, P, or C; V121 replaced with D, E, H, K, R, N, Q, F, W, Y, P, or C; A122 replaced with D, E, H, K, R, N, Q, F, W, Y, P, or C; P123 replaced with D, E, H, K, R, A, G, I, L, S, T, M, V, N, Q, F, W, Y, or C; G124 replaced with D, E, H, K, R, N, Q, F, W, Y, P, or C; E125 replaced with H, K, R, A, G, I, L, S, T, M, V, N, Q, F, W, Y, P, or C; A126 replaced with D, E, H, K, R, N, Q, F, W, Y, P, or C; A127 replaced with D, E, H, K, R, N, Q, F, W, Y, P, or C; T128 replaced with D, E, H, K, R, N, Q, F, W, Y, P, or C; Q129 replaced with D, E, H, K, R, A, G, I, L, S, T, M, V, F, W, Y, P, or C; F130 replaced with D, E, H, K, R, N, Q, A, G, I, L, S, T, M, V, P, or C; L131 replaced with D, E, H, K, R, N, Q, F, W, Y, P, or C; R132 replaced with D, E, A, G, I, L, S, T, M, V, N, Q, F, W, Y, P, or C; E133 replaced with H, K, R, A, G, I, L, S, T, M, V, N, Q, F, W, Y, P, or C; T134 replaced with D, E, H, K, R, N, Q, F, W, Y, P, or C; N135 replaced with D, E, H, K, R, A, G, I, L, S, T, M, V, F, W, Y, P, or C; R136 replaced with D, E, A, G, I, L, S, T, M, V, N, Q, F, W, Y, P, or C; L137 replaced with D, E, H, K, R, N, Q, F, W, Y, P, or C; F138 replaced with D, E, H, K, R, N, Q, A, G, I, L, S, T, M, V, P, or C; V139 replaced with D, E, H, K, R, N, Q, F, W, Y, P, or C; S140 replaced with D, E, H, K, R, N, Q, F, W, Y, P, or C; N141 replaced with D, E, H, K, R, A, G, I, L, S, T, M, V, F, W, Y, P, or C; L142 replaced with D, E, H, K, R, N, Q, F, W, Y, P, or C; L143 replaced with D, E, H, K, R, N, Q, F, W, Y, P, or C; K144 replaced with D, E, A, G, I, L, S, T, M, V, N, Q, F, W, Y, P, or C; E145 replaced with H, K, R, A, G, I, L, S, T, M, V, N, Q, F, W, Y, P, or C; C146 replaced with D, E, H, K, R, A, G, I, L, S, T, M, V, N, Q, F, W, Y, or P; I147 replaced with D, E, H, K, R, N, Q, F, W, Y, P, or C; R148 replaced with D, E, A, G, I, L, S, T, M, V, N, Q, F, W, Y, P, or C; N149 replaced with D, E, H, K, R, A, G, I, L, S, T, M, V, F, W, Y, P, or C; C150 replaced with D, E, H, K, R, A, G, I, L, S, T, M, V, N, Q, F, W, Y, or P; T151 replaced with D, E, H, K, R, N, Q, F, W, Y, P, or C; L152 replaced with D, E, H, K, R, N, Q, F, W, Y, P, or C; E153 replaced with H, K, R, A, G, I, L, S, T, M, V, N, Q, F, W, Y, P, or C; T154 replaced with D, E, H, K, R, N, Q, F, W, Y, P, or C; S155 replaced with D, E, H, K, R, N, Q, F, W, Y, P, or C; K156 replaced with D, E, A, G, I, L, S, T, M, V, N, Q, F, W, Y, P, or C; Q157 replaced with D, E, H, K, R, A, G, I, L, S, T, M, V, F, W, Y, P, or C; I158 replaced with D, E, H, K, R, N, Q, F, W, Y, P, or C; L159 replaced with D, E, H, K, R, N, Q, F, W, Y, P, or C; V160 replaced with D, E, H, K, R, N, Q, F, W, Y, P, or C; R161 replaced with D, E, A, G, I, L, S, T, M, V, N, Q, F, W, Y, P, or C; S162 replaced with D, E, H, K, R, N, Q, F, W, Y, P, or C; T163 replaced with D, E, H, K, R, N, Q, F, W, Y, P, or C; V164 replaced with D, E, H, K, R, N, Q, F, W, Y, P, or C; A165 replaced with D, E, H, K, R, N, Q, F, W, Y, P, or C; N166 replaced with D, E, H, K, R, A, G, I, L, S, T, M, V, F, W, Y, P, or C; E167 replaced with H, K, R, A, G, I, L, S, T, M, V, N, Q, F, W, Y, P, or C; S168 replaced with D, E, H, K, R, N, Q, F, W, Y, P, or C; L169 replaced with D, E, H, K, R, N, Q, F, W, Y, P, or C; V170 replaced with D, E, H, K, R, N, Q, F, W, Y, P, or C; L171 replaced with D, E, H, K, R, N, Q, F, W, Y, P, or C; D172 replaced with H, K, R, A, G, I, L, S, T, M, V, N, Q, F, W, Y, P, or C; W173 replaced with D, E, H, K, R, N, Q, A, G, I, L, S, T, M, V, P, or C; P174 replaced with D, E, H, K, R, A, G, I, L, S, T, M, V, N, Q, F, W, Y, or C; T175 replaced with D, E, H, K, R, N, Q, F, W, Y, P, or C; D176 replaced with H, K, R, A, G, I, L, S, T, M, V, N, Q, F, W, Y, P, or C; E177 replaced with H, K, R, A, G, I, L, S, T, M, V, N, Q, F, W, Y, P, or C; S178 replaced with D, E, H, K, R, N, Q, F, W, Y, P, or C; Y179 replaced with D, E, H, K, R, N, Q, A, G, I, L, S, T, M, V, P, or C; A180 replaced with D, E, H, K, R, N, Q, F, W, Y, P, or C; L181 replaced with D, E, H, K, R, N, Q, F, W, Y, P, or C; V182 replaced with D, E, H, K, R, N, Q, F, W, Y, P, or C; V183 replaced with D, E, H, K, R, N, Q, F, W, Y, P, or C; R184 replaced with D, E, A, G, I, L, S, T, M, V, N, Q, F, W, Y, P, or C; T185 replaced with D, E, H, K, R, N, Q, F, W, Y, P, or C; T186 replaced with D, E, H, K, R, N, Q, F, W, Y, P, or C; E187 replaced with H, K, R, A, G, I, L, S, T, M, V, N, Q, F, W, Y, P, or C; T188 replaced with D, E, H, K, R, N, Q, F, W, Y, P, or C; A189 replaced with D, E, H, K, R, N, Q, F, W, Y, P, or C; T190 replaced with D, E, H, K, R, N, Q, F, W, Y, P, or C; F191 replaced with D, E, H, K, R, N, Q, A, G, I, L, S, T, M, V, P, or C; V192 replaced with D, E, H, K, R, N, Q, F, W, Y, P, or C; D193 replaced with H, K, R, A, G, I, L, S, T, M, V, N, Q, F, W, Y, P, or C; I194 replaced with D, E, H, K, R, N, Q, F, W, Y, P, or C; Q195 replaced with D, E, H, K, R, A, G, I, L, S, T, M, V, F, W, Y, P, or C; A196 replaced with D, E, H, K, R, N, Q, F, W, Y, P, or C; T197 replaced with D, E, H, K, R, N, Q, F, W, Y, P, or C; T198 replaced with D, E, H, K, R, N, Q, F, W, Y, P, or C; V199 replaced with D, E, H, K, R, N, Q, F, W, Y, P, or C; Y200 replaced with D, E, H, K, R, N, Q, A, G, I, L, S, T, M, V, P, or C; G201 replaced with D, E, H, K, R, N, Q, F, W, Y, P, or C; A202 replaced with D, E, H, K, R, N, Q, F, W, Y, P, or C; R203 replaced with D, E, A, G, I, L, S, T, M, V, N, Q, F, W, Y, P, or C; H204 replaced with D, E, A, G, I, L, S, T, M, V, N, Q, F, W, Y, P, or C; A205 replaced with D, E, H, K, R, N, Q, F, W, Y, P, or C; F206 replaced with D, E, H, K, R, N, Q, A, G, I, L, S, T, M, V, P, or C; E207 replaced with H, K, R, A, G, I, L, S, T, M, V, N, Q, F, W, Y, P, or C; T208 replaced with D, E, H, K, R, N, Q, F, W, Y, P, or C; L209 replaced with D, E, H, K, R, N, Q, F, W, Y, P, or C; S210 replaced with D, E, H, K, R, N, Q, F, W, Y, P, or C; N211 replaced with D, E, H, K, R, A, G, I, L, S, T, M, V, F, W, Y, P, or C; L212 replaced with D, E, H, K, R, N, Q, F, W, Y, P, or C; V213 replaced with D, E, H, K, R, N, Q, F, W, Y, P, or C; T214 replaced with D, E, H, K, R, N, Q, F, W, Y, P, or C; G215 replaced with D, E, H, K, R, N, Q, F, W, Y, P, or C; S216 replaced with D, E, H, K, R, N, Q, F, W, Y, P, or C; L217 replaced with D, E, H, K, R, N, Q, F, W, Y, P, or C; S218 replaced with D, E, H, K, R, N, Q, F, W, Y, P, or C; N219 replaced with D, E, H, K, R, A, G, I, L, S, T, M, V, F, W, Y, P, or C; G220 replaced with D, E, H, K, R, N, Q, F, W, Y, P, or C; L221 replaced with D, E, H, K, R, N, Q, F, W, Y, P, or C; L222 replaced with D, E, H, K, R, N, Q, F, W, Y, P, or C; M223 replaced with D, E, H, K, R, N, Q, F, W, Y, P, or C; V224 replaced with D, E, H, K, R, N, Q, F, W, Y, P, or C; T225 replaced with D, E, H, K, R, N, Q, F, W, Y, P, or C; T226 replaced with D, E, H, K, R, N, Q, F, W, Y, P, or C; A227 replaced with D, E, H, K, R, N, Q, F, W, Y, P, or C; N228 replaced with D, E, H, K, R, A, G, I, L, S, T, M, V, F, W, Y, P, or C; I229 replaced with D, E, H, K, R, N, Q, F, W, Y, P, or C; T230 replaced with D, E, H, K, R, N, Q, F, W, Y, P, or C; D231 replaced with H, K, R, A, G, I, L, S, T, M, V, N, Q, F, W, Y, P, or C; R232 replaced with D, E, A, G, I, L, S, T, M, V, N, Q, F, W, Y, P, or C; P233 replaced with D, E, H, K, R, A, G, I, L, S, T, M, V, N, Q, F, W, Y, or C; A234 replaced with D, E, H, K, R, N, Q, F, W, Y, P, or C; F235 replaced with D, E, H, K, R, N, Q, A, G, I, L, S, T, M, V, P, or C; S236 replaced with D, E, H, K, R, N, Q, F, W, Y, P, or C; H237 replaced with D, E, A, G, I, L, S, T, M, V, N, Q, F, W, Y, P, or C; R238 replaced with D, E, A, G, I, L, S, T, M, V, N, Q, F, W, Y, P, or C; G239 replaced with D, E, H, K, R, N, Q, F, W, Y, P, or C; V240 replaced with D, E, H, K, R, N, Q, F, W, Y, P, or C; L241 replaced with D, E, H, K, R, N, Q, F, W, Y, P, or C; L242 replaced with D, E, H, K, R, N, Q, F, W, Y, P, or C; D243 replaced with H, K, R, A, G, I, L, S, T, M, V, N, Q, F, W, Y, P, or C; T244 replaced with D, E, H, K, R, N, Q, F, W, Y, P, or C; A245 replaced with D, E, H, K, R, N, Q, F, W, Y, P, or C; R246 replaced with D, E, A, G, I, L, S, T, M, V, N, Q, F, W, Y, P, or C; N247 replaced with D, E, H, K, R, A, G, I, L, S, T, M, V, F, W, Y, P, or C; F248 replaced with D, E, H, K, R, N, Q, A, G, I, L, S, T, M, V, P, or C; V249 replaced with D, E, H, K, R, N, Q, F, W, Y, P, or C; P250 replaced with D, E, H, K, R, A, G, I, L, S, T, M, V, N, Q, F, W, Y, or C; L251 replaced with D, E, H, K, R, N, Q, F, W, Y, P, or C; K252 replaced with D, E, A, G, I, L, S, T, M, V, N, Q, F, W, Y, P, or C; F253 replaced with D, E, H, K, R, N, Q, A, G, I, L, S, T, M, V, P, or C; I254 replaced with D, E, H, K, R, N, Q, F, W, Y, P, or C; R255 replaced with D, E, A, G, I, L, S, T, M, V, N, Q, F, W, Y, P, or C; S256 replaced with D, E, H, K, R, N, Q, F, W, Y, P, or C; T257 replaced with D, E, H, K, R, N, Q, F, W, Y, P, or C; L258 replaced with D, E, H, K, R, N, Q, F, W, Y, P, or C; D259 replaced with H, K, R, A, G, I, L, S, T, M, V, N, Q, F, W, Y, P, or C; A260 replaced with D, E, H, K, R, N, Q, F, W, Y, P, or C; M261 replaced with D, E, H, K, R, N, Q, F, W, Y, P, or C; A262 replaced with D, E, H, K, R, N, Q, F, W, Y, P, or C; A263 replaced with D, E, H, K, R, N, Q, F, W, Y, P, or C; S264 replaced with D, E, H, K, R, N, Q, F, W, Y, P, or C; K265 replaced with D, E, A, G, I, L, S, T, M, V, N, Q, F, W, Y, P, or C; L266 replaced with D, E, H, K, R, N, Q, F, W, Y, P, or C; N267 replaced with D, E, H, K, R, A, G, I, L, S, T, M, V, F, W, Y, P, or C; V268 replaced with D, E, H, K, R, N, Q, F, W, Y, P, or C; L269 replaced with D, E, H, K, R, N, Q, F, W, Y, P, or C; H270 replaced with D, E, A, G, I, L, S, T, M, V, N, Q, F, W, Y, P, or C; W271 replaced with D, E, H, K, R, N, Q, A, G, I, L, S, T, M, V, P, or C; H272 replaced with D, E, A, G, I, L, S, T, M, V, N, Q, F, W, Y, P, or C; V273 replaced with D, E, H, K, R, N, Q, F, W, Y, P, or C; V274 replaced with D, E, H, K, R, N, Q, F, W, Y, P, or C; D275 replaced with H, K, R, A, G, I, L, S, T, M, V, N, Q, F, W, Y, P, or C; T276 replaced with D, E, H, K, R, N, Q, F, W, Y, P, or C; H277 replaced with D, E, A, G, I, L, S, T, M, V, N, Q, F, W, Y, P, or C; S278 replaced with D, E, H, K, R, N, Q, F, W, Y, P, or C; F279 replaced with D, E, H, K, R, N, Q, A, G, I, L, S, T, M, V, P, or C; P280 replaced with D, E, H, K, R, A, G, I, L, S, T, M, V, N, Q, F, W, Y, or C; L281 replaced with D, E, H, K, R, N, Q, F, W, Y, P, or C; E282 replaced with H, K, R, A, G, I, L, S, T, M, V, N, Q, F, W, Y, P, or C; I283 replaced with D, E, H, K, R, N, Q, F, W, Y, P, or C; T284 replaced with D, E, H, K, R, N, Q, F, W, Y, P, or C; R285 replaced with D, E, A, G, I, L, S, T, M, V, N, Q, F, W, Y, P, or C; V286 replaced with D, E, H, K, R, N, Q, F, W, Y, P, or C; P287 replaced with D, E, H, K, R, A, G, I, L, S, T, M, V, N, Q, F, W, Y, or C; E288 replaced with H, K, R, A, G, I, L, S, T, M, V, N, Q, F, W, Y, P, or C; M289 replaced with D, E, H, K, R, N, Q, F, W, Y, P, or C; Q290 replaced with D, E, H, K, R, A, G, I, L, S, T, M, V, F, W, Y, P, or C; R291 replaced with D, E, A, G, I, L, S, T, M, V, N, Q, F, W, Y, P, or C; Y292 replaced with D, E, H, K, R, N, Q, A, G, I, L, S, T, M, V, P, or C; G293 replaced with D, E, H, K, R, N, Q, F, W, Y, P, or C; A294 replaced with D, E, H, K, R, N, Q, F, W, Y, P, or C; Y295 replaced with D, E, H, K, R, N, Q, A, G, I, L, S, T, M, V, P, or C; S296 replaced with D, E, H, K, R, N, Q, F, W, Y, P, or C; S297 replaced with D, E, H, K, R, N, Q, F, W, Y, P, or C; S298 replaced with D, E, H, K, R, N, Q, F, W, Y, P, or C; Q299 replaced with D, E, H, K, R, A, G, I, L, S, T, M, V, F, W, Y, P, or C; T300 replaced with D, E, H, K, R, N, Q, F, W, Y, P, or C; Y301 replaced with D, E, H, K, R, N, Q, A, G, I, L, S, T, M, V, P, or C; S302 replaced with D, E, H, K, R, N, Q, F, W, Y, P, or C; R303 replaced with D, E, A, G, I, L, S, T, M, V, N, Q, F, W, Y, P, or C; Q304 replaced with D, E, H, K, R, A, G, I, L, S, T, M, V, F, W, Y, P, or C; D305 replaced with H, K, R, A, G, I, L, S, T, M, V, N, Q, F, W, Y, P, or C; A306 replaced with D, E, H, K, R, N, Q, F, W, Y, P, or C; L307 replaced with D, E, H, K, R, N, Q, F, W, Y, P, or C; N308 replaced with D, E, H, K, R, A, G, I, L, S, T, M, V, F, W, Y, P, or C; L309 replaced with D, E, H, K, R, N, Q, F, W, Y, P, or C; V310 replaced with D, E, H, K, R, N, Q, F, W, Y, P, or C; K311 replaced with D, E, A, G, I, L, S, T, M, V, N, Q, F, W, Y, P, or C; Y312 replaced with D, E, H, K, R, N, Q, A, G, I, L, S, T, M, V, P, or C; A313 replaced with D, E, H, K, R, N, Q, F, W, Y, P, or C; R314 replaced with D, E, A, G, I, L, S, T, M, V, N, Q, F, W, Y, P, or C; L315 replaced with D, E, H, K, R, N, Q, F, W, Y, P, or C; R316 replaced with D, E, A, G, I, L, S, T, M, V, N, Q, F, W, Y, P, or C; G317 replaced with D, E, H, K, R, N, Q, F, W, Y, P, or C; I318 replaced with D, E, H, K, R, N, Q, F, W, Y, P, or C; R319 replaced with D, E, A, G, I, L, S, T, M, V, N, Q, F, W, Y, P, or C; I320 replaced with D, E, H, K, R, N, Q, F, W, Y, P, or C; L321 replaced with D, E, H, K, R, N, Q, F, W, Y, P, or C; I322 replaced with D, E, H, K, R, N, Q, F, W, Y, P, or C; E323 replaced with H, K, R, A, G, I, L, S, T, M, V, N, Q, F, W, Y, P, or C; I324 replaced with D, E, H, K, R, N, Q, F, W, Y, P, or C; D325 replaced with H, K, R, A, G, I, L, S, T, M, V, N, Q, F, W, Y, P, or C; G326 replaced with D, E, H, K, R, N, Q, F, W, Y, P, or C; P327 replaced with D, E, H, K, R, A, G, I, L, S, T, M, V, N, Q, F, W, Y, or C; S328 replaced with D, E, H, K, R, N, Q, F, W, Y, P, or C; H329 replaced with D, E, A, G, I, L, S, T, M, V, N, Q, F, W, Y, P, or C; A330 replaced with D, E, H, K, R, N, Q, F, W, Y, P, or C; G331 replaced with D, E, H, K, R, N, Q, F, W, Y, P, or C; N332 replaced with D, E, H, K, R, A, G, I, L, S, T, M, V, F, W, Y, P, or C; G333 replaced with D, E, H, K, R, N, Q, F, W, Y, P, or C; W334 replaced with D, E, H, K, R, N, Q, A, G, I, L, S, T, M, V, P, or C; Q335 replaced with D, E, H, K, R, A, G, I, L, S, T, M, V, F, W, Y, P, or C; W336 replaced with D, E, H, K, R, N, Q, A, G, I, L, S, T, M, V, P, or C; G337 replaced with D, E, H, K, R, N, Q, F, W, Y, P, or C; P338 replaced with D, E, H, K, R, A, G, I, L, S, T, M, V, N, Q, F, W, Y, or C; A339 replaced with D, E, H, K, R, N, Q, F, W, Y, P, or C; A340 replaced with D, E, H, K, R, N, Q, F, W, Y, P, or C; G341 replaced with D, E, H, K, R, N, Q, F, W, Y, P, or C; L342 replaced with D, E, H, K, R, N, Q, F, W, Y, P, or C; G343 replaced with D, E, H, K, R, N, Q, F, W, Y, P, or C; N344 replaced with D, E, H, K, R, A, G, I, L, S, T, M, V, F, W, Y, P, or C; M345 replaced with D, E, H, K, R, N, Q, F, W, Y, P, or C; S346 replaced with D, E, H, K, R, N, Q, F, W, Y, P, or C; V347 replaced with D, E, H, K, R, N, Q, F, W, Y, P, or C; C348 replaced with D, E, H, K, R, A, G, I, L, S, T, M, V, N, Q, F, W, Y, or P; L349 replaced with D, E, H, K, R, N, Q, F, W, Y, P, or C; N350 replaced with D, E, H, K, R, A, G, I, L, S, T, M, V, F, W, Y, P, or C; Q351 replaced with D, E, H, K, R, A, G, I, L, S, T, M, V, F, W, Y, P, or C; S352 replaced with D, E, H, K, R, N, Q, F, W, Y, P, or C; P353 replaced with D, E, H, K, R, A, G, I, L, S, T, M, V, N, Q, F, W, Y, or C; W354 replaced with D, E, H, K, R, N, Q, A, G, I, L, S, T, M, V, P, or C; R355 replaced with D, E, A, G, I, L, S, T, M, V, N, Q, F, W, Y, P, or C; R356 replaced with D, E, A, G, I, L, S, T, M, V, N, Q, F, W, Y, P, or C; F357 replaced with D, E, H, K, R, N, Q, A, G, I, L, S, T, M, V, P, or C; C358 replaced with D, E, H, K, R, A, G, I, L, S, T, M, V, N, Q, F, W, Y, or P; V359 replaced with D, E, H, K, R, N, Q, F, W, Y, P, or C; Q360 replaced with D, E, H, K, R, A, G, I, L, S, T, M, V, F, W, Y, P, or C; P361 replaced with D, E, H, K, R, A, G, I, L, S, T, M, V, N, Q, F, W, Y, or C; P362 replaced with D, E, H, K, R, A, G, I, L, S, T, M, V, N, Q, F, W, Y, or C; C363 replaced with D, E, H, K, R, A, G, I, L, S, T, M, V, N, Q, F, W, Y, or P; G364 replaced with D, E, H, K, R, N, Q, F, W, Y, P, or C; Q365 replaced with D, E, H, K, R, A, G, I, L, S, T, M, V, F, W, Y, P, or C; L366 replaced with D, E, H, K, R, N, Q, F, W, Y, P, or C; N367 replaced with D, E, H, K, R, A, G, I, L, S, T, M, V, F, W, Y, P, or C; P368 replaced with D, E, H, K, R, A, G, I, L, S, T, M, V, N, Q, F, W, Y, or C; L369 replaced with D, E, H, K, R, N, Q, F, W, Y, P, or C; N370 replaced with D, E, H, K, R, A, G, I, L, S, T, M, V, F, W, Y, P, or C; D371 replaced with H, K, R, A, G, I, L, S, T, M, V, N, Q, F, W, Y, P, or C; H372 replaced with D, E, A, G, I, L, S, T, M, V, N, Q, F, W, Y, P, or C; M373 replaced with D, E, H, K, R, N, Q, F, W, Y, P, or C; Y374 replaced with D, E, H, K, R, N, Q, A, G, I, L, S, T, M, V, P, or C; A375 replaced with D, E, H, K, R, N, Q, F, W, Y, P, or C; V376 replaced with D, E, H, K, R, N, Q, F, W, Y, P, or C; L377 replaced with D, E, H, K, R, N, Q, F, W, Y, P, or C; K378 replaced with D, E, A, G, I, L, S, T, M, V, N, Q, F, W, Y, P, or C; E379 replaced with H, K, R, A, G, I, L, S, T, M, V, N, Q, F, W, Y, P, or C; I380 replaced with D, E, H, K, R, N, Q, F, W, Y, P, or C; F381 replaced with D, E, H, K, R, N, Q, A, G, I, L, S, T, M, V, P, or C; E382 replaced with H, K, R, A, G, I, L, S, T, M, V, N, Q, F, W, Y, P, or C; D383 replaced with H, K, R, A, G, I, L, S, T, M, V, N, Q, F, W, Y, P, or C; V384 replaced with D, E, H, K, R, N, Q, F, W, Y, P, or C; A385 replaced with D, E, H, K, R, N, Q, F, W, Y, P, or C; E386 replaced with H, K, R, A, G, I, L, S, T, M, V, N, Q, F, W, Y, P, or C; V387 replaced with D, E, H, K, R, N, Q, F, W, Y, P, or C; G388 replaced with D, E, H, K, R, N, Q, F, W, Y, P, or C; A389 replaced with D, E, H, K, R, N, Q, F, W, Y, P, or C; P390 replaced with D, E, H, K, R, A, G, I, L, S, T, M, V, N, Q, F, W, Y, or C; E391 replaced with H, K, R, A, G, I, L, S, T, M, V, N, Q, F, W, Y, P, or C; E392 replaced with H, K, R, A, G, I, L, S, T, M, V, N, Q, F, W, Y, P, or C; T393 replaced with D, E, H, K, R, N, Q, F, W, Y, P, or C; L394 replaced with D, E, H, K, R, N, Q, F, W, Y, P, or C; H395 replaced with D, E, A, G, I, L, S, T, M, V, N, Q, F, W, Y, P, or C; M396 replaced with D, E, H, K, R, N, Q, F, W, Y, P, or C; G397 replaced with D, E, H, K, R, N, Q, F, W, Y, P, or C; G398 replaced with D, E, H, K, R, N, Q, F, W, Y, P, or C; D399 replaced with H, K, R, A, G, I, L, S, T, M, V, N, Q, F, W, Y, P, or C; E400 replaced with H, K, R, A, G, I, L, S, T, M, V, N, Q, F, W, Y, P, or C; V401 replaced with D, E, H, K, R, N, Q, F, W, Y, P, or C; F402 replaced with D, E, H, K, R, N, Q, A, G, I, L, S, T, M, V, P, or C; L403 replaced with D, E, H, K, R, N, Q, F, W, Y, P, or C; P404 replaced with D, E, H, K, R, A, G, I, L, S, T, M, V, N, Q, F, W, Y, or C; C405 replaced with D, E, H, K, R, A, G, I, L, S, T, M, V, N, Q, F, W, Y, or P; W406 replaced with D, E, H, K, R, N, Q, A, G, I, L, S, T, M, V, P, or C; N407 replaced with D, E, H, K, R, A, G, I, L, S, T, M, V, F, W, Y, P, or C; N408 replaced with D, E, H, K, R, A, G, I, L, S, T, M, V, F, W, Y, P, or C; T409 replaced with D, E, H, K, R, N, Q, F, W, Y, P, or C; D410 replaced with H, K, R, A, G, I, L, S, T, M, V, N, Q, F, W, Y, P, or C; E411 replaced with H, K, R, A, G, I, L, S, T, M, V, N, Q, F, W, Y, P, or C; I412 replaced with D, E, H, K, R, N, Q, F, W, Y, P, or C; R413 replaced with D, E, A, G, I, L, S, T, M, V, N, Q, F, W, Y, P, or C; D414 replaced with H, K, R, A, G, I, L, S, T, M, V, N, Q, F, W, Y, P, or C; G415 replaced with D, E, H, K, R, N, Q, F, W, Y, P, or C; M416 replaced with D, E, H, K, R, N, Q, F, W, Y, P, or C; R417 replaced with D, E, A, G, I, L, S, T, M, V, N, Q, F, W, Y, P, or C; A418 replaced with D, E, H, K, R, N, Q, F, W, Y, P, or C; R419 replaced with D, E, A, G, I, L, S, T, M, V, N, Q, F, W, Y, P, or C; G420 replaced with D, E, H, K, R, N, Q, F, W, Y, P, or C; Y421 replaced with D, E, H, K, R, N, Q, A, G, I, L, S, T, M, V, P, or C; D422 replaced with H, K, R, A, G, I, L, S, T, M, V, N, Q, F, W, Y, P, or C; L423 replaced with D, E, H, K, R, N, Q, F, W, Y, P, or C; S424 replaced with D, E, H, K, R, N, Q, F, W, Y, P, or C; E425 replaced with H, K, R, A, G, I, L, S, T, M, V, N, Q, F, W, Y, P, or C; Q426 replaced with D, E, H, K, R, A, G, I, L, S, T, M, V, F, W, Y, P, or C; S427 replaced with D, E, H, K, R, N, Q, F, W, Y, P, or C; F428 replaced with D, E, H, K, R, N, Q, A, G, I, L, S, T, M, V, P, or C; L429 replaced with D, E, H, K, R, N, Q, F, W, Y, P, or C; R430 replaced with D, E, A, G, I, L, S, T, M, V, N, Q, F, W, Y, P, or C; L431 replaced with D, E, H, K, R, N, Q, F, W, Y, P, or C; W432 replaced with D, E, H, K, R, N, Q, A, G, I, L, S, T, M, V, P, or C; S433 replaced with D, E, H, K, R, N, Q, F, W, Y, P, or C; Q434 replaced with D, E, H, K, R, A, G, I, L, S, T, M, V, F, W, Y, P, or C; F435 replaced with D, E, H, K, R, N, Q, A, G, I, L, S, T, M, V, P, or C; H436 replaced with D, E, A, G, I, L, S, T, M, V, N, Q, F, W, Y, P, or C; Q437 replaced with D, E, H, K, R, A, G, I, L, S, T, M, V, F, W, Y, P, or C; R438 replaced with D, E, A, G, I, L, S, T, M, V, N, Q, F, W, Y, P, or C; N439 replaced with D, E, H, K, R, A, G, I, L, S, T, M, V, F, W, Y, P, or C; L440 replaced with D, E, H, K, R, N, Q, F, W, Y, P, or C; N441 replaced with D, E, H, K, R, A, G, I, L, S, T, M, V, F, W, Y, P, or C; A442 replaced with D, E, H, K, R, N, Q, F, W, Y, P, or C; W443 replaced with D, E, H, K, R, N, Q, A, G, I, L, S, T, M, V, P, or C; D444 replaced with H, K, R, A, G, I, L, S, T, M, V, N, Q, F, W, Y, P, or C; E445 replaced with H, K, R, A, G, I, L, S, T, M, V, N, Q, F, W, Y, P, or C; I446 replaced with D, E, H, K, R, N, Q, F, W, Y, P, or C; N447 replaced with D, E, H, K, R, A, G, I, L, S, T, M, V, F, W, Y, P, or C; E448 replaced with H, K, R, A, G, I, L, S, T, M, V, N, Q, F, W, Y, P, or C; R449 replaced with D, E, A, G, I, L, S, T, M, V, N, Q, F, W, Y, P, or C; M450 replaced with D, E, H, K, R, N, Q, F, W, Y, P, or C; Y451 replaced with D, E, H, K, R, N, Q, A, G, I, L, S, T, M, V, P, or C; P452 replaced with D, E, H, K, R, A, G, I, L, S, T, M, V, N, Q, F, W, Y, or C; G453 replaced with D, E, H, K, R, N, Q, F, W, Y, P, or C; I454 replaced with D, E, H, K, R, N, Q, F, W, Y, P, or C; K455 replaced with D, E, A, G, I, L, S, T, M, V, N, Q, F, W, Y, P, or C; E456 replaced with H, K, R, A, G, I, L, S, T, M, V, N, Q, F, W, Y, P, or C; P457 replaced with D, E, H, K, R, A, G, I, L, S, T, M, V, N, Q, F, W, Y, or C; K458 replaced with D, E, A, G, I, L, S, T, M, V, N, Q, F, W, Y, P, or C; S459 replaced with D, E, H, K, R, N, Q, F, W, Y, P, or C; V460 replaced with D, E, H, K, R, N, Q, F, W, Y, P, or C; I461 replaced with D, E, H, K, R, N, Q, F, W, Y, P, or C; I462 replaced with D, E, H, K, R, N, Q, F, W, Y, P, or C; W463 replaced with D, E, H, K, R, N, Q, A, G, I, L, S, T, M, V, P, or C; S464 replaced with D, E, H, K, R, N, Q, F, W, Y, P, or C; S465 replaced with D, E, H, K, R, N, Q, F, W, Y, P, or C; H466 replaced with D, E, A, G, I, L, S, T, M, V, N, Q, F, W, Y, P, or C; L467 replaced with D, E, H, K, R, N, Q, F, W, Y, P, or C; T468 replaced with D, E, H, K, R, N, Q, F, W, Y, P, or C; N469 replaced with D, E, H, K, R, A, G, I, L, S, T, M, V, F, W, Y, P, or C; P470 replaced with D, E, H, K, R, A, G, I, L, S, T, M, V, N, Q, F, W, Y, or C; R471 replaced with D, E, A, G, I, L, S, T, M, V, N, Q, F, W, Y, P, or C; Y472 replaced with D, E, H, K, R, N, Q, A, G, I, L, S, T, M, V, P, or C; I473 replaced with D, E, H, K, R, N, Q, F, W, Y, P, or C; E474 replaced with H, K, R, A, G, I, L, S, T, M, V, N, Q, F, W, Y, P, or C; T475 replaced with D, E, H, K, R, N, Q, F, W, Y, P, or C; Y476 replaced with D, E, H, K, R, N, Q, A, G, I, L, S, T, M, V, P, or C; L477 replaced with D, E, H, K, R, N, Q, F, W, Y, P, or C; P478 replaced with D, E, H, K, R, A, G, I, L, S, T, M, V, N, Q, F, W, Y, or C; K479 replaced with D, E, A, G, I, L, S, T, M, V, N, Q, F, W, Y, P, or C; E480 replaced with H, K, R, A, G, I, L, S, T, M, V, N, Q, F, W, Y, P, or C; R481 replaced with D, E, A, G, I, L, S, T, M, V, N, Q, F, W, Y, P, or C; F482 replaced with D, E, H, K, R, N, Q, A, G, I, L, S, T, M, V, P, or C; I483 replaced with D, E, H, K, R, N, Q, F, W, Y, P, or C; I484 replaced with D, E, H, K, R, N, Q, F, W, Y, P, or C; Q485 replaced with D, E, H, K, R, A, G, I, L, S, T, M, V, F, W, Y, P, or C; T486 replaced with D, E, H, K, R, N, Q, F, W, Y, P, or C; W487 replaced with D, E, H, K, R, N, Q, A, G, I, L, S, T, M, V, P, or C; V488 replaced with D, E, H, K, R, N, Q, F, W, Y, P, or C; E489 replaced with H, K, R, A, G, I, L, S, T, M, V, N, Q, F, W, Y, P, or C; S490 replaced with D, E, H, K, R, N, Q, F, W, Y, P, or C; Q491 replaced with D, E, H, K, R, A, G, I, L, S, T, M, V, F, W, Y, P, or C; D492 replaced with H, K, R, A, G, I, L, S, T, M, V, N, Q, F, W, Y, P, or C; A493 replaced with D, E, H, K, R, N, Q, F, W, Y, P, or C; L494 replaced with D, E, H, K, R, N, Q, F, W, Y, P, or C; N495 replaced with D, E, H, K, R, A, G, I, L, S, T, M, V, F, W, Y, P, or C; R496 replaced with D, E, A, G, I, L, S, T, M, V, N, Q, F, W, Y, P, or C; E497 replaced with H, K, R, A, G, I, L, S, T, M, V, N, Q, F, W, Y, P, or C; L498 replaced with D, E, H, K, R, N, Q, F, W, Y, P, or C; LA99 replaced with D, E, H, K, R, N, Q, F, W, Y, P, or C; Q500 replaced with D, E, H, K, R, A, G, I, L, S, T, M, V, F, W, Y, P, or C; R501 replaced with D, E, A, G, I, L, S, T, M, V, N, Q, F, W, Y, P, or C; G502 replaced with D, E, H, K, R, N, Q, F, W, Y, P, or C; Y503 replaced with D, E, H, K, R, N, Q, A, G, I, L, S, T, M, V, P, or C; R504 replaced with D, E, A, G, I, L, S, T, M, V, N, Q, F, W, Y, P, or C; L505 replaced with D, E, H, K, R, N, Q, F, W, Y, P, or C; I506 replaced with D, E, H, K, R, N, Q, F, W, Y, P, or C; V507 replaced with D, E, H, K, R, N, Q, F, W, Y, P, or C; S508 replaced with D, E, H, K, R, N, Q, F, W, Y, P, or C; T509 replaced with D, E, H, K, R, N, Q, F, W, Y, P, or C; K510 replaced with D, E, A, G, I, L, S, T, M, V, N, Q, F, W, Y, P, or C; N511 replaced with D, E, H, K, R, A, G, I, L, S, T, M, V, F, W, Y, P, or C; A512 replaced with D, E, H, K, R, N, Q, F, W, Y, P, or C; W513 replaced with D, E, H, K, R, N, Q, A, G, I, L, S, T, M, V, P, or C; Y514 replaced with D, E, H, K, R, N, Q, A, G, I, L, S, T, M, V, P, or C; L515 replaced with D, E, H, K, R, N, Q, F, W, Y, P, or C; D516 replaced with H, K, R, A, G, I, L, S, T, M, V, N, Q, F, W, Y, P, or C; H517 replaced with D, E, A, G, I, L, S, T, M, V, N, Q, F, W, Y, P, or C; G518 replaced with D, E, H, K, R, N, Q, F, W, Y, P, or C; F519 replaced with D, E, H, K, R, N, Q, A, G, I, L, S, T, M, V, P, or C; W520 replaced with D, E, H, K, R, N, Q, A, G, I, L, S, T, M, V, P, or C; G521 replaced with D, E, H, K, R, N, Q, F, W, Y, P, or C; S522 replaced with D, E, H, K, R, N, Q, F, W, Y, P, or C; T523 replaced with D, E, H, K, R, N, Q, F, W, Y, P, or C; S524 replaced with D, E, H, K, R, N, Q, F, W, Y, P, or C; Y525 replaced with D, E, H, K, R, N, Q, A, G, I, L, S, T, M, V, P, or C; Y526 replaced with D, E, H, K, R, N, Q, A, G, I, L, S, T, M, V, P, or C; N527 replaced with D, E, H, K, R, A, G, I, L, S, T, M, V, F, W, Y, P, or C; W528 replaced with D, E, H, K, R, N, Q, A, G, I, L, S, T, M, V, P, or C; R529 replaced with D, E, A, G, I, L, S, T, M, V, N, Q, F, W, Y, P, or C; T530 replaced with D, E, H, K, R, N, Q, F, W, Y, P, or C; V531 replaced with D, E, H, K, R, N, Q, F, W, Y, P, or C; Y532 replaced with D, E, H, K, R, N, Q, A, G, I, L, S, T, M, V, P, or C; S533 replaced with D, E, H, K, R, N, Q, F, W, Y, P, or C; S534 replaced with D, E, H, K, R, N, Q, F, W, Y, P, or C; G535 replaced with D, E, H, K, R, N, Q, F, W, Y, P, or C; M536 replaced with D, E, H, K, R, N, Q, F, W, Y, P, or C; P537 replaced with D, E, H, K, R, A, G, I, L, S, T, M, V, N, Q, F, W, Y, or C; V538 replaced with D, E, H, K, R, N, Q, F, W, Y, P, or C; G539 replaced with D, E, H, K, R, N, Q, F, W, Y, P, or C; R540 replaced with D, E, A, G, I, L, S, T, M, V, N, Q, F, W, Y, P, or C; S541 replaced with D, E, H, K, R, N, Q, F, W, Y, P, or C; K542 replaced with D, E, A, G, I, L, S, T, M, V, N, Q, F, W, Y, P, or C; D543 replaced with H, K, R, A, G, I, L, S, T, M, V, N, Q, F, W, Y, P, or C; Q544 replaced with D, E, H, K, R, A, G, I, L, S, T, M, V, F, W, Y, P, or C; V545 replaced with D, E, H, K, R, N, Q, F, W, Y, P, or C; L546 replaced with D, E, H, K, R, N, Q, F, W, Y, P, or C; G547 replaced with D, E, H, K, R, N, Q, F, W, Y, P, or C; G548 replaced with D, E, H, K, R, N, Q, F, W, Y, P, or C; E549 replaced with H, K, R, A, G, I, L, S, T, M, V, N, Q, F, W, Y, P, or C; V550 replaced with D, E, H, K, R, N, Q, F, W, Y, P, or C; C551 replaced with D, E, H, K, R, A, G, I, L, S, T, M, V, N, Q, F, W, Y, or P; M552 replaced with D, E, H, K, R, N, Q, F, W, Y, P, or C; W553 replaced with D, E, H, K, R, N, Q, A, G, I, L, S, T, M, V, P, or C; S554 replaced with D, E, H, K, R, N, Q, F, W, Y, P, or C; E555 replaced with H, K, R, A, G, I, L, S, T, M, V, N, Q, F, W, Y, P, or C; Y556 replaced with D, E, H, K, R, N, Q, A, G, I, L, S, T, M, V, P, or C; V557 replaced with D, E, H, K, R, N, Q, F, W, Y, P, or C; D558 replaced with H, K, R, A, G, I, L, S, T, M, V, N, Q, F, W, Y, P, or C; Q559 replaced with D, E, H, K, R, A, G, I, L, S, T, M, V, F, W, Y, P, or C; N560 replaced with D, E, H, K, R, A, G, I, L, S, T, M, V, F, W, Y, P, or C; S561 replaced with D, E, H, K, R, N, Q, F, W, Y, P, or C; L562 replaced with D, E, H, K, R, N, Q, F, W, Y, P, or C; E563 replaced with H, K, R, A, G, I, L, S, T, M, V, N, Q, F, W, Y, P, or C; S564 replaced with D, E, H, K, R, N, Q, F, W, Y, P, or C; R565 replaced with D, E, A, G, I, L, S, T, M, V, N, Q, F, W, Y, P, or C; I566 replaced with D, E, H, K, R, N, Q, F, W, Y, P, or C; W567 replaced with D, E, H, K, R, N, Q, A, G, I, L, S, T, M, V, P, or C; P568 replaced with D, E, H, K, R, A, G, I, L, S, T, M, V, N, Q, F, W, Y, or C; R569 replaced with D, E, A, G, I, L, S, T, M, V, N, Q, F, W, Y, P, or C; A570 replaced with D, E, H, K, R, N, Q, F, W, Y, P, or C; G571 replaced with D, E, H, K, R, N, Q, F, W, Y, P, or C; A572 replaced with D, E, H, K, R, N, Q, F, W, Y, P, or C; A573 replaced with D, E, H, K, R, N, Q, F, W, Y, P, or C; A574 replaced with D, E, H, K, R, N, Q, F, W, Y, P, or C; E575 replaced with H, K, R, A, G, I, L, S, T, M, V, N, Q, F, W, Y, P, or C; R576 replaced with D, E, A, G, I, L, S, T, M, V, N, Q, F, W, Y, P, or C; M577 replaced with D, E, H, K, R, N, Q, F, W, Y, P, or C; W578 replaced with D, E, H, K, R, N, Q, A, G, I, L, S, T, M, V, P, or C; S579 replaced with D, E, H, K, R, N, Q, F, W, Y, P, or C; N580 replaced with D, E, H, K, R, A, G, I, L, S, T, M, V, F, W, Y, P, or C; P581 replaced with D, E, H, K, R, A, G, I, L, S, T, M, V, N, Q, F, W, Y, or C; K582 replaced with D, E, A, G, I, L, S, T, M, V, N, Q, F, W, Y, P, or C; S583 replaced with D, E, H, K, R, N, Q, F, W, Y, P, or C; S584 replaced with D, E, H, K, R, N, Q, F, W, Y, P, or C; A585 replaced with D, E, H, K, R, N, Q, F, W, Y, P, or C; L586 replaced with D, E, H, K, R, N, Q, F, W, Y, P, or C; L587 replaced with D, E, H, K, R, N, Q, F, W, Y, P, or C; A588 replaced with D, E, H, K, R, N, Q, F, W, Y, P, or C; Q589 replaced with D, E, H, K, R, A, G, I, L, S, T, M, V, F, W, Y, P, or C; R590 replaced with D, E, A, G, I, L, S, T, M, V, N, Q, F, W, Y, P, or C; R591 replaced with D, E, A, G, I, L, S, T, M, V, N, Q, F, W, Y, P, or C; F592 replaced with D, E, H, K, R, N, Q, A, G, I, L, S, T, M, V, P, or C; Y593 replaced with D, E, H, K, R, N, Q, A, G, I, L, S, T, M, V, P, or C; R594 replaced with D, E, A, G, I, L, S, T, M, V, N, Q, F, W, Y, P, or C; Y595 replaced with D, E, H, K, R, N, Q, A, G, I, L, S, T, M, V, P, or C; R596 replaced with D, E, A, G, I, L, S, T, M, V, N, Q, F, W, Y, P, or C; E597 replaced with H, K, R, A, G, I, L, S, T, M, V, N, Q, F, W, Y, P, or C; R598 replaced with D, E, A, G, I, L, S, T, M, V, N, Q, F, W, Y, P, or C; L599 replaced with D, E, H, K, R, N, Q, F, W, Y, P, or C; L600 replaced with D, E, H, K, R, N, Q, F, W, Y, P, or C; A601 replaced with D, E, H, K, R, N, Q, F, W, Y, P, or C; R602 replaced with D, E, A, G, I, L, S, T, M, V, N, Q, F, W, Y, P, or C; G603 replaced with D, E, H, K, R, N, Q, F, W, Y, P, or C; I604 replaced with D, E, H, K, R, N, Q, F, W, Y, P, or C; H605 replaced with D, E, A, G, I, L, S, T, M, V, N, Q, F, W, Y, P, or C; A606 replaced with D, E, H, K, R, N, Q, F, W, Y, P, or C; D607 replaced with H, K, R, A, G, I, L, S, T, M, V, N, Q, F, W, Y, P, or C; A608 replaced with D, E, H, K, R, N, Q, F, W, Y, P, or C; V609 replaced with D, E, H, K, R, N, Q, F, W, Y, P, or C; I610 replaced with D, E, H, K, R, N, Q, F, W, Y, P, or C; P611 replaced with D, E, H, K, R, A, G, I, L, S, T, M, V, N, Q, F, W, Y, or C; H612 replaced with D, E, A, G, I, L, S, T, M, V, N, Q, F, W, Y, P, or C; W613 replaced with D, E, H, K, R, N, Q, A, G, I, L, S, T, M, V, P, or C; C614 replaced with D, E, H, K, R, A, G, I, L, S, T, M, V, N, Q, F, W, Y, or P; V615 replaced with D, E, H, K, R, N, Q, F, W, Y, P, or C; L616 replaced with D, E, H, K, R, N, Q, F, W, Y, P, or C; H617 replaced with D, E, A, G, I, L, S, T, M, V, N, Q, F, W, Y, P, or C; E618 replaced with H, K, R, A, G, I, L, S, T, M, V, N, Q, F, W, Y, P, or C; G619 replaced with D, E, H, K, R, N, Q, F, W, Y, P, or C; Q620 replaced with D, E, H, K, R, A, G, I, L, S, T, M, V, F, W, Y, P, or C; C621 replaced with D, E, H, K, R, A, G, I, L, S, T, M, V, N, Q, F, W, Y, or P; and/or L622 replaced with D, E, H, K, R, N, Q, F, W, Y, P, or C.

The resulting constructs can be routinely screened for activities or functions described throughout the specification and known in the art. Preferably, the resulting constructs have loss of a hexosaminidase activity or function, while the remaining biological activities or functions are maintained. More preferably, the resulting constructs have more than one loss of biological activity or function, while the remaining biological activities or functions are maintained.

Additionally, more than one amino acid (e.g., 2, 3, 4, 5, 6, 7, 8, 9 and 10) can be replaced with the substituted amino acids as described above (either conservative or nonconservative). The substituted amino acids can occur in the full length, mature, or proprotein form of Hex-2 (SEQ ID NO:6) protein, as well as the N- and C-terminal deletion mutants, having the general formula m-n, listed below.

A further embodiment of the invention relates to a polypeptide which comprises the amino acid sequence of Hex-2 (SEQ ID NO:6) polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of Hex-2 polypeptide (SEQ ID NO:6), which contains at least one, but not more than 10, 9, 8, 7, 6, 5, 4, 3, 2 or 1 amino acid substitutions. In specific embodiments, the number of additions, substitutions, and/or deletions in the amino acid sequence of FIG. 3 or fragments thereof (e.g., the mature form and/or other fragments described herein), is 1-5, 5-10, 5-25, 5-50, 10-50 or 50-150, conservative amino acid substitutions are preferable.

Polynucleotide and Polypeptide Fragments

The present invention is also directed to polynucleotide fragments of the polynucleotides of CMP-SAT (SEQ ID NO:1). In the present invention, a “polynucleotide fragment” refers to a short polynucleotide having a nucleic acid sequence which: is a portion of that contained in the deposited clone, or encoding the polypeptide encoded by the cDNA in the deposited clone; is a portion of that shown in SEQ ID NO:1 or the complementary strands thereto, or is a portion of the polynucleotide sequence encoding the polypeptide of SEQ ID NO:2. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the cDNA sequence contained in a deposited clone or the nucleotide sequence shown in SEQ ID NO:1. In this context “about” includes the particularly recited value, a value larger or smaller by several (5, 4, 3, 2, or 1) nucleotides, at either terminus or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, or 1951 to the end of SEQ ID NO:1, or the complementary strand thereto, or the cDNA contained in the deposited clone. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Polynucleotides which hybridize to these nucleic acid molecules under stringent hybridization conditions or lower stringency conditions are also encompassed by the invention, as are polypeptides encoded by these polynucleotides. In the present invention, a “polypeptide fragment” refers to an amino acid sequence which is a portion of that contained in SEQ ID NO:2 or encoded by the cDNA contained in the deposited clone. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Even if deletion of one or more amino acids from the N-terminus of a protein results in modification or loss of one or more biological functions of the protein, other functional activities (e.g., biological activities, ability to multimerize, ability to bind substrates) may still be retained. For example, the ability of shortened CMP-SAT (SEQ ID NO:2) muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a CMP-SAT (SEQ ID NO:2) mutein with a large number of deleted N-terminal amino acid residues may retain some biological or immunogenic activities. In fact, peptides composed of as few as six CMP-SAT (SEQ ID NO:2) amino acid residues may elicit an immune response.

Preferred polypeptide fragments include the full-length protein as well as the mature form. Further preferred polypeptide fragments include the full-length protein or the mature form having a continuous series of deleted residues from the amino or the carboxy termini, or both.

Accordingly, polypeptide fragments include the full-length CMP-SAT protein (SEQ ID NO:2) as well as mature forms. Further preferred polypeptide fragments include the mature forms of CMP-SAT having a continuous series of deleted residues from the amino or the carboxy terminus, or both. For example, any number of amino acids, ranging from 1-351, can be deleted from the amino terminus of either the secreted TGF alpha HIII polypeptide or the mature form. Similarly, any number of amino acids, ranging from 1-351, can be deleted from the carboxy terminus of the secreted TGF alpha HIII protein or mature form. Furthermore, any combination of the above amino and carboxy terminus deletions are preferred. Similarly, polynucleotides encoding these polypeptide fragments are also preferred.

Particularly, N-terminal deletions of the CMP-SAT (SEQ ID NO:2) polypeptide can be described by the general formula m¹-357, where m¹ is an integer from 2 to 351, where m¹ corresponds to the position of the amino acid residue identified in SEQ ID NO:2. More in particular, the invention provides polynucleotides encoding polypeptides comprising, or alternatively consisting of, the amino acid sequence of residues of N-terminal deletions of the CMP-SAT polypeptide of the invention shown as SEQ ID NO:2 including polypeptides comprising the amino acid sequence of residues: N-2 to V-357; S-3 to V-357; I-4 to V-357; H-5 to V-357; M-6 to V-357; N-7 to V-357; A-8 to V-357; N-9 to V-357; T-10 to V-357; L-11 to V-357; K-12 to V-357; Y-13 to V-357; I-14 to V-357; S-15 to V-357; L-16 to V-357; L-17 to V-357; T-18 to V-357; L-19 to V-357; T-20 to V-357; L-21 to V-357; Q-22 to V-357; N-23 to V-357; A-24 to V-357; I-25 to V-357; L-26 to V-357; G-27 to V-357; L-28 to V-357; S-29 to V-357; M-30 to V-357; R-31 to V-357; Y-32 to V-357; A-33 to V-357; R-34 to V-357; T-35 to V-357; R-36 to V-357; P-37 to V-357; G-38 to V-357; D-39 to V-357; I-40 to V-357; F-41 to V-357; L-42 to V-357; S-43 to V-357; S-44 to V-357; T-45 to V-357; A-46 to V-357; V-47 to V-357; L-48 to V-357; M-49 to V-357; A-50 to V-357; E-51 to V-357; F-52 to V-357; A-53 to V-357; K-54 to V-357; L-55 to V-357; I-56 to V-357; T-57 to V-357; C-58 to V-357; L-59 to V-357; F-60 to V-357; L-61 to V-357; V-62 to V-357; F-63 to V-357; N-64 to V-357; E-65 to V-357; E-66 to V-357; G-67 to V-357; K-68 to V-357; D-69 to V-357; A-70 to V-357; Q-71 to V-357; K-72 to V-357; F-73 to V-357; V-74 to V-357; R-75 to V-357; S-76 to V-357; L-77 to V-357; H-78 to V-357; K-79 to V-357; T-80 to V-357; I-81 to V-357; N-82 to V-357; A-83 to V-357; N-84 to V-357; P-85 to V-357; M-86 to V-357; D-87 to V-357; T-88 to V-357; L-89 to V-357; K-90 to V-357; V-91 to V-357; C-92 to V-357; V-93 to V-357; P-94 to V-357; S-95 to V-357; L-96 to V-357; V-97 to V-357; Y-98 to V-357; I-99 to V-357; V-100 to V-357; Q-101 to V-357; N-102 to V-357; N-103 to V-357; L-104 to V-357; L-105 to V-357; Y-106 to V-357; V-107 to V-357; S-108 to V-357; A-109 to V-357; S-10 to V-357; H-111 to V-357; L-112 to V-357; D-113 to V-357; A-114 to V-357; A-115 to V-357; T-116 to V-357; Y-117 to V-357; Q-118 to V-357; V-119 to V-357; T-120 to V-357; Y-121 to V-357; Q-122 to V-357; L-123 to V-357; K-124 to V-357; I-125 to V-357; L-126 to V-357; T-127 to V-357; T-128 to V-357; A-129 to V-357; M-130 to V-357; F-131 to V-357; A-132 to V-357; V-133 to V-357; V-134 to V-357; I-135 to V-357; L-136 to V-357; R-137 to V-357; R-138 to V-357; K-139 to V-357; L-140 to V-357; L-141 to V-357; N-142 to V-357; T-143 to V-357; Q-144 to V-357; W-145 to V-357; G-146 to V-357; A-147 to V-357; L-148 to V-357; L-149 to V-357; L-150 to V-357; L-151 to V-357; V-152 to V-357; M-153 to V-357; G-154 to V-357; I-155 to V-357; V-156 to V-357; L-157 to V-357; V-158 to V-357; Q-159 to V-357; L-160 to V-357; A-161 to V-357; Q-162 to V-357; T-163 to V-357; E-164 to V-357; G-165 to V-357; P-166 to V-357; T-167 to V-357; S-168 to V-357; G-169 to V-357; S-170 to V-357; A-171 to V-357; G-172 to V-357; G-173 to V-357; A-174 to V-357; A-175 to V-357; A-176 to V-357; A-177 to V-357; A-178 to V-357; T-179 to V-357; A-180 to V-357; A-181 to V-357; S-182 to V-357; S-183 to V-357; G-184 to V-357; G-185 to V-357; A-186 to V-357; P-187 to V-357; E-188 to V-357; Q-189 to V-357; N-190 to V-357; R-191 to V-357; M-192 to V-357; L-193 to V-357; G-194 to V-357; L-195 to V-357; W-196 to V-357; A-197 to V-357; A-198 to V-357; L-199 to V-357; G-200 to V-357; A-201 to V-357; C-202 to V-357; F-203 to V-357; L-204 to V-357; S-205 to V-357; G-206 to V-357; F-207 to V-357; A-208 to V-357; G-209 to V-357; I-210 to V-357; Y-211 to V-357; F-212 to V-357; E-213 to V-357; K-214 to V-357; I-215 to V-357; L-216 to V-357; K-217 to V-357; G-218 to V-357; A-219 to V-357; E-220 to V-357; 1-221 to V-357; S-222 to V-357; V-223 to V-357; W-224 to V-357; M-225 to V-357; R-226 to V-357; N-227 to V-357; V-228 to V-357; Q-229 to V-357; L-230 to V-357; S-231 to V-357; L-232 to V-357; L-233 to V-357; S-234 to V-357; I-235 to V-357; P-236 to V-357; F-237 to V-357; G-238 to V-357; L-239 to V-357; L-240 to V-357; T-241 to V-357; C-242 to V-357; F-243 to V-357; V-244 to V-357; N-245 to V-357; D-246 to V-357; G-247 to V-357; S-248 to V-357; R-249 to V-357; I-250 to V-357; F-251 to V-357; D-252 to V-357; Q-253 to V-357; G-254 to V-357; F-255 to V-357; F-256 to V-357; K-257 to V-357; G-258 to V-357; Y-259 to V-357; D-260 to V-357; L-261 to V-357; F-262 to V-357; V-263 to V-357; W-264 to V-357; Y-265 to V-357; L-266 to V-357; V-267 to V-357; L-268 to V-357; L-269 to V-357; Q-270 to V-357; A-271 to V-357; G-272 to V-357; G-273 to V-357; G-274 to V-357; L-275 to V-357; I-276 to V-357; V-277 to V-357; A-278 to V-357; V-279 to V-357; V-280 to V-357; V-281 to V-357; K-282 to V-357; Y-283 to V-357; A-284 to V-357; D-285 to V-357; N-286 to V-357; I-287 to V-357; L-288 to V-357; K-289 to V-357; G-290 to V-357; F-291 to V-357; A-292 to V-357; T-293 to V-357; S-294 to V-357; L-295 to V-357; A-296 to V-357; I-297 to V-357; I-298 to V-357; I-299 to V-357; S-300 to V-357; C-301 to V-357; V-302 to V-357; A-303 to V-357; S-304 to V-357; I-305 to V-357; Y-306 to V-357; I-307 to V-357; F-308 to V-357; D-309 to V-357; F-310 to V-357; N-311 to V-357; L-312 to V-357; T-313 to V-357; L-314 to V-357; Q-315 to V-357; F-316 to V-357; S-317 to V-357; F-318 to V-357; G-319 to V-357; A-320 to V-357; G-321 to V-357; L-322 to V-357; V-323 to V-357; I-324 to V-357; A-325 to V-357; S-326 to V-357; I-327 to V-357; F-328 to V-357; L-329 to V-357; Y-330 to V-357; G-331 to V-357; Y-332 to V-357; D-333 to V-357; P-334 to V-357; A-335 to V-357; R-336 to V-357; S-337 to V-357; A-338 to V-357; P-339 to V-357; K-340 to V-357; P-341 to V-357; T-342 to V-357; M-343 to V-357; H-344 to V-357; G-345 to V-357; P-346 to V-357; G-347 to V-357; G-348 to V-357; D-349 to V-357; E-350 to V-357; E-351 to V-357; and/or K-352 to V-357 of SEQ ID NO:2. Polynucleotides encoding these polypeptides are also encompassed by the invention.

Also as mentioned above, even if deletion of one or more amino acids from the C-terminus of a protein results in modification or loss of one or more biological functions of the protein, other functional activities (e.g., biological activities, ability to multimerize, ability to bind substrate) may still be retained. For example the ability of the shortened CMP-SAT (SEQ ID NO:2) mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a CMP-SAT (SEQ ID NO:2) mutein with a large number of deleted C-terminal amino acid residues may retain some biological or immunogenic activities. In fact, peptides composed of as few as six CMP-SAT (SEQ ID NO:2) amino acid residues may elicit an immune response. Accordingly, the present invention further provides polypeptides having one or more residues deleted from the carboxy terminus of the amino acid sequence of the CMP-SAT polypeptide shown in FIG. 1 (SEQ ID NO:2), as described by the general formula 1-n¹, where n¹ is an integer from 6 to 351, where n¹ corresponds to the position of amino acid residue identified in SEQ ID NO:2. More in particular, the invention provides polynucleotides encoding polypeptides comprising, or alternatively consisting of, the amino acid sequence of residues of C-terminal deletions of the CMP-SAT polypeptide of the invention shown as SEQ ID NO:2 include polypeptides comprising the amino acid sequence of residues: M-1 to R-356; M-1 to P-355; M-1 to L-354; M-1 to L-353; M-1 to K-352; M-1 to E-351; M-1 to E-350; M-1 to D-349; M-1 to G-348; M-1 to G-347; M-1 to P-346; M-1 to G-345; M-1 to H-344; M-1 to M-343; M-1 to T-342; M-1 to P-341; M-1 to K-340; M-1 to P-339; M-1 to A-338; M-1 to S-337; M-1 to R-336; M-1 to A-335; M-1 to P-334; M-1 to D-333; M-1 to Y-332; M-1 to G-331; M-1 to Y-330; M-1 to L-329; M-1 to F-328; M-1 to I-327; M-1 to S-326; M-1 to A-325; M-1 to I-324; M-1 to V-323; M-1 to L-322; M-1 to G-321; M-1 to A-320; M-1 to G-319; M-1 to F-318; M-1 to S-317; M-1 to F-316; M-1 to Q-315; M-1 to L-314; M-1 to T-313; M-1 to L-312; M-1 to N-311; M-1 to F-310; M-1 to D-309; M-1 to F-308; M-1 to I-307; M-1 to Y-306; M-1 to I-305; M-1 to S-304; M-1 to A-303; M-1 to V-302; M-1 to C-301; M-1 to S-300; M-1 to I-299; M-1 to I-298; M-1 to I-297; M-1 to A-296; M-1 to L-295; M-1 to S-294; M-1 to T-293; M-1 to A-292; M-1 to F-291; M-1 to G-290; M-1 to K-289; M-1 to L-288; M-1 to I-287; M-1 to N-286; M-1 to D-285; M-1 to A-284; M-1 to Y-283; M-1 to K-282; M-1 to V-281; M-1 to V-280; M-1 to V-279; M-1 to A-278; M-1 to V-277; M-1 to I-276; M-1 to L-275; M-1 to G-274; M-1 to G-273; M-1 to G-272; M-1 to A-271; M-1 to Q-270; M-1 to L-269; M-1 to L-268; M-1 to V-267; M-1 to L-266; M-1 to Y-265; M-1 to W-264; M-1 to V-263; M-1 to F-262; M-1 to L-261; M-1 to D-260; M-1 to Y-259; M-1 to G-258; M-1 to K-257; M-1 to F-256; M-1 to F-255; M-1 to G-254; M-1 to Q-253; M-1 to D-252; M-1 to F-251; M-1 to I-250; M-1 to R-249; M-1 to S-248; M-1 to G-247; M-1 to D-246; M-1 to N-245; M-1 to V-244; M-1 to F-243; M-1 to C-242; M-1 to T-241; M-1 to L-240; M-1 to L-239; M-1 to G-238; M-1 to F-237; M-1 to P-236; M-1 to I-235; M-1 to S-234; M-1 to L-233; M-1 to L-232; M-1 to S-231; M-1 to L-230; M-1 to Q-229; M-1 to V-228; M-1 to N-227; M-1 to R-226; M-1 to M-225; M-1 to W-224; M-1 to V-223; M-1 to S-222; M-1 to I-221; M-1 to E-220; M-1 to A-219; M-1 to G-218; M-1 to K-217; M-1 to L-216; M-1 to I-215; M-1 to K-214; M-1 to E-213; M-1 to F-212; M-1 to Y-211; M-1 to I-210; M-1 to G-209; M-1 to A-208; M-1 to F-207; M-1 to G-206; M-1 to S-205; M-1 to L-204; M-1 to F-203; M-1 to C-202; M-1 to A-201; M-1 to G-200; M-1 to L-199; M-1 to A-198; M-1 to A-197; M-1 to W-196; M-1 to L-195; M-1 to G-194; M-1 to L-193; M-1 to M-192; M-1 to R-191; M-1 to N-190; M-1 to Q-189; M-1 to E-188; M-1 to P-187; M-1 to A-186; M-1 to G-185; M-1 to G-184; M-1 to S-183; M-1 to S-182; M-1 to A-181; M-1 to A-180; M-1 to T-179; M-1 to A-178; M-1 to A-177; M-1 to A-176; M-1 to A-175; M-1 to A-174; M-1 to G-173; M-1 to G-172; M-1 to A-171; M-1 to S-170; M-1 to G-169; M-1 to S-168; M-1 to T-167; M-1 to P-166; M-1 to G-165; M-1 to E-164; M-1 to T-163; M-1 to Q-162; M-1 to A-161; M-1 to L-160; M-1 to Q-159; M-1 to V-158; M-1 to L-157; M-1 to V-156; M-1 to I-155; M-1 to G-154; M-1 to M-153; M-1 to V-152; M-1 to L-151; M-1 to L-150; M-1 to L-149; M-1 to L-148; M-1 to A-147; M-1 to G-146; M-1 to W-145; M-1 to Q-144; M-1 to T-143; M-1 to N-142; M-1 to L-141; M-1 to L-140; M-1 to K-139; M-1 to R-138; M-1 to R-137; M-1 to L-136; M-1 to I-135; M-1 to V-134; M-1 to V-133; M-1 to A-132; M-1 to F-131; M-1 to M-130; M-1 to A-129; M-1 to T-128; M-1 to T-127; M-1 to L-126; M-1 to I-125; M-1 to K-124; M-1 to L-123; M-1 to Q-122; M-1 to Y-121; M-1 to T-120; M-1 to V-119; M-1 to Q-118; M-1 to Y-117; M-1 to T-116; M-1 to A-115; M-1 to A-114; M-1 to D-113; M-1 to L-112; M-1 to H-111; M-1 to S-110; M-1 to A-109; M-1 to S-108; M-1 to V-107; M-1 to Y-106; M-1 to L-105; M-1 to L-104; M-1 to N-103; M-1 to N-102; M-1 to Q-101; M-1 to V-100; M-1 to I-99; M-1 to Y-98; M-1 to V-97; M-1 to L-96; M-1 to S-95; M-1 to P-94; M-1 to V-93; M-1 to C-92; M-1 to V-91; M-1 to K-90; M-1 to L-89; M-1 to T-88; M-1 to D-87; M-1 to M-86; M-1 to P-85; M-1 to N-84; M-1 to A-83; M-1 to I-82; M-1 to I-81; M-1 to T-80; M-1 to K-79; M-1 to H-78; M-1 to L-77; M-1 to S-76; M-1 to R-75; M-1 to V-74; M-1 to F-73; M-1 to K-72; M-1 to Q-71; M-1 to A-70; M-1 to D-69; M-1 to K-68; M-1 to G-67; M-1 to E-66; M-1 to E-65; M-1 to N-64; M-1 to F-63; M-1 to V-62; M-1 to L-61; M-1 to F-60; M-1 to L-59; M-1 to C-58; M-1 to T-57; M-1 to I-56; M-1 to L-55; M-1 to K-54; M-1 to A-53; M-1 to F-52; M-1 to E-51; M-1 to A-50; M-1 to M-49; M-1 to L-48; M-1 to V-47; M-1 to A-46; M-1 to T-45; M-1 to S-44; M-1 to S-43; M-1 to L-42; M-1 to F-41; M-1 to I-40; M-1 to D-39; M-1 to G-38; M-1 to P-37; M-1 to R-36; M-1 to T-35; M-1 to R-34; M-1 to A-33; M-1 to Y-32; M-1 to R-31; M-1 to M-30; M-1 to S-29; M-1 to L-28; M-1 to G-27; M-1 to L-26; M-1 to I-25; M-1 to A-24; M-1 to N-23; M-1 to Q-22; M-1 to L-21; M-1 to T-20; M-1 to L-19; M-1 to T-18; M-1 to L-17; M-1 to L-16; M-1 to S-15; M-1 to I-14; M-1 to Y-13; M-1 to K-12; M-1 to L-11; M-1 to T-10; M-1 to N-9; M-1 to A-8; and/or M-1 to N-7 of SEQ ID NO:2. Polynucleotides encoding these polypeptides are also encompassed by the invention.

In addition, any of the above listed N- or C-terminal deletions can be combined to produce a N- and C-terminal deleted CMP-SAT (SEQ ID NO:2) polypeptides. The invention also provides polypeptides having one or more amino acids deleted from both the amino and the carboxyl termini, which may be described generally as having residues m¹-n¹ of SEQ ID NO:2, where n¹ and m¹ are integers as described above. Polynucleotides encoding these polypeptides are also encompassed by the invention.

Also included are a nucleotide sequence encoding a polypeptide consisting of a portion of the complete CMP-SAT amino acid sequence encoded by the cDNA clone contained in ATCC Deposit No. ______ (as shown in SEQ ID NO:1 and SEQ ID NO:2), where this portion excludes any integer of amino acid residues from 1 to about 351 amino acids from the amino terminus of the complete amino acid sequence encoded by the cDNA clone contained in the ATCC Deposit No. ______, or any integer of amino acid residues from 1 to 351 amino acids from the carboxy terminus, or any combination of the above amino terminal and carboxy terminal deletions, of the complete amino acid sequence encoded by the cDNA clone contained in the ATCC Deposit No. ______. Polynucleotides encoding all of the above deletion mutant polypeptide forms also are provided.

The present application is also directed to proteins containing polypeptides at least 90%, 95%, 96%, 97%, 98% or 99% identical to the CMP-SAT polypeptide sequence set forth herein. In preferred embodiments, the application is directed to proteins containing polypeptides at least 90%, 95%, 96%, 97%, 98% or 99% identical to polypeptides having the amino acid sequence of the specific CMP-SAT N- and C-terminal deletions recited herein. Polynucleotides encoding these polypeptides are also encompassed by the invention.

Additional preferred CMP-SAT (SEQ ID NO:2) polypeptide fragments comprise, or alternatively consist of, the amino acid sequence of residues: M-1 to S-15; N-2 to L-16; S-3 to L-17; I-4 to T-18; H-5 to L-19; M-6 to T-20; N-7 to L-21; A-8 to Q-22; N-9 to N-23; T-10 to A-24; L-11 to I-25; K-12 to L-26; Y-13 to G-27; I-14 to L-28; S-15 to S-29; L-16 to M-30; L-17 to R-31; T-18 to Y-32; L-19 to A-33; T-20 to R-34; L-21 to T-35; Q-22 to R-36; N-23 to P-37; A-24 to G-38; I-25 to D-39; L-26 to I-40; G-27 to F-41; L-28 to L-42; S-29 to S-43; M-30 to S-44; R-31 to T-45; Y-32 to A-46; A-33 to V-47; R-34 to L-48; T-35 to M-49; R-36 to A-50; P-37 to E-51; G-38 to F-52; D-39 to A-53; I-40 to K-54; F-41 to L-55; L-42 to I-56; S-43 to T-57; S-44 to C-58; T-45 to L-59; A-46 to F-60; V-47 to L-61; L-48 to V-62; M-49 to F-63; A-50 to N-64; E-51 to E-65; F-52 to E-66; A-53 to G-67; K-54 to K-68; L-55 to D-69; I-56 to A-70; T-57 to Q-71; C-58 to K-72; L-59 to F-73; F-60 to V-74; L-61 to R-75; V-62 to S-76; F-63 to L-77; N-64 to H-78; E-65 to K-79; E-66 to T-80; G-67 to I-81; K-68 to I-82; D-69 to A-83; A-70 to N-84; Q-71 to P-85; K-72 to M-86; F-73 to D-87; V-74 to T-88; R-75 to L-89; S-76 to K-90; L-77 to V-91; H-78 to C-92; K-79 to V-93; T-80 to P-94; I-81 to S-95; I-82 to L-96; A-83 to V-97; N-84 to Y-98; P-85 to I-99; M-86 to V-100; D-87 to Q-101; T-88 to N-102; L-89 to N-103; K-90 to L-104; V-91 to L-105; C-92 to Y-106; V-93 to V-107; P-94 to S-108; S-95 to A-109; L-96 to S-110; V-97 to H-111; Y-98 to L-112; I-99 to D-113; V-100 to A-114; Q-101 to A-115; N-102 to T-116; N-103 to Y-117; L-104 to Q-118; L-105 to V-119; Y-106 to T-120; V-107 to Y-121; S-108 to Q-122; A-109 to L-123; S-110 to K-124; H-111 to I-125; L-112 to L-126; D-113 to T-127; A-114 to T-128; A-115 to A-129; T-116 to M-130; Y-117 to F-131; Q-118 to A-132; V-119 to V-133; T-120 to V-134; Y-121 to I-135; Q-122 to L-136; L-123 to R-137; K-124 to R-138; I-125 to K-139; L-126 to L-140; T-127 to L-141; T-128 to N-142; A-129 to T-143; M-130 to Q-144; F-131 to W-145; A-132 to G-146; V-133 to A-147; V-134 to L-148; I-135 to L-149; L-136 to L-150; R-137 to L-151; R-138 to V-152; K-139 to M-153; L-140 to G-154; L-141 to I-155; N-142 to V-156; T-143 to L-157; Q-144 to V-158; W-145 to Q-159; G-146 to L-160; A-147 to A-161; L-148 to Q-162; L-149 to T-163; L-150 to E-164; L-151 to G-165; V-152 to P-166; M-153 to T-167; G-154 to S-168; I-155 to G-169; V-156 to S-170; L-157 to A-171; V-158 to G-172; Q-159 to G-173; L-160 to A-174; A-161 to A-175; Q-162 to A-176; T-163 to A-177; E-164 to A-178; G-165 to T-179; P-166 to A-180; T-167 to A-181; S-168 to S-182; G-169 to S-183; S-170 to G-184; A-171 to G-185; G-172 to A-186; G-173 to P-187; A-174 to E-188; A-175 to Q-189; A-176 to N-190; A-177 to R-191; A-178 to M-192; T-179 to L-193; A-180 to G-194; A-181 to L-195; S-182 to W-196; S-183 to A-197; G-184 to A-198; G-185 to L-199; A-186 to G-200; P-187 to A-201; E-188 to C-202; Q-189 to F-203; N-190 to L-204; R-191 to S-205; M-192 to G-206; L-193 to F-207; G-194 to A-208; L-195 to G-209; W-196 to I-210; A-197 to Y-211; A-198 to F-212; L-199 to E-213; G-200 to K-214; A-201 to I-215; C-202 to L-216; F-203 to K-217; L-204 to G-218; S-205 to A-219; G-206 to E-220; F-207 to I-221; A-208 to S-222; G-209 to V-223; I-210 to W-224; Y-211 to M-225; F-212 to R-226; E-213 to N-227; K-214 to V-228; I-215 to Q-229; L-216 to L-230; K-217 to S-231; G-218 to L-232; A-219 to L-233; E-220 to S-234; I-221 to I-235; S-222 to P-236; V-223 to F-237; W-224 to G-238; M-225 to L-239; R-226 to L-240; N-227 to T-241; V-228 to C-242; Q-229 to F-243; L-230 to V-244; S-231 to N-245; L-232 to D-246; L-233 to G-247; S-234 to S-248; I-235 to R-249; P-236 to I-250; F-237 to F-251; G-238 to D-252; L-239 to Q-253; L-240 to G-254; T-241 to F-255; C-242 to F-256; F-243 to K-257; V-244 to G-258; N-245 to Y-259; D-246 to D-260; G-247 to L-261; S-248 to F-262; R-249 to V-263; I-250 to W-264; F-251 to Y-265; D-252 to L-266; Q-253 to V-267; G-254 to L-268; F-255 to L-269; F-256 to Q-270; K-257 to A-271; G-258 to G-272; Y-259 to G-273; D-260 to G-274; L-261 to L-275; F-262 to I-276; V-263 to V-277; W-264 to A-278; Y-265 to V-279; L-266 to V-280; V-267 to V-281; L-268 to K-282; L-269 to Y-283; Q-270 to A-284; A-271 to D-285; G-272 to N-286; G-273 to I-287; G-274 to L-288; L-275 to K-289; I-276 to G-290; V-277 to F-291; A-278 to A-292; V-279 to T-293; V-280 to S-294; V-281 to L-295; K-282 to A-296; Y-283 to I-297; A-284 to I-298; D-285 to I-299; N-286 to S-300; I-287 to C-301; L-288 to V-302; K-289 to A-303; G-290 to S-304; F-291 to I-305; A-292 to Y-306; T-293 to I-307; S-294 to F-308; L-295 to D-309; A-296 to F-310; I-297 to N-311; I-298 to L-312; I-299 to T-313; S-300 to L-314; C-301 to Q-315; V-302 to F-316; A-303 to S-317; S-304 to F-318; I-305 to G-319; Y-306 to A-320; I-307 to G-321; F-308 to L-322; D-309 to V-323; F-310 to I-324; N-311 to A-325; L-312 to S-326; T-313 to I-327; L-314 to F-328; Q-315 to L-329; F-316 to Y-330; S-317 to G-331; F-318 to Y-332; G-319 to D-333; A-320 to P-334; G-321 to A-335; L-322 to R-336; V-323 to S-337; I-324 to A-338; A-325 to P-339; S-326 to K-340; I-327 to P-341; F-328 to T-342; L-329 to M-343; Y-330 to H-344; G-331 to G-345; Y-332 to P-346; D-333 to G-347; P-334 to G-348; A-335 to D-349; R-336 to E-350; S-337 to E-351; A-338 to K-352; P-339 to L-353; K-340 to L-354; P-341 to P-355; T-342 to R-356; and/or M-343 to V-357. These polypeptide fragments may retain the biological activity of CMP-SAT (SEQ ID NO:2) polypeptides of the invention and/or may be useful to generate or screen for antibodies, as described further below. Polynucleotides encoding these polypeptide fragments are also encompassed by the invention.

The present invention is also directed to polynucleotide fragments of the polynucleotides of Hex-1 (SEQ ID NO:3). In the present invention, a “polynucleotide fragment” refers to a short polynucleotide having a nucleic acid sequence which: is a portion of that contained in the deposited clone, or encoding the polypeptide encoded by the cDNA in the deposited clone; is a portion of that shown in SEQ ID NO:3 or the complementary strands thereto, or is a portion of the polynucleotide sequence encoding the polypeptide of SEQ ID NO:4. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the cDNA sequence contained in a deposited clone or the nucleotide sequence shown in SEQ ID NO:3. In this context “about” includes the particularly recited value, a value larger or smaller by several (5, 4, 3, 2, or 1) nucleotides, at either terminus or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, 2000-2050, or 2051 to the end of SEQ ID NO:3, or the complementary strand thereto, or the cDNA contained in the deposited clone. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Polynucleotides which hybridize to these nucleic acid molecules under stringent hybridization conditions or lower stringency conditions are also encompassed by the invention, as are polypeptides encoded by these polynucleotides.In the present invention, a “polypeptide fragment” refers to an amino acid sequence which is a portion of that contained in SEQ ID NO:4 or encoded by the cDNA contained in the deposited clone. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Even if deletion of one or more amino acids from the N-terminus of a protein results in modification or loss of one or more biological functions of the protein, other functional activities (e.g., biological activities, ability to multimerize, ability to bind substrates) may still be retained. For example, the ability of shortened Hex-1 (SEQ ID NO:4) muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a Hex-1 (SEQ ID NO:4) mutein with a large number of deleted N-terminal amino acid residues may retain some biological or immunogenic activities. In fact, peptides composed of as few as six Hex-1 (SEQ ID NO:4) amino acid residues may elicit an immune response.

Preferred polypeptide fragments include the full-length protein as well as the mature form. Further preferred polypeptide fragments include the full-length protein or the mature form having a continuous series of deleted residues from the amino or the carboxy termini, or both.

The present invention also provides for polypeptide fragments of the full-length Hex-1 protein as well as mature forms. Further preferred polypeptide fragments include the mature forms of Hex-1 having a continuous series of deleted residues from the amino or the carboxy terminus, or both. For example, any number of amino acids, ranging from 1-600, can be deleted from the amino terminus of either the secreted TGF alpha HIII polypeptide or the mature form. Similarly, any number of amino acids, ranging from 1-600, can be deleted from the carboxy terminus of the secreted TGF alpha HIII protein or mature form. Furthermore, any combination of the above amino and carboxy terminus deletions are preferred. Similarly,polynucleotides encoding these polypeptide fragments are also preferred.

Particularly, N-terminal deletions of the Hex-1 (SEQ ID NO:4) polypeptide can be described by the general formula m²-606, where m² is an integer from 2 to 600, 2 where m² corresponds to the position of the amino acid residue identified in SEQ ID NO:4. More in particular, the invention provides polynucleotides encoding polypeptides comprising, or alternatively consisting of, the amino acid sequence of residues of N-terminal deletions of the Hex-1 polypeptide of the invention shown as SEQ ID NO:4 including polypeptides comprising the amino acid sequence of residues: K-2 to V-606; S-3 to V-606; T-4 to V-606; R-5 to V-606; T-6 to V-606; A-7 to V-606; L-8 to V-606; G-9 to V-606; V-10 to V-606; A-11 to V-606; L-12 to V-606; L-13 to V-606; L-14 to V-606; A-15 to V-606; L-16 to V-606; V-17 to V-606; S-18 to V-606; Q-19 to V-606; L-20 to V-606; A-21 to V-606; A-22 to V-606; H-23 to V-606; S-24 to V-606; S-25 to V-606; D-26 to V-606; D-27 to V-606; L-28 to V-606; V-29 to V-606; Y-30 to V-606; G-31 to V-606; Y-32 to V-606; E-33 to V-606; C-34 to V-606; R-35 to V-606; S-36 to V-606; G-37 to V-606; Y-38 to V-606; C-39 to V-606; Q-40 to V-606; K-41 to V-606; V-42 to V-606; E-43 to V-606; L-44 to V-606; S-45 to V-606; E-46 to V-606; E-47 to V-606; N-48 to V-606; Y-49 to V-606; V-50 to V-606; K-51 to V-606; A-52 to V-606; I-53 to V-606; S-54 to V-606; L-55 to V-606; P-56 to V-606; V-57 to V-606; C-58 to V-606; R-59 to V-606; L-60 to V-606; F-61 to V-606; C-62 to V-606; G-63 to V-606; S-64 to V-606; S-65 to V-606; I-66 to V-606; G-67 to V-606; T-68 to V-606; L-69 to V-606; W-70 to V-606; P-71 to V-606; K-72 to V-606; P-73 to V-606; T-74 to V-606; G-75 to V-606; T-76 to V-606; V-77 to V-606; R-78 to V-606; L-79 to V-606; D-80 to V-606; T-81 to V-606; L-82 to V-606; M-83 to V-606; R-84 to V-606; Q-85 to V-606; V-86 to V-606; D-87 to V-606; I-88 to V-606; S-89 to V-606; F-90 to V-606; I-91 to V-606; D-92 to V-606; F-93 to V-606; N-94 to V-606; F-95 to V-606; N-96 to V-606; G-97 to V-606; I-98 to V-606; A-99 to V-606; R-100 to V-606; Q-101 to V-606; Q-102 to V-606; K-103 to V-606; L-104 to V-606; W-105 to V-606; R-106 to V-606; A-107 to V-606; V-108 to V-606; E-109 to V-606; D-110 to V-606; R-111 to V-606; F-112 to V-606; M-113 to V-606; N-114 to V-606; M-115 to V-606; L-116 to V-606; E-117 to V-606; A-118 to V-606; Q-119 to V-606; I-120 to V-606; P-121 to V-606; D-122 to V-606; R-123 to V-606; K-124 to V-606; V-125 to V-606; L-126 to V-606; A-127 to V-606; R-128 to V-606; G-129 to V-606; G-130 to V-606; Y-131 to V-606; R-132 to V-606; M-133 to V-606; S-134 to V-606; V-135 to V-606; N-136 to V-606; I-137 to V-606; N-138 to V-606; T-139 to V-606; P-140 to V-606; D-141 to V-606; E-142 to V-606; P-143 to V-606; T-144 to V-606; P-145 to V-606; A-146 to V-606; R-147 to V-606; L-148 to V-606; T-149 to V-606; L-150 to V-606; D-151 to V-606; T-152 to V-606; D-153 to V-606; E-154 to V-606; S-155 to V-606; Y-156 to V-606; T-157 to V-606; L-158 to V-606; D-159 to V-606; I-160 to V-606; D-161 to V-606; T-162 to V-606; D-163 to V-606; A-164 to V-606; S-165 to V-606; G-166 to V-606; H-167 to V-606; V-168 to V-606; L-169 to V-606; A-170 to V-606; N-171 to V-606; I-172 to V-606; T-173 to V-606; A-174 to V-606; S-175 to V-606; N-176 to V-606; F-177 to V-606; F-178 to V-606; G-179 to V-606; A-180 to V-606; R-181 to V-606; H-182 to V-606; G-183 to V-606; L-184 to V-606; E-185 to V-606; T-186 to V-606; L-187 to V-606; A-188 to V-606; Q-189 to V-606; L-190 to V-606; I-191 to V-606; V-192 to V-606; Y-193 to V-606; D-194 to V-606; D-195 to V-606; I-196 to V-606; R-197 to V-606; R-198 to V-606; E-199 to V-606; V-200 to V-606; Q-201 to V-606; V-202 to V-606; T-203 to V-606; A-204 to V-606; N-205 to V-606; A-206 to V-606; T-207 to V-606; I-208 to V-606; N-209 to V-606; D-210 to V-606; A-211 to V-606; P-212 to V-606; V-213 to V-606; Y-214 to V-606; K-215 to V-606; W-216 to V-606; R-217 to V-606; G-218 to V-606; L-219 to V-606; L-220 to V-606; L-221 to V-606; D-222 to V-606; T-223 to V-606; S-224 to V-606; R-225 to V-606; N-226 to V-606; Y-227 to V-606; Y-228 to V-606; S-229 to V-606; V-230 to V-606; K-231 to V-606; S-232 to V-606; I-233 to V-606; K-234 to V-606; R-235 to V-606; T-236 to V-606; L-237 to V-606; E-238 to V-606; G-239 to V-606; M-240 to V-606; A-241 to V-606; L-242 to V-606; V-243 to V-606; K-244 to V-606; L-245 to V-606; N-246 to V-606; T-247 to V-606; F-248 to V-606; H-249 to V-606; W-250 to V-606; H-251 to V-606; I-252 to V-606; T-253 to V-606; D-254 to V-606; S-255 to V-606; H-256 to V-606; S-257 to V-606; F-258 to V-606; P-259 to V-606; L-260 to V-606; E-261 to V-606; V-262 to V-606; K-263 to V-606; K-264 to V-606; R-265 to V-606; P-266 to V-606; E-267 to V-606; L-268 to V-606; H-269 to V-606; K-270 to V-606; L-271 to V-606; G-272 to V-606; A-273 to V-606; Y-274 to V-606; S-275 to V-606; Q-276 to V-606; R-277 to V-606; Q-278 to V-606; V-279 to V-606; Y-280 to V-606; T-281 to V-606; R-282 to V-606; R-283 to V-606; D-284 to V-606; V-285 to V-606; A-286 to V-606; E-287 to V-606; V-288 to V-606; V-289 to V-606; E-290 to V-606; Y-291 to V-606; G-292 to V-606; R-293 to V-606; V-294 to V-606; R-295 to V-606; G-296 to V-606; I-297 to V-606; R-298 to V-606; V-299 to V-606; M-300 to V-606; P-301 to V-606; E-302 to V-606; F-303 to V-606; D-304 to V-606; A-305 to V-606; P-306 to V-606; A-307 to V-606; H-308 to V-606; V-309 to V-606; G-310 to V-606; E-311 to V-606; G-312 to V-606; W-313 to V-606; Q-314 to V-606; H-315 to V-606; K-316 to V-606; N-317 to V-606; M-318 to V-606; T-319 to V-606; A-320 to V-606; C-321 to V-606; F-322 to V-606; N-323 to V-606; A-324 to V-606; Q-325 to V-606; P-326 to V-606; W-327 to V-606; K-328 to V-606; S-329 to V-606; F-330 to V-606; C-331 to V-606; V-332 to V-606; E-333 to V-606; P-334 to V-606; P-335 to V-606; C-336 to V-606; G-337 to V-606; Q-338 to V-606; L-339 to V-606; D-340 to V-606; P-341 to V-606; T-342 to V-606; V-343 to V-606; N-344 to V-606; E-345 to V-606; M-346 to V-606; Y-347 to V-606; D-348 to V-606; V-349 to V-606; L-350 to V-606; E-351 to V-606; D-352 to V-606; I-353 to V-606; Y-354 to V-606; G-355 to V-606; T-356 to V-606; M-357 to V-606; F-358 to V-606; D-359 to V-606; Q-360 to V-606; F-361 to V-606; N-362 to V-606; P-363 to V-606; D-364 to V-606; I-365 to V-606; F-366 to V-606; H-367 to V-606; M-368 to V-606; G-369 to V-606; G-370 to V-606; D-371 to V-606; E-372 to V-606; V-373 to V-606; S-374 to V-606; T-375 to V-606; S-376 to V-606; C-377 to V-606; W-378 to V-606; N-379 to V-606; S-380 to V-606; S-381 to V-606; Q-382 to V-606; P-383 to V-606; I-384 to V-606; Q-385 to V-606; Q-386 to V-606; W-387 to V-606; M-388 to V-606; K-389 to V-606; K-390 to V-606; Q-391 to V-606; G-392 to V-606; W-393 to V-606; G-394 to V-606; L-395 to V-606; E-396 to V-606; T-397 to V-606; A-398 to V-606; D-399 to V-606; F-400 to V-606; M-401 to V-606; R-402 to V-606; L-403 to V-606; W-404 to V-606; G-405 to V-606; H-406 to V-606; F-407 to V-606; Q-408 to V-606; T-409 to V-606; E-410 to V-606; A-411 to V-606; L-412 to V-606; G-413 to V-606; R-414 to V-606; V-415 to V-606; D-416 to V-606; K-417 to V-606; V-418 to V-606; A-419 to V-606; N-420 to V-606; G-421 to V-606; T-422 to V-606; H-423 to V-606; T-424 to V-606; P-425 to V-606; I-426 to V-606; I-427 to V-606; L-428 to V-606; W-429 to V-606; T-430 to V-606; S-431 to V-606; G-432 to V-606; L-433 to V-606; T-434 to V-606; E-435 to V-606; E-436 to V-606; P-437 to V-606; F-438 to V-606; I-439 to V-606; D-440 to V-606; E-441 to V-606; Y-442 to V-606; L-443 to V-606; N-444 to V-606; P-445 to V-606; E-446 to V-606; R-447 to V-606; Y-448 to V-606; I-449 to V-606; I-450 to V-606; Q-451 to V-606; I-452 to V-606; W-453 to V-606: T-454 to V-606; T-455 to V-606; G-456 to V-606; V-457 to V-606; D-458 to V-606; P-459 to V-606; K-460 to V-606; V-461 to V-606; K-462 to V-606; K-463 to V-606; I-464 to V-606; L-465 to V-606; E-466 to V-606; R-467 to V-606; G-468 to V-606; Y-469 to V-606; K-470 to V-606; I-471 to V-606; I-472 to V-606; V-473 to V-606; S-474 to V-606; N-475 to V-606; Y-476 to V-606; D-477 to V-606; A-478 to V-606; L-479 to V-606; Y-480 to V-606; L-481 to V-606; D-482 to V-606; C-483 to V-606; G-484 to V-606; G-485 to V-606; A-486 to V-606; G-487 to V-606; W-488 to V-606; V-489 to V-606; T-490 to V-606; D-491 to V-606; G-492 to V-606; N-493 to V-606; N-494 to V-606; W-495 to V-606; C-496 to V-606; S-497 to V-606; P-498 to V-606; Y-499 to V-606; I-500 to V-606; G-501 to V-606; W-502 to V-606; Q-503 to V-606; K-504 to V-606; V-505 to V-606; Y-506 to V-606; D-507 to V-606; N-508 to V-606; S-509 to V-606; L-510 to V-606; K-511 to V-606; S-512 to V-606; I-513 to V-606; A-514 to V-606; G-515 to V-606; D-516 to V-606; Y-517 to V-606; E-518 to V-606; H-519 to V-606; H-520 to V-606; V-521 to V-606; L-522 to V-606; G-523 to V-606; A-524 to V-606; E-525 to V-606; G-526 to V-606; A-527 to V-606; I-528 to V-606; W-529 to V-606; S-530 to V-606; E-531 to V-606; Q-532 to V-606; I-533 to V-606; D-534 to V-606; E-535 to V-606; H-536 to V-606; T-537 to V-606; L-538 to V-606; D-539 to V-606; N-540 to V-606; R-541 to V-606; F-542 to V-606; W-543 to V-606; P-544 to V-606; R-545 to V-606; A-546 to V-606; S-547 to V-606; A-548 to V-606; L-549 to V-606; A-550 to V-606; E-551 to V-606; R-552 to V-606; L-553 to V-606; W-554 to V-606; S-555 to V-606; N-556 to V-606; P-557 to V-606; A-558 to V-606; E-559 to V-606; G-560 to V-606; W-561 to V-606; R-562 to V-606; Q-563 to V-606; A-564 to V-606; E-565 to V-606; S-566 to V-606; R-567 to V-606; L-568 to V-606; L-569 to V-606; L-570 to V-606; H-571 to V-606; R-572 to V-606; Q-573 to V-606; R-574 to V-606; L-575 to V-606; V-576 to V-606; D-577 to V-606; N-578 to V-606; G-579 to V-606; L-580 to V-606; G-581 to V-606; A-582 to V-606; E-583 to V-606; A-584 to V-606; M-585 to V-606; Q-586 to V-606; P-587 to V-606; Q-588 to V-606; W-589 to V-606; C-590 to V-606; L-591 to V-606; Q-592 to V-606; N-593 to V-606; E-594 to V-606; H-595 to V-606; E-596 to V-606; C-597 to V-606; P-598 to V-606; I-599 to V-606; D-600 to V-606; and/or A-601 to V-606 of SEQ ID NO:4. Polynucleotides encoding these polypeptides are also encompassed by the invention.

Also as mentioned above, even if deletion of one or more amino acids from the C-terminus of a protein results in modification or loss of one or more biological functions of the protein, other functional activities (e.g., biological activities, ability to multimerize, ability to bind substrate) may still be retained. For example the ability of the shortened Hex-1 (SEQ ID NO:4) mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a Hex-1 mutein with a large number of deleted C-terminal amino acid residues may retain some biological or immunogenic activities. In fact, peptides composed of as few as six Hex-1 amino acid residues may elicit an immune response. Accordingly, the present invention further provides polypeptides having one or more residues deleted from the carboxy terminus of the amino acid sequence of the Hex-1 polypeptide shown in FIG. 2 (SEQ ID NO:4), as described by the general formula 1-n², where n² is an integer from 6 to 600, where n² corresponds to the position of amino acid residue identified in SEQ ID NO:4. More in particular, the invention provides polynucleotides encoding polypeptides comprising, or alternatively consisting of, the amino acid sequence of residues of C-terminal deletions of the Hex-1 polypeptide of the invention shown as SEQ ID NO:4 including polypeptides comprising the amino acid sequence of residues: M-1 to Q-605; M-1 to A-604; M-1 to D-603; M-1 to Y-602; M-1 to A-601; M-1 to D-600; M-1 to I-599; M-1 to P-598; M-1 to C-597; M-1 to E-596; M-1 to H-595; M-1 to E-594; M-1 to N-593; M-1 to Q-592; M-1 to L-591; M-1 to C-590; M-1 to W-589; M-1 to Q-588; M-1 to P-587; M-1 to Q-586; M-1 to M-585; M-1 to A-584; M-1 to E-583; M-1 to A-582; M-1 to G-581; M-1 to L-580; M-1 to G-579; M-1 to N-578; M-1 to D-577; M-1 to V-576; M-1 to L-575; M-1 to R-574; M-1 to Q-573; M-1 to R-572; M-1 to H-571; M-1 to L-570; M-1 to L-569; M-1 to L-568; M-1 to R-567; M-1 to S-566; M-1 to E-565; M-1 to A-564; M-1 to Q-563; M-1 to R-562; M-1 to W-561; M-1 to G-560; M-1 to E-559; M-1 to A-558; M-1 to P-557; M-1 to N-556; M-1 to S-555; M-1 to W-554; M-1 to L-553; M-1 to R-552; M-1 to E-551; M-1 to A-550; M-1 to L-549; M-1 to A-548; M-1 to S-547; M-1 to A-546; M-1 to R-545; M-1 to P-544; M-1 to W-543; M-1 to F-542; M-1 to R-541; M-1 to N-540; M-1 to D-539; M-1 to L-538; M-1 to T-537; M-1 to H-536; M-1 to E-535; M-1 to D-534; M-1 to I-533; M-1 to Q-532; M-1 to E-531; M-1 to S-530; M-1 to W-529; M-1 to I-528; M-1 to A-527; M-1 to G-526; M-1 to E-525; M-1 to A-524; M-1 to G-523; M-1 to L-522; M-1 to V-521; M-1 to H-520; M-1 to H-519; M-1 to E-518; M-1 to Y-517; M-1 to D-516; M-1 to G-515; M-1 to A-514; M-1 to I-513; M-1 to S-512; M-1 to K-511; M-1 to L-510; M-1 to S-509; M-1 to N-508; M-1 to D-507; M-1 to Y-506; M-1 to V-505; M-1 to K-504; M-1 to Q-503; M-1 to W-502; M-1 to G-501; M-1 to I-500; M-1 to Y-499; M-1 to P-498; M-1 to S-497; M-1 to C-496; M-1 to W-495; M-1 to N-494; M-1 to N-493; M-1 to G-492; M-1 to D-491; M-1 to T-490; M-1 to V-489; M-1 to W-488; M-1 to G-487; M-1 to A-486; M-1 to G-485; M-1 to G-484; M-1 to C-483; M-1 to D-482; M-1 to L-481; M-1 to Y-480; M-1 to L-479; M-1 to A-478; M-1 to D-477; M-1 to Y-476; M-1 to N-475; M-1 to S-474; M-1 to V-473; M-1 to I-472; M-1 to I-471; M-1 to K-470; M-1 to Y-469; M-1 to G-468; M-1 to R-467; M-1 to E-466; M-1 to L-465; M-1 to I-464; M-1 to K-463; M-1 to K-462; M-1 to V-461; M-1 to K-460; M-1 to P-459; M-1 to D-458; M-1 to V-457; M-1 to G-456; M-1 to T-455; M-1 to T-454; M-1 to W-453; M-1 to I-452; M-1 to Q-451; M-1 to I-450; M-1 to I-449; M-1 to Y-448; M-1 to R-447; M-1 to E-446; M-1 to P-445; M-1 to N-444; M-1 to L-443; M-1 to Y-442; M-1 to E-441; M-1 to D-440; M-1 to I-439; M-1 to F-438; M-1 to P-437; M-1 to E-436; M-1 to E-435; M-1 to T-434; M-1 to L-433; M-1 to G-432; M-1 to P-431; M-1 to T-430; M-1 to W-429; M-1 to L-428; M-1 to I-427; M-1 to I-426; M-1 to P-425; M-1 to T-424; M-1 to H-423; M-1 to T-422; M-1 to G-421; M-1 to N-420; M-1 to A-419; M-1 to V-418; M-1 to K-417; M-1 to D-416; M-1 to V-415; M-1 to R-414; M-1 to G-413; M-1 to L-412; M-1 to A-411; M-1 to E-410; M-1 to T-409; M-1 to Q-408; M-1 to F-407; M-1 to H-406; M-1 to G-405; M-1 to W-404; M-1 to L-403; M-1 to R-402; M-1 to M-401; M-1 to F-400; M-1 to D-399; M-1 to A-398; M-1 to T-397; M-1 to E-396; M-1 to L-395; M-1 to G-394; M-1 to W-393; M-1 to G-392; M-1 to Q-391; M-1 to K-390; M-1 to K-389; M-1 to M-388; M-1 to W-387; M-1 to Q-386; M-1 to Q-385; M-1 to I-384; M-1 to P-383; M-1 to Q-382; M-1 to S-381; M-1 to S-380; M-1 to N-379; M-1 to W-378; M-1 to C-377; M-1 to S-376; M-1 to T-375; M-1 to S-374; M-1 to V-373; M-1 to E-372; M-1 to D-371; M-1 to G-370; M-1 to G-369; M-1 to M-368; M-1 to H-367; M-1 to F-366; M-1 to I-365; M-1 to D-364; M-1 to P-363; M-1 to N-362; M-1 to F-361; M-1 to Q-360; M-1 to D-359; M-1 to F-358; M-1 to M-357; M-1 to T-356; M-1 to G-355; M-1 to Y-354; M-1 to I-353; M-1 to D-352; M-1 to E-351; M-1 to L-350; M-1 to V-349; M-1 to D-348; M-1 to Y-347; M-1 to M-346; M-1 to E-345; M-1 to N-344; M-1 to V-343; M-1 to T-342; M-1 to P-341; M-1 to D-340; M-1 to L-339; M-1 to Q-338; M-1 to G-337; M-1 to C-336; M-1 to P-335; M-1 to P-334; M-1 to E-333; M-1 to V-332; M-1 to C-331; M-1 to F-330; M-1 to S-329; M-1 to K-328; M-1 to W-327; M-1 to P-326; M-1 to Q-325; M-1 to A-324; M-1 to N-323; M-1 to F-322; M-1 to C-321; M-1 to A-320; M-1 to T-319; M-1 to M-318; M-1 to N-317; M-1 to K-316; M-1 to H-315; M-1 to Q-314; M-1 to W-313; M-1 to G-312; M-1 to E-311; M-1 to G-310; M-1 to V-309; M-1 to H-308; M-1 to A-307; M-1 to P-306; M-1 to A-305; M-1 to D-304; M-1 to F-303; M-1 to E-302; M-1 to P-301; M-1 to M-300; M-1 to V-299; M-1 to R-298; M-1 to I-297; M-1 to G-296; M-1 to R-295; M-1 to V-294; M-1 to R-293; M-1 to G-292; M-1 to Y-291; M-1 to E-290; M-1 to V-289; M-1 to V-288; M-1 to E-287; M-1 to A-286; M-1 to V-285; M-1 to D-284; M-1 to R-283; M-1 to R-282; M-1 to T-281; M-1 to Y-280; M-1 to V-279; M-1 to Q-278; M-1 to R-277; M-1 to Q-276; M-1 to S-275; M-1 to Y-274; M-1 to A-273; M-1 to G-272; M-1 to L-271; M-1 to K-270; M-1 to H-269; M-1 to L-268; M-1 to E-267; M-1 to P-266; M-1 to R-265; M-1 to K-264; M-1 to K-263; M-1 to V-262; M-1 to E-261; M-1 to L-260; M-1 to P-259; M-1 to F-258; M-1 to S-257; M-1 to H-256; M-1 to S-255; M-1 to D-254; M-1 to T-253; M-1 to I-252; M-1 to H-251; M-1 to W-250; M-1 to H-249; M-1 to F-248; M-1 to T-247; M-1 to N-246; M-1 to L-245; M-1 to K-244; M-1 to V-243; M-1 to L-242; M-1 to A-241; M-1 to M-240; M-1 to G-239; M-1 to E-238; M-1 to L-237; M-1 to T-236; M-1 to R-235; M-1 to K-234; M-1 to I-233; M-1 to S-232; M-1 to K-231; M-1 to V-230; M-1 to S-229; M-1 to Y-228; M-1 to Y-227; M-1 to N-226; M-1 to R-225; M-1 to S-224; M-1 to T-223; M-1 to D-222; M-1 to L-221; M-1 to L-220; M-1 to L-219; M-1 to G-218; M-1 to R-217; M-1 to W-216; M-1 to K-215; M-1 to Y-214; M-1 to V-213; M-1 to P-212; M-1 to A-211; M-1 to D-210; M-1 to N-209; M-1 to I-208; M-1 to T-207; M-1 to A-6; M-1 to N-205; M-1 to A-204; M-1 to T-203; M-1 to V-202; M-1 to Q-201; M-1 to V-200; M-1 to E-199; M-1 to R-198; M-1 to R-197; M-1 to I-196; M-1 to D-195; M-1 to D-194; M-1 to Y-193; M-1 to V-192; M-1 to I-191; M-1 to L-190; M-1 to Q-189; M-1 to A-288; M-1 to L-187; M-1 to T-186; M-1 to E-185; M-1 to L-184; M-1 to G-183; M-1 to H-182; M-1 to R-181; M-1 to A-180; M-1 to G-179; M-1 to F-178; M-1 to F-177; M-1 to N-176; M-1 to S-175; M-1 to A-174; M-1 to T-173; M-1 to I-172; M-1 to N-171; M-1 to A-170; M-1 to L-169; M-1 to V-168; M-1 to H-167; M-1 to G-166; M-1 to S-165; M-1 to A-164; M-1 to D-163; M-1 to T-162; M-1 to D-161; M-1 to I-160; M-1 to D-159; M-1 to L-158; M-1 to T-157; M-1 to Y-156; M-1 to S-155; M-1 to E-154; M-1 to D-153; M-1 to T-152; M-1 to D-151; M-1 to L-150; M-1 to T-149; M-1 to L-148; M-1 to R-147; M-1 to A-146; M-1 to P-145; M-1 to T-144; M-1 to P-143; M-1 to E-142; M-1 to D-141; M-1 to P-140; M-1 to T-139; M-1 to N-138; M-1 to I-137; M-1 to N-136; M-1 to V-135; M-1 to S-134; M-1 to M-133; M-1 to R-132; M-1 to Y-131; M-1 to G-130; M-1 to G-129; M-1 to R-128; M-1 to A-127; M-1 to L-126; M-1 to V-125; M-1 to K-124; M-1 to R-123; M-1 to D-122; M-1 to P-121; M-1 to I-120; M-1 to Q-119; M-1 to A-118; M-1 to E-117; M-1 to L-116; M-1 to M-115; M-1 to N-114; M-1 to M-113; M-1 to F-112; M-1 to R111; M-1 to D-110; M-1 to E-109; M-1 to V-108; M-1 to A-107; M-1 to R-106; M-1 to W-105; M-1 to L-104; M-1 to K-103; M-1 to Q-102; M-1 to Q-101; M-1 to R-100; M-1 to A-99; M-1 to I-98; M-1 to G-97; M-1 to N-96; M-1 to F-95; M-1 to N-94; M-1 to F-93; M-1 to D-92; M-1 to I-91; M-1 to F-90; M-1 to S-89; M-1 to I-88; M-1 to D-87; M-1 to V-86; M-1 to Q-85; M-1 to R-84; M-1 to M-83; M-1 to L-82; M-1 to T-81; M-1 to D-80; M-1 to L-79; M-1 to R-78; M-1 to V-77; M-1 to T-76; M-1 to G-75; M-1 to T-74; M-1 to P-73; M-1 to K-72; M-1 to P-71; M-1 to W-70; M-1 to L-69; M-1 to T-68; M-1 to G-67; M-1 to I-66; M-1 to S-65; M-1 to S-64; M-1 to G-63; M-1 to C-62; M-1 to F-61; M-1 to L-60; M-1 to R-59; M-1 to C-58; M-1 to V-57; M-1 to P-56; M-1 to L-55; M-1 to S-54; M-1 to I-53; M-1 to A-52; M-1 to K-51; M-1 to V-50; M-1 to Y-49; M-1 to N-48; M-1 to E-47; M-1 to E-46; M-1 to S-45; M-1 to L-44; M-1 to E-43; M-1 to V-42; M-1 to K-41; M-1 to Q-40; M-1 to C-39; M-1 to Y-38; M-1 to G-37; M-1 to S-36; M-1 to R-35; M-1 to C-34; M-1 to E-33; M-1 to Y-32; M-1 to G-31; M-1 to Y-30; M-1 to V-29; M-1 to L-28; M-1 to D-27; M-1 to D-26; M-1 to S-25; M-1 to S-24; M-1 to H-23; M-1 to A-22; M-1 to A-21; M-1 to L-20; M-1 to Q-19; M-1 to S-18; M-1 to V-17; M-1 to L-16; M-1 to A-15; M-1 to L-14; M-1 to L-13; M-1 to L-12; M-1 to A-11; M-1 to V10; M-1 to G-9; M-1 to L-8;and/or M-1 to A-7 of SEQ ID NO:4. Polynucleotides encoding these polypeptides are also encompassed by the invention.

Moreover, a signal sequence may be added to these C-terminal contructs. For example, amino acids I-23 of SEQ ID NO:4, amino acids 2-23 of SEQ ID NO:4, amino acids 3-23 of SEQ ID NO:4, amino acids 4-23 of SEQ ID NO:4, amino acids 5-23 of SEQ ID NO:4, amino acids 6-23 of SEQ ID NO:4, amino acids 7-23 of SEQ ID NO:4, amino acids 8-23 of SEQ ID NO:4, amino acids 9-23 of SEQ ID NO:4, amino acids 10-23 of SEQ ID NO:4, amino acids 11-23 of SEQ ID NO:4, amino acids 12-23 of SEQ ID NO:4, amino acids 13-23 of SEQ ID NO:4, amino acids 14-23 of SEQ ID NO:4, amino acids 15-23 of SEQ ID NO:4, amino acids 16-23 of SEQ ID NO:4, amino acids 17-23 of SEQ ID NO:4, amino acids 18-23 of SEQ ID NO:4, amino acids 19-23 of SEQ ID NO:4, amino acids 20-23 of SEQ ID NO:4, amino acids 21-23 of SEQ ID NO:4, amino acids 22-23 of SEQ ID NO:4, amino acids 23-23 of SEQ ID NO:4, amino acids 24-23 of SEQ ID NO:4, amino acids 25-23 of SEQ ID NO:4, amino acids 26-23 of SEQ ID NO:4, amino acids 27-23 of SEQ ID NO:4, amino acids 28-23 of SEQ ID NO:4, or amino acids 29-23 of SEQ ID NO:4 can be added to the N-terminus of each C-terminal constructs listed above.

In addition, any of the above listed N- or C-terminal deletions can be combined to produce a N- and C-terminal deleted Hex-1 (SEQ ID NO:4) polypeptide. The invention also provides polypeptides having one or more amino acids deleted from both the amino and the carboxyl termini, which may be described generally as having residues m²-n² of SEQ ID NO:4, where n² and m² are integers as described above. Polynucleotides encoding these polypeptides are also encompassed by the invention.

Also included are a nucleotide sequence encoding a polypeptide consisting of a portion of the complete Hex-1 amino acid sequence encoded by the cDNA clone contained in ATCC Deposit No. ______ (as shown in SEQ ID NO:3 and SEQ ID NO:4), where this portion excludes any integer of amino acid residues from 1 to about 600 amino acids from the amino terminus of the complete amino acid sequence encoded by the cDNA clone contained in the ATCC Deposit No. ______, or any integer of amino acid residues from 1 to 600 amino acids from the carboxy terminus, or any combination of the above amino terminal and carboxy terminal deletions, of the complete amino acid sequence encoded by the cDNA clone contained in the ATCC Deposit No. ______. Polynucleotides encoding all of the above deletion mutant polypeptide forms also are provided.

The present application is also directed to proteins containing polypeptides at least 90%, 95%, 96%, 97%, 98% or 99% identical to the Hex-1 polypeptide sequence set forth herein. In preferred embodiments, the application is directed to proteins containing polypeptides at least 90%, 95%, 96%, 97%, 98% or 99% identical to polypeptides having the amino acid sequence of the specific Hex-1 N- and C-terminal deletions recited herein. Polynucleotides encoding these polypeptides are also encompassed by the invention. Additional preferred Hex-1 (SEQ ID NO:4) polypeptide fragments comprise, or alternatively consist of, the amino acid sequence of residues: M-1 to A-15; K-2 to L-16; S-3 to V-17; T-4 to S-18; R-5 to Q-19; T-6 to L-20; A-7 to A-21; L-8 to A-22; G-9 to H-23; V-10 to S-24; A-11 to S-25; L-12 to D-26; L-13 to D-27; L-14 to L-28; A-15 to V-29; L-16 to Y-30; V-17 to G-31; S-18 to Y-32; Q-19 to E-33; L-20 to C-34; A-21 to R-35; A-22 to S-36; H-23 to G-37; S-24 to Y-38; S-25 to C-39; D-26 to Q-40; D-27 to K-41; L-28 to V-42; V-29 to E-43; Y-30 to L-44; G-31 to S-45; Y-32 to E-46; E-33 to E-47; C-34 to N-48; R-35 to Y-49; S-36 to V-50; G-37 to K-51; Y-38 to A-52; C-39 to I-53; Q-40 to S-54; K-41 to L-55; V-42 to P-56; E-43 to V-57; L-44 to C-58; S-45 to R-59; E-46 to L-60; E-47 to F-61; N-48 to C-62; Y-49 to G-63; V-50 to S-64; K-51 to S-65; A-52 to I-66; I-53 to G-67; S-54 to T-68; L-55 to L-69; P-56 to W-70; V-57 to P-71; C-58 to K-72; R-59 to P-73; L-60 to T-74; F-61 to G-75; C-62 to T-76; G-63 to V-77; S-64 to R-78; S-65 to L-79; I-66 to D-80; G-67 to T-81; T-68 to L-82; L-69 to M-83; W-70 to R-84; P-71 to Q-85; K-72 to V-86; P-73 to D-87; T-74 to I-88; G-75 to S-89; T-76 to F-90; V-77 to I-91; R-78 to D-92; L-79 to F-93; D-80 to N-94; T-81 to F-95; L-82 to N-96; M-83 to G-97; R-84 to I-98; Q-85 to A-99; V-86 to R-100; D-87 to Q-101; I-88 to Q-102; S-89 to K-103; F-90 to L-104; I-91 to W-105; D-92 to R-106; F-93 to A-107; N-94 to V-108; F-95 to E-109; N-96 to D-110; G-97 to R-111; I-98 to F-112; A-99 to M-113; R-100 to N-114; Q-101 to M-115; Q-102 to L-116; K-103 to E-117; L-104 to A-118; W-105 to Q-119; R-106 to I-120; A-107 to P-121; V-108 to D-122; E-109 to R-123; D-110 to K-124; R-111 to V-125; F-112 to L-126; M-113 to A-127; N-114 to R-128; M-115 to G-129; L-116 to G-130; E-117 to Y-131; A-118 to R-132; Q-119 to M-133; I-120 to S-134; P-121 to V-135; D-122 to N-136; R-123 to I-137; K-124 to N-138; V-125 to T-139; L-126 to P-140; A-127 to D-141; R-128 to E-142; G-129 to P-143; G-130 to T-144; Y-131 to P-145; R-132 to A-146; M-133 to R-147; S-134 to L-148; V-135 to T-149; N-136 to L-150; I-137 to D-151; N-138 to T-152; T-139 to D-153; P-140 to E-154; D-141 to S-155; E-142 to Y-156; P-143 to T-157; T-144 to L-158; P-145 to D-159; A-146 to I-160; R-147 to D-161; L-148 to T-162; T-149 to D-163; L-150 to A-164; D-151 to S-165; T-152 to G-166; D-153 to H-167; E-154 to V-168; S-155 to L-169; Y-156 to A-170; T-157 to N-171; L-158 to I-172; D-159 to T-173; I-160 to A-174; D-161 to S-175; T-162 to N-176; D-163 to F-177; A-164 to F-178; S-165 to G-179; G-166 to A-180; H-167 to R-181; V-168 to H-182; L-169 to G-183; A-170 to L-184; N-171 to E-185; I-172 to T-186; T-173 to L-187; A-174 to A-188; S-175 to Q-189; N-176 to L-190; F-177 to I-191; F-178 to V-192; G-179 to Y-193; A-180 to D-194; R-181 to D-195; H-182 to I-196; G-183 to R-197; L-184 to R-198; E-185 to E-199; T-186 to V-200; L-187 to Q-201; A-188 to V-202; Q-189 to T-203; L-190 to A-204; I-191 to N-205; V-192 to A-206; Y-193 to T-207; D-194 to I-208; D-195 to N-209; I-196 to D-210; R-197 to A-211; R-198 to P-212; E-199 to V-213; V-200 to Y-214; Q-201 to K-215; V-202 to W-216; T-203 to R-217; A-204 to G-218; N-205 to L-219; A-206 to L-220; T-207 to L-221; I-208 to D-222; N-209 to T-223; D-210 to S-224; A-211 to R-225; P-212 to N-226; V-213 to Y-227; Y-214 to Y-228; K-215 to S-229; W-216 to V-230; R-217 to K-231; G-218 to S-232; L-219 to I-233; L-220 to K-234; L-221 to R-235; D-222 to T-236; T-223 to L-237; S-224 to E-238; R-225 to G-239; N-226 to M-240; Y-227 to A-241; Y-228 to L-242; S-229 to V-243; V-230 to K-244; K-231 to L-245; S-232 to N-246; I-233 to T-247; K-234 to F-248; R-235 to H-249; T-236 to W-250; L-237 to H-251; E-238 to I-252; G-239 to T-253; M-240 to D-254; A-241 to S-255; L-242 to H-256; V-243 to S-257; K-244 to F-258; L-245 to P-259; N-246 to L-260; T-247 to E-261; F-248 to V-262; H-249 to K-263; W-250 to K-264; H-251 to R-265; I-252 to P-266; T-253 to E-267; D-254 to L-268; S-255 to H-269; H-256 to K-270; S-257 to L-271; F-258 to G-272; P-259 to A-273; L-260 to Y-274; E-261 to S-275; V-262 to Q-276; K-263 to R-277; K-264 to Q-278; R-265 to V-279; P-266 to Y-280; E-267 to T-281; L-268 to R-282; H-269 to R-283; K-270 to D-284; L-271 to V-285; G-272 to A-286; A-273 to E-287; Y-274 to V-288; S-275 to V-289; Q-276 to E-290; R-277 to Y-291; Q-278 to G-292; V-279 to R-293; Y-280 to V-294; T-281 to R-295; R-282 to G-296; R-283 to I-297; D-284 to R-298; V-285 to V-299; A-286 to M-300; E-287 to P-301; V-288 to E-302; V-289 to F-303; E-290 to D-304; Y-291 to A-305; G-292 to P-306; R-293 to A-307; V-294 to H-308; R-295 to V-309; G-296 to G-310; I-297 to E-311; R-298 to G-312; V-299 to W-313; M-300 to Q-314; P-301 to H-315; E-302 to K-316; F-303 to N-317; D-304 to M-318; A-305 to T-319; P-306 to A-320; A-307 to C-321; H-308 to F-322; V-309 to N-323; G-310 to A-324; E-311 to Q-325; G-312 to P-326; W-313 to W-327; Q-314 to K-328; H-315 to S-329; K-316 to F-330; N-317 to C-331; M-318 to V-332; T-319 to E-333; A-320 to P-334; C-321 to P-335; F-322 to C-336; N-323 to G-337; A-324 to Q-338; Q-325 to L-339; P-326 to D-340; W-327 to P-341; K-328 to T-342; S-329 to V-343; F-330 to N-344; C-331 to E-345; V-332 to M-346; E-333 to Y-347; P-334 to D-348; P-335 to V-349; C-336 to L-350; G-337 to E-351; Q-338 to D-352; L-339 to I-353; D-340 to Y-354; P-341 to G-355; T-342 to T-356; V-343 to M-357; N-344 to F-358; E-345 to D-359; M-346 to Q-360; Y-347 to F-361; D-348 to N-362; V-349 to P-363; L-350 to D-364; E-351 to I-365; D-352 to F-366; I-353 to H-367; Y-354 to M-368; G-355 to G-369; T-356 to G-370; M-357 to D-371; F-358 to E-372; D-359 to V-373; Q-360 to S-374; F-361 to T-375; N-362 to S-376; P-363 to C-377; D-364 to W-378; I-365 to N-379; F-366 to S-380; H-367 to S-381; M-368 to Q-382; G-369 to P-383; G-370 to I-384; D-371 to Q-385; E-372 to Q-386; V-373 to W-387; S-374 to M-388; T-375 to K-389; S-376 to K-390; C-377 to Q-391; W-378 to G-392; N-379 to W-393; S-380 to G-394; S-381 to L-395; Q-382 to E-396; P-383 to T-397; I-384 to A-398; Q-385 to D-399; Q-386 to F-400; W-387 to M-401; M-388 to R-402; K-389 to L-403; K-390 to W-404; Q-391 to G-405; G-392 to H-406; W-393 to F-407; G-394 to Q-408; L-395 to T-409; E-396 to E-410; T-397 to A-411; A-398 to L-412; D-399 to G-413; F-400 to R-414; M-401 to V-415; R-402 to D-416; L-403 to K-417; W-404 to V-418; G-405 to A-419; H-406 to N-420; F-407 to G-421; Q-408 to T-422; T-409 to H-423; E-410 to T-424; A-411 to P-425; L-412 to I-426; G-413 to I-427; R-414 to L-428; V-415 to W-429; D-416 to T-430; K-417 to S-431; V-418 to G-432; A-419 to L-433; N-420 to T-434; G-421 to E-435; T-422 to E-436; H-423 to P-437; T-424 to F-438; P-425 to I-439; I-426 to D-440; I-427 to E-441; L-428 to Y-442; W-429 to L-443; T-430 to N-444; S-431 to P-445; G-432 to E-446; L-433 to R-447; T-434 to Y-448; E-435 to I-449; E-436 to I-450; P-437 to Q-451; F-438 to I-452; I-439 to W-453; D-440 to T-454; E-441 to T-455; Y-442 to G-456; L-443 to V-457; N-444 to D-458; P-445 to P-459; E-446 to K-460; R-447 to V-461; Y-448 to K-462; I-449 to K-463; I-450 to I-464; Q-451 to L-465; I-452 to E-466; W-453 to R-467; T-454 to G-468; T-455 to Y-469; G-456 to K-470; V-457 to I-471; D-458 to I-472; P-459 to V-473; K-460 to S-474; V-461 to N-475; K-462 to Y-476; K-463 to D-477; I-464 to A-478; L-465 to L-479; E-466 to Y-480; R-467 to L-481; G-468 to D-482; Y-469 to C-483; K-470 to G-484; I-471 to G-485; I-472 to A-486; V-473 to G-487; S-474 to W-488; N-475 to V-489; Y-476 to T-490; D-477 to D-491; A-478 to G-492; L-479 to N-493; Y-480 to N-494; L-481 to W-495; D-482 to C-496; C-483 to S-497; G-484 to P-498; G-485 to Y-499; A-486 to I-500; G-487 to G-501; W-488 to W-502; V-489 to Q-503; T-490 to K-504; D-491 to V-505; G-492 to Y-506; N-493 to D-507; N-494 to N-508; W-495 to S-509; C-496 to L-510; S-497 to K-511; P-498 to S-512; Y-499 to I-513; I-500 to A-514; G-501 to G-515; W-502 to D-516; Q-503 to Y-517; K-504 to E-518; V-505 to H-519; Y-506 to H-520; D-507 to V-521; N-508 to L-522; S-509 to G-523; L-510 to A-524; K-511 to E-525; S-512 to G-526; I-513 to A-527; A-514 to I-528; G-515 to W-529; D-516 to S-530; Y-517 to E-531; E-518 to Q-532; H-519 to I-533; H-520 to D-534; V-521 to E-535; L-522 to H-536; G-523 to T-537; A-524 to L-538; E-525 to D-539; G-526 to N-540; A-527 to R-541; I-528 to F-542; W-529 to W-543; S-530 to P-544; E-531 to R-545; Q-532 to A-546; I-533 to S-547; D-534 to A-548; E-535 to L-549; H-536 to A-550; T-537 to E-551; L-538 to R-552; D-539 to L-553; N-540 to W-554; R-541 to S-555; F-542 to N-556; W-543 to P-557; P-544 to A-558; R-545 to E-559; A-546 to G-560; S-547 to W-561; A-548 to R-562; L-549 to Q-563; A-550 to A-564; E-551 to E-565; R-552 to S-566; L-553 to R-567; W-554 to L-568; S-555 to L-569; N-556 to L-570; P-557 to H-571; A-558 to R-572; E-559 to Q-573; G-560 to R-574; W-561 to L-575; R-562 to V-576; Q-563 to D-577; A-564 to N-578; E-565 to G-579; S-566 to L-580; R-567 to G-581; L-568 to A-582; L-569 to E-583; L-570 to A-584; H-571 to M-585; R-572 to Q-586; Q-573 to P-587; R-574 to Q-588; L-575 to W-589; V-576 to C-590; D-577 to L-591; N-578 to Q-592; G-579 to N-593; L-580 to E-594; G-581 to H-595; A-582 to E-596; E-583 to C-597; A-584 to P-598; M-585 to I-599; Q-586 to D-600; P-587 to A-601; Q-588 to Y-602; W-589 to D-603; C-590 to A-604; L-591 to Q-605; and/or Q-592 to V-606. These polypeptide fragments may retain the biological activity of Hex-1 (SEQ ID NO:4) polypeptides of the invention and/or may be useful to generate or screen for antibodies, as described further below. Polynucleotides encoding these polypeptide fragments are also encompassed by the invention.

The present invention is also directed to polynucleotide fragments of the polynucleotides of Hex-2 (SEQ ID NO:5). In the present invention, a “polynucleotide fragment” refers to a short polynucleotide having a nucleic acid sequence which: is a portion of that contained in the deposited clone, or encoding the polypeptide encoded by the cDNA in the deposited clone; is a portion of that shown in SEQ ID NO:5 or the complementary strands thereto, or is a portion of the polynucleotide sequence encoding the polypeptide of SEQ ID NO:5. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the cDNA sequence contained in a deposited clone or the nucleotide sequence shown in SEQ ID NO:5. In this context “about” includes the particularly recited value, a value larger or smaller by several (5, 4, 3, 2, or 1) nucleotides, at either terminus or at both ternini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, 2001-2050, 2051-2100, or 2100 to the end of SEQ ID NO:5, or the complementary strand thereto, or the cDNA contained in the deposited clone. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Polynucleotides which hybridize to these nucleic acid molecules under stringent hybridization conditions or lower stringency conditions are also encompassed by the invention, as are polypeptides encoded by these polynucleotides. In the present invention, a “polypeptide fragment” refers to an amino acid sequence which is a portion of that contained in SEQ ID NO:6 or encoded by the cDNA contained in the deposited clone. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Even if deletion of one or more amino acids from the N-terminus of a protein results in modification or loss of one or more biological functions of the protein, other functional activities (e.g., biological activities, ability to multimerize, ability to bind substrates) may still be retained. For example, the ability of shortened Hex-2 (SEQ ID NO:6) muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a Hex-2 (SEQ ID NO:6) mutein with a large number of deleted N-terminal amino acid residues may retain some biological or immunogenic activities. In fact, peptides composed of as few as six Hex-2 (SEQ ID NO:6) amino acid residues may elicit an immune response.

Preferred polypeptide fragments include the fill-length protein as well as the mature form. Further preferred polypeptide fragments include the full-length protein or the mature form having a continuous series of deleted residues from the amino or the carboxy termini, or both.

Accordingly, polypeptide fragments include the full-length Hex-2 protein (SEQ ID NO:6) as well as mature forms. Further preferred polypeptide fragments include the mature forms of Hex-2 having a continuous series of deleted residues from the amino or the carboxy terminus, or both. For example, any number of amino acids, ranging from 1-616, can be deleted from the amino terminus of either the secreted TGF alpha HIII polypeptide or the mature form. Similarly, any number of amino acids, ranging from 1-616, can be deleted from the carboxy terminus of the secreted TGF alpha HIII protein or mature form. Furthermore, any combination of the above amino and carboxy terminus deletions are preferred. Similarly,polynucleotides encoding these polypeptide fragments are also preferred.

Particularly, N-terminal deletions of the Hex-2 (SEQ ID NO:6) polypeptide can be described by the general formula m³-616, where m³ is an integer from 2 to 616, where m² corresponds to the position of the amino acid residue identified in SEQ ID NO:6. More in particular, the invention provides polynucleotides encoding polypeptides comprising, or alternatively consisting of, the amino acid sequence of residues of N-terminal deletions of the Hex-2 polypeptide of the invention shown as SEQ ID NO:6 including polypeptides comprising the amino acid sequence of residues: R-2 to L-622; F-3 to L-622; S-4 to L-622; G-5 to L-622; Y-6 to L-622; N-7 to L-622; R-8 to L-622; Y-9 to L-622; Q-10 to L-622; C-11 to L-622; F-12 to L-622; C-13 to L-622; S-14 to L-622; A-15 to L-622; V-16 to L-622; G-17 to L-622; S-18 to L-622; L-19 to L-622; L-20 to L-622; L-21 to L-622; L-22 to L-622; S-23 to L-622; L-24 to L-622; L-25 to L-622; S-26 to L-622; F-27 to L-622; A-28 to L-622; V-29 to L-622; G-30 to L-622; A-31 to L-622; A-32 to L-622; L-33 to L-622; T-34 to L-622; R-35 to L-622; A-36 to L-622; D-37 to L-622; D-38 to L-622; V-39 to L-622; G-40 to L-622; S-41 to L-622; D-42 to L-622; A-43 to L-622; D-44 to L-622; A-45 to L-622; G-46 to L-622; S-47 to L-622; A-48 to L-622; S-49 to L-622; R-50 to L-622; K-51 to L-622; W-52 to L-622; L-53 to L-622; C-54 to L-622; S-55 to L-622; R-56 to L-622; T-57 to L-622; D-58 to L-622; I-59 to L-622; C-60 to L-622; T-61 to L-622; A-62 to L-622; E-63 to L-622; G-64 to L-622; E-65 to L-622; M-66 to L-622; V-67 to L-622; A-68 to L-622; G-69 to L-622; L-70 to L-622; Q-71 to L-622; Y-72 to L-622; A-73 to L-622; P-74 to L-622; E-75 to L-622; I-76 to L-622; F-77 to L-622; E-78 to L-622; S-79 to L-622; Q-80 to L-622; R-81 to L-622; D-82 to L-622; C-83 to L-622; R-84 to L-622; L-85 to L-622; S-86 to L-622; C-87 to L-622; G-88 to L-622; K-89 to L-622; Y-90 to L-622; G-91 to L-622; A-92 to L-622; I-93 to L-622; W-94 to L-622; P-95 to L-622; M-96 to L-622; P-97 to L-622; T-98 to L-622; G-99 to L-622; K-100 to L-622; E-101 to L-622; C-102 to L-622; T-103 to L-622; I-104 to L-622; S-105 to L-622; H-106 to L-622; R-107 to L-622; R-108 to L-622; V-109 to L-622; R-110 to L-622; F-111 to L-622; D-112 to L-622; P-113 to L-622; W-114 to L-622; K-115 to L-622; V-116 to L-622; R-117 to L-622; F-118 to L-622; H-119 to L-622; V-120 to L-622; V-121 to L-622; A-122 to L-622; P-123 to L-622; G-124 to L-622; E-125 to L-622; A-126 to L-622; A-127 to L-622; T-128 to L-622; Q-129 to L-622; F-130 to L-622; L-131 to L-622; R-132 to L-622; E-133 to L-622; T-134 to L-622; N-135 to L-622; R-136 to L-622; L-137 to L-622; F-138 to L-622; V-139 to L-622; S-140 to L-622; N-141 to L-622; L-142 to L-622; L-143 to L-622; K-144 to L-622; E-145 to L-622; C-146 to L-622; I-147 to L-622; R-148 to L-622; N-149 to L-622; C-150 to L-622; T-151 to L-622; L-152 to L-622; E-153 to L-622; T-154 to L-622; S-155 to L-622; K-156 to L-622; Q-157 to L-622; I-158 to L-622; L-159 to L-622; V-160 to L-622; R-161 to L-622; S-162 to L-622; T-163 to L-622; V-164 to L-622; A-165 to L-622; N-166 to L-622; E-167 to L-622; S-168 to L-622; L-169 to L-622; V-170 to L-622; L-171 to L-622; D-172 to L-622; W-173 to L-622; P-174 to L-622; T-175 to L-622; D-176 to L-622; E-177 to L-622; S-178 to L-622; Y-179 to L-622; A-180 to L-622; L-181 to L-622; V-182 to L-622; V-183 to L-622; R-184 to L-622; T-185 to L-622; T-186 to L-622; E-187 to L-622; T-188 to L-622; A-189 to L-622; T-190 to L-622; F-191 to L-622; V-192 to L-622; D-193 to L-622; I-194 to L-622; Q-195 to L-622; A-196 to L-622; T-197 to L-622; T-198 to L-622; V-199 to L-622; Y-200 to L-622; G-201 to L-622; A-202 to L-622; R-203 to L-622; H-204 to L-622; A-205 to L-622; F-206 to L-622; E-207 to L-622; T-208 to L-622; L-209 to L-622; S-210 to L-622; N-211 to L-622; L-212 to L-622; V-213 to L-622; T-214 to L-622; G-215 to L-622; S-216 to L-622; L-217 to L-622; S-218 to L-622; N-219 to L-622; G-220 to L-622; L-221 to L-622; L-222 to L-622; M-223 to L-622; V-224 to L-622; T-225 to L-622; T-226 to L-622; A-227 to L-622; N-228 to L-622; I-229 to L-622; T-230 to L-622; D-231 to L-622; R-232 to L-622; P-233 to L-622; A-234 to L-622; F-235 to L-622; S-236 to L-622; H-237 to L-622; R-238 to L-622; G-239 to L-622; V-240 to L-622; L-241 to L-622; L-242 to L-622; D-243 to L-622; T-244 to L-622; A-245 to L-622; R-246 to L-622; N-247 to L-622; F-248 to L-622; V-249 to L-622; P-250 to L-622; L-251 to L-622; K-252 to L-622; F-253 to L-622; I-254 to L-622; R-255 to L-622; S-256 to L-622; T-257 to L-622; L-258 to L-622; D-259 to L-622; A-260 to L-622; M-261 to L-622; A-262 to L-622; A-263 to L-622; S-264 to L-622; K-265 to L-622; L-266 to L-622; N-267 to L-622; V-268 to L-622; L-269 to L-622; H-270 to L-622; W-271 to L-622; H-272 to L-622; V-273 to L-622; V-274 to L-622; D-275 to L-622; T-276 to L-622; H-277 to L-622; S-278 to L-622; F-279 to L-622; P-280 to L-622; L-281 to L-622; E-282 to L-622; I-283 to L-622; T-284 to L-622; R-285 to L-622; V-286 to L-622; P-287 to L-622; E-288 to L-622; M-289 to L-622; Q-290 to L-622; R-291 to L-622; Y-292 to L-622; G-293 to L-622; A-294 to L-622; Y-295 to L-622; S-296 to L-622; S-297 to L-622; S-298 to L-622; Q-299 to L-622; T-300 to L-622; Y-301 to L-622; S-302 to L-622; R-303 to L-622; Q-304 to L-622; D-305 to L-622; A-306 to L-622; L-307 to L-622; N-308 to L-622; L-309 to L-622; V-310 to L-622; K-311 to L-622; Y-312 to L-622 A-313 to L-622; R-314 to L-622; L-315 to L-622; K-316 to L-622; G-317 to L-622; I-318 to L-622; R-319 to L-622; I-320 to L-622; L-321 to L-622; I-322 to L-622; E-323 to L-622; I-324 to L-622; D -325 to L-622; G-326 to L-622; P-327 to L-622; S-328 to L-622; H-329 to L-622; A-330 to L-622; G-331 to L-622; N-332 to L-622; G-333 to L-622; W-334 to L-622; Q-335 to L-622; W-336 to L-622; G-337 to L-622; P-338 to L-622; A-339 to L-622; A-340 to L-622; G-341 to L-622; L-342 to L-622; G-343 to L-622; N-344 to L-622; M-345 to L-622; S-346 to L-622; V-347 to L-622; C-348 to L-622; L-349 to L-622; N-330 to L-622; Q-351 to L-622; S-352 to L-622; P-353 to L-622; W-354 to L-622; R-355 to L-622; R-356 to L-622; F-357 to L-622; C-358 to L-622; V-359 to L-622; Q-360 to L-622; P-361 to L-622; P-362 to L-622; C-363 to L-622; G-364 to L-622; Q-365 to L-622; L-366 to L-622; N-367 to L-622; P-368 to L-622; L-369 to L-622; N-370 to L-622; D-371 to L-622; H-372 to L-622; M-373 to L-622; Y-374 to L-622; A-375 to L-622; V-376 to L-622; L-377 to L-622; K-378 to L-622; E-379 to L-622; I-380 to L-622; F-381 to L-622; E-382 to L-622; D-383 to L-622; V-384 to L-622; A-385 to L-622; E-386 to L-622; V-387 to L-622; G-388 to L-622; A-389 to L-622; P-390 to L-622; E-391 to L-622; E-392 to L-622; T-393 to L-622; L-394 to L-622; H-395 to L-622; M-396 to L-622; G-397 to L-622; G-398 to L-622; D-399 to L-622; E-400 to L-622; V-401 to L-622; F-402 to L-622; L-403 to L-622; P-404 to L-622; C-405 to L-622; W-406 to L-622; N-407 to L-622; N-408 to L-622; T-409 to L-622; D-410 to L-622; E-411 to L-622; I-412 to L-622; R-413 to L-622; D-414 to L-622; G-415 to L-622; M-416 to L-622; R-417 to L-622; A-418 to L-622; R-419 to L-622; G-420 to L-622; Y-421 to L-622; D-422 to L-622; L-423 to L-622; S-424 to L-622; E-425 to L-622; Q-426 to L-622; S-427 to L-622; F-428 to L-622; L-429 to L-622; R-430 to L-622; L-431 to L-622; W-432 to L-622; S-433 to L-622; Q-434 to L-622; F-435 to L-622; H-436 to L-622; Q-437 to L-622; R-438 to L-622; N-439 to L-622; L-440 to L-622; N-441 to L-622; A-442 to L-622; W-443 to L-622; D-444 to L-622; E-445 to L-622; I-446 to L-622; N-447 to L-622; E-448 to L-622; R-449 to L-622; M-450 to L-622; Y-451 to L-622; P-452 to L-622; G-453 to L-622; I-454 to L-622; K-455 to L-622; E-456 to L-622; P-457 to L-622; K-458 to L-622; S-459 to L-622; V-460 to L-622; I-461 to L-622; I-462 to L-622; W-463 to L-622; S-464 to L-622; S-465 to L-622; H-466 to L-622; L-467 to L-622; T-468 to L-622; N-469 to L-622; P-470 to L-622; R-471 to L-622; Y-472 to L-622; I-473 to L-622; E-474 to L-622; T-475 to L-622; Y-476 to L-622; L-477 to L-622; P-478 to L-622; K-479 to L-622; E-480 to L-622; R-481 to L-622; F-482 to L-622; I-483 to L-622; I-484 to L-622; Q-485 to L-622; T-486 to L-622; W-487 to L-622; V-488 to L-622; E-489 to L-622; S-490 to L-622; Q-491 to L-622; D-492 to L-622; A-493 to L-622; L-494 to L-622; N-495 to L-622; R-496 to L-622; E-497 to L-622; L-498 to L-622; L-499 to L-622; Q-500 to L-622; R-501 to L-622; G-502 to L-622; Y-503 to L-622; R-504 to L-622; L-505 to L-622; I-506 to L-622; V-507 to L-622; S-508 to L-622; T-509 to L-622; K-510 to L-622; N-511 to L-622; A-512 to L-622; W-513 to L-622; Y-514 to L-622; L-515 to L-622; D-516 to L-622; H-517 to L-622; G-518 to L-622; F-519 to L-622; W-520 to L-622; G-521 to L-622; S-522 to L-622; T-523 to L-622; S-524 to L-622; Y-525 to L-622; Y-526 to L-622; N-527 to L-622; W-528 to L-622; R-529 to L-622; T-530 to L-622; V-531 to L-622; Y-532 to L-622; S-533 to L-622; S-534 to L-622; G-535 to L-622; M-536 to L-622; P-537 to L-622; V-538 to L-622; G-539 to L-622; R-540 to L-622; S-541 to L-622; K-542 to L-622; D-543 to L-622; Q-544 to L-622; V-545 to L-622; L-546 to L-622; G-547 to L-622; G-548 to L-622; E-549 to L-622; V-550 to L-622; C-551 to L-622; M-552 to L-622; W-553 to L-622; S-554 to L-622; E-555 to L-622; Y-556 to L-622; V-557 to L-622; D-558 to L-622; Q-559 to L-622; N-560 to L-622; S-561 to L-622; L-562 to L-622; E-563 to L-622; S-564 to L-622; R-565 to L-622; I-566 to L-622; W-567 to L-622; P-568 to L-622; R-569 to L-622; A-570 to L-622; G-571 to L-622; A-572 to L-622; A-573 to L-622; A-574 to L-622; E-575 to L-622; R-576 to L-622; M-577 to L-622; W-578 to L-622; S-579 to L-622; N-580 to L-622; P-581 to L-622; K-582 to L-622; S-583 to L-622; S-584 to L-622; A-585 to L-622; L-586 to L-622; L-587 to L-622; A-588 to L-622; Q-589 to L-622; R-590 to L-622; R-591 to L-622; F-592 to L-622; Y-593 to L-622; R-594 to L-622; Y-595 to L-622; R-596 to L-622; E-597 to L-622; R-598 to L-622; L-599 to L-622; L-600 to L-622; A-601 to L-622; R-602 to L-622; G-603 to L-622; I-604 to L-622; H-605 to L-622; A-606 to L-622; D-607 to L-622; A-608 to L-622; V-609 to L-622; I-610 to L-622; P-611 to L-622; H-612 to L-622; W-613 to L-622; C-614 to L-622; V-615 to L-622; L-616 to L-622; and/or H-617 to L-622 of SEQ ID NO:6. Polynucleotides encoding these polypeptides are also encompassed by the invention.

Also as mentioned above, even if deletion of one or more amino acids from the C-terminus of a protein results in modification or loss of one or more biological functions of the protein, other functional activities (e.g., biological activities, ability to multimerize, ability to bind substrate) may still be retained. For example the ability of the shortened Hex-2 (SEQ ID NO:6) mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a Hex-2 mutein with a large number of deleted C-terminal amino acid residues may retain some biological or immunogenic activities. In fact, peptides composed of as few as six Hex-2 amino acid residues may elicit an immune response. Accordingly, the present invention further provides polypeptides having one or more residues deleted from the carboxy terminus of the amino acid sequence of the Hex-2 polypeptide shown in FIG. 3 (SEQ ID NO:6), as described by the general formula 1-n³, where n³ is an integer from 6 to 616, where n³ corresponds to the position of amino acid residue identified in SEQ ID NO:6. More in particular, the invention provides polynucleotides encoding polypeptides comprising, or alternatively consisting of, the amino acid sequence of residues of C-terminal deletions of the Hex-2 polypeptide of the invention shown as SEQ ID NO:6 including polypeptides comprising the amino acid sequence of residues: M-1 to C-621; M-1 to Q-620; M-1 to G-619; M-1 to E-618; M-1 to H-617; M-1 to L-616; M-1 to V-615; M-1 to C-614; M-1 to W-613; M-1 to H-612; M-1 to P-611; M-1 to I-610; M-1 to V-609; M-1 to A-608; M-1 to D-607; M-1 to A-606; M-1 to H-605; M-1 to I-604; M-1 to G-603; M-1 to R-602; M-1 to A-601; M-1 to L-600; M-1 to L-599; M-1 to R-598; M-1 to E-597; M-1 to R-596; M-1 to Y-595; M-1 to R-594; M-1 to Y-593; M-1 to F-592; M-1 to R-591; M-1 to R-590; M-1 to Q-589; M-1 to A-588; M-1 to L-587; M-1 to L-586; M-1 to A-585; M-1 to S-584; M-1 to S-583; M-1 to K-582; M-1 to P-581; M-1 to N-580; M-1 to S-579; M-1 to W-578; M-1 to M-577; M-1 to R-576; M-1 to E-575; M-1 to A-574; M-1 to A-573; M-1 to A-572; M-1 to G-571; M-1 to A-570; M-1 to R-569; M-1 to P-568; M-1 to W-567; M-1 to I-566; M-1 to R-565; M-1 to S-564; M-1 to E-563; M-1 to L-562; M-1 to S-561; M-1 to N-560; M-1 to Q-559; M-1 to D-558; M-1 to V-557; M-1 to Y-556; M-1 to E-555; M-1 to S-554; M-1 to W-553; M-1 to M-552; M-1 to C-551; M-1 to V-550; M-1 to E-549; M-1 to G-548; M-1 to G-547; M-1 to L-546; M-1 to V-545; M-1 to Q-544; M-1 to D-543; M-1 to K-542; M-1 to S-541; M-1 to R-540; M-1 to G-539; M-1 to V-538; M-1 to P-537; M-1 to M-536; M-1 to G-535; M-1 to S-534; M-1 to S-533; M-1 to Y-532; M-1 to V-531; M-1 to T-530; M-1 to R-529; M-1 to W-528; M-1 to N-527; M-1 to Y-526; M-1 to Y-525; M-1 to I-524; M-1 to T-523; M-1 to S-522; M-1 to G-521; M-1 to W-520; M-1 to F-519; M-1 to G-558; M-1 to H-517; M-1 to D-516; M-1 to L-515 ; M-1 to Y-514; M-1 to W-513 ; M-1 to A-512; M-1 to N-511; M-1 to K-510; M-1 to T-509; M-1 to S-508; M-1 to V-507; M-1 to I-506; M-1 to L-505; M-1 to R-504; M-1 to Y-503; M-1 to Y-502; M-1 to R-501; M-1 to Q-500; M-1 to L-499; M-1 to L-498; M-1 to E-497; M-1 to R-496; M-1 to N-495; M-1 to L-494; M-1 to A-493; M-1 to D-492; M-1 to Q-491; M-1 to S-490; M-1 to E-489; M-1 to V-488; M-1 to W-487; M-1 to T-486; M-1 to Q-485; M-1 to I-484; M-1 to I -483; M-1 to F-482; M-1 to R-481; M-1 to E-480; M-1 to K-479; M-1 to P-478; M-1 to L-477; M-1 to Y-476; M-1 to T-475; M-1 to E-474; M-1 to I-473; M-1 to Y-472; M-1 to R-471; M-1 to P-470; M-1 to N-469; M-1 to T-468; M-1 to L-467; M-1 to H-466; M-1 to S-465; M-1 to S-464; M-1 to W-463; M-1 to I-462; M-1 to I-461; M-1 to V-460; M-1 to S-459; M-1 to K-458; M-1 to P-457; M-1 to E-456; M-1 to K-455; M-1 to I-454; M-1 to G-453; M-1 to P-452; M-1 to Y-451; M-1 to M-450; M-1 to R-449; M-1 to E-448; M-1 to N-447; M-1 to I-446; M-1 to E-445; M-1 to D-444; M-1 to W-443; M-1 to A-442; M-1 to N-441; M-1 to L-440; M-1 to N-439; M-1 to R-438; M-1 to Q-437; M-1 to H-436; M-1 to F-435; M-1 to Q-434; M-1 to S-433; M-1 to W-432; M-1 to L-431; M-1 to R-430; M-1 to L-429; M-1 to F-428; M-1 to S-427; M-1 to Q-426; M-1 to E-425; M-1 to S-424; M-1 to L-423; M-1 to D-422; M-1 to Y-421; M-1 to G-420; M-1 to R-419; M-1 to A-418; M-1 to R-417; M-1 to M-416; M-1 to G-415; M-1 to D-414; M-1 to R-413; M-1 to I-412; M-1 to E-411; M-1 to D-410; M-1 to T-409; M-1 to N-408; M-1 to N-407; M-1 to W-406; M-1 to C-405; M-1 to P-404; M-1 to L-403; M-1 to F-402; M-1 to V-401; M-1 to E-400; M-1 to D-399; M-1 to G-398; M-1 to G-397; M-1 to M-396; M-1 to H-395; M-1 to L-394; M-1 to T-393; M-1 to E-392; M-1 to E-391; M-1 to P-390; M-1 to A-389; M-1 to G-388; M-1 to V-387; M-1 to E-386; M-1 to A-385; M-1 to V-384; M-1 to D-383; M-1 to E-382; M-1 to F-381; M-1 to I-380; M-1 to E-379; M-1 to K-378; M-1 to L-377; M-1 to V-376; M-1 to A-375; M-1 to Y-374; M-1 to M-373; M-1 to H-372; M-1 to D-371; M-1 to N-370; M-1 to L-369; M-1 to P-368; M-1 to N-367; M-1 to L-366; M-1 to Q-365; M-1 to G-364; M-1 to C-363; M-1 to P-362; M-1 to P-361; M-1 to Q-360; M-1 to V-359; M-1 to C-358; M-1 to F-357; M-1 to R-356; M-1 to R-355; M-1 to W-354; M-1 to P-353; M-1 to S-352; M-1 to Q-351; M-1 to N-350; M-1 to L-349; M-1 to C-348; M-1 to V-347; M-1 to S-346; M-1 to M-345; M-1 to N-344; M-1 to G-343; M-1 to L-342; M-1 to G-341; M-1 to A-340; M-1 to A-339; M-1 to P-338; M-1 to G-337; M-1 to W-336; M-1 to Q-335; M-1 to W-334; M-1 to G-333; M-1 to N-332; M-1 to G-331; M-1 to A-330; M-1 to H-329; M-1 to S-328; M-1 to P-327; M-1 to G-326; M-1 to D-325; M-1 to I-324; M-1 to E-323; M-1 to I-322; M-1 to L-321; M-1 to I-320; M-1 to R-319; M-1 to I-318; M-1 to G-317; M-1 to R-316; M-1 to L-315;,M-1 to R-314; M-1 to A-313; M-1 to Y-312; M-1 to K-311; M-1 to V-310; M-1 to L-309; M-1 to N-308; M-1 to L-307; M-1 to A-306; M-1 to D-305; M-1 to Q-304; M-1 to R-303; M-1 to S-302; M-1 to Y-301; M-1 to T-300; M-1 to Q-299; M-1 to S-298; M-1 to S-297; M-1 to S-296; M-1 to Y-295; M-1 to A-294; M-1 to G-293; M-1 to Y-292; M-1 to R-291; M-1 to Q-290; M-1 to M-289; M-1 to E-288; M-1 to P-287; M-1 to V-286; M-1 to R-285; M-1 to T-284; M-1 to I-283; M-1 to E-282; M-1 to L-281; M-1 to P-280; M-1 to F-279; M-1 to S-278; M-1 to H-277; M-1 to T-276; M-1 to D-275; M-1 to V-274; M-1 to V-273; M-1 to H-272; M-1 to W-271; M-1 to H-270; M-1 to L-269; M-1 to V-268; M-1 to N-267; M-1 to L-266; M-1 to K-265; M-1 to S-264; M-1 to A-263; M-1 to A-262; M-1 to M-261; M-1 to A-260; M-1 to D-259; M-1 to L-258; M-1 to T-257; M-1 to S-256; M-1 to R-255; M-1 to I-254; M-1 to F-253; M-1 to K-252; M-1 to L-251; M-1 to P-250; M-1 to V-249; M-1 to F-248; M-1 to N-247; M-1 to R-246; M-1 to A-245; M-1 to T-244; M-1 to D-243; M-1 to L-242; M-1 to L-241; M-1 to V-240; M-1 to G-239; M-1 to R-238; M-1 to H-237; M-1 to S-236; M-1 to F-235; M-1 to A-234; M-1 to P-233; M-1 to R-232; M-1 to D-231; M-1 to T-230; M-1 to I-229; M-1 to N-228; M-1 to A-227; M-1 to T-226; M-1 to T-225; M-1 to V-224; M-1 to M-223; M-1 to L-222; M-1 to L-221; M-1 to G-220; M-1 to N-219; M-1 to S-218; M-1 to L-217; M-1 to S-216; M-1 to G-215; M-1 to T-214; M-1 to V-213; M-1 to L-212; M-1 to N-211; M-1 to S-210; M-1 to L-209; M-1 to T-208; M-1 to E-207; M-1 to F-206; M-1 to A-205; M-1 to H-204; M-1 to R-203; M-1 to A-202; M-1 to G-201; M-1 to Y-200; M-1 to V-199; M-1 to T-198; M-1 to T-197; M-1 to A-196; M-1 to Q-195; M-1 to I-194; M-1 to D-193; M-1 to V-192; M-1 to F-191; M-1 to T-190; M-1 to A-189; M-1 to T-188; M-1 to E-187; M-1 to T-186; M-1 to T-185; M-1 to R-184; M-1 to V-183; M-1 to V-182; M-1 to L-181; M-1 to A-180; M-1 to Y-179; M-1 to S-178; M-1 to E-177; M-1 to D-176; M-1 to T-175; M-1 to P-174; M-1 to W-173; M-1 to D-172; M-1 to L-171; M-1 to V-170; M-1 to L-169; M-1 to S-168; M-1 to E-167; M-1 to N-166; M-1 to A-165; M-1 to V-164; M-1 to T-163; M-1 to S-162; M-1 to R-161; M-1 to V-160; M-1 to L-159; M-1 to I-158; M-1 to Q-157; M-1 to K-156; M-1 to S-155; M-1 to T-154; M-1 to E-153; M-1 to L-152; M-1 to T-151; M-1 to C-150; M-1 to N-149; M-1 to R-148; M-1 to I-147; M-1 to C-146; M-1 to E-145; M-1 to K-144; M-1 to L-143; M-1 to L-142; M-1 to N-141; M-1 to S-140; M-1 to V-139; M-1 to F-138; M-1 to L-137; M-1 to R-136; M-1 to N-135; M-1 to T-134; M-1 to E-133; M-1 to R-132; M-1 to L-131; M-1 to F-130; M-1 to Q-129; M-1 to T-128; M-1 to A-127; M-1 to A-126; M-1 to E-125; M-1 to G-124; M-1 to P-123; M-1 to A-122; M-1 to V-121; M-1 to V-120; M-1 to H -119; M-1 to F-118; M-1 to R-117; M-1 to V-116; M-1 to K-115; M-1 to W-114; M-1 to P-113; M-1 to D-112; M-1 to F-111; M-1 to R-110; M-1 to V-109; M-1 to R-108; M-1 to R-107; M-1 to H-106; M-1 to S-105; M-1 to I-104; M-1 to T-103; M-1 to C-102; M-1 to E-101; M-1 to K-100; M-1 to G-99; M-1 to T-98; M-1 to P-97; M-1 to M-96; M-1 to P-95; M-1 to W-94; M-1 to I-93; M-1 to A-92; M-1 to G-91; M-1 to Y-90; M-1 to K-89; M-1 to G-88; M-1 to C-87; M-1 to S-86; M-1 to L-85; M-1 to R-84; M-1 to C-83; M-1 to D-82; M-1 to R-81; M-1 to Q-80; M-1 to S-79; M-1 to E-78; M-1 to F-77; M-1 to I-76; M-1 to E-75; M-1 to P-74; M-1 to A-73; M-1 to Y-72; M-1 to Q-71; M-1 to L-70; M-1 to G-69; M-1 to A-68; M-1 to V-67; M-1 to M-66; M-1 to E-65; M-1 to G-64; M-1 to E-63; M-1 to A-62; M-1 to T-61; M-l to C-60; M-1 to I-59; M-1 to D-58; M-1 to T-57; M-1 to R-56; M-1 to S-55; M-1 to C-54; M-1 to L-53; M-1 to W-52; M-1 to K-51; M-1 to R-50; M-1 to S-49; M-1 to A-48; M-1 to S-47; M-1 to G-46; M-1 to A-45; M-1 to D-44; M-1 to A-43; M-1 to D-42; M-1 to S-41; M-1 to G-40; M-1 to V-39; M-1 to D-38; M-1 to D-37; M-1 to A-36; M-1 to R-35; M-1 to T-34; M-1 to L-33; M-1 to A-32; M-1 to A-31; M-1 to G-30; M-1 to V-29; M-1 to A-28; M-1 to F-27; M-1 to S-26; M-1 to L-25; M-1 to L-24; M-1 to S-23; M-1 to L-22; M-1 to L-21; M-1 to L-20; M-l to L-19; M-1 to S-18; M-1 to G-17; M-1 to V-16; M-1 to A-15; M-1 to S-14; M-1 to C-13; M-1 to F-12; M-1 to C-11; M-1 to Q-10; M-1 to Y-9; M-1 to R-8; and/or M-1 to N-7 of SEQ ID NO:6. Polynucleotides encoding these polypeptides are also encompassed by the invention.

Moreover, a signal sequence may be added to these C-terminal contructs. For example, amino acids 1-32 of SEQ ID NO:6, amino acids 2-32 of SEQ ID NO:6, amino acids 3-32 of SEQ ID NO:6, amino acids 4-32 of SEQ ID NO:6, amino acids 5-32 of SEQ ID NO:6, amino acids 6-32 of SEQ ID NO:6, amino acids 7-32 of SEQ ID NO:6, amino acids 8-32 of SEQ ID NO:6, amino acids 9-32 of SEQ ID NO:6, amino acids 10-32 of SEQ ID NO:6, amino acids 11-32 of SEQ ID NO:6, amino acids 12-32 of SEQ ID NO:6, amino acids 13-32 of SEQ ID NO:6, amino acids 14-32 of SEQ ID NO:6, amino acids 15-32 of SEQ ID NO:6, amino acids 16-32 of SEQ ID NO:6, amino acids 17-32 of SEQ ID NO:6, amino acids 18-32 of SEQ ID NO:6, amino acids 19-32 of SEQ ID NO:6, amino acids 20-32 of SEQ ID NO:6, amino acids 21-32 of SEQ ID NO:6, amino acids 22-32 of SEQ ID NO:6, amino acids 32-32 of SEQ ID NO:6, amino acids 24-32 of SEQ ID NO:6, amino acids 25-32 of SEQ ID NO:6, amino acids 26-32 of SEQ ID NO:6, amino acids 27-32 of SEQ ID NO:6, amino acids 28-32 of SEQ ID NO:6, amino acids 29-32 of SEQ ID NO:6, amino acids 30-32 of SEQ ID NO:6, or amino acids 31-32 of SEQ ID NO:6 can be added to the N-terminus of each C-terminal constructs listed above.

In addition, any of the above listed N- or C-terminal deletions can be combined to produce a N- and C-terminal deleted Hex-2 polypeptide. The invention also provides polypeptides having one or more amino acids deleted from both the amino and the carboxyl termini, which may be described generally as having residues m³-n³ of SEQ ID NO:6, where n³ and m³ are integers as described above. Polynucleotides encoding these polypeptides are also encompassed by the invention.

Also included are a nucleotide sequence encoding a polypeptide consisting of a portion of the complete Hex-2 amino acid sequence encoded by the cDNA clone contained in ATCC Deposit No. ______ (as shown in SEQ ID NO:5 and SEQ ID NO:6), where this portion excludes any integer of amino acid residues from 1 to about 616 amino acids from the amino terminus of the complete amino acid sequence encoded by the cDNA clone contained in the ATCC Deposit No. ______, or any integer of amino acid residues from 1 to 616 amino acids from the carboxy terminus, or any combination of the above amino terminal and carboxy terminal deletions, of the complete amino acid sequence encoded by the cDNA clone contained in the ATCC Deposit No. ______. Polynucleotides encoding all of the above deletion mutant polypeptide forms also are provided.

The present application is also directed to proteins containing polypeptides at least 90%, 95%, 96%, 97%, 98% or 99% identical to the Hex-2 (SEQ ID NO:6) polypeptide sequence set forth herein. In preferred embodiments, the application is directed to proteins containing polypeptides at least 90%, 95%, 96%, 97%, 98% or 99% identical to polypeptides having the amino acid sequence of the specific Hex-2 N- and C-terminal deletions recited herein. Polynucleotides encoding these polypeptides are also encompassed by the invention. Additional preferred Hex-2 (SEQ ID NO:6) polypeptide fragments comprise, or alternatively consist of, the amino acid sequence of residues: M-1 to A-15; R-2 to V-16; F-3 to G-17; S-4 to S-18; G-5 to L-19; Y-6 to L-20; N-7 to L-21; R-8 to L-22; Y-9 to S-23; Q-10 to L-24; C-11 to L-25; F-12 to S-26; C-13 to F-27; S-14 to A-28; A-15 to V-29; V-16 to G-30; G-17 to A-31; S-18 to A-32; L-19 to L-33; L-20 to T-34; L-21 to R-35; L-22 to A-36; S-23 to D-37; L-24 to D-38; L-25 to V-39; S-26 to G-40; F-27 to S-41; A-28 to D-42; V-29 to A-43; G-30 to D-44; A-31 to A-45; A-32 to G-46; L-33 to S-47; T-34 to A-48; R-35 to S-49; A-36 to R-50; D-37 to K-51; D-38 to W-52; V-39 to L-53; G-40 to C-54; S-41 to S-55; D-42 to R-56; A-43 to T-57; D-44 to D-58; A-45 to I-59; G-46 to C-60; S-47 to T-61; A-48 to A-62; S-49 to E-63; R-50 to G-64; K-51 to E-65; W-52 to M-66; L-53 to V-67; C-54 to A-68; S-55 to G-69; R-56 to L-70; T-57 to Q-71; D-58 to Y-72; I-59 to A-73; C-60 to P-74; T-61 to E-75; A-62 to I-76; E-63 to F-77; G-64 to E-78; E-65 to S-79; M-66 to Q-80; V-67 to R-81; A-68 to D-82; G-69 to C-83; L-70 to R-84; Q-71 to L-85; Y-72 to S-86; A-73 to C-87; P-74 to G-88; E-75 to K-89; I-76 to Y-90; F-77 to G-91; E-78 to A-92; S-79 to I-93; Q-80 to W-94; R-81 to P-95; D-82 to M-96; C-83 to P-97; R-84 to T-98; L-85 to G-99; S-86 to K-100; C-87 to E-101; G-88 to C-102; K-89 to T-103; Y-90 to I-104; G-91 to S-105; A-92 to H-106; I-93 to R-107; W-94 to R-108; P-95 to V-109; M-96 to R-10; P-97 to F-111; T-98 to D-112; G-99 to P-113; K-100 to W-114; E-101 to K-115; C-102 to V-116; T-103 to R-117; I-104 to F-118; S-105 to H-119; H-106 to V-120; R-107 to V-121; R-108 to A-122; V-109 to P-123; R-110 to G-124; F-111 to E-125; D-112 to A-126; P-113 to A-127; W-114 to T-128; K-115 to Q-129; V-116 to F-130; R-117 to L-131; F-118 to R-132; H-119 to E-133; V-120 to T-134; V-121 to N-135; A-122 to R-136; P-123 to L-137; G-124 to F-138; E-125 to V-139; A-126 to S-140; A-127 to N-141; T-128 to L-142; Q-129 to L-143; F-130 to K-144; L-131 to E-145; R-132 to C-146; E-133 to I-147; T-134 to R-148; N-135 to N-149; R-136 to C-150; L-137 to T-151; F-138 to L-152; V-139 to E-153; S-140 to T-154; N-141 to S-155; L-142 to K-156; L-143 to Q-157; K-144 to I-158; E-145 to L-159; C-146 to V-160; I-147 to R-161; R-148 to S-162; N-149 to T-163; C-150 to V-164; T-151 to A-165; L-152 to N-166; E-153 to E-167; T-154 to S-168; S-155 to L-169; K-156 to V-170; Q-157 to L-171; I-158 to D-172; L-159 to W-173; V-160 to P-174; R-161 to T-175; S-162 to D-176; T-163 to E-177; V-164 to S-178; A-165 to Y-179; N-166 to A-180; E-167 to L-181; S-168 to V-182; L-169 to V-183; V-170 to R-184; L-171 to T-185; D-172 to T-186; W-173 to E-187; P-174 to T-188; T-175 to A-189; D-176 to T-190; E-177 to F-191; S-178 to V-192; Y-179 to D-193; A-180 to I-194; L-181 to Q-195; V-182 to A-196; V-183 to T-197; R-184 to T-198; T-185 to V-199; T-186 to Y-200; E-187 to G-201; T-188 to A-202; A-189 to R-203; T-190 to H-204; F-191 to A-205; V-192 to F-206; D-193 to E-207; I-194 to T-208; Q-195 to L-209; A-196 to S-210; T-197 to N-211; T-198 to L-212; V-199 to V-213; Y-200 to T-214; G-201 to G-215; A-202 to S-216; R-203 to L-217; H-204 to S-218; A-205 to N-219; F-206 to G-220; E-207 to L-221; T-208 to L-222; L-209 to M-223; S-210 to V-224; N-211 to T-225; L-212 to T-226; V-213 to A-227; T-214 to N-228; G-215 to I-229; S-216 to T-230; L-217 to D-231; S-218 to R-232; N-219 to P-233; G-220 to A-234; L-221 to F-235; L-222 to S-236; M-223 to H-237; V-224 to R-238; T-225 to G-239; T-226 to V-240; A-227 to L-241; N-228 to L-242; I-229 to D-243; T-230 to T-244; D-231 to A-245; R-232 to R-246; P-233 to N-247; A-234 to F-248; F-235 to V-249; S-236 to P-250; H-237 to L-251; R-238 to K-252; G-239 to F-253; V-240 to I-254; L-241 to R-255; L-242 to S-256; D-243 to T-257; T-244 to L-258; A-245 to D-259; R-246 to A-260; N-247 to M-261; F-248 to A-262; V-249 to A-263; P-250 to S-264; L-251 to K-265; K-252 to L-266; F-253 to N-267; I-254 to V-268; R-255 to L-269; S-256 to H-270; T-257 to W-271; L-258 to H-272; D-259 to V-273; A-260 to V-274; M-261 to D-275; A-262 to T-276; A-263 to H-277; S-264 to S-278; K-265 to F-279; L-266 to P-280; N-267 to L-281; V-268 to E-282; L-269 to I-283; H-270 to T-284; W-271 to R-285; H-272 to V-286; V-273 to P-287; V-274 to E-288; D-275 to M-289; T-276 to Q-290; H-277 to R-291; S-278 to Y-292; F-279 to G-293; P-280 to A-294; L-281 to Y-295; E-282 to S-296; I-283 to S-297; T-284 to S-298; R-285 to Q-299; V-286 to T-300; P-287 to Y-301; E-288 to S-302; M-289 to R-303; Q-290 to Q-304; R-291 to D-305; Y-292 to A-306; G-293 to L-307; A-294 to N-308; Y-295 to L-309; S-296 to V-310; S-297 to K-311; S-298 to Y-312; Q-299 to A-313; T-300 to R-314; Y-301 to L-315; S-302 to R-316; R-303 to G-317; Q-304 to I-318; D-305 to R-319; A-306 to I-320; L-307 to L-321; N-308 to I-322; L-309 to E-323; V-310 to I-324; K-311 to D-325; Y-312 to G-326; A-313 to P-327; R-314 to S-328; L-315 to H-329; R-316 to A-330; G-317 to G-331; I-318 to N-332; R-319 to G-333; I-320 to W-334; L-321 to Q-335; I-322 to W-336; E-323 to G-337; I-324 to P-338; D-325 to A-339; G-326 to A-340; P-327 to G-341; S-328 to L-342; H-329 to G-343; A-330 to N-344; G-331 to M-345; N-332 to S-346; G-333 to V-347; W-334 to C-348; Q-335 to L-349; W-336 to N-350; G-337 to Q-351; P-338 to S-352; A-339 to P-353; A-340 to W-354; G-341 to R-355; L-342 to R-356; G-343 to F-357; N-344 to C-358; M-345 to V-359; S-346 to Q-360; V-347 to P-361; C-348 to P-362; L-349 to C-363; N-350 to G-364; Q-351 to Q-365; S-352 to L-366; P-353 to N-367; W-354 to P-368; R-355 to L-369; R-356 to N-370; F-357 to D-371; C-358 to H-372; V-359 to M-373; Q-360 to Y-374; P-361 to A-375; P-362 to V-376; C-363 to L-377; G-364 to K-378; Q-365 to E-379; L-366 to I-380; N-367 to F-381; P-368 to E-382; L-369 to D-383; N-370 to V-384; D-371 to A-385; H-372 to E-386; M-373 to V-387; Y-374 to G-388; A-375 to A-389; V-376 to P-390; L-377 to E-391; K-378 to E-392; E-379 to T-393; I-380 to L-394; F-381 to H-395; E-382 to M-396; D-383 to G-397; V-384 to G-398; A-385 to D-399; E-386 to E-400; V-387 to V-401; G-388 to F-402; A-389 to L-403; P-390 to P-404; E-391 to C-405; E-392 to W-406; T-393 to N-407; L-394 to N-408; H-395 to T-409; M-396 to D-410; G-397 to E-411; G-398 to I-412; D-399 to R-413; E-400 to D-414; V-401 to G-415; F-402 to M-416; L-403 to R-417; P-404 to A-418; C-405 to R-419; W-406 to G-420; N-407 to Y-421; N-408 to D-422; T-409 to L-423; D-410 to S-424; E-411 to E-425; 1-412 to Q-426; R-413 to S-427; D-414 to F-428; G-415 to L-429; M-416 to R-430; R-417 to L-431; A-418 to W-432; R-419 to S-433; G-420 to Q-434; Y-421 to F-435; D-422 to H-436; L-423 to Q-437; S-424 to R-438; E-425 to N-439; Q-426 to L-440; S-427 to N-441; F-428 to A-442; L-429 to W-443; R-430 to D-444; L-431 to E-445; W-432 to I-446; S-433 to N-447; Q-434 to E-448; F-435 to R-449; H-436 to M-450; Q-437 to Y-451; R-438 to P-452; N-439 to G-453; L-440 to I-454; N-441 to K-455; A-442 to E-456; W-443 to P-457; D-444 to K-458; E-445 to S-459; I-446 to V-460; N-447 to I-461; E-448 to I-462; R-449 to W-463; M-450 to S-464; Y-451 to S-465; P-452 to H-466; G-453 to L-467; I-454 to T-468; K-455 to N-469; E-456 to P-470; P-457 to R-471; K-458 to Y-472; S-459 to I-473; V-460 to E-474; I-461 to T-475; I-462 to Y-476; W-463 to L-477; S-464 to P-478; S-465 to K-479; H-466 to E-480; L-467 to R-481; T-468 to F-482; N-469 to I-483; P-470 to I-484; R-471 to Q-485; Y-472 to T-486; I-473 to W-487; E-474 to V-488; T-475 to E-489; Y-476 to S-490; L-477 to Q-491; P-478 to D-492; K-479 to A-493; E-480 to L-494; R-481 to N-495; F-482 to R-496; I-483 to E-497; I-484 to L-498; Q-485 to L-499; T-486 to Q-500; W-487 to R-501; V-488 to G-502; E-489 to Y-503; S-490 to R-504; Q-491 to L-505; D-492 to I-506; A-493 to V-507; L-494 to S-508; N-495 to T-509; R-496 to K-510; E-497 to N-511; L-498 to A-512; L-499 to W-513; Q-500 to Y-514; R-501 to L-515; G-502 to D-516; Y-503 to H-517; R-504 to G-518; L-505 to F-519; I-506 to W-520; V-507 to G-521; S-508 to S-522; T-509 to T-523; K-510 to S-524; N-511 to Y-525; A-512 to Y-526; W-513 to N-527; Y-514 to W-528; L-515 to R-529; D-516 to T-530; H-517 to V-531; G-518 to Y-532; F-519 to S-533; W-520 to S-534; G-521 to G-535; S-522 to M-536; T-523 to P-537; S-524 to V-538; Y-525 to G-539; Y-526 to R-540; N-527 to S-541; W-528 to K-542; R-529 to D-543; T-530 to Q-544; V-531 to V-545; Y-532 to L-546; S-533 to G-547; S-534 to G-548; G-535 to E-549; M-536 to V-550; P-537 to C-551; V-538 to M-552; G-539 to W-553; R-540 to S-554; S-541 to E-555; K-542 to Y-556; D-543 to V-557; Q-544 to D-558; V-545 to Q-559; L-546 to N-560; G-547 to S-561; G-548 to L-562; E-549 to E-563; V-550 to S-564; C-551 to R-565; M-552 to I-566; W-553 to W-567; S-554 to P-568; E-555 to R-569; Y-556 to A-570; V-557 to G-571; D-558 to A-572; Q-559 to A-573; N-560 to A-574; S-561 to E-575; L-562 to R-576; E-563 to M-577; S-564 to W-578; R-565 to S-579; I-566 to N-580; W-567 to P-581; P-568 to K-582; R-569 to S-583; A-570 to S-584; G-571 to A-585; A-572 to L-586; A-573 to L-587; A-574 to A-588; E-575 to Q-589; R-576 to R-590; M-577 to R-591; W-578 to F-592; S-579 to Y-593; N-580 to R-594; P-581 to Y-595; K-582 to R-596; S-583 to E-597; S-584 to R-598; A-585 to L-599; L-586 to L-600; L-587 to A-601; A-588 to R-602; Q-589 to G-603; R-590 to I-604; R-591 to H-605; F-592 to A-606; Y-593 to D-607; R-594 to A-608; Y-595 to V-609; R-596 to I-610; E-597 to P-611; R-598 to H-612; L-599 to W-613; L-600 to C-614; A-601 to V-615; R-602 to L-616; G-603 to H-617; I-604 to E-618; H-605 to G-619; A-606 to Q-620; D-607 to C-621; A-608 to L-622. These polypeptide fragments may retain the biological activity of Hex-2 polypeptides of the invention and/or may be useful to generate or screen for antibodies, as described further below. Polynucleotides encoding these polypeptide fragments are also encompassed by the invention.

Compositions and methods for producing glycoproteins having sialylated oligosaccharides are provided. The compositions of the invention comprise enzymes involved in transport of nucleotide sugars and in carbohydrate processing, nucleotide sequences encoding such enzymes, and cells transformed with these nucleotide sequences. The compositions of the invention are useful in methods for producing complex sialylated glycoproteins in cells of interest including, but not limited to, mammalian cells and non-mammalian cells (e.g., insect cells). Further, the compositions of the invention are useful in methods for producing complex sialylated glycoproteins in cells by increasing CMP-SA transport via expression of recombinant Drosophila CMP-SAT and via inhibition of endogenous Drosophila Hex-1 and Hex-2 activity.

The sialylation process involves the post-translational addition of a donor substrate, cytidine monophosphate-sialic acid (CMP-SA) onto a specific acceptor carbohydrate (GalGlcNAcMan-R) via an enzymatic reaction catalyzed by a sialyltransferase in the Golgi apparatus. Since transport of CMP-SA to the Golgi apparatus is limiting or absent in certain cells of interest, methods are provided to enhance CMP-SA transport. Furthermore, protein sialylation requires the presence of appropriate carbohydrate acceptor substrates. Since these substrates are limited in insect cells by endogenouse glucosaminidase activity, the invention provides for methods of inhibiting Hex-1 and Hex-2 activity.

Vectors and Hosts

The present invention also encompasses recombinant vectors, which include the isolated nucleic acid molecules and polynucleotides that may be used according to the methods of the present invention, and to host cells containing the recombinant vectors and/or nucleic acid molecules, as well as to methods of making such vectors and host cells and for using them for production of polynucleotides and polypeptides by recombinant techniques. Polynucleotides and polypeptides produced by such methods are also provided.

The invention encompasses utilizing vectors for the maintenance (cloning vectors) and vectors for expression (expression vectors) of the desired polynucleotides and/or the encoded carbohydrate processing polypeptides of the invention, or those encoding proteins to be sialylated by the methods of the invention and/or by expression of the proteins in cells of the invention. The vectors can function in prokaryotic or eukaryotic cells or in both (shuttle vectors).

In one embodiment, one or more of the polynucleotide sequences used according to the methods of the invention are inserted into commercially, publicly, or otherwise available baculovirus expression vectors for enhanced expression of the corresponding enzyme. In another non-exclusive embodiment, one ore more of the polynucleotides used according to the methods of the invention are inserted into other viral vectors or for generation of stable insect cell lines. Techniques known in the art, such as, for example, HPAEC and HPLC techniques, may be routinely used to evaluate the enzymatic activity of these enzymes from both eukaryotic and bacterial sources to determine which source is best for generating SA in insect cells.

Generally, expression vectors contain cis-acting regulatory regions that are operably linked in the vector to the polynucleotide to be expressed, or other relevant polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of the polynucleotides from the vector. Alternatively, a trans-acting factor may be supplied by the host cell. Finally, a trans-acting factor can be produced from the vector itself.

It is understood, however, that in some embodiments, transcription of the polynucleotides can occur in a cell-free system.

The regulatory sequence to which the polynucleotides described herein can be operably linked include, for example, promoters for directing mRNA transcription. These promoters include, but are not limited to, baculovirus promoters including, but not limited to, 1E0, 1E1, 1E2, 39 k, 35 k, egt, ME53, ORF 142, PE38, p6.9, capsid, gp64 polyhedrin, p10, basic and core; and insect cell promoters including, but not limited to, Drosophila actin, metallothionine, and the like. Where the host cell is not an insect cell, such promoters include, but are not limited to, the left promoter from bacteriophage lambda, the lac, TRP, and TAC promoters from E. coli, promoters from Actinomycetes, including Nocardia, and Streptomyces.

Promoters may be isolated, if they have not already been isolated, by standard promoter identification and trapping methods known in the art, see, for example, in Sambrook et al., Molecular Cloning: A Laboratory Manual 2nd. ed., Cold Spring Harbor Laboratory Press, Cold Spring Harbor, N.Y., (1989).

It would be understood by a person of ordinary skill in the art that the choice of promoter would depend upon the choice of host cell. Similarly, the choice of host cell will depend upon the use of the host cell. Accordingly, host cells can be used for simply amplifying, but not expressing, the nucleic acid. However, host cells can also be used to produce desirable amounts of the desired polypeptide. In this embodiment, the host cell is simply used to express the protein per se. For example, amounts of the protein could be produced that enable its purification and subsequent use, for example, in a cell free system. In this case, the promoter is compatible with the host cell. Host cells can be chosen from virtually any of the known host cells that are manipulated by the methods of the invention to produce the desired glycosylation patterns. These could include mammalian, bacterial, yeast, filamentous fungi, or plant cells.

In addition to control regions that promote transcription, expression vectors may also include regions that modulate transcription, such as repressor binding sites and enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., cited above.

Depending on the choice of a host cell, a variety of expression vectors can be used to express the polynucleotide. Such vectors include chromosomal, episomal, and particularly virus-derived vectors, for example, AcMNPV, OpMNPV, BmNPV, HzMNPV, and RoMNPV. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The polynucleotides can be inserted into the vector nucleic acid using techniques known in the art. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

Specific expression vectors are described herein for the purposes of the invention; for example, AcMNPV. Other expression vectors listed herein are not intended to be limiting, and are merely provided by way of example. The person of ordinary skill in the art would be aware of other vectors suitable for maintenance, propagation, or expression of the polynucleotides described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Any cell type or expression system can be used for the purposes of the invention including but not limited to, for example, baculovirus systems (O'Riley et al. (1992) Baculovirus Expression Vectors, W.H. Freeman and Company, New York 1992) and Drosophila-derived systems (Johansen et al. (1989) Genes Dev 3(6):882-889).

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Where secretion of the polypeptide is desired, appropriate secretion signals known in the art are incorporated into the vector using techniques known in the art. The signal sequence can be endogenous to the polypeptides or heterologous to these polypeptides.

Where the polypeptide is not secreted into the medium, the desired protein can be isolated from the host cell by techniques known in the art, such as, for example, standard disruption procedures, including freeze thaw, sonication, mechanical disruption, use of lysing agents and the like. The polypeptide can then be recovered and purified by well-known purification methods including, but not limited to, ammonium sulfate precipitation, acid extraction, anion or cationic exchange chromatography, phosphocellulose chromatography, hydrophobic-interaction chromatography, affinity chromatography, hydroxylapatite chromatography, lectin chromatography, and high performance liquid chromatography.

### EXAMPLES

Having generally described the invention, the same will be more readily understood by reference to the following assays and examples, which are provided by way of illustration and are not intended as limiting.

Analytical bioassays are implemented to evaluate enzymatic activities in the N-glycosylation pathway of insect cells. In order to screen a larger selection of insect cells for particular oligosaccharide processing enzymes, bioassays in which multiple samples can be analyzed simultaneously are advantageous. Consequently, bioassays based on fluorescence energy transfer (FRET) and time-resolved fluorometry of europium (Eu) are designed to screen native and recombinant insect cell lines for carbohydrate processing enzymes in a format that can handle multiple samples.

Fluorescence assays are especially useful in detecting limiting steps in carbohydrate processing due to their sensitivity and specificity. FRET and Eu assays detect enzymatic activities at levels as low as 10⁻¹⁴ M, which is greater than the sensitivity obtained with ¹²⁵I. In addition, the use of substrates modified with fluorophores enables the measurement of one specific enzyme activity in an insect cell lysate, and multiple samples can be analyzed simultaneously in a microtiter plate configuration used in an appropriate fluorometer.

### Example 1

Europium (Eu⁻⁾) Fluorescence Assays.

An example of the use of Eu⁺³ fluorescence for the evaluation of Gal T activity is provided herein in the N-linked oligosaccharides from insect cells. The same techniques are used to develop enzymatic assay for transferases such as GlcNAc Ti and glycosidases such as Drosophila Hex-1 and Hex-2. Further enhancements in sensitivity are obtained with the advent of the super-sensitive Eu-chelator, BHHT (4, 4′-bis (1″,1″,1″,2″,2″,3″,3′-heptatluro-4″,6″ -hexanedione-6′-yl)-chlorosulfo-o-terphenyl) (Yuan et al. (1998) Anal. Chem. 70:596-601), which allows detection down to the lower fmol range.

### Example 2

Glucosaminidase Assay

An assay for N-acetylglucosaminidase activity utilizes the lectin GS-II which is specific for GlcNAc. The substrate is prepared by modification of the same trimannosyl core glycoside described above using in vitro purified GlcNAc T1, which results in addition of a GlcNAc_beta(1-2) residue to the Man_alpha(1-3) residue. Following incubation with insect cell lysates, enzymatic hydrolysis by N-acetylglucosaminidase removes GlcNAc from the substrate resulting in the triannosyl core product. The product is not susceptible to lectin binding and thus escapes into the filtrate. Evaluation of Eu⁺³ fluorescence in the filtrate provides a measure of the N-acetylglucosaminidase activity. Alternatively, enhanced binding of the Eu⁺³-bound trimannosyl core to the Crocus lectin described above can be used as another assay for N-acetylglucosaminidase activity.

### Example 3

Characterization of N-linked Oligosaccharides from Insect Cells

Carbohydrate structure elucidation of the N-glycans of a recombinant glycoprotein, IgG, purified from Trichoplusia ni (High Five™ cells; Invitrogen Corp., Carlsbad, Calif., USA) has been undertaken (Davis et al. (1993) In Vitro Cell. Dev. Biol. 29:842-846; Hsu et al. (1997) J. Biol. Chem. 272:9062-9070). The recombinant glycoprotein, immunoglobulin G (IgG), was purified from both intracellular and extracellular (secreted) sources and all the attached N-glycans determined using three dimensional HPLC techniques. The composition of these structures provided insights into the carbohydrate processing pathways present in insect cells and allowed a comparison of intracellular and secreted N-glycan structures.

The Trichoplusia ni cells grown in serum free medium in suspension culture were infected with a baculovirus vector encoding a murine IgG (Summers et al. (1987) A manual of methods for baculovirus vectors and insect cells culture procedures). IgG includes an N-linked oligosaccharide attachment on each of the two heavy chains.

Heterologous IgG was purified from the culture supernatant and soluble cell lysates using a Protein A-Sepharose column. N-linked oligosaccharides were isolated following protease digestion of IgG and treatment with glycoamidase A to release the N-glycans. Oligosaccharides were then derivatized with 2-aminopyridine (PA) at the reducing ends to provide fluorogenic properties for detection.

Three-dimensional HPLC analysis, was performed to elucidate the N-linked oligosaccharide structures attached to the heavy chain of IgG (Tomiya et al. (1988) Anal. Biochem. 171:73-90, Takahashi et al. (1992) Handbook of Endoglycosidases and Glycoamidases Ed. 199-332). This technique separates oligosaccharides by three successive HPLC steps and enables the identification of structures by comparison of elution conditions with those of known standards.

A DEAE column was used to separate oligosaccharides on the basis of carbohydrate acidity (first dimension). None of the oligosaccharides retained on this column were found to include sialic acid. Treatment of the acidic fractions with neuraminidase from Arthrobacter ureafaciens (known to cleave all known sialic acid linkages) failed to release any sialic acid, and ODS-chromatography of the fractions revealed several minor components different from all known sialylated oligosaccharides.

The second dimension used reverse phase HPLC with an ODS-silica column to fractionate the labeled oligosaccharides according to carbohydrate structure. Supernatant (S) and lysate (L) IgGs oligosaccharides were separated into 6 and 10 fractions, respectively, labeled A-L in FIG. 6.

Separation in the third and final dimension was accomplished using an amide column to isolate oligosaccharides on the basis of molecular size. Peak B from the ODS column was separated into two separate oligosaccharide fractions, and peak H was separated into three separate oligosaccharide fractions on the amide-column.

After oligosaccharide purification, structures of unknown oligosaccharides were determined by comparing their positions on the 3-dimensional map with the positions of over 450 known oligosaccharides. Co-elution of an unknown sample with a known PA-oligosaccharide on the ODS and amide-silica columns was used to confirm the identity of an oligosaccharide. Digestion by glycosidases with specific cleavage sites (alpha-L-fucosidase, beta-galactosidase, beta-N-acetylglucosaminidase, and alpha-mannosidase) followed by reseparation provided further confirmation.

All the oligosaccharides in the culture medium and cell lysates matched known carbohydrates except for oligosaccharide G. The structure of oligosaccharide G was elucidated by treatment of the N-glycan with alpha-L-fucosidase, known to digest Fuc_alpha1-6GIcNAc, followed by treatment with 13.5 M trifluoroacetic acid to remove the alpha1,3 linked fucose. The de-alpha1,6- and de-alpha1,3-fucosylated oligosaccharide G co-eluted with a known oligosaccharide, allowing the identification of G. The structure of oligosaccharide G is shown in FIG. 7.

The structure of oligosaccharide G was further confirmed by ¹H-NMR and electrospray ionization (ESI) mass spectrometry (Hsu et al. (1997) J. Biol. Chem. 272:9062-9070). Thus, the combination of these techniques can be used to elucidate both known and unknown oligosaccharides.

The carbohydrates attached to IgG from the culture medium and intracellular lysate were identified and the levels present in each source were quantified. These structures were then used in conjunction with previous studies of oligosaccharide processing in insect cells (Altmann et al. (1996) Trends in Glycoscience and Glycotechnology 8:101-114) to generate a detailed map of N-linked oligosaccharide processing in Trichoplitsia ni insect cells. The pathway and the levels of the oligosaccharides from secreted and intracellular sources are detailed in FIG. 8.

The initial processing in the T. ni cells appears to be similar to the mammalian pathway, including trimming of the terminal glucose and mannose residues. The trimming process follows a linear pathway with the exception of two different forms of the Man₇GlcNAc₂ (M7GN, in FIG. 8 also observed in native insect glycoproteins (Altmann et al. (1996) Trends in Glycoscience and Glycotechnology 8:101-114) and IgG₄, from NS/0 cells (Ip et al. (1994) Arch. Biochem. Biophys. 308:387-399). The presence of these two Man₇ forms suggests the possible existence of an alternative processing pathway that yields Man₇GlcNAc₂ through the action of endo-alpha-mannosidase. Following cleavage of the mannose residues, GlcNAc (GN) is added to the alpha1,3 branch of Man₅GlcNAC₂ by GIcNAc TI (N-acetylglusosaminyltransferase I) (Altmann et al. (1996) Trends in Glycoscience and Glycotechnology 8:101-114). However, GlcNAc₂ Man₅GlcNAC₂ must be a short-lived intermediate quickly processed by alpha-Man II, since this structure was not detected in the T. ni cell lysate. At the GlcNAc₁, Man₃ GlcNAc₂ oligosaccharide, several branching steps in the N-glycan processing pathway are possible in insect cells. Complex glycoforms can be generated by the action of GlcNAc TII (N-acetylglucosaminyltransferase II) and Gal T (galactosyltransferase T) to provide oligosaccharides which include terminal GlcNAc (GN) and Gal (G) residues. None of the complex oligosaccharide structures included sialic acid indicating that sialylation is negligible or non-existent in these cells.

The production of these complex glycoforms must compete with an alternative processing pathway that is catalyzed by N-acetylglucosaminidase (Altmann et al. (1995) J. Biol. Chem. 270:17344-17349) (see Branch Points in FIG. 8), leading to the production of hybrid and paucimannosidic structures. While the complex-type N-glycans represent 35% of the total secreted glycoforms (supernatant % in FIG. 8), the majority of secreted N-glycans are either paucimannosidic (35%) or hybrid structures (30%). Furthermore, those complex structures with a branch terminating in Gal represent less than 20% of the total secreted glycoforms and no structures were observed with terminal Gal on both branches of the N-glycan.

In contrast to the secreted glycoforms, the intracellular N-glycans (lysate % in FIG. 8) obtained from insect cells include more than 50% high-mannose type structures. The fraction of intracellular complex oligosaccharides is less than 15% and only 8% include a terminal Gal residue. The high level of high-mannose structures from intracellular sources indicates significantly less oligosaccharide processing for most of the intracellular immunoglobulins. Many of these intracellular immunoglobulins may not reach the compartments in which carbohydrate trimming takes place (Jarvis et al. (1989) Mol. Cell. Biol. 9:214-223) High mannose glycoforms are also observed intracellularly for mammalian cells (Jenkins et al. (1998) Cell Culture Engineering VI).

### Example 4

Evaluation of N-glycosylation Pathway Enzymes

The levels of N-linked oligosaccharide processing enzymes are measured using analytical assays to characterize carbohydrate processing in native and recombinant insect cells. These assays are used to compare the N-glycan processing capacity of different cell lines and to evaluate changes in processing and metabolite levels following metabolic engineering modifications.

### Example 4a

High Performance Anion Exchange Chromatography (HPAEC) assay for Galactose Transferase

HPAEC is used in combination with pulsed amperometric detection (HPAEC-PAD) or conductivity to detect metabolite levels in the CMP-SA pathway and to evaluate N-linked oligosaccharide processing enzymes essentially as described by (Lee et al. (1990) Anal. Biochem. 34:953-957, Lee et al. (1996) J. Chromatography A 720:137-149). Shown in FIG. 9 is an example of the use of HPAEC-PAD for measuring Gal T activity by following the lactose formation reaction:

UDP−Gal+Glc GalT Lac+UDP→

The peak labeled “Lac” indicates the formation of the product lactose (Lac). Many of the enzymes involved in N-glycosylation (e.g., aldolase, CMP-NeuAc synthetase, sialyltransferase) and metabolic intermediates (e.g., sialic acid, CMP-sialic acid, ManNAc, ManNAc-6-phosphate) in the CMP-SA production pathway are measured using this form of chromatography, essentially as described by Lee et al. (1990) Anal. Biochem. 34:953-957, Lee et al. (1996) J. Chromatography A 720:137-149, Hardy et al. (1988) Anal. Biochem. 170:54-62, Townsend et al. (1988) Anal. Biochem. 174:459-470, Kiang et al. (1997) Anal. Biochem. 245:97-101.

### Example 4b

Reverse Phase High Performance Liquid Chromatography (HPLC) for Sialyltransferase

To detect native sialyltransferase enzyme activity, Trichoplusia ni lysates were incubated in the presence of exogenously added CMP-SA and the fluorescent substrate, 4-methylumbelliferyl lactoside (Lac-MU). Negligible conversion of the substrate was observed, indicating the absence of endogenous sialyltransferase activity. However, following infection of the insect cells with a baculovirus encoding human alpha2-3-sialyltransferase, conversion of Lac-MU to the product sialyl LacMU was observed in cell lysates using Reverse Phase HPLC and a fluorescence detector (FIG. 10). For higher sensitivity, Lac-PA (PA=2-aminopyridine) or Lac-ABA (ABA=o-aminobenzamide) are used as substrates. HPLC and HPAEC is used in conjunction with other fluorometric methods detailed in the procedures to analyze the metabolites and enzymatic activities in insect cells.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:l, 3, or 5 or a polynucleotide fragment of the cDNA sequence included in ATCC Deposit No: ______;
(b) a polynucleotide encoding a polypeptide fragment of SEQ ID NO:2, 4 or 6 or the cDNA sequence included in ATCC Deposit No: ______;
(c) a polynucleotide encoding a polypeptide domain of SEQ ID NO:2, 4 or 6 or the cDNA sequence included in ATCC Deposit No: ______;
(d) a polynucleotide encoding a polypeptide epitope of SEQ ID NO:2, 4 or 6 or the cDNA sequence included in ATCC Deposit No: ______;
(e) a polynucleotide encoding a polypeptide of SEQ ID NO:2, 4 or 6 or the cDNA sequence included in ATCC Deposit No: ______ having biological activity;
(f) a polynucleotide which is a variant of SEQ ID NO: 1, 3, or 5;
(g) a polynucleotide which is an allelic variant of SEQ ID NO: 1, 3, or 5;
(h) a polynucleotide capable of hybridizing under stringent conditions to any one of the polynucleotides specified in (a)-(g), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a mature form or a secreted protein.

3. The isolated nucleic acid molecule of claim 1, wherein the polynucleotide fragment comprises a nucleotide sequence encoding the sequence identified as SEQ ID NO:2, 4 or 6 or the coding sequence included in ATCC Deposit No: ______.

4. The isolated nucleic acid molecule of claim 1, wherein the polynucleotide fragment comprises the entire nucleotide sequence of SEQ ID NO:1, 3, or 5 or the cDNA sequence included in ATCC Deposit No: ______.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. The isolated nucleic acid molecule of claim 2, wherein the nucleotide sequence comprises any 45 consecutive nucleotides.

8. The isolated nucleic acid molecule of claim 3, wherein the nucleotide sequence comprises any 45 consecutive nucleotides.

9. A recombinant vector comprising the isolated nucleic acid molecule of claim 1.

10. A method of making a recombinant host cell comprising the isolated nucleic acid molecule of claim 1.

11. A recombinant host cell produced by the method of claim 10.

12. The recombinant host cell of claim 11 comprising vector sequences.

13. An isolated polypeptide comprising an amino acid sequence at least 95% identical to a sequence selected from the group consisting of:
(a) a polypeptide fragment of SEQ ID NO:2, 4 or 6 or the encoded sequence included in ATCC Deposit No: ______;
(b) a polypeptide fragment of SEQ ID NO:2, 4 or 6 or the encoded sequence included in ATCC Deposit No: ______ having biological activity;
(c) a polypeptide domain of SEQ ID NO:2, 4 or 6 or the encoded sequence included in ATCC Deposit No: ______;
(d) a polypeptide epitope of SEQ ID NO:2, 4 or 6 or the encoded sequence included in ATCC Deposit No: ______;
(e) a mature form of a secreted protein;
(f) a full length secreted protein;
(g) a variant of SEQ ID NO:2, 4 or 6;
(h) an allelic variant of SEQ ID NO:2, 4 or 6.

14. The isolated polypeptide of claim 13, wherein the mature form or the full length secreted protein comprises sequential amino acid deletions from either the C-terminus or the N-terminus.

15. The isolated polypeptide of claim 13, wherein the polypeptide comprises any 15 consecutive amino acids.

16. An isolated antibody that binds specifically to the isolated polypeptide of claim 13.

17. A recombinant host cell that expresses the isolated polypeptide of claim 13.

18. A method of making an isolated polypeptide comprising:
(a) culturing the recombinant host cell of claim 17 under conditions such that said polypeptide is expressed; and
(b) recovering said polypeptide.

19. The polypeptide produced by claim 18.

20. The gene corresponding to the cDNA sequence of SEQ ID NO: 1, 3 or 5.


SEQUENCE LISTING (6 sequences)

SEQ ID NO: 1 - DNA, 1988 bp, Drosophila melanogaster
  misc_feature (1)..(1988): Nucleotide of Drosophila CMP-SA transporter shown in Figure 1.
cccggtccagtggagtcgtcttcatttgcgctcttctgcttttctggccaacttgaagggaacgaactgccaaacggaacggcccgccgacaactgatcttggccaggaacggagtctttgtgtacccccaactacggcgttttgtttgccttcggtttctatttggttttggagttgagacaatagagctagaagcgtagcaccatgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctagctctggaggaatccggatctgccaacagtatttgtccgtaacccggaggccgccgttcggtaattgctctggtgtatccggtgcatccggtgtccttgatattccccacgttttgtgtgtatatatattaggatggatattaacgtctggacaattcgtagcactttctgttgagacgataattgtaatgtaaagcagcgataagaatacttaatgatttatgtaatttaaatgtatttagggtattcactagcgaaactaaagtgtagttaatgaaatgatgcagcttgaatgcacgttttattgatcattttaattgttactttgcgaatgctgaggaatgccagcagtatttactggacaacaccacacataccgtgcctatccttttgtaaatatccacttgcactcaagtttagactaagatttgtaacgatttatagttttcaagttacacttgatgttcggccaattcacagccacaactacttttgttcgaattttttttttagaagatcatatatatgtatatatatttacttgaatgtgcattatttattcattgccaattactttatttttgagaaatcctaggttgagccagcccagacttatatatttgtatttgaactaaagtcgcattactcgcacagtgttttaagagtgattccatatcgatatgtttcatttcctttgaataaatattagg

SEQ ID NO: 2 - protein, 357 aa, Drosophila melanogaster
  PEPTIDE (1)..(357): Amino acid sequence of Drosophila CMP-SA transporter shown in Figure 1.
MNSIHMNANTLKYISLLTLTLQNAILGLSMRYARTRPGDIFLSSTAVLMAEFAKLITCLFLVFNEEGKDAQKFVRSLHKTIIANPMDTLKVCVPSLVYIVQNNLLYVSASHLDAATYQVTYQLKILTTAMFAVVILRRKLLNTQWGALLLLVMGIVLVQLAQTEGPTSGSAGGAAAAATAASSGGAPEQNRMLGLWAALGACFLSGFAGIYFEKILKGAEISVWMRNVQLSLLSIPFGLLTCFVNDGSRIFDQGFFKGYDLFVWYLVLLQAGGGLIVAVVVKYADNILKGFATSLAIIISCVASIYIFDFNLTLQFSFGAGLVIASIFLYGYDPARSAPKPTMHGPGGDEEKLLPRV

SEQ ID NO: 3 - DNA, 2079 bp, Drosophila melanogaster
  misc_feature (1)..(2079): Nucleotide sequence of Drosophila Hexosaminidase-1 shown in Figure 2.
ctgaacagcagccaagttcagttggaatcgatttggaataacctatattttgcattcgccagttggttttgagagtttctgtgctgaggttgacgaaatgaagtcgacgcggactgcacttggagtggccctgctcctggccctggtgtctcaactggcggcccacagctcggatgacttggtttacggctacgagtgccgcagtggctactgccagaaggtagagctcagcgaggagaactacgtcaaggccatcagtctgcccgtgtgccggctcttctgcggcagctccatcggcaccttgtggcccaaaccgacgggcactgtgcgtctggacacgttgatgcgccaagtggacatctccttcattgatttcaatttcaatggaattgcccgccagcagaagctatggcgagcggttgaagaccgtttcatgaacatgctcgaagcccagattccggatcgcaaggttctggcacgaggtggctaccgtatgtctgtgaacatcaatactccggatgagccgacaccggccagactcaccctggatacggatgagagctatacgctggacattgatacggatgcctcgggtcatgtgctggccaacataaccgcgtccaacttctttggagcccgtcatggcctggagacactagcccagttgattgtctacgatgacatccgccgcgaggttcaggtgactgccaatgccaccatcaacgatgctcccgtgtacaaatggcgtggattgctcctggacacctcccgtaactactactctgtgaagtccatcaagaggacgttggagggcatggccttggtcaaactgaacaccttccactggcacatcacggactcgcacagcttcccattggaggtgaagaagcgtccggagctgcacaagctgggagcctattcccaacggcaggtgtacacccgtcgggacgtggccgaggttgtggagtacggccgcgtgcgaggcatccgtgttatgccagagttcgatgcccctgcccatgtgggtgagggctggcagcacaagaacatgaccgcctgcttcaatgcccagccatggaagtcgttctgcgttgagccaccgtgtggccaactcgatcccactgtgaacgaaatgtacgatgtgctggaggacatttacggcaccatgttcgatcagttcaacccggatatcttccacatgggcggcgatgaagtgtcaaccagctgctggaacagcagccagcccatccagcagtggatgaagaaacagggttggggcctggagaccgccgactttatgcgcttgtggggacacttccagacggaggctcttggtcgcgtggacaaggtggccaatggtacgcacacgcccatcattctgtggactagtggactcactgaggagcccttcattgacgagtatctgaacccagagcgttacatcattcagatctggaccactggtgtagatccgaaggtaaagaagattctggagcggggctacaagatcattgtgtccaactacgatgccctgtacttggattgcggcggagctggctgggtgaccgatggaaacaactggtgctccccctacattggctggcagaaggtgtacgacaatagtttgaaatccattgccggcgactacgagcatcatgtattgggcgcggagggagccatttggtcagagcagatcgacgagcacacactggacaaccgcttctggcccagggccagtgccctggccgaacgactttggtcaaatcctgccgaaggttggcgccaggcggagtcacgtctgctgctccaccgccagcgactggtggacaatggtctgggcgcagaggccatgcagccgcagtggtgcctgcaaaacgagcacgagtgtcccattgacgcgtacgatgcacaagtttgaccagctattaaaccatttctcttaacggtcgctatccttgtagatgtagtcggggcagcgggcggcttgggttgatcgtcctgttgcttctcacaacgctctctgcctgaaaataaagtacagctcaaaggtggccttacgcaaaaaaaaaaaaaaaaaaa

SEQ ID NO: 4 - protein, 606 aa, Drosophila melanogaster
  PEPTIDE (1)..(606): Amino acid sequence of Drosophila Hexosaminidase-1 shown in Figure 2.
MKSTRTALGVALLLALVSQLAAHSSDDLVYGYECRSGYCQKVELSEENYVKAISLPVCRLFCGSSIGTLWPKPTGTVRLDTLMRQVDISFIDFNFNGIARQQKLWRAVEDRFMNMLEAQIPDRKVLARGGYRMSVNINTPDEPTPARLTLDTDESYTLDIDTDASGHVLANITASNFFGARHGLETLAQLIVYDDIRREVQVTANATINDAPVYKWRGLLLDTSRNYYSVKSIKRTLEGMALVKLNTFHWHITDSHSFPLEVKKRPELHKLGAYSQRQVYTRRDVAEVVEYGRVRGIRVMPEFDAPAHVGEGWQHKNMTACFNAQPWKSFCVEPPCGQLDPTVNEMYDVLEDIYGTMFDQFNPDIFHMGGDEVSTSCWNSSQPIQQWMKKQGWGLETADFMRLWGHFQTEALGRVDKVANGTHTPIILWTSGLTEEPFIDEYLNPERYIIQIWTTGVDPKVKKILERGYKIIVSNYDALYLDCGGAGWVTDGNNWCSPYIGWQKVYDNSLKSIAGDYEHHVLGAEGAIWSEQIDEHTLDNRFWPRASALAERLWSNPAEGWRQAESRLLLHRQRLVDNGLGAEAMQPQWCLQNEHECPIDAYDAQV

SEQ ID NO: 5 - DNA, 2136 bp, Drosophila melanogaster
  misc_feature (1)..(2136): Nucleotide sequence of Drosophila Hexosaminidase-2 shown in Figure 3.
cggagcgggtgggcggccggttgaaaatagcgaatacaccgtacacacccccctaacacaacaatatagcaaataccgaacagaaattataaaagcgagcggcgcagccaaagaagccgttagaaagaaacgatgcggttcagcggctacaatcgttatcagtgcttttgctctgctgttggatcgctgctgctattatccctattatccttcgcggtcggcgctgcgctgacgcgggcagatgacgtcggcagcgacgccgacgccggcagcgccagcaggaaatggctgtgcagccggacggatatctgcaccgcggagggcgagatggtggccggactgcagtacgcgccggagatcttcgagagccagcgggattgccgactgtcgtgcggcaagtacggagccatctggcccatgcccaccggcaaggagtgcaccatatcgcacaggcgggtacgattcgatccgtggaaggtgcgtttccatgttgtggcgcccggcgaggcggccacccagtttctcagggagacgaaccggctgttcgtctcgaatctgctgaaggagtgcatacgaaactgcacgctggagaccagcaagcagattctggtcagatccacggtggccaacgagagcctcgttctggactggcccaccgacgagagctacgccctggtggtgcgaaccactgaaacggccacctttgtggacatccaagcgacgacggtgtacggtgcacgtcatgccttcgagacgctgagcaacctggtcaccggcagcctgtccaatggcctgctgatggtcaccacggccaacatcaccgatcgtccggccttttcgcatcgcggcgtccttctggatacggcccgtaactttgtgccgctcaagtttatacgcagcaccttggatgcgatggcggccagtaagctgaatgtgctccactggcatgtggtggacacgcacagttttccgctggagatcaccagggtgccggagatgcagcgttacggagcgtactcctcatcgcagacgtactcgcgccaggatgcactgaatcttgtgaaatatgcccgactgcggggcatacggatactgatcgagatcgatggaccctcgcatgcgggcaatggctggcaatggggtcctgccgccggactgggcaacatgtccgtgtgcctgaatcaatcgccctggaggagattctgtgtacagccaccgtgcggccaactgaatcccctgaacgatcatatgtacgcagtgctcaaggagatcttcgaggacgtagccgaggtgggcgctcccgaggagaccctgcacatgggcggcgacgaggtgttccttccctgctggaataacaccgacgagattcgggatgggatgcgggcacgtggctacgatctcagcgagcagagcttcctgcggctgtggtcgcaattccatcagcggaacctcaatgcatgggacgagattaacgagcgtatgtatccgggcatcaaggagccgaagtcggtgatcatctggtccagtcacctcaccaatccccggtacatcgagacctacctgcccaaggagcggttcataatccagacatgggtggagtcacaggatgccctcaatcgggagctattgcagcgaggctaccgactgattgtgtccacgaagaatgcctggtacctggatcacggcttctggggcagcacatcgtactacaactggcgcaccgtgtactccagtggcatgcccgttggtcgcagcaaggatcaggttctcggcggtgaggtgtgcatgtggagcgagtatgtggatcagaactcactggaatcccgaatctggccgcgagctggagcagcagccgagcgaatgtggtcaaatccaaagtcttcagccctgttggcccaaagaagattctaccgctaccgggagcgactcctggcgcgcggaatccatgcggatgcagttattccgcactggtgcgtcctccacgaaggacagtgcctctaaatcggtcgttccgaacctagttttagcaccagcacataccatgtacatacctttcaaactatgttattttattcatcagttattgcccataaagatctgtttttaatccaaaaaaaaaaaaaaaaaaaaaaaaaa

SEQ ID NO: 6 - protein, 622 aa, Drosophila melanogaster
  PEPTIDE (1)..(622): Amino acid sequence of Drosophila Hexosaminidase-2 shown in Figure 3.
MRFSGYNRYQCFCSAVGSLLLLSLLSFAVGAALTRADDVGSDADAGSASRKWLCSRTDICTAEGEMVAGLQYAPEIFESQRDCRLSCGKYGAIWPMPTGKECTISHRRVRFDPWKVRFHVVAPGEAATQFLRETNRLFVSNLLKECIRNCTLETSKQILVRSTVANESLVLDWPTDESYALVVRTTETATFVDIQATTVYGARHAFETLSNLVTGSLSNGLLMVTTANITDRPAFSHRGVLLDTARNFVPLKFIRSTLDAMAASKLNVLHWHVVDTHSFPLEITRVPEMQRYGAYSSSQTYSRQDALNLVKYARLRGIRILIEIDGPSHAGNGWQWGPAAGLGNMSVCLNQSPWRRFCVQPPCGQLNPLNDHMYAVLKEIFEDVAEVGAPEETLHMGGDEVFLPCWNNTDEIRDGMRARGYDLSEQSFLRLWSQFHQRNLNAWDEINERMYPGIKEPKSVIIWSSHLTNPRYIETYLPKERFIIQTWVESQDALNRELLQRGYRLIVSTKNAWYLDHGFWGSTSYYNWRTVYSSGMPVGRSKDQVLGGEVCMWSEYVDQNSLESRIWPRAGAAAERMWSNPKSSALLAQRRFYRYRERLLARGIHADAVIPHWCVLHEGQCL